SEQUENCE LISTING (164 sequences)

SEQ ID NO: 1 - protein, 159 aa, Streptomyces avidinii
  REGION 1..159: note = Streptavidin (UniProt No. P22629)
DPSKDSKAQVSAAEAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAASIDAAKKAGVNNGNPLDAVQQ

SEQ ID NO: 2 - protein, 127 aa, synthetic construct
  REGION 1..127: note = Minimal streptavidin
MEAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAAS

SEQ ID NO: 3 - protein, 159 aa, synthetic construct
  REGION 1..159: note = Mutein Streptavidin Val44-Thr45-Ala46-Arg47
DPSKDSKAQVSAAEAGITGTWYNQLGSTFIVTAGADGALTGTYVTARGNAESRYVLTGRYDSAPATDGSGTALGWTVAWKNNYRNAHSATTWSGQYVGGAEARINTQWLLTSGTTEANAWKSTLVGHDTFTKVKPSAASIDAAKKAGVNNGNPLDAVQQ

SEQ ID NO: 4 - protein, 127 aa, synthetic construct
  REGION 1..127: note = Mutein Streptavidin Val44-Thr45-Ala46-Arg47
MEAGITGTWYNQLGSTFIVTAGADGALTGTYVTARGNAESRYVLTGRYDSAPATDGSGTALGWTVAWKNNYRNAHSATTWSGQYVGGAEARINTQWLLTSGTTEANAWKSTLVGHDTFTKVKPSAAS

SEQ ID NO: 5 - protein, 159 aa, synthetic construct
  REGION 1..159: note = Mutein Streptavidin Ile44-Gly45-Ala46-Arg47
DPSKDSKAQVSAAEAGITGTWYNQLGSTFIVTAGADGALTGTYIGARGNAESRYVLTGRYDSAPATDGSGTALGWTVAWKNNYRNAHSATTWSGQYVGGAEARINTQWLLTSGTTEANAWKSTLVGHDTFTKVKPSAASIDAAKKAGVNNGNPLDAVQQ

SEQ ID NO: 6 - protein, 127 aa, synthetic construct
  REGION 1..127: note = Mutein Streptavidin Ile44-Gly45-Ala46-Arg47
MEAGITGTWYNQLGSTFIVTAGADGALTGTYIGARGNAESRYVLTGRYDSAPATDGSGTALGWTVAWKNNYRNAHSATTWSGQYVGGAEARINTQWLLTSGTTEANAWKSTLVGHDTFTKVKPSAAS

SEQ ID NO: 7 - protein, 8 aa, synthetic construct
  REGION 1..8: note = Streptavidin-binding peptide, Strep-tag
WRHPQFGG

SEQ ID NO: 8 - protein, 8 aa, synthetic construct
  REGION 1..8: note = Strep-tag II
WSHPQFEK

SEQ ID NO: 9 - skipped

SEQ ID NO: 10 - protein, 4 aa, synthetic construct
  REGION 1..4: note = Streptavidin-binding peptide
HPQF

SEQ ID NO: 11 - protein, 8 aa, synthetic construct
  REGION 1..8: note = Streptavidin-binding peptide
  VARIANT 1: note = X is Trp, Lys, or Arg
  VARIANT 2: note = X is any amino acid
  VARIANT 7: note = X is Gly or Glu
  VARIANT 8: note = X is Gly, Lys, or Arg
XXHPQFXX

SEQ ID NO: 12 - protein, 8 aa, synthetic construct
  REGION 1..8: note = Streptavidin-binding peptide
  VARIANT 2: note = X is any amino acid
  VARIANT 7: note = X is Gly or Glu
  VARIANT 8: note = X is Gly, Lys, or Arg
WXHPQFXX

SEQ ID NO: 13 - protein, 24 aa, synthetic construct
  REGION 1..24: note = Sequential modules of streptavidin-binding peptide
  VARIANT 9..16: note = X is any amino acid
WSHPQFEKXXXXXXXXWSHPQFEK

SEQ ID NO: 14 - protein, 24 aa, synthetic construct
  REGION 1..24: note = Sequential modules of streptavidin-binding peptide
WSHPQFEKGGGSGGGSWSHPQFEK

SEQ ID NO: 15 - protein, 30 aa, synthetic construct
  REGION 1..30: note = Twin-Strep-tag
SAWSHPQFEKGGGSGGGSGGGSWSHPQFEK

SEQ ID NO: 16 - protein, 30 aa, synthetic construct
  REGION 1..30: note = Twin-Strep-tag
SAWSHPQFEKGGGSGGGSGGSAWSHPQFEK

SEQ ID NO: 17 - protein, 28 aa, synthetic construct
  REGION 1..28: note = Twin-Strep-tag
WSHPQFEKGGGSGGGSGGGSWSHPQFEK

SEQ ID NO: 18 - protein, 24 aa, synthetic construct
  REGION 1..24: note = Twin-Strep-tag
WSHPQFEKGGGSGGGSWSHPQFEK

SEQ ID NO: 19 - protein, 28 aa, synthetic construct
  REGION 1..28: note = Twin-Strep-tag
WSHPQFEKGGGSGGGSGGSAWSHPQFEK

SEQ ID NO: 20 - protein, 9 aa, synthetic construct
  REGION 1..9: note = HA-tag
YPYDVPDYA

SEQ ID NO: 21 - protein, 11 aa, synthetic construct
  REGION 1..11: note = VSV-G-tag
YTDIEMNRLGK

SEQ ID NO: 22 - protein, 11 aa, synthetic construct
  REGION 1..11: note = HSV-tag
QPELAPEDPED

SEQ ID NO: 23 - protein, 10 aa, synthetic construct
  REGION 1..10: note = T7 epitope
ASMTGGQQMG

SEQ ID NO: 24 - protein, 11 aa, synthetic construct
  REGION 1..11: note = HSV epitope
QPELAPEDPED

SEQ ID NO: 25 - protein, 10 aa, synthetic construct
  REGION 1..10: note = Myc epitope
EQKLISEEDL

SEQ ID NO: 26 - protein, 14 aa, synthetic construct
  REGION 1..14: note = V5-tag
GKPIPNPLLGLDST

SEQ ID NO: 27 - protein, 126 aa, synthetic construct
  REGION 1..126: note = Mutein Streptavidin Val44-Thr45-Ala46-Arg47 and Glu117, Gly120, Tyr121 (mutein m1-9)
EAGITGTWYNQLGSTFIVTAGADGALTGTYVTARGNAESRYVLTGRYDSAPATDGSGTALGWTVAWKNNYRNAHSATTWSGQYVGGAEARINTQWLLTSGTTEENAGYSTLVGHDTFTKVKPSAAS

SEQ ID NO: 28 - protein, 139 aa, synthetic construct
  REGION 1..139: note = Mutein Streptavidin Val44-Thr45-Ala46-Arg47 and Glu117, Gly120, Tyr121 (mutein m1-9)
DPSKDSKAQVSAAEAGITGTWYNQLGSTFIVTAGADGALTGTYVTARGNAESRYVLTGRYDSAPATDGSGTALGWTVAWKNNYRNAHSATTWSGQYVGGAEARINTQWLLTSGTTEENAGYSTLVGHDTFTKVKPSAAS

SEQ ID NO: 29 - protein, 253 aa, synthetic construct
  REGION 1..253: note = Variable Heavy chain of Fab fragment m13B8.2
AMQVQLKQSGPGLVQPSQSLSITCTVSGFSLTTFGVHWVRQSPGKGLEWLGVIWASGITDYNVPFMSRLSITKDNSKSQVFFKLNSLQPDDTAIYYCAKNDPGTGFAYWGQGTLVTVSAGSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGSAWSHPQFEKGGGSGGGSGGSAWSHPQFEK

SEQ ID NO: 30 - protein, 218 aa, synthetic construct
  REGION 1..218: note = Variable Light chain of Fab Fragment m13B8.2
AMDIQMTQSPASLSASVGETVTFTCRASEMIYSYLAWYQQKQGKSPQLLVHDAKTLAEGVPSRFSGGGSGTQFSLKINTLQPEDFGTYYCQAHYGNPPTFGGGTKLEIKRGIAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGS

SEQ ID NO: 31 - protein, 119 aa, synthetic construct [sequence omitted]

SEQ ID NO: 32 - protein, 106 aa, synthetic construct [sequence omitted]

SEQ ID NO: 33 - protein, 116 aa, synthetic construct
  REGION 1..116: note = Variable Heavy chain of anti-CD28 antibody CD28.3
LQQSGAELVKPGASVRLSCKASGYTFTEYIIHWIKLRSGQGLEWIGWFYPGSNDIQYNAKFKGKATLTADKSSSTVYMELTGLTSEDSAVYFCARRDDFSGYDALPYWGQGTMVTV

SEQ ID NO: 34 - protein, 108 aa, synthetic construct
  REGION 1..108: note = Variable Light chain of anti-CD28 antibody CD28.3
DIQMTQSPASLSVSVGETVTITCRTNENIYSNLAWYQQKQGKSPQLLIYAATHLVEGVPSRFSGSGSGTQYSLKITSLQSEDFGNYYCQHFWGTPCTFGGGTKLEIKR

SEQ ID NO: 35 - protein, 11 aa, synthetic construct
  REGION 1..11: note = MAT tag
HNHRHKHGGGC

SEQ ID NO: 36 - protein, 118 aa, synthetic construct
  source 1..118: note = Variable Heavy chain of huOKT8
  REGION 1..118: note = Variable Heavy chain of huOKT8
QVQLVQSGAEVKKPGATVKISCKVSGFNIKDTYIHWVQQAPGKGLEWMGRIDPANDNTLYASKFQGRVTITADTSTDTAYMELSSLRSEDTAVYYCARGYGYYVFDHWGQGTLVTVSS

SEQ ID NO: 37 - protein, 107 aa, synthetic construct
  REGION 1..107: note = Variable Light chain of huOKT8
DVQITQSPSSLSASVGDRVTITCRTSRSISQYLAWYQQKPGKVPKLLIYSGSTLQSGVPSRFSGSGSGTDFTLTISSLQPEDVATYYCQQHNENPLTFGGGTKVEIK

SEQ ID NO: 38 - protein, 30 aa, synthetic construct
  REGION 1..30: note = Linker
PGGGSGGGGSGGGGSGGGGSGGGGSGGGGP

SEQ ID NO: 39 - protein, 17 aa, synthetic construct
  REGION 1..17: note = Linker
GSADDAKKDAAKKDGKS

SEQ ID NO: 40 - protein, 22 aa, synthetic construct
  REGION 1..22: note = GMCSFR alpha chain
MLLLVTSLLLCELPHPAFLLIP

SEQ ID NO: 41 - DNA, 66 bp, synthetic construct
  source 1..66: mol_type = other DNA
  misc_feature 1..66: note = GMCSFR alpha chain
atgcttctcctggtgacaagccttctgctctgtgagttaccacacccagcattcctcctgatccca

SEQ ID NO: 42 - protein, 18 aa, synthetic construct
  REGION 1..18: note = CD8 alpha signal peptide
MALPVTALLLPLALLLHA

SEQ ID NO: 43 - protein, 357 aa, synthetic construct
  REGION 1..357: note = truncated epidermal growth factor receptor (tEGFR)
MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 44 - protein, 335 aa, synthetic construct
  REGION 1..335: note = truncated epidermal growth factor receptor (tEGFR)
RKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 45 - protein, 22 aa, synthetic construct
  REGION 1..22: note = F2A
VKQTLNFDLLKLAGDVESNPGP

SEQ ID NO: 46 - protein, 20 aa, synthetic construct
  REGION 1..20: note = E2A
QCTNYALLKLAGDVESNPGP

SEQ ID NO: 47 - protein, 24 aa, synthetic construct
  REGION 1..24: note = T2A
LEGGGEGRGSLLTCGDVEENPGPR

SEQ ID NO: 48 - protein, 18 aa, synthetic construct
  REGION 1..18: note = T2A
EGRGSLLTCGDVEENPGP

SEQ ID NO: 49 - protein, 22 aa, synthetic construct
  REGION 1..22: note = P2A
GSGATNFSLLKQAGDVEENPGP

SEQ ID NO: 50 - protein, 19 aa, synthetic construct
  REGION 1..19: note = P2A
ATNFSLLKQAGDVEENPGP

SEQ ID NO: 51 - protein, 5 aa, synthetic construct
  REGION 1..5: note = CDR H1
DYGVS

SEQ ID NO: 52 - protein, 16 aa, synthetic construct
  REGION 1..16: note = CDR H2
VIWGSETTYYNSALKS

SEQ ID NO: 53 - protein, 7 aa, synthetic construct
  REGION 1..7: note = CDR H3
YAMDYWG

SEQ ID NO: 54 - protein, 12 aa, synthetic construct
  REGION 1..12: note = CDR H3
HYYYGGSYAMDY

SEQ ID NO: 55 - protein, 11 aa, synthetic construct
  REGION 1..11: note = CDR L1
RASQDISKYLN

SEQ ID NO: 56 - protein, 7 aa, synthetic construct
  REGION 1..7: note = CDR L2
SRLHSGV

SEQ ID NO: 57 - protein, 7 aa, synthetic construct
  REGION 1..7: note = CDR L2
HTSRLHS

SEQ ID NO: 58 - protein, 9 aa, synthetic construct
  REGION 1..9: note = CDR L3
GNTLPYTFG

SEQ ID NO: 59 - protein, 9 aa, synthetic construct
  REGION 1..9: note = CDR L3
QQGNTLPYT

SEQ ID NO: 60 - protein, 120 aa, synthetic construct
  REGION 1..120: note = VH
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 61 - protein, 107 aa, synthetic construct
  REGION 1..107: note = VL
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 62 - protein, 18 aa, synthetic construct
  REGION 1..18: note = Linker
GSTSGSGKPGSGEGSTKG

SEQ ID NO: 63 - DNA, 735 bp, synthetic construct
  source 1..735: mol_type = other DNA
  misc_feature 1..735: note = Sequence encoding scFv
gacatccagatgacccagaccacctccagcctgagcgccagcctgggcgaccgggtgaccatcagctgccgggccagccaggacatcagcaagtacctgaactggtatcagcagaagcccgacggcaccgtcaagctgctgatctaccacaccagccggctgcacagcggcgtgcccagccggtttagcggcagcggctccggcaccgactacagcctgaccatctccaacctggaacaggaagatatcgccacctacttttgccagcagggcaacacactgccctacacctttggcggcggaacaaagctggaaatcaccggcagcacctccggcagcggcaagcctggcagcggcgagggcagcaccaagggcgaggtgaagctgcaggaaagcggccctggcctggtggcccccagccagagcctgagcgtgacctgcaccgtgagcggcgtgagcctgcccgactacggcgtgagctggatccggcagccccccaggaagggcctggaatggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgagcagc

SEQ ID NO: 64 - protein, 245 aa, synthetic construct
  REGION 1..245: note = scFv
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 65 - protein, 5 aa, synthetic construct
  REGION 1..5: note = CDR H1
SYWMN

SEQ ID NO: 66 - protein, 17 aa, synthetic construct
  REGION 1..17: note = CDR H2
QIYPGDGDTNYNGKFKG

SEQ ID NO: 67 - protein, 13 aa, synthetic construct
  REGION 1..13: note = CDR H3
KTISSVVDFYFDY

SEQ ID NO: 68 - protein, 9 aa, synthetic construct
  REGION 1..9: note = CDR L3
QQYNRYPYT

SEQ ID NO: 69 - protein, 5 aa, synthetic construct
  REGION 1..5: note = CDR H1
SYWMN

SEQ ID NO: 70 - protein, 17 aa, synthetic construct
  REGION 1..17: note = CDR H2
QIYPGDGDTNYNGKFKG

SEQ ID NO: 71 - protein, 122 aa, synthetic construct
  REGION 1..122: note = VH
EVKLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTVTVSS

SEQ ID NO: 72 - protein, 108 aa, synthetic construct
  REGION 1..108: note = VL
DIELTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEIKR

SEQ ID NO: 73 - protein, 11 aa, synthetic construct
  REGION 1..11: note = CDR L1
KASQNVGTNVA

SEQ ID NO: 74 - protein, 7 aa, synthetic construct
  REGION 1..7: note = CDR L2
SATYRNS

SEQ ID NO: 75 - protein, 9 aa, synthetic construct
  REGION 1..9: note = CDR L3
QQYNRYPYT

SEQ ID NO: 76 - protein, 5 aa, synthetic construct
  REGION 1..5: note = CDR H1
SYWMN

SEQ ID NO: 77 - protein, 17 aa, synthetic construct
  REGION 1..17: note = CDR H2
QIYPGDGDTNYNGKFKG

SEQ ID NO: 78 - protein, 13 aa, synthetic construct
  REGION 1..13: note = CDR H3
KTISSVVDFYFDY

SEQ ID NO: 79 - protein, 15 aa, synthetic construct
  REGION 1..15: note = Linker
GGGGSGGGGSGGGGS

SEQ ID NO: 80 - protein, 245 aa, synthetic construct
  REGION 1..245: note = scFv
EVKLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTVTVSSGGGGSGGGGSGGGGSDIELTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEIKR

SEQ ID NO: 81 - protein, 12 aa, Homo sapiens
  REGION 1..12: note = spacer (IgG4hinge)
ESKYGPPCPPCP

SEQ ID NO: 82 - DNA, 36 bp, Homo sapiens
  source 1..36: mol_type = genomic DNA
  misc_feature 1..36: note = spacer (IgG4hinge)
gaatctaagtacggaccgccctgccccccttgccct

SEQ ID NO: 83 - protein, 119 aa, Homo sapiens
  REGION 1..119: note = Hinge-CH3 spacer
ESKYGPPCPPCPGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 84 - protein, 229 aa, Homo sapiens
  REGION 1..229: note = Hinge-CH2-CH3 spacer
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 85 - protein, 282 aa, Homo sapiens
  REGION 1..282: note = IgD-hinge-Fc
RWPESPKAQASSVPTAQPQAEGSLAKATTAPATTRNTGRGGEEKKKEKEKEEQEERETKTPECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREPAAQAPVKLSLNLLASSDPPEAASWLLCEVSGFSPPNILLMWLEDQREVNTSGFAPARPPPQPGSTTFWAWSVLRVPAPPSPQPATYTCVVSHEDSRTLLNASRSLEVSYVTDH

SEQ ID NO: 86 - protein, 14 aa, synthetic construct
  REGION 1..14: note = Exemplary IgG Hinge
EVVVKYGPPCPPCP

SEQ ID NO: 87 - skipped

SEQ ID NO: 88 - protein, 15 aa, synthetic construct
  REGION 1..15: note = Exemplary IgG Hinge
EPKSCDKTHTCPPCP

SEQ ID NO: 89 - protein, 12 aa, synthetic construct
  REGION 1..12: note = Exemplary IgG Hinge
ERKCCVECPPCP

SEQ ID NO: 90 - protein, 61 aa, synthetic construct
  REGION 1..61: note = Exemplary IgG Hinge
ELKTPLGDTHTCPRCPEPKSCDTPPPCPRCPEPKSCDTPPPCPRCPEPKSCDTPPPCPRCP

SEQ ID NO: 91 - protein, 12 aa, synthetic construct
  REGION 1..12: note = Exemplary IgG Hinge
ESKYGPPCPSCP

SEQ ID NO: 92 - protein, 12 aa, synthetic construct
  REGION 1..12: note = Exemplary IgG Hinge
ESKYGPPCPPCP

SEQ ID NO: 93 - protein, 9 aa, synthetic construct
  REGION 1..9: note = Exemplary IgG Hinge
YGPPCPPCP

SEQ ID NO: 94 - protein, 10 aa, synthetic construct
  REGION 1..10: note = Exemplary IgG Hinge
KYGPPCPPCP

SEQ ID NO: 95 - protein, 27 aa, Homo sapiens
  REGION 1..27: note = CD28 (amino acids 153-179 of Accession No. P10747)
FWVLVVVGGVLACYSLLVTVAFIIFWV

SEQ ID NO: 96 - protein, 66 aa, Homo sapiens
  REGION 1..66: note = CD28 (amino acids 114-179 of Accession No. P10747)
IEVMYPPPYLDNEKSNGTIIHVKGKHLCPSPLFPGPSKPFWVLVVVGGVLACYSLLVTVAFIIFWV

SEQ ID NO: 97 - protein, 41 aa, Homo sapiens
  REGION 1..41: note = CD28 (amino acids 180-220 of P10747)
RSKRSRLLHSDYMNMTPRRPGPTRKHYQPYAPPRDFAAYRS

SEQ ID NO: 98 - protein, 41 aa, Homo sapiens
  REGION 1..41: note = CD28 (LL to GG)
RSKRSRGGHSDYMNMTPRRPGPTRKHYQPYAPPRDFAAYRS

SEQ ID NO: 99 - protein, 42 aa, Homo sapiens
  REGION 1..42: note = 4-1BB (amino acids 214-255 of Q07011.1)
KRGRKKLLYIFKQPFMRPVQTTQEEDGCSCRFPEEEEGGCEL

SEQ ID NO: 100 - protein, 112 aa, Homo sapiens
  REGION 1..112: note = CD3 zeta
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 101 - protein, 112 aa, Homo sapiens
  REGION 1..112: note = CD3 zeta
RVKFSRSAEPPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 102 - protein, 112 aa, Homo sapiens
  REGION 1..112: note = CD3 zeta
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 103 - protein, 126 aa, synthetic construct
  REGION 1..126: note = Minimal streptavidin without initial methionine
EAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAAS

SEQ ID NO: 104 - protein, 126 aa, synthetic construct
  REGION 1..126: note = Mutein Streptavidin Val44-Thr45-Ala46-Arg47 without initial methionine
EAGITGTWYNQLGSTFIVTAGADGALTGTYVTARGNAESRYVLTGRYDSAPATDGSGTALGWTVAWKNNYRNAHSATTWSGQYVGGAEARINTQWLLTSGTTEANAWKSTLVGHDTFTKVKPSAAS

SEQ ID NO: 105 - protein, 126 aa, synthetic construct
  REGION 1..126: note = Mutein Streptavidin Ile44-Gly45-Ala-46-Arg47 without initial methionine
EAGITGTWYNQLGSTFIVTAGADGALTGTYIGARGNAESRYVLTGRYDSAPATDGSGTALGWTVAWKNNYRNAHSATTWSGQYVGGAEARINTQWLLTSGTTEANAWKSTLVGHDTFTKVKPSAAS

SEQ ID NO: 106 - protein, 326 aa, Homo sapiens
  REGION 1..326: note = Human IgG2 Fc (Uniprot P01859)
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 107 - protein, 327 aa, Homo sapiens
  REGION 1..327: note = Human IgG4 Fc (Uniprot P01861)
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 108 - protein, 107 aa, synthetic construct
  REGION 1..107: note = FKBP
GVQVETISPGDGRTFPKRGQTCVVHYTGMLEDGKKMDSSRDRNKPFKFMLGKQEVIRGWEEGVAQMSVGQRAKLTISPDYAYGATGHPGIIPPHATLVFDVELLKLE

SEQ ID NO: 109 - protein, 107 aa, synthetic construct
  REGION 1..107: note = FKBP12v36
GVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLE

SEQ ID NO: 110 - protein, 16 aa, synthetic construct
  REGION 1..16: note = Modified acylation motif
MGSNKSKPKDASQRRR

SEQ ID NO: 111 - protein, 5 aa, synthetic construct
  REGION 1..5: note = dual acylation motif
  VARIANT 4: note = X is any amino acid
MGCXC

SEQ ID NO: 112 - skipped

SEQ ID NO: 113 - protein, 5 aa, synthetic construct
  REGION 1..5: note = CDR-H1
DYSIN

SEQ ID NO: 114 - protein, 17 aa, synthetic construct
  REGION 1..17: note = CDR-H2
WINTETREPAYAYDFRG

SEQ ID NO: 115 - protein, 8 aa, synthetic construct
  REGION 1..8: note = CDR-H3
DYSYAMDY

SEQ ID NO: 116 - protein, 15 aa, synthetic construct
  REGION 1..15: note = CDR-L1
RASESVTILGSHLIH

SEQ ID NO: 117 - protein, 7 aa, synthetic construct
  REGION 1..7: note = CDR-L2
LASNVQT

SEQ ID NO: 118 - protein, 9 aa, synthetic construct
  REGION 1..9: note = CDR-L3
LQSRTIPRT

SEQ ID NO: 119 - protein, 117 aa, synthetic construct
  REGION 1..117: note = Variable heavy (VH) Anti-BCMA
QIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSS

SEQ ID NO: 120 - protein, 111 aa, synthetic construct
  REGION 1..111: note = Variable light (VL) Anti-BCMA
DIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIK

SEQ ID NO: 121 - protein, 246 aa, synthetic construct
  REGION 1..246: note = scFv
DIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSS

SEQ ID NO: 122 - protein, 472 aa, synthetic construct
  REGION 1..472: note = anti-BCMA CAR
DIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 123 - protein, 5 aa, synthetic construct
  REGION 1..5: note = CDR-H1
DYYVY

SEQ ID NO: 124 - protein, 17 aa, synthetic construct
  REGION 1..17: note = CDR-H2
WINPNSGGTNYAQKFQG

SEQ ID NO: 125 - protein, 9 aa, synthetic construct
  REGION 1..9: note = CDR-H3
SQRDGYMDY

SEQ ID NO: 126 - protein, 8 aa, synthetic construct
  REGION 1..8: note = CDR-L1
TGTSSDVG

SEQ ID NO: 127 - protein, 7 aa, synthetic construct
  REGION 1..7: note = CDR-L2
EDSKRPS

SEQ ID NO: 128 - protein, 10 aa, synthetic construct
  REGION 1..10: note = CDR-L3
SSNTRSSTLV

SEQ ID NO: 129 - protein, 118 aa, synthetic construct
  REGION 1..118: note = Variable heavy (VH) Anti-BCMA
EVQLVQSGAEMKKPGASLKLSCKASGYTFIDYYVYWMRQAPGQGLESMGWINPNSGGTNYAQKFQGRVTMTRDTSISTAYMELSRLRSDDTAMYYCARSQRDGYMDYWGQGTLVTVSS

SEQ ID NO: 130 - protein, 105 aa, synthetic construct
  REGION 1..105: note = Variable light (VL) Anti-BCMA
QSALTQPASVSASPGQSIAISCTGTSSDVGWYQQHPGKAPKLMIYEDSKRPSGVSNRFSGSKSGNTASLTISGLQAEDEADYYCSSNTRSSTLVFGGGTKLTVLG

SEQ ID NO: 131 - protein, 244 aa, synthetic construct
  REGION 1..244: note = scFv
QSALTQPASVSASPGQSIAISCTGTSSDVGWYQQHPGKAPKLMIYEDSKRPSGVSNRFSGSKSGNTASLTISGLQAEDEADYYCSSNTRSSTLVFGGGTKLTVLGSRGGGGSGGGGSGGGGSLEMAEVQLVQSGAEMKKPGASLKLSCKASGYTFIDYYVYWMRQAPGQGLESMGWINPNSGGTNYAQKFQGRVTMTRDTSISTAYMELSRLRSDDTAMYYCARSQRDGYMDYWGQGTLVTVSS

SEQ ID NO: 132 - protein, 654 aa, synthetic construct
  REGION 1..654: note = anti-BCMA CAR
QSALTQPASVSASPGQSIAISCTGTSSDVGWYQQHPGKAPKLMIYEDSKRPSGVSNRFSGSKSGNTASLTISGLQAEDEADYYCSSNTRSSTLVFGGGTKLTVLGSRGGGGSGGGGSGGGGSLEMAEVQLVQSGAEMKKPGASLKLSCKASGYTFIDYYVYWMRQAPGQGLESMGWINPNSGGTNYAQKFQGRVTMTRDTSISTAYMELSRLRSDDTAMYYCARSQRDGYMDYWGQGTLVTVSSESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 133 - protein, 271 aa, synthetic construct
  REGION 1..271: note = Heparin binding domain II of fibronectin (FN12-14)
AIPAPTDLKFTQVTPTSLSAQWTPPNVQLTGYRVRVTPKEKTGPMKEINLAPDSSSVVVSGLMVATKYEVSVYALKDTLTSRPAQGVVTTLENVSPPRRARVTDATETTITISWRTKTETITGFQVDAVPANGQTPIQRTIKPDVRSYTITGLQPGTDYKIYLYTLNDNARSSPVVIDASTAIDAPSNLRFLATTPNSLLVSWQPPRARITGYIIKYEKPGSPPREVVPRPRPGVTEATITGLEPGTEYTIYVIALKNNQKSEPLIGRKKT

SEQ ID NO: 134 - protein, 10 aa, synthetic construct
  REGION 1..10: note = Linker
GGGGSGGGGS

SEQ ID NO: 135 - protein, 585 aa, synthetic construct
  REGION 1..585: note = Viral binding protein
MAIPAPTDLKFTQVTPTSLSAQWTPPNVQLTGYRVRVTPKEKTGPMKEINLAPDSSSVVVSGLMVATKYEVSVYALKDTLTSRPAQGVVTTLENVSPPRRARVTDATETTITISWRTKTETITGFQVDAVPANGQTPIQRTIKPDVRSYTITGLQPGTDYKIYLYTLNDNARSSPVVIDASTAIDAPSNLRFLATTPNSLLVSWQPPRARITGYIIKYEKPGSPPREVVPRPRPGVTEATITGLEPGTEYTIYVIALKNNQKSEPLIGRKKTGGGGSGGGGSAIPAPTDLKFTQVTPTSLSAQWTPPNVQLTGYRVRVTPKEKTGPMKEINLAPDSSSVVVSGLMVATKYEVSVYALKDTLTSRPAQGVVTTLENVSPPRRARVTDATETTITISWRTKTETITGFQVDAVPANGQTPIQRTIKPDVRSYTITGLQPGTDYKIYLYTLNDNARSSPVVIDASTAIDAPSNLRFLATTPNSLLVSWQPPRARITGYIIKYEKPGSPPREVVPRPRPGVTEATITGLEPGTEYTIYVIALKNNQKSEPLIGRKKTSAWSHPQFEKGGGGSGGGGSGGSAWSHPQFEK

SEQ ID NO: 136 - DNA, 1755 bp, synthetic construct
  source 1..1755: mol_type = other DNA
  misc_feature 1..1755: note = Sequence encoding viral binding protein
atggcgattccggcgccgaccgatctgaaatttacccaggtgaccccgaccagcctgagcgcgcagtggaccccgccgaacgtgcagctgaccggctatcgcgtgcgcgtgaccccgaaagaaaaaaccggcccgatgaaagaaattaacctggcgccggatagcagcagcgtggtggtgagcggcctgatggtggcgaccaaatatgaagtgagcgtgtatgcgctgaaagataccctgaccagccgcccggcgcagggcgtggtgaccaccctggaaaacgtgagcccgccgcgccgcgcgcgcgtgaccgatgcgaccgaaaccaccattaccattagctggcgcaccaaaaccgaaaccattaccggctttcaggtggatgcggtgccggcgaacggccagaccccgattcagcgcaccattaaaccggatgtgcgcagctataccattaccggcctgcagccgggcaccgattataaaatttatctgtataccctgaacgataacgcgcgcagcagcccggtggtgattgatgcgagcaccgcgattgatgcgccgagcaacctgcgctttctggcgaccaccccgaacagcctgctggtgagctggcagccgccgcgcgcgcgcattaccggctatattattaaatatgaaaaaccgggcagcccgccgcgcgaagtggtgccgcgcccgcgcccgggcgtgaccgaagcgaccattaccggcctggaaccgggcaccgaatataccatttatgtgattgcgctgaaaaacaaccagaaaagcgaaccgctgattggccgcaaaaaaaccggcggcggcggcagcggcggcggcggcagcgcgattccggcgccgaccgatctgaaatttacccaggtgaccccgaccagcctgagcgcgcagtggaccccgccgaacgtgcagctgaccggctatcgcgtgcgcgtgaccccgaaagaaaaaaccggcccgatgaaagaaattaacctggcgccggatagcagcagcgtggtggtgagcggcctgatggtggcgaccaaatatgaagtgagcgtgtatgcgctgaaagataccctgaccagccgcccggcgcagggcgtggtgaccaccctggaaaacgtgagcccgccgcgccgcgcgcgcgtgaccgatgcgaccgaaaccaccattaccattagctggcgcaccaaaaccgaaaccattaccggctttcaggtggatgcggtgccggcgaacggccagaccccgattcagcgcaccattaaaccggatgtgcgcagctataccattaccggcctgcagccgggcaccgattataaaatttatctgtataccctgaacgataacgcgcgcagcagcccggtggtgattgatgcgagcaccgcgattgatgcgccgagcaacctgcgctttctggcgaccaccccgaacagcctgctggtgagctggcagccgccgcgcgcgcgcattaccggctatattattaaatatgaaaaaccgggcagcccgccgcgcgaagtggtgccgcgcccgcgcccgggcgtgaccgaagcgaccattaccggcctggaaccgggcaccgaatataccatttatgtgattgcgctgaaaaacaaccagaaaagcgaaccgctgattggccgcaaaaaaaccagcgcgtggagccatccgcagtttgaaaaaggcggcggcggcagcggcggcggcggcagcggcggcagcgcgtggagccatccgcagtttgaaaaa

SEQ ID NO: 137-140 - skipped (4 entries)

SEQ ID NO: 141 - protein, 6 aa, synthetic construct
  REGION 1..6: note = Heparin-Binding Sequence
PRRARV

SEQ ID NO: 142 - protein, 6 aa, synthetic construct
  REGION 1..6: note = Heparin-Binding Sequence
LIGRKK

SEQ ID NO: 143 - protein, 4 aa, synthetic construct
  REGION 1..4: note = Sequence of mutein streptavidin at sequence positions corresponding to positions 44 to 47 of streptavidin
IGAR

SEQ ID NO: 144 - protein, 4 aa, synthetic construct
  REGION 1..4: note = Sequence of mutein streptavidin at sequence positions corresponding to positions 44 to 47 of streptavidin
VTAR

SEQ ID NO: 145-146 - skipped (2 entries)

SEQ ID NO: 147 - protein, 6 aa, synthetic construct
  REGION 1..6: note = Linker
GGSGGS

SEQ ID NO: 148 - protein, 9 aa, synthetic construct
  REGION 1..9: note = Linker
GGSGGSGGS

SEQ ID NO: 149 - protein, 5 aa, synthetic construct
  REGION 1..5: note = Linker
GGGGS

SEQ ID NO: 150 - protein, 7 aa, synthetic construct
  REGION 1..7: note = Heparin-Binding Sequence
KNNQKSE

SEQ ID NO: 151 - protein, 7 aa, synthetic construct
  REGION 1..7: note = Heparin-Binding Sequence
LIGRKKT

SEQ ID NO: 152 - protein, 6 aa, synthetic construct
  REGION 1..6: note = Heparin-Binding Sequence
PRRGRV

SEQ ID NO: 153 - protein, 7 aa, synthetic construct
  REGION 1..7: note = Heparin-Binding Sequence
LIGKKKT

SEQ ID NO: 154 - protein, 7 aa, synthetic construct
  REGION 1..7: note = Heparin-Binding Sequence
LIGRKTG

SEQ ID NO: 155 - protein, 7 aa, synthetic construct
  REGION 1..7: note = Heparin-Binding Sequence
LIGRKTG

SEQ ID NO: 156 - protein, 7 aa, synthetic construct
  REGION 1..7: note = Heparin-Binding Sequence
LIGRKTG

SEQ ID NO: 157 - protein, 7 aa, synthetic construct
  REGION 1..7: note = Heparin-Binding Sequence
LIGRKFD

SEQ ID NO: 158 - protein, 7 aa, synthetic construct
  REGION 1..7: note = Heparin-Binding Sequence
LIGRKFE

SEQ ID NO: 159 - protein, 7 aa, synthetic construct
  REGION 1..7: note = Heparin-Binding Sequence
LIGRKFD

SEQ ID NO: 160 - protein, 7 aa, synthetic construct
  REGION 1..7: note = Heparin-Binding Sequence
LIGRKYD

SEQ ID NO: 161 - protein, 7 aa, synthetic construct
  REGION 1..7: note = Heparin-Binding Sequence
LIGRKFN

SEQ ID NO: 162 - protein, 127 aa, synthetic construct
  REGION 1..127: note = Minimal streptavidin
AEAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAAS

SEQ ID NO: 163 - protein, 127 aa, synthetic construct
  REGION 1..127: note = Mutein Streptavidin Val44-Thr45-Ala46-Arg47 and Glu117, Gly120, Tyr121 (mutein m1-9)
MEAGITGTWYNQLGSTFIVTAGADGALTGTYVTARGNAESRYVLTGRYDSAPATDGSGTALGWTVAWKNNYRNAHSATTWSGQYVGGAEARINTQWLLTSGTTEENAGYSTLVGHDTFTKVKPSAAS

SEQ ID NO: 164 - protein, 28 aa, synthetic construct
  REGION 1..28: note = Sequential modules of streptavidin-binding peptide; note = Sequential modules of streptavidin-binding peptide
  VARIANT 9..20: note = X is any amino acid
WSHPQFEKXXXXXXXXXXXXWSHPQFEK

(19) United States
(12) Patent Application Publication
(10) Pub. No.: US 2025/0297282 A1
(43) Pub. Date: Sep. 25, 2025

(54) VIRAL-BINDING PROTEIN AND RELATED REAGENTS, ARTICLES, AND METHODS OF USE

(71) Applicant: Juno Therapeutics GmbH, Munich (DE)

(72) Inventors: Mateusz Pawel POLTORAK, Grasbrunn (DE); Manuel EFFENBERGER, Villingen-Schwenningen (DE); Simon FRAESSLE, Kirchzarten (DE)

(73) Assignee: Juno Therapeutics GmbH, Munich (DE)

(21) Appl. No.: 18/862,153

(22) PCT Filed: May 4, 2023
(86) PCT No.: PCT/EP2023/061854

§ 371 Date: Oct. 31, 2024

Related U.S. Application Data
(60) Provisional application No. 63/338,872, filed on May 5, 2022.

(51) Int. Cl. C12N 15/86; B01D 15/32; B01D 15/38; B01D 15/42; C07K 16/14; C07K 16/18; C12N 5/0783

(52) U.S. Cl. CPC C12N 15/86; B01D 15/327; B01D 15/3823; B01D 15/426; C07K 16/14; C07K 16/18; C12N 5/0636; C12N 2501/2302; C12N 2501/2307; C12N 2501/2315; C12N 2510/00; C12N 2740/15043; C12N 2740/15051

(57) ABSTRACT

Provided herein are proteins comprising a viral-binding domain and reagents comprising same. In some aspects, the provided proteins and reagents can be used for the purification of viral particles. In some aspects, the provided proteins and reagents improve the efficiency with which target cells can be transduced. Also provided herein are methods using the provided proteins and reagents, including for purifying viral particles or for transducing cells. Related kits and articles of manufacture are also provided.

### CROSS-REFERENCE TO RELATED APPLICATIONS

This application claims priority to U.S. Provisional Application No. 63/338,872, filed May 5, 2022, the contents of which are hereby incorporated by reference in their entirety for all purposes.

### INCORPORATION BY REFERENCE OF SEQUENCE LISTING

The present application is being filed along with a Sequence Listing in electronic format. The Sequence Listing is provided as a file entitled 735042025640SegList.xml, created May 2, 2023, which is 212,078 bytes in size. The information in the electronic format of the Sequence Listing is incorporated by reference in its entirety.

### FIELD

The present disclosure relates to proteins comprising a viral-binding domain and reagents comprising same. In some aspects, the provided proteins and reagents can be used for the purification of viral particles. In some aspects, the provided proteins and reagents improve the efficiency with which target cells can be transduced. Also provided herein are methods using the provided proteins and reagents, including for purifying viral particles or for transducing cells. Related kits and articles of manufacture are also provided.

### BACKGROUND

Various cell therapy methods are available for treating diseases and conditions. Among cell therapy methods are methods involving immune cells, such as T cells (e.g., CD4+ and CD8+ T cells), which may be genetically engineered with a recombinant receptor, such as a chimeric antigen receptor. Improved methods for generating cell populations suitable for use in, for example, cell therapy, are needed. Provided are proteins, reagents, methods, and articles of manufacture that meet such needs.

### SUMMARY

Provided herein in some embodiments is a protein comprising a viral-binding domain and a streptavidin-binding partner, wherein the streptavidin-binding partner is connected to the viral-binding domain. In some embodiments, the viral-binding domain is a heparin-binding domain.

Also provided herein in some embodiments is a protein comprising two heparin-binding domains and a streptavidin-binding partner, wherein the streptavidin-binding partner is connected to one of the heparin-binding domains.

In some of any embodiments, the protein comprises between 2 and 10 heparin-binding domains, 2 and 9 heparin-binding domains, 2 and 8 heparin-binding domains, 2 and 7 heparin-binding domains, 2 and 6 heparin-binding domains, 2 and 5 heparin-binding domains, 2 and 4 heparin-binding domains, or 2 and 3 heparin-binding domains, each inclusive. In some of any embodiments, the protein comprises no more than two heparin-binding domains.

Also provided herein in some embodiments is a protein set forth by the formula (heparin-binding domain)ₙ-(streptavidin-binding partner), wherein n is between 2 and 10, inclusive.

In some of any embodiments, n is between 2 and 9, 2 and 8, 2 and 7, 2 and 6, 2 and 5, 2 and 4, or 2 and 3, each inclusive. In some of any embodiments, n is 2.

In some of any embodiments, the heparin-binding domains are different from one another. In some of any embodiments, the heparin-binding domains are identical to one another.

In some of any embodiments, one of the heparin-binding domains comprises an amino acid sequence that binds to heparin and exhibits at least 85% sequence identity to a heparin-binding domain of fibronectin. In some of any embodiments, each of the heparin-binding domains comprises an amino acid sequence that binds to heparin and exhibits at least 85% sequence identity to a heparin-binding domain of fibronectin. In some of any embodiments, one of the heparin-binding domains comprises the amino acid sequence of a heparin-binding domain of fibronectin. In some of any embodiments, each of the heparin-binding domains comprises the amino acid sequence of a heparin-binding domain of fibronectin. In some of any embodiments, the fibronectin is human fibronectin. In some of any embodiments, the heparin-binding domain of fibronectin is heparin-binding domain II of fibronectin (FN12-14).

In some of any embodiments, one of the heparin-binding domains comprises an amino acid sequence selected from X-B-B-X-B-X (SEQ ID NO: 137), X-B-B-B-X-X-B-X (SEQ ID NO: 138), X-B-X-B-B-X (SEQ ID NO: 139), and X-B-X-X-B-B-B-X (SEQ ID NO: 140), wherein each X is independently selected from any hydrophobic amino acid, and each B is independently selected from any basic amino acid. In some of any embodiments, each of the heparin-binding domains comprises an amino acid sequence selected from X-B-B-X-B-X (SEQ ID NO: 137), X-B-B-B-X-X-B-X (SEQ ID NO: 138), X-B-X-B-B-X (SEQ ID NO: 139), and X-B-X-X-B-B-B-X (SEQ ID NO: 140), wherein each X is independently selected from any hydrophobic amino acid, and each B is independently selected from any basic amino acid. In some of any embodiments, one of the heparin-binding domains comprises the amino acid sequence X-B-B-X-B-X (SEQ ID NO: 137), wherein each X is independently selected from any hydrophobic amino acid, and each B is independently selected from any basic amino acid. In some of any embodiments, each of the heparin-binding domains comprises the amino acid sequence X-B-B-X-B-X (SEQ ID NO: 137), wherein each X is independently selected from any hydrophobic amino acid, and each B is independently selected from any basic amino acid. In some of any embodiments, each B is independently selected from arginine and lysine.

In some of any embodiments, one of the heparin-binding domains comprises the amino acid sequence set forth in SEQ ID NO: 141. In some of any embodiments, each of the heparin-binding domains comprises the amino acid sequence set forth in SEQ ID NO: 141.

In some of any embodiments, one of the heparin-binding domains comprises the amino acid sequence set forth in SEQ ID NO: 150. In some of any embodiments, each of the heparin-binding domains comprises the amino acid sequence set forth in SEQ ID NO: 150.

In some of any embodiments, one of the heparin-binding domains comprises the amino acid sequence set forth in SEQ ID NO: 142. In some of any embodiments, each of the heparin-binding domains comprises the amino acid sequence set forth in SEQ ID NO: 142.

In some of any embodiments, one of the heparin-binding domains comprises an amino acid sequence that binds to heparin and exhibits at least 85% sequence identity to the amino acid sequence set forth in SEQ ID NO: 133. In some of any embodiments, each of the heparin-binding domains comprises an amino acid sequence that binds to heparin and exhibits at least 85% sequence identity to the amino acid sequence set forth in SEQ ID NO: 133. In some of any embodiments, one of the heparin-binding domains comprises the amino acid sequence set forth in SEQ ID NO: 133. In some of any embodiments, each of the heparin-binding domains comprises the amino acid sequence set forth in SEQ ID NO: 133. In some of any embodiments, one of the heparin-binding domains consists of the amino acid sequence set forth in SEQ ID NO: 133. In some of any embodiments, each of the heparin-binding domains consists of the amino acid sequence set forth in SEQ ID NO: 133.

In some of any embodiments, the heparin-binding domains are connected to one another via a peptide linker. In some of any embodiments, the peptide linker is a GS-linker. In some of any embodiments, the peptide linker comprises the amino acid sequence set forth in SEQ ID NO: 134. In some of any embodiments, the peptide linker consists of the amino acid sequence set forth in SEQ ID NO: 134.

In some of any embodiments, the streptavidin-binding partner is directly connected to one of the heparin-binding domains. In some of any embodiments, the streptavidin-binding partner is directly connected to the C-terminus of one of the heparin-binding domains. In some of any embodiments, the streptavidin-binding partner is at the C-terminus of the protein.

In some of any embodiments, the streptavidin-binding partner binds to a molecule that is streptavidin, avidin, a streptavidin analog or mutein, or an avidin analog or mutein. In some of any embodiments, the streptavidin-binding partner binds to a molecule that is streptavidin or a streptavidin mutein. In some of any embodiments, the streptavidin-binding partner binds to a biotin-binding site of the molecule. In some of any embodiments, the streptavidin mutein comprises the amino acid sequence set forth in any of SEQ ID NO: 3-6, 27, 28, 104, 105, and 163.

In some of any embodiments, the streptavidin-binding partner comprises biotin, a biotin analog or derivative, or a streptavidin-binding peptide. In some of any embodiments, the streptavidin-binding partner comprises biotin. In some of any embodiments, the streptavidin-binding partner comprises a biotin analog. In some of any embodiments, the streptavidin-binding partner comprises a biotin derivative. In some of any embodiments, the streptavidin-binding partner comprises a streptavidin-binding peptide. In some of any embodiments, the streptavidin-binding partner is a streptavidin-binding peptide.

In some of any embodiments, the streptavidin-binding peptide comprises the amino acid sequence set forth SEQ ID NO: 7 or SEQ ID NO: 8.

In some of any embodiments, the streptavidin-binding peptide comprises a sequential arrangement of two streptavidin-binding modules. In some of any embodiments, the streptavidin-binding modules are separated from one another by no more than 50 amino acids. In some of any embodiments, one of the streptavidin-binding modules comprises an amino acid sequence independently selected from the amino acid sequences set forth in SEQ ID NO: 7 and SEQ ID NO: 8. In some of any embodiments, each of the streptavidin-binding modules comprises an amino acid sequence independently selected from the amino acid sequences set forth in SEQ ID NO: 7 and SEQ ID NO: 8. In some of any embodiments, each of the streptavidin-binding modules comprises the amino acid sequence set forth in SEQ ID NO: 8.

In some of any embodiments, the streptavidin-binding peptide comprises the amino acid sequence set forth in any of SEQ ID NO: 15-19. In some of any embodiments, the streptavidin-binding peptide consists of the amino acid sequence set forth in any of SEQ ID NO: 15-19. In some of any embodiments, the streptavidin-binding peptide comprises the amino acid sequence set forth in SEQ ID NO: 16. In some of any embodiments, the streptavidin-binding peptide consists of the amino acid sequence set forth in SEQ ID NO: 16.

In some of any embodiments, the protein comprises the amino acid sequence set forth in SEQ ID NO: 135. In some of any embodiments, the protein consists of the amino acid sequence set forth in SEQ ID NO: 135.

Also provided herein in some embodiments is a heparin-binding reagent comprising (i) a protein reagent comprising one or a plurality of a molecule that is streptavidin, avidin, a streptavidin analog or mutein, or an avidin analog or mutein, and (ii) any of the provided proteins, wherein the streptavidin-binding partner of the protein is bound to at least one of the one or plurality of molecules.

In some of any embodiments, the heparin-binding reagent is capable of being immobilized on a solid support.

In some of any embodiments, the heparin-binding reagent is in soluble form. In some of any embodiments, the heparin-binding reagent is not immobilized on a solid support.

In some of any embodiments, the heparin-binding reagent comprises a plurality of the molecule, and the streptavidin-binding partner is bound to at least one of the plurality of molecules.

In some of any embodiments, the protein reagent is an oligomer of the plurality of molecules.

In some of any embodiments, the plurality of molecules comprises between or between about 500 and 5000 tetramers, 1000 and 4000 tetramers, or 2000 and 3000 tetramers, each inclusive, of the molecule of the protein reagent or a mixture of molecules each independently selected from streptavidin, avidin, a streptavidin analog or mutein, or an avidin analog or mutein. In some of any embodiments, the plurality of molecules comprises about 2500 tetramers of the molecule of the protein reagent or a mixture of molecules each independently selected from streptavidin, avidin, a streptavidin analog or mutein, or an avidin analog or mutein.

In some of any embodiments, the molecule of the protein reagent is streptavidin. In some of any embodiments, the molecule of the protein reagent is a streptavidin mutein. In some of any embodiments, the streptavidin mutein comprises the amino acid sequence IGAR (SEQ ID NO: 143) or VTAR (SEQ ID NO: 144) at sequence positions corresponding to positions 44 to 47 of the sequence of amino acids set forth in SEQ ID NO: 1. In some of any embodiments, the streptavidin mutein begins N-terminally in the region of amino acid positions 10 to 16 of SEQ ID NO: 1 and terminates C-terminally in the region of amino acid positions 133 to 142 of SEQ ID NO: 1.

Also provided herein in some embodiments is a heparin-binding reagent comprising a protein reagent and any of the provided proteins, wherein the protein reagent is an oligomer of a plurality of a molecule that is a streptavidin mutein, wherein the streptavidin mutein (i) comprises the amino acid sequence IGAR (SEQ ID NO: 143) or VTAR (SEQ ID NO: 144) at sequence positions corresponding to positions 44 to 47 of the sequence of amino acids set forth in SEQ ID NO: 1, (ii) begins N-terminally in the region of amino acid positions 10 to 16 of SEQ ID NO: 1, and (iii) terminates C-terminally in the region of amino acid positions 133 to 142 of SEQ ID NO: 1; and the streptavidin-binding partner of the protein (i) is a streptavidin-binding peptide comprising the amino acid sequence set forth in SEQ ID NO: 7 or SEQ ID NO: 8, and (ii) is bound to at least one of the plurality of molecules.

In some of any embodiments, the plurality of molecules comprises between or between about 500 and 5000 tetramers, 1000 and 4000 tetramers, or 2000 and 3000 tetramers, each inclusive, of the molecule of the protein reagent. In some of any embodiments, the plurality of molecules comprises about 2500 tetramers of the molecule of the protein reagent.

In some of any embodiments, the streptavidin mutein further comprises the residues Glu117, Gly120, and Tyr121 with reference to positions of the sequence of amino acids set forth in SEQ ID NO: 1.

In some of any embodiments, the streptavidin mutein comprises the sequence of amino acids set forth in any of SEQ ID NOs: 3-6, 27, 28, 104, and 105. In some of any embodiments, the streptavidin mutein comprises the sequence of amino acids set forth in SEQ ID NO: 6.

In some of any embodiments, molecules of the plurality of molecules are crosslinked to one another by a polysaccharide or a bifunctional linker. In some of any embodiments, molecules of the plurality of molecules are crosslinked to one another by an amine-to-thiol crosslinker.

In some of any embodiments, the streptavidin-binding partner is bound to a biotin-binding site of the at least one molecule.

In some of any embodiments, the streptavidin-binding partner is reversibly bound to the at least one molecule. In some of any embodiments, the binding affinity of the streptavidin-binding partner to the at least one molecule is reduced compared to the binding affinity of biotin to streptavidin. In some of any embodiments, the binding of the streptavidin-binding partner to the at least one molecule is disrupted by the presence of biotin.

In some of any embodiments, the heparin-binding reagent further comprises one or more binding agents that are each bound to the protein reagent. In some of any embodiments, one of the one or more binding agents is reversibly bound to the protein reagent. In some of any embodiments, each of the one or more binding agents is reversibly bound to the protein reagent.

In some of any embodiments, each of the one or more binding agents comprises a streptavidin-binding partner that is bound to at least one of the plurality of molecules. In some of any embodiments, the streptavidin-binding partner of one of the one or more binding agents is bound to a biotin-binding site of the at least one molecule. In some of any embodiments, the streptavidin-binding partner of each of the one or more binding agents is bound to a biotin-binding site of the at least one molecule.

In some of any embodiments, the streptavidin-binding partner of one of the one or more binding agents comprises biotin, a biotin analog, or a streptavidin-binding peptide. In some of any embodiments, the streptavidin-binding partner of each of the one or more binding agents comprises biotin, a biotin analog, or a streptavidin-binding peptide.

In some of any embodiments, the streptavidin-binding partner of one of the one or more binding agents comprises a biotin derivative. In some of any embodiments, the streptavidin-binding partner of each of the one or more binding agents comprises a biotin derivative.

In some of any embodiments, the streptavidin-binding partner of one of the one or more binding agents is reversibly bound to the at least one molecule. In some of any embodiments, the streptavidin-binding partner of each of the one or more binding agents is reversibly bound to the at least one molecule. In some of any embodiments, the binding affinity of the streptavidin-binding partner of one of the one or more binding agents to the at least one molecule is reduced compared to the binding affinity of biotin to streptavidin. In some of any embodiments, the binding affinity of the streptavidin-binding partner of each of the one or more binding agents to the at least one molecule is reduced compared to the binding affinity of biotin to streptavidin. In some of any embodiments, the binding of the streptavidin-binding partner of one of the one or more binding agents to the at least one molecule is disrupted by the presence of biotin. In some of any embodiments, the binding of the streptavidin-binding partner of each of the one or more binding agents to the at least one molecule is disrupted by the presence of biotin.

In some of any embodiments, the at least one molecule is streptavidin, and the streptavidin-binding partner of one of the one or more binding agents comprises a biotin analog or a streptavidin-binding peptide. In some of any embodiments, the at least one molecule is streptavidin, and the streptavidin-binding partner of each of the one or more binding agents comprises a biotin analog or a streptavidin-binding peptide.

In some of any embodiments, the at least one molecule is streptavidin, and the streptavidin-binding partner of one of the one or more binding agents comprises a biotin derivative. In some of any embodiments, the at least one molecule is streptavidin, and the streptavidin-binding partner of each of the one or more binding agents comprises a biotin derivative.

In some of any embodiments, the streptavidin-binding partner of one of the one or more binding agents comprises a streptavidin-binding peptide. In some of any embodiments, the streptavidin-binding partner of each of the one or more binding agents comprises a streptavidin-binding peptide. In some of any embodiments, the streptavidin-binding partner of one of the one or more binding agents consists of a streptavidin-binding peptide. In some of any embodiments, the streptavidin-binding partner of each of the one or more binding agents consists of a streptavidin-binding peptide.

In some of any embodiments, the streptavidin-binding peptide of one of the one or more binding agents comprises an amino acid sequence independently selected from the amino acid sequences set forth in SEQ ID NO: 7 and SEQ ID NO: 8. In some of any embodiments, the streptavidin-binding peptide of each of the one or more binding agents comprises an amino acid sequence independently selected from the amino acid sequences set forth in SEQ ID NO: 7 and SEQ ID NO: 8.

In some of any embodiments, the streptavidin-binding peptide of one of the one or more binding agents comprises a sequential arrangement of two streptavidin-binding modules. In some of any embodiments, the streptavidin-binding peptide of each of the one or more binding agents comprises a sequential arrangement of two streptavidin-binding modules. In some of any embodiments, the streptavidin-binding modules are separated from one another by no more than 50 amino acids. In some of any embodiments, one of the streptavidin-binding modules comprises an amino acid sequence independently selected from the amino acid sequences set forth in SEQ ID NO: 7 and SEQ ID NO: 8. In some of any embodiments, each of the streptavidin-binding modules comprises an amino acid sequence independently selected from the amino acid sequences set forth in SEQ ID NO: 7 and SEQ ID NO: 8. In some of any embodiments, each of the streptavidin-binding modules comprises the amino acid sequence set forth in SEQ ID NO: 8.

In some of any embodiments, the streptavidin-binding peptide of one of the one or more binding agents comprises an amino acid sequence independently selected from the amino acid sequences set forth in SEQ ID NO: 15-19. In some of any embodiments, the streptavidin-binding peptide of each of the one or more binding agents comprises an amino acid sequence independently selected from the amino acid sequences set forth in SEQ ID NO: 15-19. In some of any embodiments, the streptavidin-binding peptide of one of the one or more binding agents consists of an amino acid sequence independently selected from the amino acid sequences set forth in SEQ ID NO: 15-19. In some of any embodiments, the streptavidin-binding peptide of each of the one or more binding agents consists of an amino acid sequence independently selected from the amino acid sequences set forth in SEQ ID NO: 15-19. In some of any embodiments, the streptavidin-binding peptide of one of the one or more binding agents comprises the amino acid sequence set forth in SEQ ID NO: 16. In some of any embodiments, the streptavidin-binding peptide of each of the one or more binding agents comprises the amino acid sequence set forth in SEQ ID NO: 16. In some of any embodiments, the streptavidin-binding peptide of one of the one or more binding agents consists of the amino acid sequence set forth in SEQ ID NO: 16. In some of any embodiments, the streptavidin-binding peptide of each of the one or more binding agents consists of the amino acid sequence set forth in SEQ ID NO: 16.

In some of any embodiments, one of the one or more binding agents comprises an antibody or an antibody fragment. In some of any embodiments, each of the one or more binding agents comprises an antibody or an antibody fragment. In some of any embodiments, one of the one or more binding agents comprises an antibody fragment. In some of any embodiments, each of the one or more binding agents comprises an antibody fragment. In some of any embodiments, the antibody fragment is a monovalent antibody fragment. In some of any embodiments, the antibody fragment is a Fab.

In some of any embodiments, one of the one or more binding agents is a binding agent that binds to a molecule expressed on the surface of a target cell.

In some of any embodiments, the target cell is an immune cell. In some of any embodiments, the target cell is a T cell.

In some of any embodiments, the molecule expressed on the surface of the target cell is a member of a TCR/CD3 complex. In some of any embodiments, the molecule expressed on the surface of the target cell is CD3.

In some of any embodiments, the molecule expressed on the surface of the target cell is a costimulatory molecule.

In some of any embodiments, the molecule expressed on the surface of the target cell is a co-receptor.

In some of any embodiments, the binding agent is a first binding agent, the molecule expressed on the surface of the target cell is a first molecule, and one of the one or more binding agents is a second binding agent that binds to a second molecule expressed on the surface of the target cell.

In some of any embodiments, the second molecule expressed on the surface of the target cell is a costimulatory molecule.

In some of any embodiments, the costimulatory molecule is CD28, CD90 (Thy-1), CD95 (Apo−/Fas), CD137 (4-1BB), CD154 (CD40L), ICOS, LAT, CD27, OX40, or HVEM. In some of any embodiments, the costimulatory molecule is CD28.

In some of any embodiments, the first binding agent comprises the streptavidin-binding partner and an anti-CD3 antibody or antibody fragment, and the second binding agent comprises the streptavidin-binding partner and an anti-CD28 antibody or antibody fragment. In some of any embodiments, the first binding agent comprises the streptavidin-binding partner and an anti-CD3 Fab, and the second binding agent comprises the streptavidin-binding partner and an anti-CD28 Fab.

In some of any embodiments, the second molecule expressed on the surface of the target cell is a co-receptor.

In some of any embodiments, the co-receptor is CD4 or CD8.

In some of any embodiments, the first binding agent comprises the streptavidin-binding partner and an anti-CD4 antibody or antibody fragment, and the second binding agent comprises the streptavidin-binding partner and an anti-CD8 antibody or antibody fragment. In some of any embodiments, the first binding agent comprises the streptavidin-binding partner and an anti-CD4 Fab, and the second binding agent comprises the streptavidin-binding partner and an anti-CD8 Fab.

Also provided herein in some embodiments is a method for purifying viral particles, comprising (a) adding a sample comprising viral particles to an internal cavity of a chromatography column, wherein the internal cavity comprises a stationary phase comprising a chromatography matrix and any of the provided proteins, and the protein is immobilized on the chromatography matrix, thereby immobilizing a viral particle from the sample on the stationary phase; and (b) eluting the viral particle from the internal cavity.

In some of any embodiments, the eluting comprises disrupting the binding between the protein and the viral particle.

In some of any embodiments, the protein is reversibly bound to the chromatography matrix.

In some of any embodiments, the eluting comprises disrupting the binding between the protein and the chromatography matrix.

In some of any embodiments, the protein is immobilized on the chromatography matrix via a selection reagent comprising a molecule that is streptavidin, avidin, a streptavidin analog or mutein, or an avidin analog or mutein, wherein the selection reagent is immobilized on the chromatography matrix, and the streptavidin-binding partner of the protein is bound to the molecule. In some of any embodiments, the streptavidin-binding partner is bound to a biotin-binding site of the molecule of the selection reagent.

In some of any embodiments, the streptavidin-binding partner is reversibly bound to the molecule of the selection reagent.

In some of any embodiments, the molecule of the selection reagent is a streptavidin mutein. In some of any embodiments, the streptavidin mutein comprises the amino acid sequence IGAR (SEQ ID NO: 143) or VTAR (SEQ ID NO: 144) at sequence positions corresponding to positions 44 to 47 of the sequence of amino acids set forth in SEQ ID NO: 1. In some of any embodiments, the streptavidin mutein begins N-terminally in the region of amino acid positions 10 to 16 of SEQ ID NO: 1 and terminates C-terminally in the region of amino acid positions 133 to 142 of SEQ ID NO: 1. In some of any embodiments, the streptavidin mutein comprises the sequence of amino acids set forth in any of SEQ ID NOs: 3-6, 27, 28, 104, and 105. In some of any embodiments, the streptavidin mutein comprises the sequence of amino acids set forth in SEQ ID NO: 6.

In some of any embodiments, the eluting comprises adding, to the internal cavity, a composition comprising a substance that disrupts the immobilization of the viral particle on the stationary phase. In some of any embodiments, the substance comprises biotin or a biotin analog. In some of any embodiments, the substance comprises biotin. In some of any embodiments, the substance comprises a biotin analog. In some of any embodiments, the substance comprises a biotin derivative.

Also provided herein in some embodiments is a method for transducing cells, comprising incubating one or more target cells in the simultaneous presence of a viral particle and any of the provided heparin-binding reagents, thereby producing a transduced target cell.

In some of any embodiments, at least a portion of the incubating occurs in an internal cavity of a chromatography column.

In some of any embodiments, the one or more target cells are immobilized on a solid support during at least a portion of the incubating. In some of any embodiments, the solid support is a stationary phase for column chromatography. In some of any embodiments, the stationary phase is comprised in an internal cavity of a chromatography column during the at least a portion of the incubating.

Also provided herein in some embodiments is a method for on-column transduction of cells, comprising incubating, in an internal cavity of a chromatography column, one or more target cells in the simultaneous presence of a viral particle and any of the provided heparin-binding reagents, thereby producing a transduced target cell, wherein the internal cavity comprises a stationary phase, and the one or more target cells are immobilized on the stationary phase during at least a portion of the incubation.

In some of any embodiments, prior to the incubating, the method comprises adding a sample comprising a plurality of the target cells to the stationary phase, thereby immobilizing the one or more target cells on the stationary phase.

In some of any embodiments, the stationary phase comprises a selection agent that specifically binds to a selection marker expressed on the surface of the one or more target cells, wherein specific binding of the selection agent to the selection marker effects the immobilization of the one or more target cells on the stationary phase.

Also provided herein in some embodiments is a method for on-column transduction of cells, comprising (a) adding a sample comprising a plurality of target cells to an internal cavity of a chromatography column, wherein the internal cavity comprises a stationary phase comprising a selection agent that specifically binds to a selection marker expressed on the surface of one or more of the plurality of target cells, thereby immobilizing the one or more target cells on the stationary phase; and (b) incubating the one or more target cells in the simultaneous presence of a viral particle and any of the provided heparin-binding reagents, thereby producing a transduced target cell, wherein the one or more target cells are immobilized on the stationary phase during at least a portion of the incubation.

In some of any embodiments, prior to the incubating, the method comprises contacting the one or more target cells with one or more compositions each comprising one or both of the heparin-binding reagent and the viral particle. In some of any embodiments, prior to the incubating, the method comprises adding, to the stationary phase, one or more compositions each comprising one or both of the heparin-binding reagent and the viral particle, thereby contacting the one or more target cells with the one or more compositions. In some of any embodiments, the one or more compositions each comprise a cell medium.

In some of any embodiments, the one or more compositions are a first composition comprising the heparin-binding reagent and a second, separate composition comprising the viral particle. In some of any embodiments, the one or more target cells are simultaneously contacted with the first composition and the second composition. In some of any embodiments, the one or more compositions is a composition comprising both of the heparin-binding reagent and the viral particle. In some of any embodiments, the method comprises mixing the heparin-binding reagent and the viral particle to form the composition comprising both of the heparin-binding reagent and the viral particle.

In some of any embodiments, prior to the incubating, the one or more target cells are incubated in the presence of one or more binding agents, one of the one or more binding agents being a binding agent that binds to a molecule expressed on the surface of the one or more target cells.

In some of any embodiments, at least a portion of the incubating is further in the presence of one or more binding agents, one of the one or more binding agents being a binding agent that binds to a molecule expressed on the surface of the one or more target cells. In some of any embodiments, all of the incubating is further in the presence of one or more binding agents, one of the one or more binding agents being a binding agent that binds to a molecule expressed on the surface of the one or more target cells.

In some of any embodiments, the one or more binding agents are comprised in a second reagent comprising a second protein reagent comprising one or a plurality of a molecule that is streptavidin, avidin, a streptavidin analog or mutein, or an avidin analog or mutein, wherein the one or more binding agents are each bound to the second protein reagent of the second reagent. In some of any embodiments, one of the one or more binding agents is reversibly bound to the second protein reagent. In some of any embodiments, each of the one or more binding agents is reversibly bound to the second protein reagent.

In some of any embodiments, each of the one or more binding agents comprises a streptavidin-binding partner that is bound to at least one of the plurality of molecules of the second protein reagent.

In some of any embodiments, the second reagent is a stimulatory reagent.

In some of any embodiments, the method comprises contacting the one or more target cells with a composition comprising the second reagent. In some of any embodiments, the method comprises adding, to the stationary phase, a composition comprising the second reagent, thereby contacting the one or more target cells with the composition comprising the second reagent. In some of any embodiments, the one or more target cells are simultaneously contacted with the composition comprising the second reagent and the one or more compositions each comprising one or both of the heparin-binding reagent and the viral particle. In some of any embodiments, the composition comprising both of the heparin-binding reagent and the viral particle further comprises the second reagent.

In some of any embodiments, the heparin-binding reagent comprises the one or more binding agents, wherein the one or more binding agents are each bound to the protein reagent of the heparin-binding reagent. In some of any embodiments, one of the one or more binding agents is reversibly bound to the protein reagent. In some of any embodiments, each of the one or more binding agents is reversibly bound to the protein reagent.

In some of any embodiments, each of the one or more binding agents comprises a streptavidin-binding partner that is bound to at least one of the plurality of molecules of the protein reagent.

In some of any embodiments, the streptavidin-binding partner of one of the one or more binding agents comprises biotin, a biotin analog, or a streptavidin-binding peptide. In some of any embodiments, the streptavidin-binding partner of each of the one or more binding agents comprises biotin, a biotin analog, or a streptavidin-binding peptide. In some of any embodiments, the streptavidin-binding partner of one of the one or more binding agents comprises a streptavidin-binding peptide. In some of any embodiments, the streptavidin-binding partner of each of the one or more binding agents comprises a streptavidin-binding peptide. In some of any embodiments, the streptavidin-binding peptide of one of the one or more binding agents comprises the amino acid sequence set forth in any of SEQ ID NO: 7, 8, and 15-19. In some of any embodiments, the streptavidin-binding peptide of each of the one or more binding agents comprises the amino acid sequence set forth in any of SEQ ID NO: 7, 8, and 15-19. In some of any embodiments, the streptavidin-binding peptide of one of the one or more binding agents comprises the amino acid sequence set forth in SEQ ID NO: 16. In some of any embodiments, the streptavidin-binding peptide of each of the one or more binding agents comprises the amino acid sequence set forth in SEQ ID NO: 16.

In some of any embodiments, the streptavidin-binding partner of one of the one or more binding agents comprises a biotin derivative. In some of any embodiments, the streptavidin-binding partner of each of the one or more binding agents comprises a biotin derivative.

In some of any embodiments, one of the one or more binding agents comprises an antibody or an antibody fragment. In some of any embodiments, each of the one or more binding agents comprises an antibody or an antibody fragment. In some of any embodiments, one of the one or more binding agents comprises an antibody fragment. In some of any embodiments, each of the one or more binding agents comprises an antibody fragment. In some of any embodiments, the antibody fragment is a monovalent antibody fragment. In some of any embodiments, the antibody fragment is a Fab.

In some of any embodiments, the binding agent binds to the molecule expressed on the one or more target cells and thereby provides a primary activation signal to the one or more target cells.

In some of any embodiments, the one or more target cells are immune cells. In some of any embodiments, the one or more target cells are T cells.

In some of any embodiments, the molecule expressed on the surface of the one or more target cells is a member of a TCR/CD3 complex. In some of any embodiments, the molecule expressed on the surface of the one or more target cells is CD3.

In some of any embodiments, the molecule expressed on the surface of the one or more target cells is a costimulatory molecule. In some of any embodiments, the binding agent binds to the costimulatory molecule and thereby provides a costimulatory signal to the one or more target cells.

In some of any embodiments, the molecule expressed on the surface of the one or more target cells is a co-receptor.

In some of any embodiments, the binding agent is a first binding agent, the molecule expressed on the surface of the one or more target cells is a first molecule, and one of the one or more binding agents is a second binding agent that binds to a second molecule expressed on the surface of the one or more target cells.

In some of any embodiments, the second molecule expressed on the surface of the one or more target cells is a costimulatory molecule. In some of any embodiments, the second binding agent binds to the costimulatory molecule and thereby provides a costimulatory signal to the one or more target cells.

In some of any embodiments, the costimulatory molecule is CD28, CD90 (Thy-1), CD95 (Apo−/Fas), CD137 (4-1BB), CD154 (CD40L), ICOS, LAT, CD27, OX40, or HVEM. In some of any embodiments, the costimulatory molecule is CD28.

In some of any embodiments, the first binding agent comprises a streptavidin-binding partner and an anti-CD3 antibody or antibody fragment, and the second binding agent comprises a streptavidin-binding partner and an anti-CD28 antibody or antibody fragment. In some of any embodiments, the first binding agent comprises a streptavidin-binding partner and an anti-CD3 Fab, and the second binding agent comprises a streptavidin-binding partner and an anti-CD28 Fab.

In some of any embodiments, the second molecule expressed on the surface of the target cell is a co-receptor.

In some of any embodiments, the co-receptor is CD4 or CD8.

In some of any embodiments, the first binding agent comprises a streptavidin-binding partner and an anti-CD4 antibody or antibody fragment, and the second binding agent comprises a streptavidin-binding partner and an anti-CD8 antibody or antibody fragment. In some of any embodiments, the first binding agent comprises a streptavidin-binding partner and an anti-CD4 Fab, and the second binding agent comprises a streptavidin-binding partner and an anti-CD8 Fab.

In some of any embodiments, the incubating is initiated within or within about 10 minutes, within or within about 20 minutes, within or within about 30 minutes, within or within about 45 minutes, within or within about 60 minutes, within or within about 90 minutes, or within or within about 120 minutes after adding the sample to the internal cavity.

In some of any embodiments, the incubating is carried out in a cell medium. In some of any embodiments, the cell medium is a serum free medium. In some of any embodiments, the cell medium comprises a recombinant cytokine. In some of any embodiments, the recombinant cytokine is selected from IL-2, IL-15, and IL-7. In some of any embodiments, the cell medium comprises recombinant IL-2, IL-15, and IL-7.

In some of any embodiments, the incubating is in the presence of between or between about 0.1 μg and 20 μg, inclusive; between or between about 0.1 μg and 12 μg, inclusive; or between or between about 0.5 μg and 8 μg, inclusive, of the heparin-binding reagent; each per 10⁶ cells of the one or more target cells or of an estimated cell count thereof. In some of any embodiments, the incubating is in the presence of between or between about 3 μg and 5 μg, inclusive, of the heparin-binding reagent per 10⁶ cells of the one or more target cells or of an estimated cell count thereof.

In some of any embodiments, the composition of the one or more compositions that comprises the heparin-binding reagent comprises between or between about 0.1 μg and 20 μg, inclusive; between or between about 0.1 μg and 12 μg, inclusive; or between or between about 0.5 μg and 8 μg, inclusive, of the heparin-binding reagent; each per 10⁶ cells of the one or more target cells or of an estimated cell count thereof. In some of any embodiments, the composition of the one or more compositions that comprises the heparin-binding reagent comprises between or between about 3 μg and 5 μg, inclusive, of the heparin-binding reagent per 10⁶ cells of the one or more target cells or of an estimated cell count thereof.

In some of any embodiments, the incubating is in the presence of between or between about 0.1 μL and 100 μL, inclusive; between or between about 0.5 μL and 50 μL, inclusive; between or between about 1 μL and 25 μL, inclusive; or between or between about 2 μL and 10 μL, inclusive, of a preparation of the viral particle per 10⁶ cells of the one or more target cells or of an estimated cell count thereof. In some of any embodiments, the incubating is in the presence of about 6 μL per 10⁶ cells of the one or more target cells or of an estimated cell count thereof.

In some of any embodiments, the composition of the one or more compositions that comprises the viral particle comprises between or between about 0.1 μL and 100 μL, inclusive; between or between about 0.5 μL and 50 μL, inclusive; between or between about 1 μL and 25 μL, inclusive; or between or between about 2 μL and 10 μL, inclusive, of a preparation of the viral particle per 10⁶ cells of the one or more target cells or of an estimated cell count thereof. In some of any embodiments, the composition of the one or more compositions that comprises the viral particle comprises about 6 μL per 10⁶ cells of the one or more target cells or of an estimated cell count thereof.

In some of any embodiments, the preparation of the viral particle has a titer of between or between about 1×10⁶ TU/mL and 1×10⁹ TU/mL, between or between about 1×10⁶ TU/mL and 1×10⁸ TU/mL, between or between about 1×10⁶ TU/mL and 1×10⁷ TU/mL, between or between about 1×10⁷ TU/mL and 1×10⁹ TU/mL, between or between about 1×10⁷ TU/mL and 1×10⁸ TU/mL, or between or between about 1×10⁸ TU/mL and 1×10⁹ TU/mL.

In some of any embodiments, the stationary phase has a binding capacity of between or between about 0.5 billion and 5 billion cells, 0.5 billion and 4 billion cells, 0.5 billion and 3 billion cells, 0.5 billion and 2 billion cells, 1 billion and 5 billion cells, 1 billion and 4 billion cells, 1 billion and 3 billion cells, or 1 billion and 2 billion cells, each inclusive. In some of any embodiments, the stationary phase has a binding capacity of between or between about 1 billion and 2 billion cells, inclusive.

In some of any embodiments, at least a portion of the incubating is carried out at a temperature between about 35° C. and about 39° C. In some of any embodiments, all of the incubating is carried out at a temperature between about 35° C. and about 39° C. In some of any embodiments, at least a portion of the incubating is carried out at a temperature of or of about 37° C. In some of any embodiments, all of the incubating is carried out at a temperature of or of about 37° C. In some of any embodiments, the temperature of the stationary phase is regulated by one or more heating elements configured to provide heat to the stationary phase.

In some of any embodiments, the method comprises collecting the transduced target cell. In some of any embodiments, the collecting comprises eluting the transduced target cell from the chromatography column.

In some of any embodiments, the collecting is carried out within 24 hours, 22 hours, 20 hours, 18 hours, 16 hours, 16 hours, 14 hours, 12 hours, 10 hours, 9 hours, 8 hours, 7 hours, 6 hours, or 5 hours after the initiation of the incubating. In some of any embodiments, the collecting is carried out between or between about 2 hours and 24 hours, 2 hours and 22 hours, 2 hours and 20 hours, 2 hours and 18 hours, 2 hours and 16 hours, 2 hours and 14 hours, 2 hours and 12 hours, 2 hours and 10 hours, 2 hours and 9 hours, 2 hours and 8 hours, 2 hours and 7 hours, 2 hours and 6 hours, 2 hours and 5 hours, 3 hours and 6 hours, 3 hours and 5 hours, 4 hours and 6 hours, or 4 hours and 5 hours, each inclusive, after the initiation of the incubating. In some of any embodiments, the collecting is carried out at or about 4.5 hours after the initiation of the incubating.

In some of any embodiments, the collecting comprises adding a wash buffer to the chromatography column to collect the transduced target cell. In some of any embodiments, the wash buffer comprises a cell medium. In some of any embodiments, the cell medium is a serum free medium. In some of any embodiments, the wash buffer comprises a recombinant cytokine. In some of any embodiments, the recombinant cytokine for the wash buffer is selected from IL-2, IL-15, and IL-7. In some of any embodiments, the wash buffer comprises recombinant IL-2, IL-15, and IL-7.

In some of any embodiments, the wash buffer does not comprise a competition agent to elute the transduced target cell from the stationary phase. In some of any embodiments, the wash buffer comprises a competition agent to elute the transduced target cell from the stationary phase.

In some of any embodiments, the competition agent facilitates detachment of the one or more target cells from the stationary phase. In some of any embodiments, the competition agent comprises biotin or a biotin analog. In some of any embodiments, the competition agent comprises biotin. In some of any embodiments, the competition agent comprises a biotin analog. In some of any embodiments, the competition agent comprises a biotin derivative.

In some of any embodiments, the method comprises further incubating the collected transduced target cell.

In some of any embodiments, the further incubating is carried at a temperature of at or about 37°±2° C.

In some of any embodiments, the further incubating is carried out for no more than 14 days, no more than 12 days, no more than 10 days, no more than 8 days, no more than 6 days, or no more than 5 days.

In some of any embodiments, the further incubating is carried out under conditions to induce expansion of the collected transduced target cell.

In some of any embodiments, the further incubating is carried out in cell medium comprising a recombinant cytokine. In some of any embodiments, the recombinant cytokine for the further incubating is selected from IL-2, IL-15, and IL-7. In some of any embodiments, the further incubating is carried out in cell medium comprising recombinant IL-2, IL-15, and IL-7.

In some of any embodiments, the further incubating is carried out under conditions in which there is minimal or no further expansion of the collected transduced target cell.

In some of any embodiments, the further incubating is carried out in a basal medium without any recombinant cytokines.

In some of any embodiments, the stationary phase comprises a chromatography matrix, and the selection agent is immobilized on the chromatography matrix. In some of any embodiments, the selection agent is immobilized on the chromatography matrix via a selection reagent comprising a molecule that is streptavidin, avidin, a streptavidin analog or mutein, or an avidin analog or mutein, wherein the selection reagent is immobilized on the chromatography matrix, and the selection agent comprises a streptavidin-binding partner that is bound to the molecule. In some of any embodiments, the streptavidin-binding partner of the selection agent is bound to a biotin-binding site of the molecule of the selection reagent.

In some of any embodiments, the molecule of the selection reagent is a streptavidin mutein. In some of any embodiments, the streptavidin mutein comprises the amino acid sequence IGAR (SEQ ID NO: 143) or VTAR (SEQ ID NO: 144) at sequence positions corresponding to positions 44 to 47 of the sequence of amino acids set forth in SEQ ID NO: 1. In some of any embodiments, the streptavidin mutein begins N-terminally in the region of amino acid positions 10 to 16 of SEQ ID NO: 1 and terminates C-terminally in the region of amino acid positions 133 to 142 of SEQ ID NO: 1. In some of any embodiments, the streptavidin mutein comprises the sequence of amino acids set forth in any of SEQ ID NOs: 3-6, 27, 28, 104, 105, and 163. In some of any embodiments, the streptavidin mutein comprises the sequence of amino acids set forth in SEQ ID NO: 6.

In some of any embodiments, the streptavidin-binding partner of the selection agent comprises biotin, a biotin analog, or a streptavidin-binding peptide. In some of any embodiments, the streptavidin-binding partner of the selection agent comprises biotin. In some of any embodiments, the streptavidin-binding partner of the selection agent comprises a biotin analog. In some of any embodiments, the streptavidin-binding partner of the selection agent comprises a biotin derivative. In some of any embodiments, the streptavidin-binding partner of the selection agent comprises a streptavidin-binding peptide.

In some of any embodiments, the binding of the streptavidin-binding partner of the selection agent to the molecule of the selection reagent is reversible.

In some of any embodiments, the streptavidin-binding partner of the selection agent comprises a streptavidin-binding peptide. In some of any embodiments, the streptavidin-binding peptide of the selection agent comprises the amino acid sequence set forth in any of SEQ ID NO: 7, 8, and 15-19. In some of any embodiments, the streptavidin-binding peptide comprises the amino acid sequence set forth in SEQ ID NO: 16.

In some of any embodiments, the selection agent comprises an antibody or antibody fragment that binds to the selection marker. In some of any embodiments, the selection agent comprises an antibody fragment that binds to the selection marker. In some of any embodiments, the antibody fragment is a monovalent antibody fragment. In some of any embodiments, the antibody fragment is a Fab.

In some of any embodiments, the selection marker is a T cell coreceptor or a member of a T cell antigen receptor complex. In some of any embodiments, the selection marker is selected from the group consisting of CD3, CD4, CD8, CD45RA, CD27, CD28, and CCR7. In some of any embodiments, the selection marker is CD3.

In some of any embodiments, the one or more target cells are primary cells from a human subject. In some of any embodiments, the sample is an apheresis or leukapheresis product.

In some of any embodiments, the viral particle comprises a nucleic acid sequence encoding a recombinant protein. In some of any embodiments, the recombinant protein is an antigen receptor. In some of any embodiments, the recombinant protein is a chimeric antigen receptor (CAR). In some of any embodiments, the recombinant protein is a T cell receptor (TCR).

In some of any embodiments, the viral particle is a viral vector. In some of any embodiments, the viral vector is a retroviral vector. In some of any embodiments, the viral vector is a lentiviral vector. In some of any embodiments, the viral vector is pseudotyped with VSV-G.

In some of any embodiments, the method comprises harvesting the collected transduced target cell. In some of any embodiments, the harvesting is carried out after the further incubating.

In some of any embodiments, the method comprises formulating the harvested transduced target cell for cryopreservation or administration to a subject. In some of any embodiments, the harvested transduced target cell is formulated in the presence of a pharmaceutically acceptable excipient or a cryoprotectant.

In some of any embodiments, one of the steps of the method is carried out in a closed system. In some of any embodiments, each of the steps of the method is carried out in a closed system.

In some of any embodiments, one of the steps of the method is automated. In some of any embodiments, each of the steps of the method is automated.

Also provided herein in some embodiments is a kit for purifying viral particles, comprising any of the provided proteins, a chromatography matrix suitable for viral purification using column chromatography, and a selection reagent, the selection reagent comprising a molecule that is streptavidin, avidin, a streptavidin analog or mutein, or an avidin analog or mutein, wherein the selection reagent is capable of being immobilized on the chromatography matrix.

In some of any embodiments, the streptavidin-binding partner of the protein is bound to the molecule.

Also provided herein in some embodiments is a kit for transducing cells, comprising any of the provided heparin-binding reagents, a chromatography matrix suitable for cell separation using column chromatography, and a selection agent that specifically binds to a selection marker expressed on the surface of a target cell, wherein the selection agent is capable of being immobilized on the chromatography matrix.

In some of any embodiments, the kit comprises a viral particle.

In some of any embodiments, the kit comprises a stimulatory reagent.

In some of any embodiments, the selection agent is immobilized on the chromatography matrix.

In some of any embodiments, the selection agent comprises a streptavidin-binding partner.

In some of any embodiments, the kit comprises a selection reagent, the selection reagent comprising a molecule that is streptavidin, avidin, a streptavidin analog or mutein, or an avidin analog or mutein, wherein the selection reagent is capable of being immobilized on the chromatography matrix.

In some of any embodiments, the selection reagent is immobilized on the chromatography matrix.

In some of any embodiments, the streptavidin-binding partner of the selection agent is bound to the molecule.

In some of any embodiments, the kit comprises a chromatography column. In some of any embodiments, the chromatography matrix is comprised in an internal cavity of the chromatography column.

Also provided herein in some embodiments is a stationary phase for purifying viral particles, comprising any of the provided proteins, a chromatography matrix suitable for viral purification using column chromatography, and a selection reagent, the selection reagent comprising a molecule that is streptavidin, avidin, a streptavidin analog or mutein, or an avidin analog or mutein, wherein the selection agent is immobilized on the chromatography matrix, and the streptavidin-binding partner of the protein is bound to the molecule.

Also provided herein in some embodiments is an article of manufacture for purifying viral particles, comprising any of the provided stationary phases and a chromatography column, wherein the stationary phase is comprised in an internal cavity of the chromatography column.

Also provided herein in some embodiments is a viral particle purified by any of the provided methods.

Also provided herein in some embodiments is a target cell transduced by any of the provided methods.

### BRIEF DESCRIPTION OF THE DRAWINGS

FIG. 1 shows GFP expression of T cells five days after transduction using flow through (FT) or elution fractions collected from columns functionalized with viral binding protein (+VB) or columns functionalized without viral binding protein (−VB).

FIG. 2A and FIG. 2B show transduction efficiency of CD3+ T cells (FIG. 2A) and yield of transduced CD3+ T cells (CAR+, FIG. 2B) following transduction with a lentiviral vector encoding a CAR in the presence (+VBP) or absence (−VBP) of VBP-functionalized reagent and five days of culture.

FIG. 3A-3D show transduction efficiency of CD3+ T cells (FIG. 3A), yield of transduced CD3+ T cells (CAR+) (FIG. 3B), transduction efficiency fold-increase (FIG. 3C), and yield fold-increase (FIG. 3D) following transduction with a lentiviral vector encoding a CAR in the presence of VBP-functionalized reagent or anti-CD4/anti-CD8 VBP-functionalized reagent and five days of culture.

FIG. 4A and FIG. 4B show transduction efficiency fold-increase (FIG. 4A) and yield fold-increase (FIG. 4B) following transduction of CD3+ T cells with a lentiviral vector encoding a CAR in the presence of anti-CD3/anti-CD28 VBP-functionalized stimulatory reagent and five days of culture.

### DETAILED DESCRIPTION

Provided herein in some embodiments is a protein that contains a viral-binding domain. In some embodiments, the protein may be also referred to herein as a viral-binding protein (VBP). In some embodiments, the viral-binding domain binds to a viral particle. In some embodiments, the viral-binding domain binds to a viral vector.

Also provided herein in some embodiments is a protein that contains a heparin-binding domain. In some embodiments, the protein binds to a viral particle. In some embodiments, the heparin-binding domain of the protein binds to the viral particle. In some embodiments, the heparin-binding domain is a viral-binding domain. In some embodiments, the viral particle is a viral vector. In some embodiments, the protein that contains a heparin-binding domain is an example of a VBP.

In some embodiments, the VBP can be used for the purification of viral particles, for instance via binding of the VBP to viral particles contained in a sample. Also provided herein in some embodiments are methods for purifying viral particles, for instance those using any of the provided VBPs.

In some embodiments, the VBP contains a binding partner, such as a streptavidin-binding partner. In some embodiments, the binding partner of the VBP can bind to a target binding site of a protein. In some aspects, the binding allows for binding of the VBP to the protein via interactions between the binding partner and the binding site. In some embodiments, this binding allows for the detection of the VBP. In some embodiments, the binding partner of the VBP can facilitate purification of the VBP from a sample, for example using methods that involve selecting for proteins having the binding partner, e.g., via binding to the binding partner. In some embodiments, the binding partner can be used to bind the VBP to a reagent having a binding site for the binding partner. Such functionalization can provide or improve the viral binding ability of the reagent. In some embodiments, the VBP is multimerized on the reagent, which in some aspects further increases the viral binding ability of the reagent.

Also provided herein in some embodiments are VBP-functionalized reagents (also referred to herein as VBP reagents), including those containing any of the provided VBPs.

Also provided herein in some embodiments are heparin-binding reagents, including those containing any of the provided proteins containing a heparin-binding domain. In some embodiments, the heparin-binding reagent binds to a viral particle. In some embodiments, the protein containing the heparin-binding domain binds to a viral particle. In some embodiments, the heparin-binding domain binds to a viral particle. In some embodiments, the heparin-binding reagent is an example of a VBP reagent.

While the provided proteins and reagents are sometimes referred to herein as being viral-binding proteins or reagents, one of ordinary skill in the art would recognize that the provided proteins and reagents may be capable of binding to targets other than viral particles, such as targets in addition to a viral particle. For instance, the provided proteins and reagents may be used to promote interaction with any heparin-containing molecule. One of ordinary skill in the art would recognize that the provided proteins and reagents may be used for a variety of purposes, such as in addition to for binding to viral particles, and all such uses are among the provided embodiments.

In some embodiments, the VBP reagent also contains a binding agent. In some embodiments, the binding agent binds to a cell surface molecule, such as a cell surface molecule on target cells for which transduction is desired. In some aspects, the targeting of cell surface molecules and viral particles by one reagent, e.g., the VBP reagent, may increase the co-localization of target cells and viral particles. As shown herein, the presence of such VBP reagents during transduction can increase the number of target cells transduced with a viral particle.

In some embodiments, the binding agent is capable of stimulating target cells. In some embodiments, the VBP reagent is a combined stimulation and transduction reagent capable of both stimulating target cells and facilitating transduction thereof, as demonstrated herein.

Also provided herein in some embodiments are methods of transducing cells, for instance using any of the provided VBPs and VBP reagents. In some embodiments, the provided methods are methods of transducing cells contained in a chromatography column, for instance while the cells are immobilized on-column following selection.

Also provided herein are related stationary phases, kits, and articles of manufacturing, such as those that can be used to perform the provided methods, and for instance those including any of the provided VBPs or VBP reagents.

The provided embodiments offer various advantages. Certain available methods for generating engineered cell populations, such as those for use in cell therapies, often require separate selection, stimulation, and engineering steps, which can prolong manufacturing. Multiple processing steps may result in cell stress, potentially affecting downstream cell processing or even cell biology, in addition to requiring considerable time to complete. Certain available methods may also result in inefficient transduction of cells or low yield of transduced cells. Additional methods for generating engineered cell populations are needed.

In some aspects, the provided embodiments address these and other needs. In some aspects, and as shown herein, transduction efficiency is improved by the presence during transduction of the VBP reagents provided herein, thereby increasing the yield of engineered cells. In some embodiments, the provided methods, which in some embodiments involve use of the provided VBP reagents, are those with improved yield of engineered cells.

In some embodiments, the use of the VBP in a stimulatory reagent allows for the provision of a single reagent for stimulation and improved transduction of cells, thereby simplifying manufacturing. As also shown herein, the VBP can also improve transduction when provided on a separate reagent from the stimulatory reagent. Thus, the provided VBPs and VBP reagents offer flexibility in how they can be used in methods for engineering cells, including those involving the simultaneous stimulation and transduction of cells, as demonstrated herein.

In some embodiments, the cells are simultaneously stimulated and transduced following selection, including while cells are immobilized on the stationary phase of a chromatography column used for selection. Thus, in some aspects, the provided embodiments reduce and/or minimize cell handling, contamination, and processing time in a manufacturing process. Further, the provided embodiments enable the use of a fully closed system that unifies on-column operations such as selection, stimulation, and genetic engineering of cells. In some aspects, steps of the provided methods can be automated, or the methods can be fully automated. In some aspects, the provided embodiments allow for more rapid manufacturing with less manipulation of cells, for instance leading to the retention of broader cell properties, improved cell production turn-around times, reduction in hands-on failures, and ultimately reduced manufacturing costs for cell therapies. In some aspects, the provided embodiments are advantageous in that they allow for condensing multiple processing steps (e.g., selection, stimulation, and transduction) and allow the condensed process to occur within the same container, for instance also in a closed system, which can provide increased efficiency and sterility.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. Viral-Binding Proteins (VBPs)

In some embodiments, the provided viral-binding protein (VBP) contains a viral-binding domain. In some embodiments, the viral-binding domain binds to a viral particle. In some embodiments, the viral-binding domain binds to a viral vector. In some embodiments, the viral-binding domain is an amino acid sequence that bind to a viral particle. In some embodiments, the viral-binding domain is any of the viral-binding domains described herein, for instance any described in Section I-A. In some embodiments, the viral-binding domain is any of the heparin-binding domains described herein.

In some embodiments, the provided viral-binding protein (VBP) contains a heparin-binding domain. In some embodiments, the heparin-binding domain is an amino acid sequence that bind to heparin. In some embodiments, the heparin-binding domain binds to a viral particle. In some embodiments, the heparin-binding domain binds to a viral vector. In some embodiments, the heparin-binding domain is any of the heparin-binding domains described herein, for instance any described in Section I-A.

In some embodiments, the VBP contains a binding partner. In some embodiments, the binding partner can bind to a target binding site of a protein. In some aspects, the binding allows for binding of the VBP to the protein via interactions between the binding partner and the binding site. In some embodiments, this binding allows for the detection of the VBP. In some embodiments, the binding partner facilitates purification of the VBP from a sample, for example using methods that involve selecting for proteins having the binding partner, e.g., via binding to the binding partner. In some embodiments, the binding partner can be used to detect or purify VBPs containing particular viral-binding or heparin-binding domains.

In some embodiments, the VBP contains a binding partner by which a reagent that is suitable for use during the transduction of target cells with viral particles, or is suitable for use in one or more steps of a method involving the transduction of target cells with viral particles, can incorporate the VBP therein. In some embodiments, the binding partner binds to a molecule of the reagent, thereby functionalizing the reagent with the VBP. In some aspects, this functionalization imparts viral binding ability to the functionalized reagent. In some embodiments, the reagent prior to functionalization possesses limited or no viral binding ability. In some embodiments, the reagent possesses viral binding ability prior to functionalization, which in some aspects can be augmented by the functionalization of the reagent with the VBP. In some embodiments, the functionalized reagent retains its previous properties, in some instances such that the reagent has viral binding and other abilities following functionalization. As an example, in some embodiments, a stimulatory reagent can be functionalized with the VBP via binding of the binding partner to a molecule of the stimulatory reagent. In some aspects, the functionalized reagent has the ability both to stimulate cells and to bind to viral particles via the incorporation of the VBP therein, for instance via the reagent's binding to the binding partner of the VBP.

In some embodiments, the binding partner is any of the binding partners described herein, for instance any as described in Section I-B. In some embodiments, the binding partner is any of the streptavidin or avidin binding partners described herein. In some embodiments, the binding partner is any of the streptavidin-binding partners described herein. In some embodiments, the binding partner contains biotin, a biotin analog or derivative, or a streptavidin-binding peptide. In some embodiments, the binding partner contains any of the streptavidin-binding peptides described herein. In some aspects, the binding partner allows for the functionalization of reagents having a binding site for the binding partner, for instance reagents containing streptavidin, avidin, a streptavidin analog or mutein, or an avidin analog or mutein.

In some embodiments, the VBP contains two or more viral-binding domains. In some embodiments, the viral-binding domains of the VBP are different from one another. In some embodiments, the viral-binding domains of the VBP are identical to one another.

In some embodiments, the VBP contains between 2 and 20, 2 and 18, 2 and 16, 2 and 14, 2 and 12, 2 and 10, 2 and 8, 2 and 6, 2 and 4, 4 and 20, 4 and 18, 4 and 16, 4 and 14, 4 and 12, 4 and 10, 4 and 8, 4 and 6, 6 and 20, 6 and 18, 6 and 16, 6 and 14, 6 and 12, 6 and 10, 6 and 8, 8 and 20, 8 and 18, 8 and 16, 8 and 14, 8 and 12, 8 and 10, 10 and 20, 10 and 18, 10 and 16, 10 and 14, 10 and 12, 12 and 20, 12 and 18, 12 and 16, 12 and 14, 14 and 20, 14 and 18, 14 and 16, 16 and 20, 16 and 18, or 18 and 20 viral-binding domains, each inclusive. In some embodiments, the VBP contains between 2 and 10, 2 and 9, 2 and 8, 2 and 7, 2 and 6, 2 and 5, 2 and 4, 2 and 3, 3 and 10, 3 and 9, 3 and 8, 3 and 7, 3 and 6, 3 and 5, 3 and 4, 4 and 10, 4 and 9, 4 and 8, 4 and 7, 4 and 6, 4 and 5, 5 and 10, 5 and 9, 5 and 8, 5 and 7, 5 and 6, 6 and 10, 6 and 9, 6 and 8, 6 and 7, 7 and 10, 7 and 9, 7 and 8, 8 and 10, 8 and 9, or 9 and 10 viral-binding domains, each inclusive. In some embodiments, the VBP contains between 2 and 10 viral-binding domains, inclusive. In some embodiments, the VBP contains between 2 and 9 viral-binding domains, inclusive. In some embodiments, the VBP contains between 2 and 8 viral-binding domains, inclusive. In some embodiments, the VBP contains between 2 and 7 viral-binding domains, inclusive. In some embodiments, the VBP contains between 2 and 6 viral-binding domains, inclusive. In some embodiments, the VBP contains between 2 and 5 viral-binding domains, inclusive. In some embodiments, the VBP contains between 2 and 4 viral-binding domains, inclusive. In some embodiments, the VBP contains between 2 and 3 viral-binding domains, inclusive. In some embodiments, the VBP contains exactly, e.g., no more or less than, two viral-binding domains. In some embodiments, the VBP contains exactly three viral-binding domains. In some embodiments, the VBP contains exactly four viral-binding domains. In some embodiments, the VBP contains exactly five viral-binding domains. In some embodiments, the VBP contains exactly six viral-binding domains. In some embodiments, the VBP contains exactly seven viral-binding domains. In some embodiments, the VBP contains exactly eight viral-binding domains. In some embodiments, the VBP contains exactly nine viral-binding domains. In some embodiments, the VBP contains exactly 10 viral-binding domains.

In some embodiments, the VBP is set forth by the formula (viral-binding domain)ₙ-(binding partner), wherein n is at least one and is the number of viral-binding domains contained in the VBP. In some embodiments, n is at least two. In some embodiments, n is between 2 and 20, 2 and 18, 2 and 16, 2 and 14, 2 and 12, 2 and 10, 2 and 8, 2 and 6, 2 and 4, 4 and 20, 4 and 18, 4 and 16, 4 and 14, 4 and 12, 4 and 10, 4 and 8, 4 and 6, 6 and 20, 6 and 18, 6 and 16, 6 and 14, 6 and 12, 6 and 10, 6 and 8, 8 and 20, 8 and 18, 8 and 16, 8 and 14, 8 and 12, 8 and 10, 10 and 20, 10 and 18, 10 and 16, 10 and 14, 10 and 12, 12 and 20, 12 and 18, 12 and 16, 12 and 14, 14 and 20, 14 and 18, 14 and 16, 16 and 20, 16 and 18, or 18 and 20, each inclusive. In some embodiments, n is between 2 and 10, 2 and 9, 2 and 8, 2 and 7, 2 and 6, 2 and 5, 2 and 4, 2 and 3, 3 and 10, 3 and 9, 3 and 8, 3 and 7, 3 and 6, 3 and 5, 3 and 4, 4 and 10, 4 and 9, 4 and 8, 4 and 7, 4 and 6, 4 and 5, 5 and 10, 5 and 9, 5 and 8, 5 and 7, 5 and 6, 6 and 10, 6 and 9, 6 and 8, 6 and 7, 7 and 10, 7 and 9, 7 and 8, 8 and 10, 8 and 9, or 9 and 10, each inclusive. In some embodiments, n is between 2 and 10, inclusive. In some embodiments, n is between 2 and 9, inclusive. In some embodiments, n is between 2 and 8, inclusive. In some embodiments, n is between 2 and 7, inclusive. In some embodiments, n is between 2 and 6, inclusive. In some embodiments, n is between 2 and 5, inclusive. In some embodiments, n is between 2 and 4, inclusive. In some embodiments, n is between 2 and 3, inclusive. In some embodiments, n is 2. In some embodiments, n is 3. In some embodiments, n is 4. In some embodiments, n is 5. In some embodiments, n is 6. In some embodiments, n is 7. In some embodiments, n is 8. In some embodiments, n is 9. In some embodiments, n is 10.

In some embodiments, the viral-binding domains are connected to one another. In some embodiments, the viral-binding domains are connected to the N- and C-termini of one another. In some embodiments, the viral-binding domains are connected to one another in a sequential arrangement. In some embodiments, the viral-binding domains are directly connected to one another. In some embodiments, the viral-binding domains are indirectly connected to one another. In some embodiments, the viral-binding domains are connected to one another via a linker.

In some embodiments, the viral-binding domains are conjugated to one another. In some embodiments, the viral-binding domains are chemically conjugated to one another. In some embodiments, the viral-binding domains are connected to one another via a chemical linker. Methods and chemical linkers for conjugating the viral-binding domains to one another can be identified and selected by one of ordinary skill in the art. For instance, in some embodiments, the viral-binding domains are connected to one another via an N-(α-Maleimidoacetoxy)-succinimide ester, N-5-Azido-2-nitrobenzyloxy-succinimide, 1,4-Bis-Maleimidobutane, 1,4-Bis-Maleimmidyl-2,3-dihydroxy-butane, Bis-Maleimidohexane, Bis-Maleimidoethane, N-(β-Maleimidopropionic acid)hydrazide·TFA, N-(β-Maleimidopropyloxy)succinimide ester, 1,8-Bis-Maleimidodiethylene-glycol, 1,11-Bis-Maleimidotriethyleneglycol, Bis (sulfosuccinimidyl)suberate, Bis (sulfosuccinimidyl)glutarate-d0, Bis (sulfosuccinimidyl)2,2,4,4-glutarate-d4, Bis (sulfosuccinimidyl)suberate-d0, Bis (sulfosuccinimidyl)2,2,7,7-suberate-d4, Bis (NHS)PEG5, Bis (NHS)PEG9, Bis (2-[succinimidoxycarbonyloxy]ethyl)sulfone, N,N-Dicyclohexylcarbodiimide, 1-5-Difluoro-2,4-dinitrobenzene, Dimethyl adipimidate·2HCI, Dimethyl pimelimidate·2HCI, Dimethyl suberimidate·2HCl, Disuccinimidyl glutarate, Dithiobis(succimidylpropionate) (Lomant's Reagent), Disuccinimidyl suberate, Disuccinimidyl tartarate, Dimethyl 3,3′-dithiobispropionimidate·2HCI, Dithiobis-maleimidoethane, 3,3′-Dithiobis (sulfosuccinimidylpropionate), 1-Ethyl-3-(3-dimethylaminopropyl) carbodiimide hydrochloride, Ethylene glycol bis (succinimidylsuccinate), N-ε-Maleimidocaproic acid, N-(ε-Maleimidocaproic acid)hydrazide, N-(ε-Maleimidocaproyloxy)succinimide ester, N-(γ-Maleimidobutyryloxy)succinimide ester, N-(κ-Maleimidoundecanoic acid)hydrazide, NHS-LC-Diazirine, Succinimidyl 4-(N-maleimidomethyl) cyclohexane-1-carboxy-(6-amidocaproate), Succinimidyl 6-(3′-[2-pyridyldithio]propionamido)hexanoate, L-Photo-Leucine, L-Photo-Methionine, m-Maleimidobenzoyl-N-hydroxysuccinimide ester, 4-(4-N-Maleimidophenyl)-butyric acid hydrazide·HCI, 2-[N2-(4-Azido-2,3,5,6-tetrafluorobenzoyl)-N6-(6-biotinamidocaproyl)-L-lysinyl]ethylmethanethiosulfate, 2-{N2-[N6-(4-Azido-2,3,5,6-tetrafluorobenzoyl)-N6-(6-biotinamidocaproyl)-L-lysinyl]}ethylmethanethiosulfate, N-Hydroxysuccinimide, N-hydroxysuccinimide ester ethane azide, N-hydroxysuccinimide ester tetraoxapentadecane azide, N-hydroxysuccinimide ester dodecaoxanonatriacontane azide, NHS-Phosphine, 3-(2-Pyridyldithio)propionylhydrazide, 2-pyridyldithiol-tetraoxatetradecane-N-hydroxysuccinimide, 2-pyridyldithiol-tetraoxaoctatriacontane-N-hydroxysuccinimide, N-(p-Maleimidophenyl)isocyanate, Succinimdyl 3-(bromoacetamido)propionate, NHS-Diazirine, NHS-SS-Diazirine, N-succinimidyl iodoacetate, N-Succinimidyl(4-iodoacetyl)aminobenzoate, Succinimidyl 4-(N-maleimido-methyl)cyclohexane-1-carboxylate, NHS-PEG2-Maliemide, NHS-PEG4-Maliemide, NHS-PEG6-Maleimide, NHS-PEG8-Maliemide, NHS-PEG12-Maliemide, NHS-PEG24-Maleimide, Succinimidyl 4-(p-maleimido-phenyl)butyrate, Succinimidyl-6-(β-maleimidopropionamido)hexanoate, 4-Succinimidyloxycarbonyl-methyl-α-(2-pyridyldithio)toluene, Succinimidyl-(4-psoralen-8-yloxy)butyrate, N-Succinimidyl 3-(2-pyridyldithio)propionate, Ethylene glycol bis (sulfo-succinimidyl succinate), N-(ε-Maleimidocaproyloxy)sulfosuccinimide ester, N-(γ-Maleimidobutryloxy)sulfosuccinimide ester, N-(κ-Maleimidoundecanoyloxy)sulfosuccinimide ester, Sulfo-NHS-LC-Diazirine, Sulfosuccinimidyl 6-(3′-[2-pyridyldithio]propionamido)hexanoate, m-Maleimidobenzoyl-N-hydroxysulfosuccinimide ester, N-Hydroxysuccinimide, Sulfo-NHS-Phosphine, Sulfosuccinimidyl 6-(4′-azido-2′-nitrophenylamino)hexanoate, Sulfo-NHS-(2-6-[Biotinamido]-2-(p-azidobezamido), Sulfo-NHS-Diazirine, Sulfo-NHS-SS-Diazirine, Sulfosuccinimidyl(4-iodo-acetyl)aminobenzoate, Sulfosuccinimidyl 4-(N-maleimidomethyl)cyclohexane-1-carboxylate, Sulfosuccinimidyl 4-(p-maleimidophenyl)butyrate, Tris-(2-Maleimidoethyl)amine, or Tris-(succimimidyl aminotricetate) crosslinker.

In some embodiments, the viral-binding domains are connected to one another as part of a fusion protein sequence. In some embodiments, the fusion protein sequence contains a sequential arrangement of the viral-binding domains. In some embodiments, the viral-binding domains are directly connected to the N- and C-termini of one another. In some embodiments, the viral-binding domains are connected to one another by a peptide linker. In some embodiments, the peptide linker is a flexible linker. In some embodiments, the peptide linker is a GS-linker. In some embodiments, the peptide linker contains the amino acid sequence set forth in any of SEQ ID NO: 134 and 145-149. In some embodiments, the peptide linker is set forth in any of SEQ ID NO: 134 and 145-149. In some embodiments, the peptide linker contains the amino acid sequence set forth in SEQ ID NO: 134. In some embodiments, the sequence of the peptide linker is set forth in SEQ ID NO: 134.

In some embodiments, the binding partner is connected to the viral-binding domain. In some embodiments, the binding partner is indirectly connected to the viral-binding domain. In some embodiments, the binding partner is directly connected to the viral-binding domain. In some embodiments, the binding partner is connected to the N-terminus of the viral-binding domain. In some embodiments, the binding partner is connected to the C-terminus of the viral-binding domain. In some embodiments, the binding partner is positioned between the N- and C-terminus of the viral-binding domain.

In some embodiments, the binding partner is connected to one of the viral-binding domains. In some embodiments, the binding partner is connected to exactly one viral-binding domain. In some embodiments, the binding partner is connected to multiple of the viral-binding domains.

In some embodiments, the binding partner is indirectly connected to one of the viral-binding domains. In some embodiments, the binding partner is directly connected to one of the viral-binding domains. In some embodiments, the binding partner is connected to the N-terminus of one of the viral-binding domains. In some embodiments, the binding partner is connected to the C-terminus of one of the viral-binding domains. In some embodiments, the binding partner is positioned between the N- and C-terminus of one of the viral-binding domains. In some embodiments, the binding partner is positioned between two of the viral-binding domains.

In some embodiments, the binding partner is at the N-terminus of the VBP. In some embodiments, the binding partner is at the C-terminus of the VBP.

In some embodiments, the binding partner is conjugated to the viral-binding domain. In some embodiments, the binding partner is conjugated to one of the viral-binding domains. In some embodiments, the binding partner is chemically conjugated. In some embodiments, the binding partner is conjugated via a chemical linker. Methods and linkers for conjugating the binding partner to the viral-binding domain or one of the viral-binding domains can be identified and selected by one of ordinary skill in the art, for instance based on the binding partner being used. In some embodiments, the binding partner is connected via any of the crosslinkers described herein, for instance any of those described for connecting the viral-binding domains to one another. As a further example, in some embodiments, the binding partner contains biotin, and the binding partner is conjugated to one of the viral-binding domains using biotinylation reagents, such as Sulfo-NHS—SS-Biotin, Sulfo-NHS-Biotin, Biotin Hydrazide, Sulfo-NHS-LC-Biotin, or NHS-Biotin. Other conjugation methods for binding partners other than biotin, such as for binding partners that contain a biotin analog or derivative, e.g., any of the biotin analogs or derivatives described herein, can be identified and selected by one of ordinary skill in the art as well.

In some embodiments, the binding partner is connected to the viral-binding domain as part of a fusion protein sequence. In some embodiments, the VBP is a fusion protein containing the viral-binding domain and the binding partner. In some embodiments, the fusion protein contains a sequential arrangement of the viral-binding domain and the binding partner. In some embodiments, the binding partner is connected to the viral-binding domain via a peptide linker. In some embodiments, the peptide linker is a flexible linker. In some embodiments, the peptide linker is a GS-linker. In some embodiments, the peptide linker contains the amino acid sequence set forth in any of SEQ ID NO: 134 and 145-149. In some embodiments, the sequence of the peptide linker is set forth in any of SEQ ID NO: 134 and 145-149. In some embodiments, the binding partner is directly connected to the N-terminus of the viral-binding domain. In some embodiments, the binding partner is directly connected to the C-terminus of the viral-binding domain. In some embodiments, the binding partner is at the N-terminus of the VBP. In some embodiments, the binding partner is at the C-terminus of the VBP.

In some embodiments, the binding partner is connected to one of the viral-binding domains as part of a fusion protein sequence. In some embodiments, the VBP is a fusion protein containing the binding partner and the viral-binding domains. In some embodiments, the fusion protein contains a sequential arrangement of the viral-binding domains and the binding partner. In some embodiments, the binding partner is connected to one of the viral-binding domains via a peptide linker. In some embodiments, the peptide linker is a flexible linker. In some embodiments, the peptide linker is a GS-linker. In some embodiments, the peptide linker contains the amino acid sequence set forth in any of SEQ ID NO: 134 and 145-149. In some embodiments, the sequence of the peptide linker is set forth in any of SEQ ID NO: 134 and 145-149. In some embodiments, the binding partner is directly connected to the N-terminus of one of the viral-binding domains. In some embodiments, the binding partner is directly connected to the C-terminus of one of the viral-binding domains. In some embodiments, the binding partner is at the N-terminus of the VBP. In some embodiments, the binding partner is at the C-terminus of the VBP.

Any suitable method for producing the provided VBP can be used. Examples of such methods are described in Section I-C.

### A. Viral-Binding Domains

Exemplary viral-binding domains for the provided VBP are described in this section. In some embodiments, the viral-binding domain is any as described herein. In some embodiments, each viral-binding domain of the VBP is individually selected from among the described viral-binding domains. In some embodiments, each viral-binding domain of the VBP is the same and is any one of the viral-binding domains described herein.

Exemplary heparin-binding domains for the provided VBP are also described in this section. In some embodiments, the heparin-binding domain is any as described herein. In some embodiments, each heparin-binding domain of the VBP is individually selected from among the described heparin-binding domains. In some embodiments, each heparin-binding domain of the VBP is the same and is any one of the heparin-binding domains described herein. In some embodiments, the heparin-binding domain is any of the viral-binding domains described herein that binds to heparin.

In some embodiments, the viral-binding domain binds to a molecule present on the surface of a viral particle. In some embodiments, the molecule is a viral capsid protein. In some embodiments, the molecule is a viral matrix protein. In some embodiments, the molecule is a viral envelop protein. In some embodiments, the molecule is a viral envelope glycoprotein. In some embodiments, the molecule is a VSV glycoprotein (VSV-G), a Sindbis glycoprotein, a MMLV glycoprotein, an HSV glycoprotein, an MMTV glycoprotein, a Measles virus glycoprotein, an HTLV glycoprotein, an SIV glycoprotein, a GALV glycoprotein, an HIV glycoprotein, or an RSV glycoprotein.

In some embodiments, the molecule is one that is native to the strain or species of the viral particle. In some embodiments, the molecule is not native to the strain or species of the viral particle. For instance, in some embodiments, the viral particle, such as a viral vector, is pseudotyped to contain an envelope glycoprotein derived from a different virus. In some embodiments, the molecule contains a synthetic moiety. In some embodiments, the synthetic moiety contains an affinity tag. In some embodiments, the viral particle is engineered to express the synthetic moiety on its surface. In some embodiments, the viral particle is engineered to express the synthetic moiety, such as a synthetic peptide, as part of a fusion protein containing the synthetic moiety and a molecule present on the surface of the viral particle, such as a viral glycoprotein. In some embodiments, the synthetic moiety contains any of the binding partners described herein, for instance any as described in Section I-B. In some embodiments, the synthetic moiety contains any of the streptavidin or avidin binding partners described herein. In some embodiments, the synthetic moiety contains any of the streptavidin-binding peptides described herein.

In some embodiments, the viral-binding domain contains all or a portion of a cell surface molecule or an extracellular matrix (ECM) component involved in viral entry. Such cell surface molecules and ECM components can be identified and selected by one of ordinary skill in the art. In some embodiments, the cell surface molecule or ECM component is a human cell surface molecule or ECM component. In some embodiments, the viral-binding domain contains all or a portion of an adhesion molecule, an integrin, a lectin, a growth factor receptor, a glycan (e.g., heparan sulfate), a proteoglycan (e.g., heparan sulfate proteoglycan), fibronectin, vitronectin, heparin sulfate, collagen, thrombospondin, or laminin. In some embodiments, the viral-binding domain contains all or a portion of any of the foregoing that is from a human. In some embodiments, the viral-binding domain domains all or a portion of an ECM component. In some embodiments, the ECM component is a human ECM component. In some embodiments, the viral-binding domain contains all or a portion of fibronectin. In some embodiments, the viral-binding domain contains a portion of fibronectin. In some embodiments, the fibronectin is a human fibronectin. In some embodiments, the viral-binding domain is a heparin-binding domain from any of the foregoing.

In some embodiments, the viral-binding domain is a heparin-binding domain. In some embodiments, each viral-binding domain of the VBP is a heparin-binding domain.

Amino acid sequences that exhibit heparin binding can be identified and selected by one of ordinary skill in the art; include those described in Barkalow and Schwarzbauer (1991), Journal of Biological Chemistry 266(12): 7812-7818; Zhong et al., (2018), Biochemistry 57(42): 6045-6049; Hansen et al. (1995), Biochim Biophys Acta 1252(1): 135-45; Leahy et al. (1992), Science 258(5084): 987-91; Sharma et al. (1999), The EMBO Journal 18: 1468-1479; Busby et al. (1995), Protein Chemistry and Structure 270(31): P18558-18562; Munoz and Linhardt (2004), Arterioscler Thromb Vasc Biol 24(9): 1549 1557; Drake et al. (1993), Journal of Biological Chemistry 268(21): 15859-15867; Sipes et al. (1993), The Journal of Cell Biology 121(2): 469-477; Moritz et al. (1993), J Clin Invest 93: 1451-1457; and U.S. Pat. No. 7,790,849; and include sequences present in fibronectin (e.g., heparin-binding domain I or domain II [FN12-14]), thrombospondin, the 70 kDA heat-shock family of proteins (HSPs), tenascin, L-selectin (LAM-1), cytochrome c, vitronectin, apolipoproteins E and B-100, and platelet factor 4. In some embodiments, the heparin-binding domain is from a human heparin-binding protein.

In some embodiments, the heparin-binding domain contains an amino acid sequence selected from X-B-B-X-B-X (SEQ ID NO: 137), X-B-B-B-X-X-B-X (SEQ ID NO: 138), X-B-X-B-B-X (SEQ ID NO: 139), and X-B-X-X-B-B-B-X (SEQ ID NO: 140), wherein each X is independently selected from any hydrophobic amino acid, and each B is independently selected from any basic amino acid. In some embodiments, the heparin-binding domain contains the amino acid sequence X₁-B₁-B₂-X₂-B₃-X₃ (SEQ ID NO: 137), wherein each X is independently selected from any hydrophobic amino acid, and each B is independently selected from any basic amino acid. In some embodiments, each B is independently selected from arginine and lysine. In some embodiments, X₁ is P. In some embodiments, B₁ is R. In some embodiments, B₂ is R. In some embodiments, X₂ is A. In some embodiments, X₂ is G. In some embodiments, B₃ is R. In some embodiments, X₃ is V. In some embodiments, the heparin-binding domain contains the amino acid sequence set forth in SEQ ID NO: 152. In some embodiments, the heparin-binding domain contains the amino acid sequence set forth in SEQ ID NO: 141.

In some embodiments, the heparin-binding domain contains the amino acid sequence set forth in SEQ ID NO: 150.

In some embodiments, the heparin-binding domain contains any of the amino acid sequences set forth in SEQ ID NO: 151 and 153-161. In some embodiments, the heparin-binding domain contains the amino acid sequence set forth in SEQ ID NO: 142. In some embodiments, the heparin-binding domain contains the amino acid sequence set forth in SEQ ID NO: 151.

In some embodiments, the heparin-binding domain contains (i) the amino acid sequence set forth in SEQ ID NO: 141 and (ii) the amino acid sequence set forth in SEQ ID NO: 142. In some embodiments, the heparin-binding domain contains (i) the amino acid sequence set forth in SEQ ID NO: 141 and (ii) the amino acid sequence set forth in SEQ ID NO: 151. In some embodiments, the heparin-binding domain contains (i) the amino acid sequence set forth in SEQ ID NO: 141 and (ii) the amino acid sequence set forth in SEQ ID NO: 150.

In some embodiments, the heparin-binding domain contains (i) the amino acid sequence set forth in SEQ ID NO: 141; (ii) the amino acid sequence set forth in SEQ ID NO: 150; and (iii) the amino acid sequence set forth in SEQ ID NO: 142. In some embodiments, the heparin-binding domain contains (i) the amino acid sequence set forth in SEQ ID NO: 141; (ii) the amino acid sequence set forth in SEQ ID NO: 150; and (iii) the amino acid sequence set forth in SEQ ID NO: 151.

In some embodiments, the heparin-binding domain contains the amino acid sequence of a heparin-binding domain of fibronectin or an amino acid sequence that binds to heparin and exhibits at least 85% sequence identity to a heparin-binding domain of fibronectin. In some embodiments, the heparin-binding domain contains an amino acid sequence that binds to heparin and exhibits at least 85%, 86%, 87%, 88%, 89%, 90%, 91%, 92%, 93%, 94%, 95%, 96%, 97%, 98%, or 99% sequence identity to a heparin-binding domain of fibronectin. In some embodiments, the viral-binding domain contains the amino acid sequence of a heparin-binding domain of fibronectin. In some embodiments, the heparin-binding domain of fibronectin is the heparin-binding domain I of fibronectin. In some embodiments, the heparin-binding domain of fibronectin is the heparin-binding domain II of fibronectin (FN12-14).

In some embodiments, the heparin-binding domain contains the amino acid sequence set forth in SEQ ID NO: 133 or an amino acid sequence that binds to heparin and exhibits at least 85% sequence identity to the sequence of amino acids set forth in SEQ ID NO: 133.

In some embodiments, the heparin-binding domain contains an amino acid sequence that binds to heparin and exhibits at least 85%, 86%, 87%, 88%, 89%, 90%, 91%, 92%, 93%, 94%, 95%, 96%, 97%, 98%, or 99% sequence identity to the sequence of amino acids set forth in SEQ ID NO: 133. In some embodiments, the amino acid sequence contains the amino acid sequence set forth in SEQ ID NO: 141. In some embodiments, the amino acid sequence contains the amino acid sequence set forth in SEQ ID NO: 150. In some embodiments, the amino acid sequence contains the amino acid sequence set forth in SEQ ID NO: 142. In some embodiments, the amino acid sequence contains the amino acid sequence set forth in SEQ ID NO: 151. In some embodiments, the amino acid sequence contains the amino acid sequence set forth in SEQ ID NO: 141 and the amino acid sequence set forth in SEQ ID NO: 150. In some embodiments, the amino acid sequence contains the amino acid sequence set forth in SEQ ID NO: 141 and the amino acid sequence set forth in SEQ ID NO: 142. In some embodiments, the amino acid sequence contains the amino acid sequence set forth in SEQ ID NO: 141 and the amino acid sequence set forth in SEQ ID NO: 151. In some embodiments, the amino acid sequence contains (i) the amino acid sequence set forth in SEQ ID NO: 141; (ii) the amino acid sequence set forth in SEQ ID NO: 150; and (iii) the amino acid sequence set forth in SEQ ID NO: 142. In some embodiments, the amino acid sequence contains (i) the amino acid sequence set forth in SEQ ID NO: 141; (ii) the amino acid sequence set forth in SEQ ID NO: 150; and (iii) the amino acid sequence set forth in SEQ ID NO: 151.

In some embodiments, the heparin-binding domain contains the amino acid sequence set forth in SEQ ID NO: 133. In some embodiments, the sequence of the heparin-binding domain is set forth in SEQ ID NO: 133.

In some embodiments, each of the heparin-binding domains of the VBP contains the amino acid sequence set forth in SEQ ID NO: 133. In some embodiments, the sequence of each heparin-binding domain of the VBP is set forth in SEQ ID NO: 133. In some embodiments, the VBP contains exactly two of the heparin-binding domains.

### B. Binding Partners

In some embodiments, the VBP contains a binding partner. In some embodiments, the binding partner is a molecule that binds to a target binding site of a protein. In some embodiments, the VBP can be detected via binding of the binding partner to the target binding site. In some embodiments, the VBP can be purified via binding of the binding partner to the target binding site. In some embodiments, the VBP can be incorporated into a reagent via binding of the binding partner to the target binding site. In some embodiments, the reagent contains a molecule having a target binding site for the binding partner of the VBP. In some embodiments, the binding partner binds to the target binding site contained by the reagent. In some aspects, this binding allows for the functionalization of the reagent with the VBP, including the multimerization of the VBP onto the reagent, in the case of a reagent containing multiple binding sites each capable of binding to the binding partner of the VBP. In some embodiments, the reagent is a protein reagent. In some embodiments, the protein reagent is any as described herein, for instance any as described in Section II-A.

In some embodiments, the choice of binding partner can be informed by the detection or purification methods that are readily available to one of ordinary skill in the art. In some embodiments, such methods can involve detecting or purifying proteins via binding to particular molecules recognized by the detection or purification reagents being employed. For instance, in some embodiments, a method can involve the purification of proteins using affinity chromatography, which in some aspects can leverage specific binding interactions between ligand and binding partner pairs. In some embodiments, the methods can involve binding to an affinity tag recognized by the detection or purification reagents. In some embodiments, the affinity tag is a chemical structure recognized by the detection or purification reagents. For instance, the methods may be suitable for binding to biotinylated proteins. In some embodiments, the affinity tag is a peptide sequence recognized by the detection or purification reagents. In some embodiments, the affinity tag can be selected and used as the binding partner of the VBP, for instance so that following preparation of the VBP, such methods can be used for the detection or purification of the VBP thereafter. Suitable structures for the binding partner of the provided VBP can be selected and identified by one of ordinary skill in the art, based on these or any other considerations.

In some embodiments, the choice of binding partner can be informed by the reagent for which incorporation of the VBP is desired. In some embodiments, the reagent is one that is suitable for use in one or more steps of a method involving the transduction of target cells with viral particles. In some embodiments, the reagent is one that is suitable for use during the transduction of target cells with viral particles. In some embodiments, the reagent contains particular molecules or moieties by which additional agents, such as a VBP, can be incorporated into the reagent. In some embodiments, additional agents can be added via binding of the additional agents to the particular molecules or moieties. In some embodiments, suitable binding partners for the VBP can be selected and identified based on these particular molecules or moieties of the reagent for which incorporation of the VBP is desired. Suitable structures for the binding partner of the provided VBP can be selected and identified by one of ordinary skill in the art, based on these or any other considerations.

In some embodiments, the VBP contains between 1 and 5, 1 and 4, 1 and 3, or 1 and 2 binding partners, each inclusive. In some embodiments, the VBP contains exactly one binding partner. In some embodiments, the VBP contains exactly two binding partners. In some embodiments, the VBP contains exactly three binding partners. In some embodiments, the VBP contains exactly four binding partners. In some embodiments, the VBP contains exactly five binding partners.

Exemplary binding partners are described in this section. In some embodiments, the binding partner is any as described herein. In some embodiments, each binding partner of a VBP containing multiple binding partners is individually selected from among the described binding partners. In some embodiments, each binding partner of a VBP containing multiple binding partners is the same and is any one of the binding partners described herein.

Im some embodiments, the binding partner is hydrocarbon-based (including polymeric) and contains nitrogen-, phosphorus-, sulphur-, carben-, halogen- or pseudohalogen groups. In some embodiments, the binding partner is an alcohol, an organic acid, an inorganic acid, an amine, a phosphine, a thiol, a disulfide, an alkane, an amino acid, a peptide, an oligopeptide, a polypeptide, a protein, a nucleic acid, a lipid, a saccharide, an oligosaccharide, or a polysaccharide. As further examples, in some embodiments, the binding partner is a cation, an anion, a polycation, a polyanion, a polycation, an electrolyte, a polyelectrolyte, a carbon nanotube, or carbon nanofoam. As yet further examples, in some embodiments, the binding partner is a crown ether, an immunoglobulin or a fragment thereof, or a proteinaceous binding molecule with antibody-like functions.

In some embodiments, the binding partner includes a moiety known to one of ordinary skill in the art as an affinity tag. In some embodiments, the reagent includes a corresponding binding partner, for example an antibody or an antibody fragment known to bind to the affinity tag. As a few illustrative examples of known affinity tags, in some embodiments, the affinity tag includes dinitrophenol or digoxigenin, oligohistidine, polyhistidine, an immunoglobulin domain, glutathione-S-transferase (GST), chitin binding protein (CBP) or thioredoxin, calmodulin binding peptide (CBP), FLAG′-peptide, the HA-tag (SEQ ID NO: 20), the VSV-G-tag (SEQ ID NO: 21), the HSV-tag (SEQ ID NO: 22), the T7 epitope (SEQ ID NO: 23), maltose binding protein (MBP), the HSV epitope (SEQ ID NO: 24) of herpes simplex virus glycoprotein D, the “myc” epitope of the transcription factor c-myc (SEQ ID NO: 25), or the V5-tag (SEQ ID NO: 26). In some embodiments, the complex formed between the binding site of the reagent and the affinity tag, for instance between the corresponding binding partner of the reagent, e.g., an antibody or antibody fragment, and the affinity tag, can be disrupted competitively by contacting the complex with a free binding partner, e.g., an unbound affinity tag.

In some embodiments, the affinity tag includes an oligonucleotide tag. In some some embodiments, the oligonucleotide tag hybridizes to an oligonucleotide linked to or included in the reagent with a complementary sequence.

In some embodiments, the binding partner is a lectin, protein A, protein G, a metal, a metal ion, nitrilo triacetic acid derivatives (NT A), RGD-motifs, a dextrane, polyethyleneimine (PEI), a redox polymer, a glycoprotein, an aptamer, a dye, amylose, maltose, cellulose, chitin, glutathione, calmodulin, gelatine, polymyxin, heparin, NAD, NADP, lysine, arginine, benzamidine, poly U, or oligo-dT. Lectins such as Concavalin A are known to bind to polysaccharides and glycosylated proteins. An illustrative example of a dye is a triazine dye, such as Cibacron blue F3G-A (CB) or Red HE-3B, which specifically binds NADH-dependent enzymes. Green A is known to bind to Co A proteins, human serum albumin, and dehydrogenases. The dyes 7-aminoactinomycin D and 4′,6-diamidino-2-phenylindole are known to bind to DNA. Cations of metals such as Ni, Cd, Zn, Co, or Cu can also be used to bind affinity tags, such as an oligohistidine-containing sequence, including the hexahistidine or the MAT tag (SEQ ID NO: 35), and N-methacryloyl-(L)-cysteine methyl ester.

In some embodiments, the binding between the binding partner and the binding site of the reagent occurs in the presence of a divalent, a trivalent, or a tetravalent cation. In some embodiments, the reagent includes a divalent, a trivalent, or a tetravalent cation, for instance held, e.g., complexed, by means of a suitable chelator. In some embodiments, the binding partner includes a moiety that complexes with a divalent, a trivalent, or a tetravalent cation. Examples of metal chelators include ethylenediamine, ethylene-diaminetetraacetic acid (EDTA), ethylene glycol tetraacetic acid (EGTA), diethylenetri-aminepentaacetic acid (DTPA), N,N-bis(carboxymethyl)glycine (also called nitrilotriacetic acid, NTA), 1,2-bis(o-aminophenoxy)ethane-N,N,N′,N′-tetraacetic acid (BAPTA), 2,3-dimer-capto-l-propanol (dimercaprol), porphine, and heme. As an example, EDTA can form a complex with most monovalent, divalent, trivalent, and tetravalent metal ions, such as silver (Ag⁺), calcium (Ca²⁺), manganese (Mn²⁺), copper (Cu²⁺), iron (Fe²⁺), cobalt (Co⁺), and zirconium (Zr⁴⁺) while BAPTA is specific for Ca²⁺. As an illustrative example, one of ordinary skill in the art can use methods involving the formation of a complex between an oligohistidine tag and copper (Cu²⁺), nickel (Ni²⁺), cobalt (Co²⁺), or zinc (Zn²⁺) ions, which are presented by means of the chelator nitrilotriacetic acid (NTA).

In some embodiments, the binding partner includes a calmodulin-binding peptide, and the reagent includes multimeric calmodulin, for instance as described in U.S. Pat. No. 5,985,658. In some embodiments, the binding partner includes a FLAG peptide, and the reagent includes an antibody that binds to the FLAG peptide. For instance, in some embodiments, the reagent includes the monoclonal antibody 4E11 that binds to the FLAG peptide, for instance as described in U.S. Pat. No. 4,851,341. In some embodiments, the binding partner includes an oligohistidine tag, and the reagent includes an antibody or a transition metal ion that binds the oligohistidine tag. In some embodiments, calmodulin, antibodies such as 4E11, chelated metal ions, and free chelators may be multimerized by methods involving, for example, biotinylation and complexation with streptavidin, avidin, or oligomers thereof, or by the introduction of carboxyl residues into a polysaccharide, e.g., dextran, for instance as described in Noguchi et al. (1992), Bioconjugate Chemistry 3: 132-137, in a first step, and linking calmodulin, antibodies, chelated metal ions, or free chelators via primary amino groups to the carboxyl groups in the polysaccharide, e.g. dextran, using carbodiimide chemistry in a second step. In some embodiments, the binding between the binding partner and the binding site of the reagent can be disrupted by metal ion chelation. The metal chelation may be accomplished by, for example, addition of EGTA or EDTA.

In some embodiments, the binding partner binds to a biotin-binding molecule. In some embodiments, the binding partner binds to the biotin-binding site of the molecule.

In some embodiments, the binding partner is a streptavidin or avidin binding partner. In some embodiments, the binding partner is a streptavidin-binding partner. In some embodiments, the streptavidin-binding partner is also an avidin-binding partner.

In some embodiments, the binding partner binds to a molecule that is streptavidin, avidin, a streptavidin analog or mutein, or an avidin analog or mutein. In some embodiments, the molecule is any of the streptavidin, avidin, streptavidin analog or mutein, and avidin analog or mutein molecules described herein, for instance any as described in Section II-A. In some embodiments, the reagent contains the molecule. In some embodiments, the binding partner binds to a biotin-binding site of the molecule. In some embodiments, the binding partner binds to the natural biotin-binding site of the molecule (see, e.g., Qureshi et al. (2001), Journal of Biological Chemistry 276(49): 46422-46428; and Livnah et al. (1993), Proc Natl Acad Sci 90: 5076-5080; which describe the interactions of biotin with streptavidin and avidin, respectively). In some embodiments, the binding partner allows for the functionalization of reagents containing streptavidin, avidin, a streptavidin analog or mutein, or an avidin analog or mutein.

Binding partners that bind to streptavidin, avidin, a streptavidin analog or mutein, or an avidin analog or mutein, including that bind to the biotin-binding sites of these molecules, can be identified and selected by one of ordinary skill in the art. In some embodiments, the binding partner binds to a molecule that is streptavidin.

In some embodiments, the binding partner contains biotin. In some embodiments, the binding partner is biotin. In some embodiments, the biotin is D-biotin. In some embodiments, the binding partner contains a biotin analog or derivate. In some embodiments, the binding partner is a biotin analog or derivate. In some embodiments, the biotin analog or derivative is a structural analog of biotin. In some embodiments, the biotin analog or derivative binds to the biotin-binding site of streptavidin, avidin, a streptavidin analog or mutein, or an avidin analog or mutein. In some embodiments, the biotin analog or derivative binds to the biotin-binding site of streptavidin. In some embodiments, the biotin analog or derivative is desthiobiotin, iminobiotin, guanidinobiotin, diaminobiotin, lipoic acid, HABA (hydroxyazobenzene-benzoic acid), dimethyl-HABA, biotin sulfone, caproylamidobiotin, or biocytin (or any of the biotin analogs and derivatives described in, e.g., International Published PCT Appl. No. WO2008140573).

In some embodiments, the binding partner contains a streptavidin-binding peptide. In some embodiments, the binding partner is a streptavidin-binding peptide. In some embodiments, the streptavidin-binding peptide binds to the biotin-binding site of streptavidin, avidin, a streptavidin analog or mutein, or an avidin analog or mutein. In some embodiments, the streptavidin-binding peptide binds to the biotin-binding site of streptavidin. In some embodiments, the streptavidin-binding peptide contains an amino acid sequence with the formula set forth in SEQ ID NO: 9, such as contains the amino acid sequence set forth in SEQ ID NO: 10. In some embodiments, the streptavidin-binding peptide contains an amino acid sequence with the formula set forth in SEQ ID NO: 11, such as set forth in SEQ ID NO: 12. In some embodiments, the streptavidin-binding peptide contains the amino acid sequence set forth in SEQ ID NO: 7, also called Strep-Tag®. In some embodiments, the sequence of the streptavidin-binding peptide is set forth in SEQ ID NO: 7. In some embodiments, the streptavidin-binding peptide contains the amino acid sequence set forth in SEQ ID NO: 8, also called Strep-Tag® II. In some embodiments, the sequence of the streptavidin-binding peptide is set forth in SEQ ID NO: 8.

In some embodiments, the streptavidin-binding peptide may be further modified. In some embodiments, the streptavidin-binding peptide contains the amino acid sequence set forth in SEQ ID NO: 8 that is conjugated to a nickel charged trisNTA, also called His-STREPPER or His/Strep-Tag®II Adapter.

In some embodiments, the streptavidin-binding peptide contains a sequential arrangement of two streptavidin-binding modules. In some embodiments, the streptavidin-binding peptide contains a sequential arrangement of exactly two streptavidin-binding modules. In some embodiments, the streptavidin-binding modules are separated from one another by no more than 50 amino acids, for instance for no more than 45, 40, 35, 30, 25, 20, 15, 10, or 5 amino acids. In some embodiments, the streptavidin-binding modules are directly connected to one another. In some embodiments, one streptavidin-binding module has three to eight amino acids and contains at least the sequence His-Pro-Xaa (SEQ ID NO: 9), where Xaa is glutamine, asparagine, or methionine. In some embodiments, another streptavidin-binding module has the same or different sequence from the first streptavidin-binding module, such as set forth in SEQ ID NO: 11 (see, e.g., International Published PCT Appl. No. WO02/077018; and U.S. Pat. No. 7,981,632). In some embodiments, one of the streptavidin-binding modules contains the amino acid sequence set forth in SEQ ID NO: 7. In some embodiments, each of the streptavidin-binding modules contains the amino acid sequence set forth in SEQ ID NO: 7. In some embodiments, one of the streptavidin-binding modules contains the amino acid sequence set forth in SEQ ID NO: 8. In some embodiments, each of the streptavidin-binding modules contains the amino acid sequence set forth in SEQ ID NO: 8. In some embodiments, the streptavidin-binding peptide contains an amino acid sequence having the formula set forth in any of SEQ ID NO 13, 14, 17, and 164. In some embodiments, the streptavidin-binding peptide contains the amino acid sequence set forth in any of SEQ ID NO: 15-19. In some embodiments, the sequence of the streptavidin-binding peptide is set forth in any of SEQ ID NO: 15-19. In some embodiments, the streptavidin-binding peptide contains the amino acid sequence set forth in SEQ ID NO: 16, also called Twin-Strep-Tag®. In some embodiments, the sequence of the streptavidin-binding peptide is set forth in SEQ ID NO: 16.

In some embodiments, the VBP contains the amino acid sequence set forth in SEQ ID NO: 135. In some embodiments, the sequence of the VBP is set forth in SEQ ID NO: 135.

### C. Methods of Producing VBPs

For production of any of the recombinant polypeptides described herein, including any of the provided VBPs, such as any of the provided VBPs that are fusion proteins or contain fusion protein sequences, a nucleic acid encoding the recombinant polypeptide can be isolated or designed in silico and inserted into a replicable vector for further cloning (e.g., amplification of DNA) or for expression. Nucleic acids encoding the recombinant polypeptide can be readily isolated and sequenced or designed in silico using conventional procedures. In some embodiments, a nucleic acid encoding the recombinant polypeptide is designed in silico and inserted into a replicable vector for further cloning (e.g., amplification of DNA) or for expression. Any suitable vector can be used. The choice of vector can depend in part on the host cell to be used. Host cells can include prokaryotic and eukaryotic (e.g., mammalian) cells.

Expression vectors containing regulatory elements from eukaryotic viruses can be used in eukaryotic expression vectors, e.g., SV40 vectors, papilloma virus vectors, and vectors derived from Epstein-Barr virus. Other exemplary eukaryotic vectors include pMSG, pAV009/A+, pMTO10/A+, pMAMneo-5, baculovirus pDSVE, and any other vector allowing expression of proteins under the direction of the CMV promoter, SV40 early promoter, SV40 later promoter, metallothionein promoter, murine mammary tumor virus promoter, Rous sarcoma virus promoter, polyhedrin promoter, or other promoters shown effective for expression in eukaryotic cells.

Some expression systems have markers that provide gene amplification, such as thymidine kinase and dihydrofolate reductase. Alternatively, high-yield expression systems not involving gene amplification can also be used, such as using a baculovirus vector in insect cells, with a nucleic acid sequence encoding the recombinant polypeptide under the direction of the polyhedrin promoter or other strong baculovirus promoters.

Nucleic acid sequences encoding components of the recombinant polypeptide can be obtained using standard recombinant techniques. In some embodiments, nucleic acid sequences can be synthesized using nucleotide synthesizer or PCR techniques. Once obtained, the nucleic acid sequences can be inserted into a recombinant vector capable of replicating and expressing heterologous polynucleotides in prokaryotic hosts. Any suitable vector can be used. Selection of an appropriate vector can depend on the size of the nucleic acid sequences to be inserted into the vector and the particular host cell to be transformed with the vector. Each vector can contain an origin of replication, a selection marker gene, a promoter, a ribosome binding site (RBS), a signal sequence, the encoding nucleic acid sequence, and a transcription termination sequence.

Plasmid vectors containing replicon and control sequences which are derived from species compatible with the host cell can be used in connection with these hosts. The vector can carry a replication site, as well as marking sequences which are capable of providing phenotypic selection in transformed cells. For example, E. coli can be transformed using pBR322, a plasmid derived from an E. coli species. pBR322 contains genes encoding ampicillin (Amp) and tetracycline (Tet) resistance and thus can provide means for identifying transformed cells. pBR322, its derivatives, or other microbial plasmids or bacteriophage can also contain, or be modified to contain, promoters which can be used by the microbial organism for expression of proteins. Examples of pBR322 derivatives used for expression have been described in, e.g., U.S. Pat. No. 5,648,237.

In addition, phage vectors containing replicon and control sequences that are compatible with the host microorganism can be used as transforming vectors in connection with these hosts. For example, bacteriophage such as λGEM™-11 can be utilized in making a recombinant vector which can be used to transform susceptible host cells, such as E. coli LE392.

A large number of promoters recognized by a variety of potential host cells can be used. The selected promoter can be operably linked to cistron DNA encoding the recombinant polypeptide by removing the promoter from the source DNA via restriction enzyme digestion and inserting the isolated promoter sequence into the vectors described herein. Both the native promoter sequence and many heterologous promoters can be used to direct amplification or expression of the recombinant polypeptides. In some embodiments, heterologous promoters are utilized, as they can permit greater transcription and higher yields of expressed recombinant polypeptides as compared to the native target polypeptide promoter.

Promoters suitable for use with prokaryotic hosts include an ara B promoter, a PhoA promoter, β-galactamase and lactose promoter systems, a tryptophan (trp) promoter system, and hybrid promoters such as the tac or the trc promoter. However, other promoters that are functional in bacteria (such as other known bacterial or phage promoters) are suitable as well. Their nucleotide sequences have been published, thereby enabling one of ordinary skill in the art to operably ligate them to cistrons encoding the recombinant polypeptides (see, e.g., Siebenlist et al. (1980) Cell 20: 269) using linkers or adaptors to supply any required restriction sites.

Bacterial expression systems for expressing recombinant polypeptides are available in, e.g., E. coli, Bacillus sp., and Salmonella (see, e.g., Palva et al., Gene, 22:229 235 (1983); and Mosbach et al., Nature, 302:543-545 (1983)).

In some embodiments, each cistron within the recombinant vector contains a secretion signal sequence component that directs translocation of the expressed recombinant polypeptide across a membrane. The signal sequence can be a component of the vector, or it can be a part of the encoding nucleic acid sequence that is inserted into the vector. The signal sequence can be one that is recognized and processed (e.g., cleaved by a signal peptidase) by the host cell. For prokaryotic host cells that do not recognize and process the signal sequences native to the recombinant polypeptide, the signal sequence can be substituted by a prokaryotic signal sequence, for example PelB, OmpA, alkaline phosphatase, penicillinase, Ipp, heat-stable enterotoxin II (STII) leaders, LamB, PhoE, or MBP. In some embodiments, the production of the recombinant polypeptide occurs in the cytoplasm of the host cell, and a secretion signal sequence is not present within each cistron.

Suitable host cells for cloning or expression of recombinant polypeptide-encoding vectors include prokaryotic or eukaryotic cells described herein. In some embodiments, the host cell is eukaryotic, e.g., a Chinese Hamster Ovary (CHO) cell, Human Embryonic Kidney (HEK) cell, or lymphoid cell (e.g., YO, NSO, or Sp20 cell). In some embodiments, the recombinant polypeptide is produced in bacteria. For expression of recombinant polypeptides in bacteria, see, e.g., U.S. Pat. Nos. 5,648,237, 5,789,199, and 5,840,523 (see also Charlton, Methods in Molecular Biology, Vol. 248 (B. K. C. Lo, ed., Humana Press, Totowa, N.J., 2003), pp. 245-254, describing expression of recombinant polypeptides in E. coli). After expression, the recombinant polypeptide can be isolated from the bacterial cell paste in a soluble fraction and can be further purified. In addition to prokaryotes, eukaryotic microbes such as filamentous fungi or yeast are suitable cloning or expression hosts for recombinant polypeptide-encoding vectors. Suitable host cells for the expression of recombinant polypeptides can also be derived from multicellular organisms (invertebrates and vertebrates). Examples of invertebrate cells include plant and insect cells. Numerous baculoviral strains have been identified which can be used in conjunction with insect cells, for instance for transfection of Spodoptera frugiperda cells. Methods for constructing derivatives of any of the described bacteria having defined genotypes are known in the art and described in, for example, Bass et al., Proteins, 8:309-314 (1990). Selection of the host bacteria can take into consideration the replicability of the replicon in the cells of the bacteria. For example, E. coli, Serratia, or Salmonella species can be suitably used as the host when plasmids such as pBR322, pBR325, pACYC177, or pKN410 are used to supply the replicon.

In some embodiments, the host cell secretes minimal amounts of proteolytic enzymes, and additional protease inhibitors can be incorporated in the cell culture. To minimize proteolysis of expressed recombinant polypeptides (for instance those that are proteolytically sensitive), certain host strains deficient for proteolytic enzymes can be used. For example, host cell strains can be modified to effect genetic mutations in the genes encoding bacterial proteases, such as Protease III, OmpT, DegP, Tsp, Protease I, Protease Mi, Protease V, Protease VI, and combinations thereof. Some E. coli protease-deficient strains are available (see, e.g., U.S. Pat. Nos. 5,264,365 and 5,508,192; and Hara et al., Microbial Drug Resistance, 2:63-72 (1996)). E. coli strains deficient for proteolytic enzymes and transformed with plasmids overexpressing one or more chaperone proteins can be used as host cells.

Plant cell cultures can also be utilized as hosts (see, e.g. U.S. Pat. Nos. 5,959,177, 6,040,498, 6,420,548, 7,125,978, and 6,417,429). Vertebrate cells can also be used as hosts. For example, mammalian cell lines that are adapted to grow in suspension can be used. Other examples of mammalian host cell lines include monkey kidney CV1 line transformed by SV40 (COS-7); human embryonic kidney line (293 or 293 cells, for instance as described in Graham et al., Gen VlI′01. 36:59 (1977)); baby hamster kidney cells (BHK); mouse sertoli cells (TM4 cells as described in, e.g., Mather, Biol. Reprod. 23:243-251 (1980)); monkey kidney cells (CV1); African green monkey kidney cells (V ERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TR1 cells, as described in, e.g., Mather et al., Annals NI'. Acad. Sci. 383:44 68 (1982); MRC 5 cells; and FS4 cells. Other mammalian host cell lines include Chinese hamster ovary (CHO) cells, including DHFR′ CHO cells (see, e.g., Urlaub et al., Proc. Natl. Acad. Sci. USA 77:4216 (1980)); and myeloma cell lines, such as YO, NSO and Sp2/0. For a review of mammalian host cell lines, see, e.g., Yazaki and Wu, Methods in Molecular Biology, Vol. 248 (B. K. C. Lo, ed., Humana Press, Totowa, N.]), pp. 255-268 (2003).

Depending on the host cell used, transformation can be done using standard techniques appropriate to such cells. The calcium treatment employing calcium chloride can be used for bacterial cells that contain substantial cell-wall barriers. Another method for transformation employs polyethylene glycol/DMSO. Yet another technique used is electroporation.

The expressed recombinant polypeptides can be secreted into and recovered from the periplasm of the host cells or transported into the culture medium. Protein recovery from the periplasm can involve disrupting the microorganism, such as by means as osmotic shock, sonication, or lysis. Once cells are disrupted, cell debris or whole cells can be removed by centrifugation or filtration. The recombinant polypeptides can be further purified by, for example, affinity resin chromatography. Alternatively, recombinant polypeptides that are transported into the culture medium can be isolated therein. Cells can be removed from the culture, and the culture supernatant can be filtered and concentrated for further purification of the produced recombinant polypeptide. The expressed recombinant polypeptide can be further isolated and identified using methods such as polyacrylamide gel electrophoresis (PAGE) and Western blot assay.

Any suitable protein purification method can be employed, such as fractionation on immunoaffinity or ion-exchange columns, ethanol precipitation, reverse phase HPLC, chromatography on silica or on a cation-exchange resin, such as DEAE, chromatofocusing, SDS-PAGE, ammonium sulfate precipitation, and gel filtration using, for example, Sephadex G-75. In some embodiments, the binding partner of the VBP is a streptavidin or avidin binding partner, and the VBP can be isolated and purified by chromatography using, e.g., streptavidin, avidin, streptavidin analog or mutein, or avidin analog or mutein columns binding to the streptavidin or avidin binding partner of the VBP. Such columns can be identified and selected by one of ordinary skill in the art and include the StrepTactin® XT gravity flow column available from IBA Lifesciences GmbH. Following isolation, the VBP can be eluted from the column under conditions. In some embodiments, the VBP can be eluted by addition of a substance having greater binding affinity for the column, e.g., for the streptavidin, avidin, streptavidin analog or mutein, or avidin analog or mutein, than the streptavidin or avidin binding partner of the VBP. These substances can be identified and selected by one of ordinary skill in the art and include biotin, e.g., D-biotin, and any of the biotin analogs or derivatives described herein.

### II. Viral-Binding Protein (VBP)-Functionalized Reagents

In some embodiments, the provided viral-binding protein (VBP)-functionalized reagent (also referred to herein as a VBP reagent) contains any of the provided VBPs, such as any described in Section I.

In some embodiments, the provided heparin-binding reagent contains any of the provided VBPs, such as any described in Section I. In some embodiments, the heparin-binding reagent binds to a viral particle, such as via the heparin-binding domain or domains contained in the heparin-binding reagent. In some embodiments, the heparin-binding reagent may be also referred to herein as a VBP reagent.

In some embodiments, the VBP reagent contains a reagent having a binding site for the binding partner of the VBP. In some embodiments, the binding partner is bound to the reagent. In some embodiments, the binding partner is reversibly bound to the reagent.

In some embodiments, the reagent is a protein reagent. In some embodiments, the protein reagent is any as described herein, for instance any as described in Section II-A.

In some embodiments, the protein reagent contains a plurality of binding sites for the binding partner of the VBP. Thus, in some embodiments, the protein reagent allows for the multimerization of the VBP thereon, in some aspects for causing an avidity effect for the binding to viral particles. The plurality of binding sites can be the same or different across the protein reagent. In some embodiments, the protein reagent is multimerized using multiple copies of one provided VBP. In some embodiments, the protein reagent is multimerized with a plurality of different provided VBPs, for instance VBPs differing in their binding partners, viral-binding domain or domains, or both.

In some embodiments, the protein reagent contains a molecule to which the binding partner of the VBP is bound. In some embodiments, the protein reagent contains a plurality of molecules to which the binding partner can bind. In some embodiments, the binding partner is bound to one of the plurality of molecules. In some embodiments, the binding partner is bound to two of the plurality of molecules.

In some embodiments, the molecule is any described herein that can bind to the binding partner of the VBP, for instance any of the molecules described in Section II-A. In some embodiments, the molecule is any of the streptavidin, avidin, streptavidin analog or mutein, and avidin analog or mutein molecules described herein. In some embodiments, the molecule is streptavidin. In some embodiments, the molecule is any of the streptavidin mutein molecules described herein.

In some embodiments, each molecule of the protein reagent is individually selected from among any of the streptavidin, avidin, streptavidin analog or mutein, and avidin analog or mutein molecules described herein. In some embodiments, the protein reagent contains a mixture of any of the streptavidin, avidin, streptavidin analog or mutein, and avidin analog or mutein molecules described herein.

In some embodiments, each molecule of the protein reagent is individually selected from among any of the streptavidin and streptavidin analog or mutein molecules described herein. In some embodiments, the protein reagent contains a mixture of any of the streptavidin and streptavidin analog or mutein molecules described herein.

In some embodiments, each molecule of the protein reagent is the same and is any one of the streptavidin, avidin, streptavidin analog or mutein, and avidin analog or mutein molecules described herein. In some embodiments, each molecule of the protein reagent is the same and is streptavidin. In some embodiments, each molecule of the protein reagent is the same and is any one of the streptavidin mutein molecules described herein.

In some embodiments, the VBP reagent contains a weight ratio of protein reagent to VBP that is between about 10:1 and 2:1, 9:1 and 2:1, 8:1 and 2:1, 7:1 and 2:1, 6:1 and 2:1, 5:1 and 2:1, 4:1 and 2:1, 3:1 and 2:1, 10:1 and 3:1, 9:1 and 3:1, 8:1 and 3:1, 7:1 and 3:1, 6:1 and 3:1, 5:1 and 3:1, 4:1 and 3:1, 10:1 and 4:1, 9:1 and 4:1, 8:1 and 4:1, 7:1 and 4:1, 6:1 and 4:1, 5:1 and 4:1, 10:1 and 5:1, 9:1 and 5:1, 8:1 and 5:1, 7:1 and 5:1, 6:1 and 5:1, 10:1 and 6:1, 9:1 and 6:1, 8:1 and 6:1, 7:1 and 6:1, 10:1 and 7:1, 9:1 and 7:1, 8:1 and 7:1, 10:1 and 8:1, 9:1 and 8:1, or 10:1 and 9:1, each inclusive. In some embodiments, the VBP reagent contains a weight ratio of protein reagent to VBP that is between about 10:1 and 2:1, inclusive. In some embodiments, the VBP reagent contains a weight ratio of protein reagent to VBP that is between about 8:1 and 2:1, inclusive. In some embodiments, the VBP reagent contains a weight ratio of protein reagent to VBP that is between about 8:1 and 4:1, inclusive. In some embodiments, the VBP reagent contains a weight ratio of protein reagent to VBP that is about 6:1.

In some embodiments, the VBP reagent is prepared by mixing the protein reagent and the VBP at a weight ratio to each other of between about 10:1 and 2:1, 9:1 and 2:1, 8:1 and 2:1, 7:1 and 2:1, 6:1 and 2:1, 5:1 and 2:1, 4:1 and 2:1, 3:1 and 2:1, 10:1 and 3:1, 9:1 and 3:1, 8:1 and 3:1, 7:1 and 3:1, 6:1 and 3:1, 5:1 and 3:1, 4:1 and 3:1, 10:1 and 4:1, 9:1 and 4:1, 8:1 and 4:1, 7:1 and 4:1, 6:1 and 4:1, 5:1 and 4:1, 10:1 and 5:1, 9:1 and 5:1, 8:1 and 5:1, 7:1 and 5:1, 6:1 and 5:1, 10:1 and 6:1, 9:1 and 6:1, 8:1 and 6:1, 7:1 and 6:1, 10:1 and 7:1, 9:1 and 7:1, 8:1 and 7:1, 10:1 and 8:1, 9:1 and 8:1, or 10:1 and 9:1, each inclusive. In some embodiments, the VBP reagent is prepared by mixing the protein reagent and the VBP at a weight ratio to each other of between about 10:1 and 2:1, inclusive. In some embodiments, the VBP reagent is prepared by mixing the protein reagent and the VBP at a weight ratio to each other of between about 8:1 and 2:1, inclusive. In some embodiments, the VBP reagent is prepared by mixing the protein reagent and the VBP at a weight ratio to each other of between about 8:1 and 4:1, inclusive. In some embodiments, the VBP reagent is prepared by mixing the protein reagent and the VBP at a weight ratio to each other of about 6:1. In some embodiments, the mixing is performed at room temperature.

In some embodiments, the VBP reagent contains one or more binding agents, e.g., one or more binding agents in addition to the VBP. In some embodiments, the protein reagent contains one or more binding sites for the one or more binding agents. In some embodiments, the one or more binding agents are bound to the protein reagent. In some embodiments, the one or more binding agents are any as described herein, for instance any as described in Section II-B.

In some embodiments, the one or more binding agents include other viral-binding agents. In some embodiments, the other viral-binding agents are any of the provided VBPs.

In some embodiments, the one or more binding agents include one or more binding agents that bind to cell surface molecules. Thus, in some embodiments, the presence of binding agents in addition to the VBP on the VBP reagent may increase the co-localization of viral particles and target cells for engineering.

In some embodiments, the one or more binding agents each contain a binding partner. In some embodiments, the binding partner is the same across the one or more binding agents of the VBP reagent. In some embodiments, the binding partner is different across the one or more binding agents of the VBP.

In some embodiments, the binding partner of each of the one or more binding agents is individually selected from any of the binding partners described herein, for instance any of the binding partners described in Section I-B. In some embodiments, the binding partner of each of the one or more binding agents is individually selected from any of the binding partners that can be used for the VBP. In some embodiments, the binding partner of any of the one or more binding agents is the same as the binding partner of the VBP. In some embodiments, the binding partner of each of the one or more binding agents is the same as the binding partner of the VBP.

In some embodiments, the binding partner of each of the one or more binding agents is individually selected from any of the streptavidin or avidin binding partners described herein. In some embodiments, the binding partner of each of the one or more binding agents is individually selected from any of the streptavidin-binding partners described herein. In some embodiments, the binding partner of the one or more binding agents contains biotin. In some embodiments, the binding partner of the one or more binding agents is biotin. In some embodiments, the biotin is D-biotin. In some embodiments, the binding partner of the one or more binding agents contains any of the biotin analogs or derivates described herein. In some embodiments, the binding partner of the one or more binding agents is any of the biotin analogs or derivates described herein. In some embodiments, the binding partner of the one or more binding agents contains any of the streptavidin-binding peptides described herein. In some embodiments, the binding partner of the one or more binding agents is any of the streptavidin-binding peptides described herein. In some embodiments, the streptavidin-binding peptide contains the sequence of amino acids set forth in any of SEQ ID NO: 7, 8, and 15-19. In some embodiments, the sequence of the streptavidin-binding peptide is set forth in any of SEQ ID NO: 7, 8, and 15-19. In some embodiments, the streptavidin-binding peptide contains the sequence of amino acids set forth in SEQ ID NO: 16. In some embodiments, the sequence of the streptavidin-binding peptide is set forth in SEQ ID NO: 16.

In some embodiments, the protein reagent contains a plurality of binding sites for the binding partner or partners of the one or more binding agents. Thus, in some embodiments, the protein reagent allows for the multimerization of the one or more binding agents thereon, in some aspects for causing an avidity effect for the binding of targets of the one or more binding agents. The plurality of binding sites can be the same or different across the protein reagent. In some embodiments, the protein reagent is multimerized using multiple copies of one binding agent. In some embodiments, the protein reagent is multimerized with a plurality of different binding agents, for instance binding agents differing in their binding partners, targets, or both.

In some embodiments, the protein reagent contains a molecule to which the one or more binding agents are bound. In some embodiments, the protein reagent contains a plurality of molecules to which the one or more binding agents can bind. In some embodiments, the binding partner of each of the one or more binding agents is bound to one of the plurality of molecules. In some embodiments, the binding partner of each of the one or more binding agents is bound to two of the plurality of molecules.

In some embodiments, the molecule to which the binding partner of each of the one or more binding agents binds is any described herein that can bind to the binding partner of the one or more binding agents, for instance any of the molecules described in Section II-A. In some embodiments, the molecule is any of the streptavidin, avidin, streptavidin analog or mutein, and avidin analog or mutein molecules described herein. In some embodiments, the molecule is streptavidin. In some embodiments, the molecule is any of the streptavidin mutein molecules described herein.

In some embodiments, the VBP reagent contains a weight ratio of protein reagent to each of the one or more binding agents that is between about 10:1 and 2:1, 9:1 and 2:1, 8:1 and 2:1, 7:1 and 2:1, 6:1 and 2:1, 5:1 and 2:1, 4:1 and 2:1, 3:1 and 2:1, 10:1 and 3:1, 9:1 and 3:1, 8:1 and 3:1, 7:1 and 3:1, 6:1 and 3:1, 5:1 and 3:1, 4:1 and 3:1, 10:1 and 4:1, 9:1 and 4:1, 8:1 and 4:1, 7:1 and 4:1, 6:1 and 4:1, 5:1 and 4:1, 10:1 and 5:1, 9:1 and 5:1, 8:1 and 5:1, 7:1 and 5:1, 6:1 and 5:1, 10:1 and 6:1, 9:1 and 6:1, 8:1 and 6:1, 7:1 and 6:1, 10:1 and 7:1, 9:1 and 7:1, 8:1 and 7:1, 10:1 and 8:1, 9:1 and 8:1, or 10:1 and 9:1, each inclusive. In some embodiments, the VBP reagent contains a weight ratio of protein reagent to each of the one or more binding agents that is between about 10:1 and 2:1, inclusive. In some embodiments, the VBP reagent contains a weight ratio of protein reagent to each of the one or more binding agents that is between about 8:1 and 2:1, inclusive. In some embodiments, the VBP reagent contains a weight ratio of protein reagent to each of the one or more binding agents that is between about 8:1 and 4:1, inclusive. In some embodiments, the weight ratio to protein reagent is different across the one or more binding agents. In some embodiments, the weight ratio to protein reagent is the same across the one or more binding agents. In some embodiments, the VBP reagent contains a weight ratio of protein reagent to each of the one or more binding agents that is about 6:1.

In some embodiments, the VBP reagent is prepared by mixing the protein reagent and each of the one or more binding agents at a weight ratio to each other of between about 10:1 and 2:1, 9:1 and 2:1, 8:1 and 2:1, 7:1 and 2:1, 6:1 and 2:1, 5:1 and 2:1, 4:1 and 2:1, 3:1 and 2:1, 10:1 and 3:1, 9:1 and 3:1, 8:1 and 3:1, 7:1 and 3:1, 6:1 and 3:1, 5:1 and 3:1, 4:1 and 3:1, 10:1 and 4:1, 9:1 and 4:1, 8:1 and 4:1, 7:1 and 4:1, 6:1 and 4:1, 5:1 and 4:1, 10:1 and 5:1, 9:1 and 5:1, 8:1 and 5:1, 7:1 and 5:1, 6:1 and 5:1, 10:1 and 6:1, 9:1 and 6:1, 8:1 and 6:1, 7:1 and 6:1, 10:1 and 7:1, 9:1 and 7:1, 8:1 and 7:1, 10:1 and 8:1, 9:1 and 8:1, or 10:1 and 9:1, each inclusive. In some embodiments, the VBP reagent is prepared by mixing the protein reagent and each of the one or more binding agents at a weight ratio to each other of between about 10:1 and 2:1, inclusive. In some embodiments, the VBP reagent is prepared by mixing the protein reagent and each of the one or more binding agents at a weight ratio to each other of between about 8:1 and 2:1, inclusive. In some embodiments, the VBP reagent is prepared by mixing the protein reagent and each of the one or more binding agents at a weight ratio to each other of between about 8:1 and 4:1, inclusive. In some embodiments, the weight ratio to protein reagent is different across the one or more binding agents. In some embodiments, the weight ratio to protein reagent is the same across the one or more binding agents. In some embodiments, the VBP reagent is prepared by mixing the protein reagent and each of the one or more binding agents at a weight ratio to each other of about 6:1. In some embodiments, the mixing is performed at room temperature.

In some embodiments, the weight ratio of the VBP to each of the one or more binding agents in the VBP reagent is 4:1 to 1:1. In some embodiments, the weight ratio of the VBP to each of the one or more binding agents in the VBP reagent is 3:1 to 1:1. In some embodiments, the weight ratio of the VBP to each of the one or more binding agents in the VBP reagent is 2:1 to 1:1. In some embodiments, the weight ratio to VBP is different across the one or more binding agents. In some embodiments, the weight ratio to VBP is the same across the one or more binding agents. In some embodiments, the weight ratio of the VBP to each of the one or more binding agents in the VBP reagent is about 1:1, e.g., equal parts by weight of the VBP and each of the one or more binding agents.

In some embodiments, the VBP reagent is prepared by mixing the VBP and each of the one or more binding agents at a weight ratio to each other of 4:1 to 1:1. In some embodiments, the VBP reagent is prepared by mixing the VBP and each of the one or more binding agents at a weight ratio to each other of 3:1 to 1:1. In some embodiments, the VBP reagent is prepared by mixing the VBP and each of the one or more binding agents at a weight ratio to each other of 2:1 to 1:1. In some embodiments, the weight ratio to VBP is different across the one or more binding agents. In some embodiments, the weight ratio to VBP is the same across the one or more binding agents. In some embodiments, the VBP reagent is prepared by mixing the VBP and each of the one or more binding agents at a weight ratio to each other of about 1:1, e.g., equal parts by weight of the VBP and each of the one or more binding agents.

In some embodiments, the one or more binding agents include one binding agent. In some embodiments, the one or more binding agents include exactly one binding agent. In some embodiments, the one or more binding agents include two binding agents. In some embodiments, the one or more binding agents include exactly two binding agents.

In some embodiments, the VBP reagent contains the two bindings agents at a weight ratio to each other of 4:1 to 1:1. In some embodiments, the VBP reagent contains the two bindings agents at a weight ratio to each other of 3:1 to 1:1. In some embodiments, the VBP reagent contains the two bindings agents at a weight ratio to each other of 2:1 to 1:1. In some embodiments, the VBP reagent contains thetwo bindings agents at a weight ratio to each other of about 1:1, e.g., equal parts by weight of the two binding agents.

In some embodiments, the VBP reagent is prepared by mixing the two bindings agents at a weight ratio to each other of 4:1 to 1:1. In some embodiments, the VBP reagent is prepared by mixing the two bindings agents at a weight ratio to each other of 3:1 to 1:1. In some embodiments, the VBP reagent is prepared by mixing the two bindings agents at a weight ratio to each other of 2:1 to 1:1. In some embodiments, the VBP reagent is prepared by mixing the two bindings agents at a weight ratio to each other of about 1:1, e.g., equal parts by weight of the two binding agents.

In some embodiments, the VBP reagent contains the VBP, a first binding agent, and a second binding agent. In some embodiments, the VBP, first binding agent, and second binding agent are present at about a 1:1:1 weight ratio in the VBP reagent. In some embodiments, the VBP reagent contains about a 6:1:1:1 weight ratio of the protein reagent, VBP, first binding agent, and second binding agent.

In some embodiments, the binding partner (e.g., that of the VBP or the one or more binding agents) is bound to a biotin-binding site of the molecule or molecules of the protein reagent to which it is bound. In some embodiments, the biotin-binding site is the natural biotin-binding site of the molecule or molecules (see, e.g., Qureshi et al. (2001), Journal of Biological Chemistry 276(49): 46422-46428; and Livnah et al. (1993), Proc Natl Acad Sci 90: 5076-5080; which describe the interactions of biotin with streptavidin and avidin, respectively).

In some embodiments, the complex formed between the binding partner (e.g., that of the VBP or the one or more binding agents) and the protein reagent can be of any desired strength and affinity. In some embodiments, the complex is reversible. In some embodiments, the binding partner is reversibly bound to the molecule or molecules of the protein reagent to which it is bound. Exemplary binding partners and molecules for reversible binding are described herein as well as in, e.g., U.S. Pat. Nos. 5,168,049; 5,506,121; 6,103,493; 7,776,562; 7,981,632; 8,298,782; 8,735,540; and 9,023,604; and International Published PCT Appl. Nos. WO2013/124474 and WO2014/076277.

In some embodiments, the binding affinity of the binding partner (e.g., that of the VBP or the one or more binding agents) to the molecule or molecules of the protein reagent to which it is bound is reduced compared to the binding affinity of biotin to streptavidin, which has a dissociation constant (K_{d}) on the order of ˜10⁻¹⁴ mol/L. Binding affinity can be determined by any suitable method. In some embodiments, the binding affinity of the binding partner to the molecule or molecules of the protein reagent to which it is bound is greater than 1×10⁻¹³ M, 1×10⁻¹² M, or 1×10⁻¹¹ M and less than 1×10⁻⁴M, 5×10⁻⁴ M, 1×10⁻⁵ M, 5×10⁻⁵ M, 1×10⁻⁶ M, 5×10⁻⁶ M, or 1×10⁻⁷ M.

In some embodiments, the binding partner contains biotin, e.g., D-biotin, and the molecule or molecules of the protein reagent to which it is bound are analogs or muteins of streptavidin or avidin that have reduced affinity for biotin, compared to streptavidin or avidin.

In some embodiments, the binding partner contains a biotin analog or derivative, e.g., any as described herein, having reduced affinity for streptavidin or avidin compared to biotin, and the molecule or molecules of the protein reagent to which it is bound are streptavidin or avidin. In some embodiments, the binding partner contains a biotin analog or derivative, e.g., any as described herein, and the molecule or molecules of the protein reagent to which it is bound are analogs or muteins of streptavidin or avidin that have reduced affinity for the biotin analog or derivative, compared to biotin.

In some embodiments, the binding partner contains a streptavidin-binding peptide, e.g., any as described herein, having reduced affinity for streptavidin or avidin compared to biotin, and the molecule or molecules of the protein reagent to which it is bound are streptavidin or avidin. In some embodiments, the binding partner contains a streptavidin-binding peptide, e.g., any as described herein, and the molecule or molecules of the protein reagent to which it is bound are analogs or muteins of streptavidin or avidin that have reduced affinity for the streptavidin-binding peptide, compared to biotin. In some embodiments, the binding partner contains a streptavidin-binding peptide, e.g., any as described herein, and the molecule or molecules of the protein reagent to which it is bound are muteins of streptavidin that have reduced affinity for the streptavidin-binding peptide, compared to biotin.

In some embodiments, the binding of the binding partner (e.g., that of the VBP or the one or more binding agents) to the molecule or molecules of the protein reagent is disrupted by the presence of biotin, e.g., D-biotin. In some embodiments, the binding of the binding partner to the molecule or molecules of the protein reagent is disrupted by the presence of a biotin analog or derivative, e.g., any as described herein. For example, binding of the streptavidin-binding peptides known as Strep-Tag®, Strep-Tag® II, and Twin-Strep-Tag® to streptavidin muteins known as StrepTactin® m1 or m2 or StrepTactin XT® are disrupted by the presence of biotin, e.g., D-biotin, iminobiotin, lipoic acid, desthiobiotin, diaminobiotin, HABA, and dimethyl-HABA (see, e.g., U.S. Pat. Nos. 5,506,121 and 6,103,493, and International Published PCT Appl. No. WO2014/076277). Other combinations of molecules and binding partners whose binding can be disrupted by the presence of biotin or a biotin analog or derivative can be identified and selected by one of ordinary skill in the art.

In some embodiments, the VBP reagent is capable of being immobilized on a solid support. In some embodiments, the VBP reagent is not immobilized on a solid support. In some embodiments, the VBP reagent is in soluble form. In some embodiments, the VBP reagent is soluble in culture media.

In some embodiments, the solid support is a chromatography matrix. In some embodiments, the chromatography matrix is any as described herein, for instance any as described in Section VI-A. In some embodiments, the solid support is a stationary phase. In some embodiments, the stationary phase is any as described herein, for instance any as described in Section VI.

### A. Protein Reagents

In some cases, the protein reagent contains at least two chelating groups K that may be capable of binding to a transition metal ion. In some embodiments, the protein reagent may be capable of binding to an oligohistidine affinity tag, a glutathione-S-transferase, calmodulin or an analog thereof, calmodulin binding peptide (CBP), a FLAG-peptide, an HA-tag, maltose binding protein (MBP), an HSV epitope, a myc epitope, or a biotinylated carrier protein.

In some embodiments, the protein reagent contains a molecule to which the binding partner of the VBP can bind. In some embodiments, the protein reagent contains a molecule to which the one or more binding agents can bind. In some embodients, the molecule can bind to the binding partner of each of the one or more binding agents.

In some embodiments, the molecule is avidin, e.g., wild-type avidin. In some embodiments, the molecule is an avidin analog. In some embodiments, an avidin analog is a variant of wild-type avidin having one or more modified functional groups, but that contains a biotin-binding site. In some embodiments, the molecule is an avidin mutein. In some embodiments, an avidin mutein is a polypeptide distinguished from the sequence of wild-type avidin by one or more amino acid substitutions, deletions, or additions, but that contains a biotin-binding site. In some embodiments, the avidin analog is neutravidin, a deglycosylated avidin with modified arginines that can exhibit a more neutral pi and is available as an alternative to wild-type avidin. In some embodiments, the avidin analog is any of those commercially available as ExtrAvidin®, available through Sigma Aldrich, NeutrAvidin, available from Thermo Scientific or Invitrogen, and CaptAvidin™, available from Molecular Probes. In some embodiments, the avidin analog or mutein is any as described in International Published PCT Appl. No. WO2008/140573.

In some embodiments, the molecule is streptavidin, e.g., wild-type streptavidin. In some embodiments, streptavidin has the amino acid sequence disclosed by Argarana et al., Nucleic Acids Res. 14 (1986) 1871-1882 and set forth in SEQ ID NO: 1, or has an amino acid sequence that is a sequence present in homologs thereof from other Streptomyces species. In some embodiments, streptavidin has the amino acid sequence set forth in SEQ ID NO: 1.

In some embodiments, the molecule is a streptavidin analog. In some embodiments, a streptavidin analog is a variant of wild-type streptavidin having one or more modified functional groups, but that contains a biotin-binding site. In some embodiments, the molecule is a streptavidin mutein. In some embodiments, a streptavidin mutein is a polypeptide distinguished from the sequence of wild-type streptavidin by one or more amino acid substitutions, deletions, or additions, but that contains a biotin-binding site.

In some embodiments, the streptavidin mutein binds to a streptavidin-binding peptide, for instance any as described herein. In some embodiments, the streptavidin mutein binds to any of the streptavidin-binding peptides set forth in SEQ ID NO: 7, 8, and 15-19. In some embodiments, the binding affinity of the streptavidin-binding peptide to the streptavidin mutein is greater than 1×10⁻¹³ M, 1×10⁻¹² M, or 1×10⁻¹¹ M and less than 1×10⁻⁴M, 5×10⁻⁴ M, 1×10⁻⁵ M, 5×10⁻⁵M, 1×10⁻⁶ M, 5×10⁻⁶ M, or 1×10⁻⁷ M. In some embodiments, the streptavidin mutein binds to biotin, e.g., D-biotin. In some embodiments, the streptavidin mutein binds to a biotin analog or derivative, e.g., any as described herein. In some embodiments, the streptavidin mutein binds to biotin or to the biotin analog or derivative with greater affinity than to the streptavidin-binding peptide. In some embodiments, binding of the streptavidin-binding peptide to the streptavidin mutein, e.g., to the biotin-binding site of the streptavidin mutein, can be disrupted by the presence of biotin or the biotin analog or derivative. In some embodiments, the binding of the streptavidin mutein to the streptavidin-binding peptide of any of SEQ ID NO: 7, 8, and 15-19 is disrupted by the presence of biotin, e.g., D-biotin.

In some embodiments, the streptavidin mutein contains only a part of wild-type streptavidin. In some embodiments, the streptavidin mutein is a minimal streptavidin (in some instances referred to as a recombinant core streptavidin) wherein wild-type streptavidin is shortened at the N- and/or C-terminus. In some embodiments, the streptavidin mutein is any of the recombinant core streptavidins described in Sano et al. (1995), Journal of Biological Chemistry 270(47): 28204-28209. In some embodiments, the streptavidin mutein begins N-terminally in the region of amino acid positions 10 to 16 of SEQ ID NO: 1 and terminates C-terminally in the region of amino acid positions 133 to 142 of SEQ ID NO: 1. Reference to the position of residues in streptavidin or streptavidin muteins is with reference to the numbering of residues in SEQ ID NO: 1. In some embodiments, the sequence of the streptavidin mutein is set forth in any of SEQ ID NO: 2, 103, and 162. In some embodiments, the streptavidin mutein is an amino acid sequence from position Ala13 to Ser139 of SEQ ID NO: 1. In some embodiments, the sequence of the streptavidin mutein is set forth in SEQ ID NO: 162. In some embodiments, the streptavidin mutein contains an N-terminal methionine and an amino acid sequence from position Glu14 to Ser139 of SEQ ID NO: 1. In some embodiments, the sequence of the streptavidin mutein is set forth in SEQ ID NO: 2.

In some embodiments, the streptavidin mutein contains one or more amino acid substitutions compared to wild-type streptavidin, such as compared to the wild-type streptavidin sequence set forth in SEQ ID NO: 1. In some embodiments, the streptavidin mutein contains one or more amino acid substitutions compared to a streptavidin mutein that is a minimal streptavidin. In some embodiments, the streptavidin contains one or more amino acid substitutions compared to a streptavidin mutein, e.g., a minimal streptavidin, that begins N-terminally in the region of amino acid positions 10 to 16 of SEQ ID NO: 1 and terminates C-terminally in the region of amino acid positions 133 to 142 of SEQ ID NO: 1. In some embodiments, the streptavidin contains one or more amino acid substitutions compared to the streptavidin mutein set forth in any of SEQ ID NO: 2, 103, and 162.

In some embodiments, the streptavidin mutein binds to biotin and contains at least 1, 2, 3, 4, 5, 6, 7, 8, 9, 10, 11, 12, 13, 14, 15, 16, 17, 18, 19, or 20 amino acid differences compared to the sequence of amino acids set forth in SEQ ID NO: 1, 2, 103, or 162. In some embodiments, the streptavidin mutein binds to biotin and contains an amino acid sequence that exhibits at least 85%, 86%, 87%, 88%, 89%, 90%, 91%, 92%, 93%, 94%, 95%, 96%, 97%, 98%, 99% or more sequence identity to the sequence of amino acids set forth in SEQ ID NO: 1, 2, 103, or 162. In some embodiments, the amino acid substitutions are conservative or non-conservative mutations. In some embodiments, the streptavidin mutein is any as described in U.S. Pat. Nos. 5,168,049; 5,506,121; 6,022,951; 6,156,493; 6,165,750; 6,103,493; 6,368,813; and Internation Published PCT Appl. Nos. WO2014/076277, WO2008/140573, WO 86/02077, WO 98/40396, and WO 96/24606. In some embodiments, the streptavidin mutein is any as described in DE 19641876 A1; Howarth et al. (2006) Nat. Methods, 3:267-73; Zhang et al. (2015) Biochem. Biophys. Res. Commun., 463:1059-63; Fairhead et al. (2013) J Mol. Biol., 426:199-214; Wu et al. (2005) J. Biol. Chem., 280:23225-31; Lim et al. (2010) Biochemistry, 50:8682-91); and Qureshi et al. (2001), Journal of Biological Chemistry 276(49): 46422-46428.

In some embodiments, the streptavidin mutein is any as described in U.S. Pat. No. 6,103,493. In some embodiments, the streptavidin mutein contains at least one mutation within the region corresponding to amino acid positions 44 to 53 of wild-type streptavidin, such as set forth in SEQ ID NO: 1. In some embodiments, “corresponding to” references amino acid positions with reference to the amino acid sequence of wild-type streptavidin, such as set forth in SEQ ID NO: 1. One of ordinary skill in the art would be able to identify these residues with methods involving, e.g., the alignment of sequences. In some embodiments, the streptavidin mutein contains a mutation at one or more of residues 44, 45, 46, and 47 of wild-type streptavidin. In some embodiments, the streptavidin mutein contains a replacement of Glu at position 44 with a hydrophobic aliphatic amino acid, e.g., Val, Ala, Ile, or Leu. In some embodiments, the streptavidin mutein contains any amino acid at position 45. In some embodiments, the streptavidin mutein contains an aliphatic amino acid, such as a hydrophobic aliphatic amino acid, at position 46. In some embodiments, the streptavidin mutein contains a replacement of Val at position 47 with a basic amino acid, e.g., Arg or Lys, such as Arg. In some embodiments, Ala is at position 46, Arg is at position 47, and Val or Ile is at position 44. In some embodiments, the streptavidin mutein contains residues Val⁴⁴-Thr⁴⁵-Ala⁴⁶-Arg⁴⁷ (SEQ ID NO: 144) at sequence positions corresponding to positions 44 to 47 of the sequence of amino acids set forth in SEQ ID NO: 1, such as set forth in exemplary streptavidin muteins containing the sequence of amino acids set forth in SEQ ID NO: 3, 4, or 104. In some embodiments, the streptavidin mutein contains residues Ile⁴⁴-Gly⁴⁵-Ala⁴⁶-Arg⁴⁷ (SEQ ID NO: 143) at sequence positions corresponding to positions 44 to 47 of the sequence of amino acids set forth in SEQ ID NO: 1, such as set forth in exemplary streptavidin muteins containing the sequence of amino acids set forth in SEQ ID NO: 5, 6, or 104. In some embodiments, the streptavidin mutein contains the amino acid sequence set forth in any of SEQ ID NO: 3-6, 104, and 105. In some embodiments, the streptavidin mutein is commercially available under the trademark Strep-Tactin® m1. In some embodiments, the streptavidin mutein is commercially available under the trademark Strep-Tactin® m2. In some embodiments, the streptavidin mutein contains the amino acid sequence set forth in SEQ ID NO: 4. In some embodiments, the streptavidin mutein contains the amino acid sequence set forth in SEQ ID NO: 6.

In some embodiment, the streptavidin mutein is any as described in International Published PCT Appl. No. WO 2014/076277. In some embodiments, the streptavidin mutein contains at least two cysteine residues in the region corresponding to amino acid positions 44 to 53 of the amino acid sequence set forth in SEQ ID NO: 1. In some embodiments, the cysteine residues are present at positions 45 and 52 to create a disulfide bridge connecting these amino acids. In some embodiments, amino acid 44 is glycine or alanine; amino acid 46 is alanine or glycine; and amino acid 47 is arginine. In some embodiments, the streptavidin mutein contains at least one mutation in the region corresponding to amino acids residues 115 to 121 of the amino acid sequence set forth in SEQ ID NO: 1. In some embodiments, the streptavidin mutein contains at least one mutation at amino acid position 117, 120, or 121 and/or a deletion of amino acids 118 and 119 and substitution of at least amino acid position 121.

In some embodiments, the streptavidin mutein contains a mutation at a position corresponding to position 117, which mutation can be to a large hydrophobic residue like Trp, Tyr, or Phe; to a charged residue like Glu, Asp, or Arg; to a hydrophilic residue like Asn or Gin; to the hydrophobic residues Leu, Met, or Ala; or the polar residues Thr, Ser, or His. In some embodiments, the mutation at position 117 is combined with a mutation at a position corresponding to position 120, which mutation can be to a small residue like Ser, Ala, or Gly, and a mutation at a position corresponding to position 121, which mutation can be to a hydrophobic residue, such as a bulky hydrophobic residue like Trp, Tyr, or Phe. In some embodiments, the mutation at position 117 is combined with a mutation at a position corresponding to position 120 of wild-type streptavidin set forth in SEQ ID NO: 1, which mutation can be a hydrophobic residue such as Leu, Ile, Met, or Val; or Tyr or Phe, and a mutation at a position corresponding to position 121 of SEQ ID NO: 1, which mutation can be to a small residue like Gly, Ala, or Ser, or with Gln, or with a hydrophobic residue like Leu, Val, Ile, Trp, Tyr, Phe, or Met. In some embodiments, the streptavidin mutein contains the residues Glu117, Gly120, and Tyr121 with reference to positions of the sequence of amino acids set forth in SEQ ID NO: 1. In some embodiments, the streptavidin mutein also contains residues Val⁴⁴-Thr⁴⁵-Ala⁴⁶-Arg⁴⁷ or residues Ile⁴⁴-Gly⁴⁵-Ala⁴⁶-Arg⁴⁷ at sequence positions corresponding to positions 44 to 47 of the sequence of amino acids set forth in SEQ ID NO: 1. In some embodiments, the streptavidin mutein contains the residues Val44, Thr45, Ala46, Arg47, Glu117, Gly120, and Tyr121. In some embodiments, the mutein streptavidin contains the sequence of amino acids set forth in any of SEQ ID NO: 27, 28, and 163, or a sequence of amino acids that exhibits at least 85%, 86%, 87%, 88%, 89%, 90%, 91%, 92%, 93%, 94%, 95%, 96%, 97%, 98%, 99% or more sequence identity to the sequence of amino acids set forth in any of SEQ ID NO: 27, 28, and 163, contains the residues Val44, Thr45, Ala46, Arg47, Glu117, Gly120 and Tyr121, and binds to biotin. In some embodiments, the streptavidin mutein contains the sequence of amino acids set forth in SEQ ID NO: 27. In some embodiments, the streptavidin mutein contains the sequence of amino acids set forth in SEQ ID NO: 28. In some embodiments, the streptavidin mutein contains the sequence of amino acids set forth in SEQ ID NO: 163.

In some embodiments, the streptavidin mutein contains the sequence of amino acids set forth in any of SEQ ID NO: 3-6, 27, 28, 104, 105, and 163, and the binding partner (e.g., that of the VBP or the one or more binding agents) contains a streptavidin-binding peptide, wherein the sequence of the streptavidin-binding peptide is set forth in any of SEQ ID NO: 7, 8, and 15-19. In some embodiments, the streptavidin mutein contains the sequence of amino acids set forth in SEQ ID NO: 6, and the binding partner (e.g., that of the VBP or the one or more binding agents) contains a streptavidin-binding peptide, wherein the sequence of the streptavidin-binding peptide is set forth in any of SEQ ID NO: 7, 8, and 15-19. In some embodiments, the streptavidin mutein contains the sequence of amino acids set forth in any of SEQ ID NO: 3-6, 27, 28, 104, 105, and 163, and the binding partner (e.g., that of the VBP or the one or more binding agents) contains a streptavidin-binding peptide, wherein the sequence of the streptavidin-binding peptide is set forth in SEQ ID NO: 16. In some embodiments, the streptavidin mutein contains the sequence of amino acids set forth in SEQ ID NO: 6, and the binding partner (e.g., that of the VBP or the one or more binding agents) contains a streptavidin-binding peptide, wherein the sequence of the streptavidin-binding peptide is set forth in SEQ ID NO: 16.

In some embodiments, the protein reagent contains a plurality of molecules, for instance a plurality of any of the described streptavidin, avidin, streptavidin analog or mutein, and avidin analog or mutein molecules. In some embodiments, the plurality of molecules is a mixture of molecules each independently selected from any of the streptavidin, avidin, streptavidin analog or mutein, and avidin analog or mutein molecules described herein. In some embodiments, the plurality of molecules is a mixture of any of the streptavidin and streptavidin mutein molecules described herein.

In some embodiments, each of the plurality of molecules is the same and is any one of the streptavidin, avidin, streptavidin analog or mutein, and avidin analog or mutein molecules described herein. In some embodiments, each of the plurality of molecules is the same and is streptavidin. In some embodiments, each of the plurality of molecules is the same and is any one of the streptavidin mutein molecules described herein.

In some embodiments, the plurality of molecules contains between 100 and 50,000, between 500 and 10,000, between 1,000 and 20,000, between 500 and 5,000, between 300 and 7,500, between 1,500 and 7,500, between 500 and 3,500, between 1,000 and 5,000, between 1,500 and 2,500, between 1,500 and 2,500, between 2,000 and 3,000, between 2,500 and 3,500, between 2,000 and 4,000, or between 2,000 and 5,000 tetramers, each inclusive, of the molecule or mixture of molecules. In some embodiments, the plurality of molecules contains between or between about 500 and 7500 tetramers, inclusive, of the molecule or mixture of molecules. In some embodiments, the plurality of molecules contains between or between about 500 and 5000 tetramers, 1000 and 4000 tetramers, or 2000 and 3000 tetramers, each inclusive, of the molecule or mixture of molecules. In some embodiments, the plurality of molecules contains between or between about 500 and 5000 tetramers, inclusive, of the molecule or mixture of molecules. In some embodiments, the plurality of molecules contains between or between about 1000 and 4000 tetramers, inclusive, of the molecule or mixture of molecules. In some embodiments, the plurality of molecules contains between or between about 2000 and 3000 tetramers, inclusive, of the molecule or mixture of molecules. In some embodiments, the plurality of molecules contains about 2500 tetramers of the molecule or mixture of molecules. In any of the foregoing embodiments, the number of tetramers is the number of tetramers of the molecule. In any of the foregoing embodiments, the number of tetramers is the number of tetramers of the mixture of molecules.

In some embodiments, the protein reagent has a radius of between 5 nm and 150 nm, between 25 nm and 150 nm, between 50 nm and 150 nm, between 75 nm and 125 nm, between 80 nm and 140 nm, between 85 nm and 135 nm, between 80 nm and 120 nm, between 80 nm and 115 nm, or between 90 nm and 110 nm, inclusive. In some embodiments, the protein reagent has a radius of between 50 nm and 150 nm, inclusive. In some embodiments, the protein reagent has a radius of between 75 nm and 125 nm, inclusive. In some embodiments, the protein reagent has a radius of between 80 nm and 120 nm, inclusive. In some embodiments, the protein reagent has a radius of between 90 nm and 110 nm, inclusive.

In some embodiments, the radius is the hydrodynamic radius, radius of gyration, Stokes radius, Stokes-Einstein radius, or the effective hydrated radius in solution. In some embodiments, the radius is the hydrodynamic radius. In some embodiments, the radius is the Stokes radius.

In some embodiments, the protein reagent is an oligomer of the plurality of molecules. In some embodiments, the oligomer is generated by linking individual molecules of the protein reagent. In some embodiments, the oligomer is generated by linking monomers, dimers, trimers, or tetramers of the molecule. In some embodiments, the molecules are directly linked to one another. In some embodiments, the molecules are indirectly linked to one another. Oligomers can be generated using any suitable method, such as any described in U.S. Pat. No. 7,776,562 and Published U.S. Patent Appl. No. 2021/0032297. In some embodiments, molecules of the plurality of molecules are crosslinked by a polysaccharide or a bifunctional linker.

In some cases, molecules of the plurality of molecules are crosslinked by a polysaccharide. In some embodiments, the oligomer is prepared by the introduction of carboxyl residues into a polysaccharide, e.g., dextran, for instance as described in Noguchi et al, Bioconjugate Chemistry (1992) 3, 132-137 in a first step. In some embodiments, the molecules of the protein reagent, e.g., the streptavidin, avidin, streptavidin analog or mutein, or avidin analog or mutein molecules, may then be linked via primary amino groups of internal lysine residues and/or the free N-terminus to the carboxyl groups in the dextran backbone using carbodiimide chemistry in a second step.

In some embodiments, molecules of the plurality of molecules are crosslinked by a bifunctional linker. Suitable bifunctional linkers can be identified and selected by one of ordinary skill in the art. In some embodiments, the linker is a heterobifunctional linker. In some embodiments, molecules of the plurality of molecules, e.g., the streptavidin, avidin, streptavidin analog or mutein, or avidin analog or mutein molecules, such as the streptavidin mutein molecules, are crosslinked by an amine-to-thiol crosslinker. Exemplary crosslinking reagents include sulfosuccinimidyl 4-(N-maleimidomethyl)cyclohexane-1-carboxylate (sulfo SMCC) or Succinimidyl-6-[(β-maleimidopropionamido)hexanoate (SMPH), and their use in generating oligomers is described in, e.g., Published U.S. Patent Appl. No. 2021/0032297.

### B. Binding Agents

In some embodiments, the VBP reagent further includes one or more binding agents. In some embodiments, the one or more binding agents are immobilized on the reagent, e.g., protein reagent, of the VBP reagent. Thus, in some embodiments, the VBP reagent contains the VBP and the one or more binding agents. Exemplary binding agents of the one or more binding agents of the VBP reagent are described in this section. In some embodiments, the one or more binding agents include any of the binding agents described herein. In some embodiments, such as in the case of more than one binding agent for the VBP reagent, the binding agents are each individually selected from among any of the binding agents described herein. In some embodiments, the one or more binding agents include 2, 3, 4, 5, 6, 7, 8, 9, or 10 different binding agents, which can target the same or different molecules. For instance, in some embodiments, the one or more binding agents include a first binding agent and a second binding agent that target different molecules from one another.

In some embodiments, the binding agent binds to a molecule expressed on the surface of a target cell. A wide variety of, for example, antibodies or antibody fragments that target cell surface molecules are available and suitable for use as part of the binding agents herein and can be identified and selected by one of ordinary skill in the art for use accordingly.

In some embodiments, the binding agent is monovalent. In some embodiments, the binding agent contains two or more binding sites for binding to the molecule expressed on the surface of the target cell (also referred to herein as the cell surface molecule). In some embodiments, the binding agent is divalent.

In some embodiments, the dissociation constant (K_{D}) of the binding between the binding agent and the cell surface molecule is from about 10⁻² M to about 10⁻¹³ M, from about 10⁻³ M to about 10⁻¹² M, from about 10⁻⁴ M to about 10⁻¹¹ M, or from about 10⁻⁵M to about 10⁻¹⁰ M. In some embodiments, the dissociation constant (K_{D}) for the binding between the binding agent and the cell surface molecule is from about 10⁻³ to about 10⁻⁷M, e.g., is of low affinity. In some embodiments, the dissociation constant (K_{D}) for the binding between the binding agent and the cell surface molecule is from about 10⁻⁷ to about 1×10⁻¹⁰ M, e.g., is of high affinity.

In some embodiments, the dissociation of the binding between the binding agent and the cell surface molecule occurs sufficiently fast to, for example, allow the target cell to be only transiently associated with the binding agent after disruption of the reversible bond between the protein reagent and the binding agent. In some embodiments, when expressed in terms of the k_{off} rate (also called dissociation rate constant) for the binding between the binding agent and the cell surface molecule, the k_{off} rate is about 0.5×10⁻⁴ sec⁻¹ or greater, about 1×10⁻⁴ sec⁻¹ or greater, about 2×10⁻⁴ sec⁻¹ or greater, about 3×10⁻⁴ sec⁻¹ or greater, about 4×10⁻⁴ sec⁻¹ of greater, about 5×10⁻⁴ sec⁻¹ or greater, about 1×10⁻³ sec⁻¹ or greater, about 1.5×10⁻³ sec⁻¹ or greater, about 2×10⁻³ sec⁻¹ or greater, about 3×10⁻³ sec⁻¹ or greater, about 4×10⁻³ sec⁻¹, about 5×10⁻³ sec⁻¹ or greater, about 1×10⁻² sec⁻¹ or greater, or about 5×10⁻¹ sec⁻¹ or greater. It is within the level of one of ordinary skill in the art to empirically determine the k_{off} rate range suitable for a particular binding agent and cell surface molecule interaction (see, e.g., U.S. Pat. No. 9,023,604). For example, a binding agent with a higher k_{off} rate of, for example, greater than 4.0×10⁻⁴ sec⁻¹ may be used so that after the disruption of the binding to the protein reagent, most of the binding agent can be removed or dissociated from the target cell within one hour. In other cases, a binding agent with a lower k_{off} rate of, for example, 1.0×10⁻⁴ sec⁻¹, may be used so that after the disruption of the binding to the protein reagent, most of the binding agent may be removed or dissociated from the target cell within about 3 and a half hours.

The K_{D}, k_{off}, and kₒₙ rate of the bond formed between the binding agent and the cell surface molecule can be determined by any suitable means, for example by fluorescence titration, equilibrium dialysis, or surface plasmon resonance.

In some embodiments, the receptor is a lipid, a polysaccharide, or a nucleic acid. In some embodiments, the cell surface molecule is a peptide or a protein, such as a receptor, e.g., a membrane receptor protein. In some embodiments, the cell surface molecule is a peripheral membrane protein or an integral membrane protein. The cell surface molecule can in some embodiments have one or more domains that span the membrane. As a few illustrative examples, a membrane protein with a transmembrane domain may be a G-protein coupled receptor, such as an odorant receptors, a rhodopsin receptor, a rhodopsin pheromone receptor, a peptide hormone receptor, a taste receptor, a GABA receptor, an opiate receptor, a serotonin receptor, a Ca2+ receptor, melanopsin, a neurotransmitter receptor, such as a ligand gated, a voltage gated or a mechanically gated receptor, including the acetylcholine, the nicotinic, the adrenergic, the norepinephrine, the catecholamines, the L-DOPA-, a dopamine and serotonin (biogenic amine, endorphin/enkephalin) neuropeptide receptor, a receptor kinase such as serine/threonine kinase, a tyrosine kinase, a porin/channel such as a chloride channel, a potassium channel, a sodium channel, an OMP protein, an ABC transporter (ATP-Binding Cassette-Transporter) such as amino acid transporter, the Na-glucose transporter, the Na/iodide transporter, an ion transporter such as Light Harvesting Complex, cytochrome c oxidase, ATPase Na/K, H/K, Ca, a cell adhesion receptor such as metalloprotease, an integrin, or a catherin.

In some embodiments, the cell surface molecule is a molecule expressed by or defining a cell population, for instance a population or subpopulation of blood cells, e.g., lymphocytes (e.g., T cells, B cells, or NK cells), monocytes, or stem cells (e.g., CD34 positive peripheral stem cells or Nanog or Oct-4 expressing stem cells). In some embodiments, the cell surface molecule is expressed on the surface of a target cell, e.g., a cell targeted for genetic engineering, e.g., transduction. In some embodiments, the cell surface molecule is a molecule expressed on the surface of immune cells. In some embodiments, the cell surface molecule is a molecule expressed on the surface of lymphocytes. In some embodiments, the cell surface molecule is a molecule expressed on the surface of T cells, B cells, or NK cells. In some embodiments, the cell surface molecule is a molecule expressed on the surface of T cells. Examples of T cells include cells such as CMV-specific CD8+ T cells, cytotoxic T cells, memory T cells, and regulatory T-cells (Treg). An illustrative example of Treg includes CD4 CD25 CD45RA Treg cells, and an illustrative example of memory T cells includes CD62L CD8+ specific central memory T cells. In some embodiments, the cell surface molecule is CD25, CD28, CD62L, CCR7, CD27, CD127, CD3, CD4, CD8, CD57, CD45RA, or CD45RO. In some embodiments, the cell surface molecule is CD3. In some embodiments, the cell surface molecule is CD28. In some embodiments, the cell surface molecule is CD4. In some embodiments, the cell surface molecule is CD8.

In some embodiments, the binding agent contains an antibody, an antibody fragment, a proteinaceous molecule with antibody-like binding properties, a molecule containing Ig domains, a cytokine, a chemokine, an MHC molecules, an MHC-peptide complex, a receptor ligand, or a binding fragment of any of the foregoing, that specifically binds to the cell surface molecule. In some embodiments, the binding agent contains an antibody. In some embodiments, the binding agent contains an antibody fragment. In some embodiments, the antibody fragment is selected from Fab fragments, Fv fragments, single-chain Fv fragments (scFv), divalent antibody fragments such as F(ab′)₂-fragments, diabodies, triabodies (Iliades, P., et al., FEBS Lett (1997) 409, 437-441), decabodies (Stone, E., et al., Journal of Immunological Methods (2007) 318, 88-94), and other domain antibodies (Holt, L. J., et al., Trends Biotechnol. (2003), 21, 11, 484-490).

In some embodiments, the binding agent binds to the cell surface molecule in a monovalent manner. In some embodiments, the binding agent contains a monovalent antibody fragment, a proteinaceous binding molecule with antibody-like binding properties, an aptamer, or an MHC molecule. In some embodiments, the binding agent contains a monovalent antibody fragment. In some embodiments, the monovalent antibody fragment is a Fab fragment, Fv fragment, or single-chain Fv fragment (scFv). In some embodiments, the monovalent antibody fragment is a Fab fragment.

In some embodiments, the binding agent contains an antibody fragment that is a divalent antibody fragment. In some embodiments, the divalent antibody fragment is an F(ab′)₂-fragment or a divalent single-chain Fv fragment.

In some embodiments, the binding agent contains a proteinaceous molecule with antibody-like binding properties. In some embodiments, the proteinaceous molecule with antibody-like binding properties is an aptamer, a mutein based on a polypeptide of the lipocalin family, a glubody, a protein based on the ankyrin scaffold, a protein based on the crystalline scaffold, an adnectin, or an avimer. Other exemplary proteinaceous molecules include an EGF-like domain, a Kringle-domain, a fibronectin type I domain, a fibronectin type II domain, a fibronectin type III domain, a PAN domain, a Gla domain, a SRCR domain, a Kunitz/Bovine pancreatic trypsin Inhibitor domain, tendamistat, a Kazal-type serine protease inhibitor domain, a Trefoil (P-type) domain, a von Willebrand factor type C domain, an Anaphylatoxin-like domain, a CUB domain, a thyroglobulin type I repeat, LDL-receptor class A domain, a Sushi domain, a Link domain, a Thrombospondin type I domain, an immunoglobulin domain or a an immunoglobulin-like domain (for example, domain antibodies or camel heavy chain antibodies), a C-type lectin domain, a MAM domain, a von Willebrand factor type A domain, a Somatomedin B domain, a WAP-type four disulfide core domain, a F5/8 type C domain, a Hemopexin domain, an SH2 domain, an SH3 domain, a Laminin-type EGF-like domain, a C2 domain, “Kappabodies” (cf. Ill. Et al., Protein Eng (1997) 10, 949-57, a so called “minibody” (Martin et al., EMBO J (1994) 13, 5303-5309), a diabody (cf. Holliger et al., PNAS USA (1993)90, 6444-6448), a so called “Janusis” (cf. Traunecker et al., EMBO J (1991) 10, 3655-3659, or Traunecker et al., Int J Cancer (1992) Suppl 7, 51-52), a nanobody, a microbody, an affilin, an affibody, a knottin, ubiquitin, a zinc-finger protein, an autofluorescent protein, and a leucine-rich repeat protein. In some embodiments, the binding agent is a bivalent proteinaceous artificial binding molecule such as a dimeric lipocalin mutein that is also known as “duocalin”.

In some embodiments, the cell surface molecule is a co-receptor. In some embodiments, the cell surface molecule is a T cell co-receptor. In some embodiments, the cell surface molecule is CD4. In some embodiments, the binding agent contains an anti-CD4 antibody, a divalent antibody fragment of an anti-CD4 antibody, a monovalent antibody fragment of an anti-CD4-antibody, or a proteinaceous CD4 binding molecule with antibody-like binding properties. In some embodiments, the anti-CD4 antibody, divalent antibody fragment of an anti-CD4 antibody, or monovalent antibody fragment of an anti-CD4 antibody (e.g., anti-CD4 Fab fragment) is derived from antibody 13B8.2 or a functionally active mutant of 13B8.2 that retains specific binding for CD4. Exemplary mutants of antibody 13B8.2 or m13B8.2 are described in U.S. Pat. No. 7,482,000, U.S. Patent Appl. No. US2014/0295458, International Patent Application No. WO2013/124474, and Bes, C, et al. J Biol Chem 278, 14265-14273 (2003). The mutant Fab fragment termed “ml3B8.2” carries the variable domain of the CD4 binding murine antibody 13B8.2 and a constant domain containing constant human CH1 domain of type gamma for the heavy chain and the constant human light chain domain of type kappa, as described in U.S. Pat. No. 7,482,000. In some embodiments, the anti-CD4 antibody, e.g., a mutant of antibody 13B8.2, contains the amino acid replacement H91A in the variable light chain, the amino acid replacement Y92A in the variable light chain, the amino acid replacement H35A in the variable heavy chain, and/or the amino acid replacement R53A in the variable heavy chain, each by Kabat numbering. In some embodiments, compared to variable domains of the 13B8.2 Fab fragment in m13B8.2, the His residue at position 91 of the light chain (position 93 in SEQ ID NO: 30) is mutated to Ala, and the Arg residue at position 53 of the heavy chain (position 55 in SEQ ID NO: 29) is mutated to Ala. In some embodiments, the binding agent contains an anti-CD4 Fab. In some embodiments, the anti-CD4 Fab contains a variable heavy chain having the sequence set forth in SEQ ID NO: 29 and a variable light chain having the sequence set forth in SEQ ID NO: 30. In some embodiments, the anti-CD4 Fab contains the CDRs of the variable heavy chain having the sequence set forth in SEQ ID NO: 29 and the CDRs of the variable light chain having the sequence set forth in SEQ ID NO: 30.

In some embodiments, the cell surface molecule is CD8. In some embodiments, the binding agent contains an anti-CD8 antibody, a divalent antibody fragment of an anti-CD8 antibody, a monovalent antibody fragment of an anti-CD8 antibody, or a proteinaceous CD8 binding molecule with antibody-like binding properties. In some embodiments, the anti-CD8 antibody, divalent antibody fragment of an anti-CD8 antibody, or monovalent antibody fragment of an anti-CD8 antibody (e.g., anti-CD8 Fab fragment) is derived from antibody OKT8 (e.g., ATCC CRL-8014) or a functionally active mutant thereof that retains specific binding for CD8. In some embodiments, the binding agent contains an anti-CD8 Fab. In some embodiments, the anti-CD8 Fab contains a variable heavy chain having the sequence set forth in SEQ ID NO: 36 and a variable light chain having the sequence set forth in SEQ ID NO: 37. In some embodiments, the anti-CD8 Fab contains the CDRs of the variable heavy chain having the sequence set forth in SEQ ID NO: 36 and the CDRs of the variable light chain having the sequence set forth in SEQ ID NO: 37.

In some embodiments, the cell surface molecule is a molecule containing an immunoreceptor tyrosine-based activation motif (ITAM). In some embodiments, the cell surface molecule is a member of a T cell antigen receptor complex. In some embodiments, the cell surface molecule is a member of a TCR/CD3 complex. In some embodiments, the cell surface molecule is CD3. In some embodiments, the cell surface molecule is a CD3 chain. In some embodiments, the cell surface molecule is a CD3 zeta chain.

In some embodiments, the cell surface molecule is CD3. In some embodiments, the binding agent contains an anti-CD3 antibody, a divalent antibody fragment of an anti-CD3 antibody, a monovalent antibody fragment of an anti-CD3 antibody, or a proteinaceous CD3 binding molecule with antibody-like binding properties. In some embodiments, the anti-CD3 antibody, divalent antibody fragment of an anti-CD3 antibody, or monovalent antibody fragment of an anti-CD3 antibody (e.g., anti-CD3 Fab fragment) is derived from antibody OKT3 (e.g., ATCC CRL-8001; see, e.g., Stemberger et al. PloS One. 2012; 7(4): e35798) or a functionally active mutant thereof that retains specific binding for CD3. In some embodiments, the binding agent contains an anti-CD3 Fab. In some embodiments, the anti-CD3 Fab contains a variable heavy chain having the sequence set forth in SEQ ID NO: 31 and a variable light chain having the sequence set forth in SEQ ID NO: 32. In some embodiments, the anti-CD3 Fab contains the CDRs of the variable heavy chain having the sequence set forth in SEQ ID NO: 31 and the CDRs of the variable light chain having the sequence set forth in SEQ ID NO: 32.

In some embodiments, the cell surface molecule is CD25. In some embodiments, the binding agent contains an anti-CD25 antibody, a divalent antibody fragment of an anti-CD25 antibody, a monovalent antibody fragment of an anti-CD25 antibody, or a proteinaceous CD25 binding molecule with antibody-like binding properties. In some embodiments, the binding agent contains an anti-CD25 Fab. In some embodiments, the anti-CD25 antibody, divalent antibody fragment of an anti-CD25 antibody, or monovalent antibody fragment of an anti-CD25 antibody (e.g., anti-CD25 Fab) is derived from antibody FRT5 (see, e.g., Stemberger et al. 2012. PloS One. 2012; 7(4):e35798) or a functionally active mutant thereof that retains specific binding for CD25.

In some embodiments, the cell surface molecule is CD62L. In some embodiments, the binding agent contains an anti-CD62L antibody, a divalent antibody fragment of an anti-CD62L antibody, a monovalent antibody fragment of an anti-CD62L antibody, or a proteinaceous CD62L binding molecule with antibody-like binding properties. In some embodiments, the binding agent contains an anti-CD62L Fab. In some embodiments, the anti-CD62L antibody, divalent antibody fragment of an anti-CD62L antibody, or monovalent antibody fragment of an anti-CD62L antibody (e.g., anti-CD62L Fab) is derived from antibody DREG56 (e.g., ATCC HB300; see, e.g., Stemberger et al. 2012, PloS One. 2012; 7(4):e35798) or a functionally active mutant thereof that retains specific binding for CD62L.

In some embodiments, the cell surface molecule is CD45RA. In some embodiments, the binding agent contains an anti-CD45RA antibody, a divalent antibody fragment of an anti-CD45RA antibody, a monovalent antibody fragment of an anti-CD45RA antibody, or a proteinaceous CD45RA binding molecule with antibody-like binding properties. In some embodiments, the binding agent contains an anti-CD45RA Fab. In some embodiments, the anti-CD45RA antibody, divalent antibody fragment of an anti-CD45RA antibody, or monovalent antibody fragment of an anti-CD45RA antibody (e.g., anti-CD45RA Fab fragment) is derived from antibody MEM56 (e.g., Millipore 05-1413; see, e.g., Stemberger et al. 2012, PloS One. 2012; 7(4):e35798) or a functionally active mutant thereof that retains specific binding for CD45RA.

In some embodiments, the cell surface molecule is a costimulatory molecule, an accessory molecule, a cytokine receptor, a chemokine receptor, an immune checkpoint molecule, or a member of the TNF family or TNF receptor family. In some embodiments, the cell surface molecule is a costimulatory molecule. In some embodiments, the costimulatory molecule is CD28, CD90 (Thy-1), CD95 (Apo−/Fas), CD137 (4-1BB), CD154 (CD40L), ICOS, LAT, CD27, OX40, or HVEM.

In some embodiments, the cell surface molecule is CD28. In some embodiments, the binding agent contains an anti-CD28 antibody, a divalent antibody fragment of an anti-CD28 antibody, a monovalent antibody fragment of an anti-CD28 antibody, or a proteinaceous CD28 binding molecule with antibody-like binding properties. In some embodiments, the anti-CD28 antibody, divalent antibody fragment of an anti-CD28 antibody, or monovalent antibody fragment of an anti-CD28 antibody (e.g., anti-CD28 Fab fragment) is derived from antibody CD28.3 (deposited as a synthetic single chain Fv construct under GenBank Accession No. AF451974.1; see also Vanhove et al, BLOOD, 15 Jul. 2003, Vol. 102, No. 2, pages 564-570), the variable heavy and light chains of which contain the amino acid sequences set forth in SEQ ID NO: 33 and 34, respectively. In some embodiments, the binding agent contains an anti-CD28 Fab. In some embodiments, the anti-CD28 Fab contains a variable heavy chain having the sequence set forth in SEQ ID NO: 33 and a variable light chain having the sequence set forth in SEQ ID NO: 34. In some embodiments, the anti-CD28 Fab contains the CDRs of the variable heavy chain having the sequence set forth in SEQ ID NO: 33 and the CDRs of the variable light chain having the sequence set forth in SEQ ID NO: 34.

In some embodiments, the cell surface molecule is CD90. In some embodiments, the binding agent contains an anti-CD90 antibody, a divalent antibody fragment of an anti-CD90 antibody, a monovalent antibody fragment of an anti-CD90 antibody, or a proteinaceous CD90 binding molecule with antibody-like binding properties. In some embodiments, the binding agent contains an anti-CD90 Fab. In some embodiments, the anti-CD90 antibody, divalent antibody fragment of an anti-CD90 antibody, or monovalent antibody fragment of an anti-CD90 antibody (e.g., anti-CD90 Fab fragment) is derived from the anti-CD90 antibody G7 (Biolegend, cat. No. 105201).

In some embodiments, the cell surface molecule is CD95. In some embodiments, the binding agent contains an anti-CD95 antibody, a divalent antibody fragment of an anti-CD95 antibody, a monovalent antibody fragment of an anti-CD95 antibody, or a proteinaceous CD95 binding molecule with antibody-like binding properties. In some embodiments, the binding agent contains an anti-CD95 Fab. In some embodiments, the anti-CD95 antibody, divalent antibody fragment of an anti-CD95 antibody, or monovalent antibody fragment of an anti-CD95 antibody (e.g., anti-CD95 Fab fragment) is derived from monoclonal mouse anti-human CD95 CH11 (Upstate Biotechnology, Lake Placid, NY), anti-CD95 mAb 7C11, or anti-APO-1, such as described in Paulsen et al. Cell Death & Differentiation 18.4 (2011): 619-631.

In some embodiments, the cell surface molecule is CD137. In some embodiments, the binding agent contains an anti-CD137 antibody, a divalent antibody fragment of an anti-CD137 antibody, a monovalent antibody fragment of an anti-CD137 antibody, or a proteinaceous CD137 binding molecule with antibody-like binding properties. In some embodiments, the binding agent contains an anti-CD137 Fab. In some embodiments, the anti-CD137 antibody, divalent antibody fragment of an anti-CD137 antibody, or monovalent antibody fragment of an anti-CD137 antibody (e.g., anti-CD137 Fab fragment) is derived from LOB12, IgG2a or LOB12.3, IgG1 as described in Taraban et al. Eur J Immunol. 2002 December; 32(12):3617-27. See also, e.g., U.S. Pat. Nos. 6,569,997, 6,303,121, and Mittler et al. Immunol Res. 2004; 29(1-3):197-208.

In some embodiments, the cell surface molecule is CD40. In some embodiments, the binding agent contains an anti-CD40 antibody, a divalent antibody fragment of an anti-CD40 antibody, a monovalent antibody fragment of an anti-CD40 antibody, or a proteinaceous CD40 binding molecule with antibody-like binding properties. In some embodiments, the binding agent contains an anti-CD40 Fab.

In some embodiments, the cell surface molecule is CD40L. In some embodiments, the binding agent contains an anti-CD40L antibody, a divalent antibody fragment of an anti-CD40L antibody, a monovalent antibody fragment of an anti-CD40L antibody, or a proteinaceous CD40L binding molecule with antibody-like binding properties. In some embodiments, the binding agent contains an anti-CD40L Fab. In some embodiments, the anti-CD40L antibody, divalent antibody fragment of an anti-CD40L antibody, or monovalent antibody fragment of an anti-CD40L antibody (e.g., anti-CD40L Fab fragment) is derived from Hu5C8, as described in Blair et al. JEM vol. 191 no. 4 651-660. See also, e.g., WO1999061065, US20010026932, U.S. Pat. No. 7,547,438, and WO2001056603.

In some embodiments, the cell surface molecule is ICOS. In some embodiments, the binding agent contains an anti-ICOS antibody, a divalent antibody fragment of an anti-ICOS antibody, a monovalent antibody fragment of an anti-ICOS antibody, or a proteinaceous ICOS binding molecule with antibody-like binding properties. In some embodiments, the binding agent contains an anti-ICO Fab. In some embodiments, the anti-ICOS antibody, divalent antibody fragment of an anti-ICOS antibody, or monovalent antibody fragment of an anti-ICOS antibody (e.g., anti-ICOS Fab fragment) is derived from any of the antibodies described in US20080279851 and Deng et al. Hybrid Hybridomics. 2004 June; 23(3):176-82.

In some embodiments, the cell surface molecule is Linker for Activation of T cells (LAT). In some embodiments, the binding agent contains an anti-LAT antibody, a divalent antibody fragment of an anti-LAT antibody, a monovalent antibody fragment of an anti-LAT antibody, or a proteinaceous LAT binding molecule with antibody-like binding properties. In some embodiments, the binding agent contains an anti-LAT Fab.

In some embodiments, the cell surface molecule is CD27. In some embodiments, the binding agent contains an anti-CD27 antibody, a divalent antibody fragment of an anti-CD27 antibody, a monovalent antibody fragment of an anti-CD27 antibody, or a proteinaceous CD27 binding molecule with antibody-like binding properties. In some embodiments, the binding agent contains an anti-CD27 Fab. In some embodiments, the anti-CD27 antibody, divalent antibody fragment of an anti-CD27 antibody, or monovalent antibody fragment of an anti-CD27 antibody (e.g., anti-CD27 Fab fragment) is derived from any of the antibodies described in WO2008051424.

In some embodiments, the cell surface molecule is OX40. In some embodiments, the binding agent contains an anti-OX40 antibody, a divalent antibody fragment of an anti-OX40 antibody, a monovalent antibody fragment of an anti-OX40 antibody, or a proteinaceous OX40 binding molecule with antibody-like binding properties. In some embodiments, the binding agent contains an anti-OX40 Fab. In some embodiments, the anti-OX40 antibody, divalent antibody fragment of an anti-OX40 antibody, or monovalent antibody fragment of an anti-OX40 antibody (e.g., anti-OX40 Fab fragment) is derived from any of the antibodies described in WO2013038191 and Melero et al. Clin Cancer Res. 2013 Mar. 1; 19(5):1044-53.

In some embodiments, the cell surface molecule is HVEM. In some embodiments, the binding agent contains an anti-HVEM antibody, a divalent antibody fragment of an anti-HVEM antibody, a monovalent antibody fragment of an anti-HVEM antibody, or a proteinaceous HVEM binding molecule with antibody-like binding properties. In some embodiments, the binding agent contains an anti-HVEM Fab. In some embodiments, the anti-HVEM antibody, divalent antibody fragment of an anti-HVEM antibody, or monovalent antibody fragment of an anti-HVEM antibody (e.g., anti-HVEM Fab fragment) is derived from any of the antibodies described in WO2006054961, WO2007001459, and Park et al. Cancer Immunol Immunother. 2012 February; 61(2):203-14.

In some embodiments, one of the one or more binding agents includes a binding agent that binds to a member of a TCR/CD3 complex. In some embodiments, one of the one or more binding agents includes a binding agent that binds to CD3. In some embodiments, one of the one or more binding agents includes a binding agent that binds to a costimulatory molecule. In some embodiments, the costimulatory molecule is CD28, CD90 (Thy-1), CD95 (Apo−/Fas), CD137 (4-1BB), CD154 (CD40L), ICOS, LAT, CD27, OX40, or HVEM. In some embodiments, the costimulatory molecule is CD28. In some embodiments, one of the one or more binding agents includes a binding agent that binds to a co-receptor. In some embodiments, the co-receptor is CD4 or CD8. In some embodiments, such binding agents are any as described herein.

In some embodiments, the binding agent contains a binding partner, e.g., any as described herein, such as any of the streptavidin-binding peptides described herein, and an anti-CD4 antibody or antibody fragment, e.g., any as described herein. In some embodiments, the binding agent contains a binding partner, e.g., any as described herein, such as any of the streptavidin-binding peptides described herein, and an anti-CD4 Fab, e.g., any as described herein. In some embodiments, the VBP reagent contains a VBP, e.g., any as described herein, and the anti-CD4 binding agent.

In some embodiments, the binding agent contains a binding partner, e.g., any as described herein, such as any of the streptavidin-binding peptides described herein, and an anti-CD8 antibody or antibody fragment, e.g., any as described herein. In some embodiments, the binding agent contains a binding partner, e.g., any as described herein, such as any of the streptavidin-binding peptides described herein, and an anti-CD8 Fab, e.g., any as described herein. In some embodiments, the VBP reagent contains a VBP, e.g., any as described herein, and the anti-CD8 binding agent.

In some embodiments, the binding agent is a first binding agent, the cell surface molecule is a first molecule, and one of the one or more binding agents is a second binding agent that binds to a second molecule expressed on the surface of the target cell. In some embodiments, the second binding agent binds to a member of a TCR/CD3 complex. In some embodiments, the second binding agent binds to CD3. In some embodiments, the second binding agent binds to a costimulatory molecule. In some embodiments, the costimulatory molecule is CD28, CD90 (Thy-1), CD95 (Apo−/Fas), CD137 (4-1BB), CD154 (CD40L), ICOS, LAT, CD27, OX40, or HVEM. In some embodiments, the costimulatory molecule is CD28. In some embodiments, the second binding agent binds to a co-receptor. In some embodiments, the co-receptor is CD4 or CD8. In some embodiments, such binding agents are any as described herein.

In some embodiments, the first binding agent contains a binding partner, e.g., any as described herein, such as any of the streptavidin-binding peptides described herein, and an anti-CD3 antibody or antibody fragment, e.g., any as described herein, and the second binding agent contains a binding partner, e.g., any as described herein, such as any of the streptavidin-binding peptides described herein, and an anti-CD28 antibody or antibody fragment e.g., any as described herein. In some embodiments, the first binding agent contains a binding partner, e.g., any as described herein, such as any of the streptavidin-binding peptides described herein, and an anti-CD3 Fab, e.g., any as described herein, and the second binding agent contains a binding partner, e.g., any as described herein, such as any of the streptavidin-binding peptides described herein, and an anti-CD28 Fab, e.g., any as described herein. In some embodiments, the VBP reagent contains a VBP, e.g., any as described herein, the anti-CD3 first binding agent, and the anti-CD28 second binding agent.

In some embodiments, the first binding agent contains a binding partner, e.g., any as described herein, such as any of the streptavidin-binding peptides described herein, and an anti-CD4 antibody or antibody fragment, e.g., any as described herein, and the second binding agent contains a binding partner, e.g., any as described herein, such as any of the streptavidin-binding peptides described herein, and an anti-CD8 antibody or antibody fragment e.g., any as described herein. In some embodiments, the first binding agent contains a binding partner, e.g., any as described herein, such as any of the streptavidin-binding peptides described herein, and an anti-CD4 Fab, e.g., any as described herein, and the second binding agent contains a binding partner, e.g., any as described herein, such as any of the streptavidin-binding peptides described herein, and an anti-CD8 Fab, e.g., any as described herein. In some embodiments, the VBP reagent contains a VBP, e.g., any as described herein, the anti-CD4 first binding agent, and the anti-CD8 second binding agent.

### III. Methods for Viral Particle Purification

In some embodiments, the provided methods involve the purification of viral particles, for instance using any of the provided VBPs or VBP reagents. In some embodiments, the viral particles are purified from a sample containing the viral particles.

In some embodiments, a sample containing viral particles is added to an internal cavity of a chromatography column. The provided methods can be used to purify viral particles from any suitable sample. In some embodiments, the internal cavity contains a stationary phase for column chromatography. In some embodiments, the stationary phase contains a chromatography matrix.

In some embodiments, the stationary phase contains any of the provided VBPs, such as any described in Section I. In some embodiments, the VBP is immobilized on the chromatography matrix. In some embodiments, the VBP is reversibly immobilized on the chromatography matrix. In some embodiments, the addition of the sample to the internal cavity immobilizes a viral particle from the sample on the stationary phase, e.g., via binding of the VBP to the viral particle.

In some embodiments, the stationary phase contains any of the provided VBP reagents, such as any described in Section II. In some embodiments, the VBP reagent is immobilized on the chromatography matrix. In some embodiments, the VBP reagent is reversibly immobilized on the chromatography matrix. In some embodiments, the VBP is reversibly bound to the protein reagent of the VBP reagent. In some embodiments, the addition of the sample to the internal cavity immobilizes a viral particle from the sample on the stationary phase, e.g., via binding of the VBP of the VBP reagent to the viral particle.

In some embodiments, the viral particles are any as described herein, for instance any as described in Section V.

In some embodiments, the chromatography column is any as described herein, for instance any as described in Section VI. In some embodiments, the stationary phase is any as described herein, for instance any as described in Section VI. In some embodiments, the chromatography matrix is any as described herein, for instance any as described in Section VI-A.

In some embodiments, the VBP is immobilized on the chromatography matrix. In some embodiments, the VBP is immobilized via binding of the binding partner of the VBP to a molecule immobilized on the chromatography matrix. In some embodiments, the molecule is any of the streptavidin, avidin, streptavidin analog or mutein, and avidin analog or mutein molecules described herein, for instance any in Section II-A. In some embodiments, the chromatography matrix has a selection reagent immobilized thereon. In some embodiments, the selection reagent contains a molecule that is any of the streptavidin, avidin, streptavidin analog or mutein, and avidin analog or mutein molecules described herein, for instance any in Section II-A. In some embodiments, the molecule is any of the streptavidin mutein molecules described herein. In some embodiments, the streptavidin mutein contains the sequence of amino acids set forth in any of SEQ ID NO: 3-6, 27, 28, 104, 105, and 163. In some embodiments, the binding partner of the VBP is bound to the molecule. In some embodiments, the binding partner of the VBP is reversibly bound to the molecule. In some embodiments, the binding partner of the VBP is bound to the biotin-binding site of the molecule. In some embodiments, the binding partner of the VBP is any of the streptavidin-binding peptides described herein. In some embodiments, the streptavidin-binding peptide is set forth in any of SEQ ID NO: 7, 8, and 15-19.

In some embodiments, the provided methods involve eluting the viral particle immobilized on the stationary phase from the internal cavity of the chromatography column. In some embodiments, the eluting includes disrupting the binding between the VBP and the viral particle. In some embodiments, the eluting includes disrupting the binding between the VBP and the chromatography matrix. In some embodiments, the VBP is reversibly bound to the chromatography matrix. In some embodiments, the VBP is reversibly bound to the molecule of the selection reagent immobilized on the chromatography matrix.

In some embodiments, the eluting includes adding a composition containing a substance to the internal cavity. In some embodiments, the substance disrupts the immobilization of the viral particle on the stationary phase. In some embodiments, the substance disrupts the immobilization of the VBP on the chromatography matrix. For instance, in some embodiments, the stationary phase contains a molecule that is any of the streptavidin, avidin, streptavidin analog or mutein, and avidin analog or mutein molecules described herein, and the binding partner of the VBP is any of the binding partners, e.g., streptavidin or avidin binding partners, such as streptavidin-binding peptides, described herein that reversibly binds to the molecule, for instance with reduced binding affinity compared to that of streptavidin to biotin, or such that the binding is disrupted in the presence of biotin. In some embodiments, the substance has higher binding affinity for the molecule than does the binding partner of the VBP. In some embodiments, the substance disrupts the binding of the binding partner of the VBP to the molecule. In some embodiments, the substance is biotin, e.g., D-biotin. In some embodiments, the substance is a biotin analog or derivative, e.g., any as described herein.

### Iv. Methods for Transduction

In some embodiments, the provided methods involve the transduction of target cells, for instance using any of the provided VBP reagents, such as any described in Section II.

In some embodiments, the provided methods involve the on-column transduction of target cells. In some embodiments, “on-column” refers to one or more target cells being immobilized on a stationary phase contained in an internal cavity of a chromatography column during at least a portion of incubation in the presence of a viral particle. For instance, in some embodiments, the stationary phase contains a selection agent that specifically binds to a selection marker expressed on the surface of the one or more target cells. In some embodiments, the specific binding of the selection agent to the selection marker effects the immobilization of the one or more target cells on the stationary phase. In some embodiments, the one or more target cells are not immobilized or become no longer immobilized on the stationary phase during a portion of the incubation in the presence of the viral particle. In some embodiments, a portion of the incubation in the presence of the viral particle is performed while the one or more target cells are present in the internal cavity, though not necessarily immobilized on the stationary phase. In some embodiments, transduction can also be continued following elution of the one or more target cells from the chromatography column, for instance by continuing to incubate the one or more target cells in the presence of the viral particle following elution.

In some embodiments, the provided methods involve incubating the one or more target cells in the presence of a viral particle. In some embodiments, the provided methods involve incubating one or more target cells in the presence of the VBP reagent and the viral particle. In some embodiments, the provided methods involve incubating one or more target cells in the simultaneous presence of the VBP reagent and the viral particle. In some embodiments, “simultaneous” refers to that the target cells, the viral particle, and the VBP are all present at the same time, for instance all present at the same time in the same container, e.g., chromatography column, in which the incubating is taking place. In some embodiments, the incubating produces one or more transduced target cells. In some embodiments, the target cells are immune cells. In some embodiments, the target cells are lymphocytes. In some embodiments, the target cells are T cells, B cells, or NK cells, In some embodiments, the target cells are T cells. In some embodiments, the T cells include CD4+ T cells and/or CD8+ T cells.

In some embodiments, the viral particles are any as described herein, for instance any as described in Section V.

In some embodiments, at least a portion of the incubating occurs in an internal cavity of a chromatography column. In some embodiments, the chromatography column is any as described herein, for instance any as described in Section VI.

In some embodiments, one or more of the target cells are immobilized on a solid support during at least a portion of the incubating. Exemplary solid supports include plates, e.g., culture or well plates, beads, e.g., magnetic beads, and chromatography matrices and stationary phases for, e.g., column chromatography. In some embodiments, the solid support is any as described herein. In some embodiments, the solid support is a chromatography matrix for column chromatography. In some embodiments, the chromatography matrix is any as described herein, including in Section VI-A. In some embodiments, the solid support is a stationary phase for column chromatography. In some embodiments, the stationary phase is any as described herein, including in Section VI. In some embodiments, the stationary phase is contained in the internal cavity of the chromatography column during the at least a portion of the incubating.

In some embodiments, the provided methods involve contacting the target cells with one or more compositions containing one or both of the VBP reagent and the viral particle. In some embodiments, the provided methods involve adding, to the internal cavity, one or more compositions containing one or both of the VBP reagent and the viral particle. In some embodiments, the one or more compositions is a composition containing both of the VBP reagent and the viral particle. In some embodiments, the target cells are contacted with the composition, for instance while they are immobilized on the stationary phase. In some embodiments, the provided methods involve mixing the VBP reagent and the viral particle to form the composition containing both. In some embodiments, the one or more compositions is a first composition containing the VBP reagent and a second composition that is separate from the first composition and contains the viral particle. In some embodiments, the target cells are contacted with the first and second compositions, for instance while they are immobilized on the stationary phase. In some embodiments, the target cells are simultaneously contacted with the first and second compositions. In some embodiments, “simultaneous contact” means with a time separation of no more than about 15 minutes, such as no more than about 10 minutes, 5 minutes, or 1 minute. In some embodiments, the target cells are contacted with the first and second compositions no more than 15 minutes, 10 minutes, 5 minutes, or 1 minute apart from each other, for instance while the target cells are immobilized on the stationary phase.

Sections IV-A to IV-F describe exemplary steps of the provided methods. In some embodiments, the provided methods involve one or more steps of selecting, stimulating, and transducing target cells. Such steps are any as described herein, for instance any as described in Sections IV-A to IV-C. In some embodiments, the provided methods involve steps of selecting and transducing target cells. In some embodiments, the provided methods involve steps of stimulating and transducing target cells. In some embodiments, the provided methods involve steps of selecting, stimulating, and transducing cells. In some embodiments, some of these steps occur in the internal cavity of the chromatography column. In some embodiments, all of these steps occur in the internal cavity of the chromatography column. In some embodiments, the steps of stimulating and transducing target cells are initiated while target cells are immobilized on the stationary phase.

In some embodiments, a step of selecting target cells is performed prior to steps of stimulating or transducing cells. In some embodiments, a step of selecting target cells is performed prior to the incubation of the target cells in the simultaneous presence of the viral particle and VBP reagent. In some embodiments, the step of selecting target cells is based on expression of a surface molecule expressed by the target cells. In some embodiments, the step of selecting target cells effects the immobilization of the target cells on the stationary phase, for instance so that the target cells are so immobilized during one or both of steps of stimulating and transducing target cells. In some embodiments, a step of selecting target cells is performed subsequent to steps of stimulating or transducing cells.

In some embodiments, a step of selecting target cells is performed subsequent to a step of stimulating target cells. For instance, in some embodiments, the target cells are stimulated using a stimulatory reagent prior to the addition of the stimulated target cells to a chromatography column. In some embodiments, the stimulatory reagent is any reagent described herein that is suitable for the stimulation of the target cells, for instance any as described in Section IV-B. In some embodiments, a step of stimulating target cells is performed subsequent to a step of selecting target cells. In some embodiments, the target cells are stimulated using the stimulatory reagent while the target cells are contained in the chromatography column used for selection. In some embodiments, stimulation with the stimulatory reagent is initiated while the target cells are immobilized on the stationary phase.

In some embodiments, a step of stimulating target cells, e.g., using the stimulatory reagent, is performed prior to a step of transducing target cells, e.g., the incubation of the target cells in the presence of the viral particle, for instance simultaneously with the VBP reagent. In some embodiments, a step of stimulating target cells, e.g., using the stimulatory reagent, is performed subsequent to a step of transducing target cells, e.g., the incubation of the target cells in the presence of the viral particle, for instance simultaneously with the VBP reagent.

In some embodiments, one or more of these steps are combined. For instance, in some embodiments, the target cells are simultaneously stimulated and transduced. In some embodiments, the target cells are in the simultaneous presence of the stimulatory reagent and the viral particle. In some embodiments, the target cells are in the simultaneous presence of the stimulatory reagent and the viral particle while they are immobilized on the stationary phase. In some embodiments, the target cells are simultaneously contacted with the stimulatory reagent and the viral particle, for instance while they are immobilized on the stationary phase. In some embodiments, “simultaneous contact” means with a time separation of no more than about 15 minutes, such as no more than about 10 minutes, 5 minutes, or 1 minute. In some embodiments, the target cells are contacted with the stimulatory reagent and the viral particle no more than 15 minutes, 10 minutes, 5 minutes, or 1 minute apart from each other, for instance while the target cells are immobilized on the stationary phase. In some embodiments, the stimulatory reagent and viral particle are contained in the same composition (e.g., a mixture containing both the stimulatory reagent and viral particle), and the target cells are contacted with the composition, for instance while they are immobilized on the stationary phase. In some embodiments, the stimulatory reagent and viral particle are contained in separate compositions (e.g., the stimulatory reagent in one composition and the viral vector in another composition). In some embodiments, the target cells are simultaneously contacted with the separate compositions, for instance while the target cells are immobilized on the stationary phase. In some embodiments, the target cells are contacted with the separate compositions with a time separation of no more than about 15 minutes, 10 minutes, 5 minutes, or 1 minute apart from one another, for instance while the target cells are immobilized on the stationary phase.

In some embodiments, during at least a portion of the incubating, the target cells are incubated in the simultaneous presence of the VBP reagent, the stimulatory reagent, and the viral particle. In some embodiments, the target cells are incubated in the simultaneous presence of the VBP reagent, the stimulatory reagent, and the viral particle while they are immobilized on the stationary phase. In some embodiments, the target cells are contacted with a single composition containing all of the VBP reagent, the stimulatory reagent, and the viral particle, for instance while the target cells are immobilized on the stationary phase. In some embodiments, the target cells are contacted with multiple compositions containing any combination of the VBP reagent, the stimulatory reagent, and the viral particle, for instance while the target cells are immobilized on the stationary phase. In some embodiments, the target cells are contacted with the multiple compositions simultaneously, for instance while the target cells are immobilized on the stationary phase.

In some embodiments, the VBP reagent contains one or more binding agents. In some embodiments, the one or more binding agents are any as described herein, for instance in Section II-B, that are suitable for the stimulation of cells. Thus, in some embodiments, the VBP reagent is itself a stimulatory reagent.

In some embodiments, the one or more binding agents are suitable for the stimulation of T cells. In some embodiments, one of the one or more binding agents binds to a molecule expressed on the surface of the plurality of target cells and thereby provides a primary activation signal to the plurality of target cells. In some embodiments, the molecule is a member of a TCR/CD3 complex. In some embodiments, the molecule is CD3. In some embodiments, the binding agent is any of the anti-CD3 binding agents described herein herein, for instance any in Section II-B.

In some embodiments, the binding agent is a first binding agent, the molecule is a first molecule, and one of the one or more binding agents is a second binding agent that binds to a second molecule expressed on the surface of the plurality of target cells. In some embodiments, the second molecule is a costimulatory molecule. In some embodiments, the second binding agent binds and thereby provides a costimulatory signal to the plurality of target cells. In some embodiments, the costimulatory molecule is CD28, CD90 (Thy-1), CD95 (Apo−/Fas), CD137 (4-1BB), CD154 (CD40L), ICOS, LAT, CD27, OX40, or HVEM. In some embodiments, the second binding agent is any as described herein, for instance any in Section II-B, that binds to these costimulatory molecules. In some embodiments, the costimulatory molecule is CD28. In some embodiments, the second binding agent is any of the anti-CD28 binding agents described herein herein, for instance any in Section II-B.

In some embodiments, the first binding agent contains an anti-CD3 antibody or antibody fragment, and the second binding agent contains an anti-CD28 antibody or antibody fragment. In some embodiments, the first binding agent contains an anti-CD3 Fab, and the second binding agent contains an anti-CD28 Fab.

In some embodiments, some or all of the steps of selecting, stimulating, and transducing cells are carried out at a temperature that is above room temperature, for instance at a physiological temperature. In some embodiments, target cells contained in the chromatography column are maintained at a temperature that is a physiological temperature. For instance, in some embodiments, at least a portion of the incubating is carried out a temperature between about 35° C. and about 39° C., such as at or about 37° C. In some embodiments, the temperature of the stationary phase is regulated by one or more heating elements configured to provide heat to the stationary phase, for instance according to any of the methods or with any of the devices described in U.S. Patent Publication No. 2022/0002669 and International Published PCT Appl. No. WO 2021/084050.

In some embodiments, the provided methods also involve one or more steps of collecting, culturing, and harvesting cells, for instance one or more steps of collecting the transduced target cells from the chromatography column, further incubating (e.g., culturing) the collected cells, for instance outside of the chromatography column, and harvesting the transduced target cells after the further incubating (e.g., culturing) of the collected transduced target cells. In some embodiments, such steps are any as described herein, for instance those described in Sections IV-D to IV-F.

In some embodiments, any number of the steps of the provided method are performed in a closed system. In some embodiments, any number of the steps of the provided method are automated.

In some embodiments, the target cells are contained in a sample. In some embodiments, the sample is any as described herein, and suitable samples can be selected and identified by one of ordinary skill in the art. In some embodiments, the provided methods involve adding the sample to the internal cavity of the chromatography column. In some embodiments, the adding immobilizes one or more of the target cells on the stationary phase, for instance via binding of a selection agent contained by the stationary phase to a selection marker expressed on the surface of the plurality of target cells.

In some embodiments, the incubating is initiated within or within about 120 minutes after adding the sample to the internal cavity. In some embodiments, the incubating is initiated within or within about 90 minutes after adding the sample to the internal cavity. In some embodiments, the incubating is initiated within or within about 60 minutes after adding the sample to the internal cavity. In some embodiments, the incubating is initiated within or within about 45 minutes after adding the sample to the internal cavity. In some embodiments, the incubating is initiated within or within about 30 minutes after adding the sample to the internal cavity. In some embodiments, the incubating is initiated within or within about 20 minutes after adding the sample to the internal cavity. In some embodiments, the incubating is initiated within or within about 15 minutes after adding the sample to the internal cavity. In some embodiments, the incubating is initiated within or within about 10 minutes after adding the sample to the internal cavity.

In some embodiments, target cells of the sample are allowed sufficient time to penetrate the stationary phase prior to the initiation of incubation, for instance prior to the addition of the one or more compositions containing the VBP reagent, stimulatory reagent, and/or viral particle. In some embodiments, target cells of the sample are allowed sufficient time to become immobilized on the stationary phase, for instance via binding to the selection agent of the stationary phase, prior to the initiation of incubation. In some embodiments, the incubating is initiated at least 5, 10, or 15 minutes following the addition of the sample. In some embodiments, the stationary phase is washed at least one time following the addition of the sample and prior to the initiation of incubation.

In some embodiments, the sample is a biological sample. In some embodiments, the plurality of target cells are primary cells. In some embodiments, the primary cells are primary cells from a human subject. The samples can include tissue, fluid, and other samples taken directly from the subject. The biological sample can be a sample obtained directly from a biological source or a sample that is processed. Biological samples include body fluids, such as blood, plasma, serum, cerebrospinal fluid, synovial fluid, urine, and sweat; tissue; and organ samples, including processed samples derived therefrom.

In some aspects, the sample is blood or a blood-derived sample, or is or is derived from an apheresis or leukapheresis product. Exemplary samples include whole blood, peripheral blood mononuclear cells (PBMCs), leukocytes, bone marrow, thymus, tissue biopsy, tumor, leukemia, lymphoma, lymph node, gut associated lymphoid tissue, mucosa associated lymphoid tissue, spleen, other lymphoid tissues, liver, lung, stomach, intestine, colon, kidney, pancreas, breast, bone, prostate, cervix, testes, ovaries, tonsil, or other organ, or cells derived therefrom.

In some examples, cells from the circulating blood of the subject are obtained by, e.g., apheresis or leukapheresis. The samples can contain lymphocytes, including T cells, monocytes, granulocytes, B cells, other nucleated white blood cells, red blood cells, and/or platelets, and in some aspects contains cells other than red blood cells and platelets.

In some embodiments, the sample is a sample containing T cells. In some embodiments, the sample is a whole blood sample, a buffy coat sample, a peripheral blood mononuclear cell (PBMC) sample, an unfractionated T cell sample, a lymphocyte sample, a white blood cell sample, an apheresis product, or a leukapheresis product. In some embodiments, the sample is an apheresis product. In some embodiments, the sample is a leukaphresis product.

In some embodiments, the blood cells collected from the subject are washed to, e.g., remove the plasma fraction and to place the cells in an appropriate buffer or medium for subsequent processing steps. In some embodiments, the cells are washed with phosphate buffered saline (PBS). In some embodiments, the wash solution lacks calcium, magnesium, and/or many or all divalent cations. In some aspects, a washing step is accomplished using a semi-automated “flow-through” centrifuge (for example, the Cobe 2991 cell processor, Baxter) according to the manufacturer's instructions. In some aspects, a washing step is accomplished by tangential flow filtration (TFF) according to the manufacturer's instructions. In some embodiments, the cells are resuspended in a variety of biocompatible buffers after washing, such as Ca²⁺/Mg²⁺ free PBS. In some embodiments, components of a blood cell sample are removed, and the cells directly resuspended in culture media. In some embodiments, the sample (e.g., an apheresis product or a leukapheresis product) is washed in order to remove one or more anti-coagulants, such as heparin, added during apheresis or leukapheresis.

### A. Selection

In some embodiments, the provided methods involve a step of selecting target cells. In some embodiments, the selecting of target cells is by column chromatography. In some embodiments, the selecting effects the immobilization of target cells on the stationary phase during the incubation in the presence of the VBP reagent, stimulatory reagent, and/or viral particle.

In some embodiments, the stationary phase, e.g., any as described herein, for instance in Section VI, contains a selection agent that specifically binds to a selection marker expressed on the surface of the target cells. In some embodiments, the sample containing the target cells contains additional cells that are devoid of the selection marker. For example, in some embodiments, T cells are selected from a sample containing multiple cells types, e.g., red blood cells or B cells.

In some embodiments, specific binding of the selection agent to the selection marker effects the immobilization of the target cells on the stationary phase. In some embodiments, the selection of target cells, e.g., the immobilization of target cells on the stationary phase, is performed using any of the methods described in PCT Application Nos. WO2013124474, WO2015164675, WO2017/068425, and WO2021/084050 and U.S. Patent Publication No. 2022/0002669. In some embodiments, specific binding of the selection agent to the selection marker effects the immobilization of the target cells on the stationary phase, for instance during the incubation in the presence of the VBP reagent, stimulatory reagent, and/or viral particle.

In some embodiments, the binding agents used in the VBP reagent can also be used as selection agents in the provided methods. In some embodiments, the selection agent is any of the binding agents described herein, e.g., any described in Section II-B, suitable for binding to cell surface molecules. For instance, in some embodiments, the selection marker is a T cell coreceptor or a member of a T cell antigen receptor complex. In some embodiments, the selection marker is selected from the group consisting of CD3, CD4, CD8, CD45RA, CD27, CD28, and CCR7. In some embodiments, the selection marker is CD4. In some embodiments, the selection marker is CD8. In some embodiments, the selection marker is CD3. In some embodiments, the selection agent is any of the binding agents described herein, e.g., any described in Section II-B, that binds to the aforementioned selection markers.

In some embodiments, the selection agent contains a binding partner. In some embodiments, the binding partner is any of the binding partners of the VBP described herein. For instance, in some embodiments, the binding partner of the selection agent is any of the streptavidin or avidin binding partners described herein. In some embodiments, the binding partner of the selection agent is any of the streptavidin-binding partners described herein. In some embodiments, the binding partner of the selection agent is any of the biotin, biotin analogs or derivatives, or streptavidin-binding peptides described herein. In some embodiments, the binding partner of the selection agent is any of the streptavidin-binding peptides described herein. In some embodiments, the streptavidin-binding peptide is set forth in any of SEQ ID NO: 7, 8, and 15-19.

In some embodiments, the stationary phase contains a chromatography matrix. In some embodiments, the chromatography matrix is any as described herein, for instance in Section VI-A. In some embodiments, the selection agent is immobilized on the chromatography matrix. In some embodiments, the chromatography matrix has a selection reagent immobilized thereon that contains a molecule that is any of the streptavidin, avidin, streptavidin analog or mutein, and avidin analog or mutein molecules described herein. In some embodiments, the binding partner of the selection agent is bound to the molecule. In some embodiments, the binding partner of the selection agent is reversibly bound to the molecule. In some embodiments, the binding partner of the selection agent is bound to the biotin-binding site of the molecule. In some embodiments, the molecule is any of the streptavidin mutein molecules described herein. In some embodiments, the streptavidin mutein contains the sequence of amino acids set forth in any of SEQ ID NO: 3-6, 27, 28, 104, 105, and 163.

### B. Stimulation

In some embodiments, the provided methods involve a step of stimulating target cells. In some embodiments, the target cells are incubated in the presence of a stimulatory reagent used for stimulating the target cells. In some embodiments, the target cells are stimulated with the stimulatory reagent while immobilized on the stationary phase, e.g., by the selection agent bound to the selection marker expressed by the target cells. In some embodiments, the target cells are incubated in the simultaneous presence of the VBP reagent, the stimulatory reagent, and the viral particle, for instance while the target cells are immobilized on the stationary phase.

In some aspects, the stimulation is carried out in accordance with techniques such as those described in U.S. Pat. No. 6,040,177 to Riddell et al., Klebanoff et al. (2012) J Immunother. 35(9): 651-660, Terakura et al. (2012) Blood.1:72-82, and Wang et al. (2012) J Immunother. 35(9):689-701.

In some embodiments, the stimulatory reagent is any as described in U.S. Pat. No. 11,274,278 and U.S. Patent Appl. No. 2021/0032297, for instance any of the soluble multimerization reagent or oligomeric particle reagents described therein that are suitable for stimulating cells.

In some embodiments, the stimulatory reagent contains any of the protein reagents described herein, e.g., any as described in Section II-A. In some embodiments, the protein reagent can be the same as those used for the VBP reagent. In some embodiments, the stimulatory reagent contains one or more binding agents, e.g., any as described in herein, for instance in Section II-B, suitable for the stimulation of cells. In some embodiments, the one or more binding agents can be the same as those used for the VBP reagent.

In some embodiments, the protein reagent contains a molecule that is any of the streptavidin, avidin, streptavidin analog or mutein, and avidin analor or mutein molecules described herein. In some embodiments, the molecule is any of the streptavidin mutein molecules described herein. In some embodiments, the streptavidin mutein contains the sequence of amino acids set forth in any of SEQ ID NO: 3-6, 27, 28, 104, 105, and 163.

In some embodiments, the one or more binding agents each contain a binding partner. In some embodiments, the binding partner is any of the binding partners of the VBP described herein. For instance, in some embodiments, the binding partner of the one or more binding agents is any of the streptavidin or avidin binding partners described herein. In some embodiments, the binding partner of the one or more binding agents is any of the streptavidin-binding partners described herein. In some embodiments, the binding partner of the one or more binding agents is any of the biotin, biotin analogs or derivatives, or streptavidin-binding peptides described herein. In some embodiments, the binding partner of the one or more binding agents is any of the streptavidin-binding peptides described herein. In some embodiments, the streptavidin-binding peptide is set forth in any of SEQ ID NO: 7, 8, and 15-19.

In some embodiments, the one or more binding agents are bound to the protein reagent of the stimulatory reagent. In some embodiments, the one or more binding agents are bound to the molecule of the protein reagent. In some embodiments, the one or more binding agents are reversibly bound to the molecule of the protein reagent. In some embodiments, the one or more binding agents are reversibly bound to a biotin-binding site of the molecule of the protein reagent.

In some embodiments, one of the one or more binding agents binds to a molecule expressed on the surface of the plurality of target cells and thereby provides a primary activation signal to the plurality of target cells. In some embodiments, the molecule is a member of a TCR/CD3 complex. In some embodiments, the molecule is CD3. In some embodiments, the binding agent is any of the anti-CD3 binding agents described herein herein, for instance any in Section II-B.

In some embodiments, the binding agent is a first binding agent, the molecule is a first molecule, and one of the one or more binding agents is a second binding agent that binds to a second molecule expressed on the surface of the plurality of target cells. In some embodiments, the second molecule is a costimulatory molecule. In some embodiments, the second binding agent binds and thereby provides a costimulatory signal to the plurality of target cells. In some embodiments, the costimulatory molecule is CD28, CD90 (Thy-1), CD95 (Apo−/Fas), CD137 (4-1BB), CD154 (CD40L), ICOS, LAT, CD27, OX40, or HVEM. In some embodiments, the second binding agent is any as described herein, for instance any in Section II-B, that binds to these costimulatory molecules. In some embodiments, the costimulatory molecule is CD28. In some embodiments, the second binding agent is any of the anti-CD28 binding agents described herein herein, for instance any in Section II-B.

In some embodiments, the stimulatory reagent is a soluble reagent containing the protein reagent, the anti-CD3 first binding agent, and the anti-CD28 second binding agent. In some embodiments, the protein reagent, the anti-CD3 first binding agent, and the anti-CD28 second binding agent can be the same as those used for the VBP reagent. In some embodiments, the first binding agent contains an anti-CD3 antibody or antibody fragment, and the second binding agent contains an anti-CD28 antibody or antibody fragment. In some embodiments, the first binding agent contains an anti-CD3 Fab, and the second binding agent contains an anti-CD28 Fab.

In some embodiments, the incubating is in the presence of between or between about 0.1 μg and 20 μg of the stimulatory reagent per 10⁶ cells of the target cells, of the target cells immobilized on the stationary phase, or of estimated cell counts of any of the foregoing. In some embodiments, the incubating is in the presence of between or between about 0.1 μg and 16 μg of the stimulatory reagent per 10⁶ cells of the target cells, of the target cells immobilized on the stationary phase, or of estimated cell counts of any of the foregoing. In some embodiments, the incubating is in the presence of between or between about 0.1 μg and 12 μg of the stimulatory reagent per 10⁶ cells of the target cells, of the target cells immobilized on the stationary phase, or of estimated cell counts of any of the foregoing. In some embodiments, the incubating is in the presence of between or between about 0.1 μg and 8 μg of the stimulatory reagent per 10⁶ cells of the target cells, of the target cells immobilized on the stationary phase, or of estimated cell counts of any of the foregoing. In some embodiments, the incubating is in the presence of between or between about 0.1 μg and 6 μg of the stimulatory reagent per 10⁶ cells of the target cells, of the target cells immobilized on the stationary phase, or of estimated cell counts of any of the foregoing. In some embodiments, the incubating is in the presence of between or between about 0.5 μg and 20 μg of the stimulatory reagent per 10⁶ cells of the target cells, of the target cells immobilized on the stationary phase, or of estimated cell counts of any of the foregoing. In some embodiments, the incubating is in the presence of between or between about 0.5 μg and 16 μg of the stimulatory reagent per 10⁶ cells of the target cells, of the target cells immobilized on the stationary phase, or of estimated cell counts of any of the foregoing. In some embodiments, the incubating is in the presence of between or between about 0.5 μg and 12 μg of the stimulatory reagent per 10⁶ cells of the target cells, of the target cells immobilized on the stationary phase, or of estimated cell counts of any of the foregoing. In some embodiments, the incubating is in the presence of between or between about 0.5 μg and 8 μg of the stimulatory reagent per 10⁶ cells of the target cells, of the target cells immobilized on the stationary phase, or of estimated cell counts of any of the foregoing. In some embodiments, the incubating is in the presence of between or between about 0.5 μg and 6 μg of the stimulatory reagent per 10⁶ cells of the target cells, of the target cells immobilized on the stationary phase, or of estimated cell counts of any of the foregoing. In some embodiments, the incubating is in the presence of between or between about 1 μg and 20 μg of the stimulatory reagent per 10⁶ cells of the target cells, of the target cells immobilized on the stationary phase, or of estimated cell counts of any of the foregoing. In some embodiments, the incubating is in the presence of between or between about 1 μg and 16 μg of the stimulatory reagent per 10⁶ cells of the target cells, of the target cells immobilized on the stationary phase, or of estimated cell counts of any of the foregoing. In some embodiments, the incubating is in the presence of between or between about 1 μg and 12 μg of the stimulatory reagent per 10⁶ cells of the target cells, of the target cells immobilized on the stationary phase, or of estimated cell counts of any of the foregoing. In some embodiments, the incubating is in the presence of between or between about 1 μg and 8 μg of the stimulatory reagent per 10⁶ cells of the target cells, of the target cells immobilized on the stationary phase, or of estimated cell counts of any of the foregoing. In some embodiments, the incubating is in the presence of between or between about 1 μg and 6 μg of the stimulatory reagent per 10⁶ cells of the target cells, of the target cells immobilized on the stationary phase, or of estimated cell counts of any of the foregoing. In some embodiments, the incubating is in the presence of between or between about 2 μg and 20 μg of the stimulatory reagent per 10⁶ cells of the target cells, of the target cells immobilized on the stationary phase, or of estimated cell counts of any of the foregoing. In some embodiments, the incubating is in the presence of between or between about 2 μg and 16 μg of the stimulatory reagent per 10⁶ cells of the target cells, of the target cells immobilized on the stationary phase, or of estimated cell counts of any of the foregoing. In some embodiments, the incubating is in the presence of between or between about 2 μg and 12 μg of the stimulatory reagent per 10⁶ cells of the target cells, of the target cells immobilized on the stationary phase, or of estimated cell counts of any of the foregoing. In some embodiments, the incubating is in the presence of between or between about 2 μg and 8 μg of the stimulatory reagent per 10⁶ cells of the target cells, of the target cells immobilized on the stationary phase, or of estimated cell counts of any of the foregoing. In some embodiments, the incubating is in the presence of between or between about 2 μg and 6 μg of the stimulatory reagent per 10⁶ cells of the target cells, of the target cells immobilized on the stationary phase, or of estimated cell counts of any of the foregoing. n some embodiments, the incubating is in the presence of between or between about 3 μg and 5 μg of the stimulatory reagent per 10⁶ cells of the target cells, of the target cells immobilized on the stationary phase, or of estimated cell counts of any of the foregoing. n some embodiments, the incubating is in the presence of between or between about 3.5 μg and 4.5 μg of the stimulatory reagent per 10⁶ cells of the target cells, of the target cells immobilized on the stationary phase, or of estimated cell counts of any of the foregoing. In some embodiments, the incubating is in the presence of about 4 μg of the stimulatory reagent per 10⁶ cells of the target cells, of the target cells immobilized on the stationary phase, or of estimated cell counts of any of the foregoing. In any of the foregoing embodiments, the amount of the stimulatory reagent is per 10⁶ cells of the target cells. In any of the foregoing embodiments, the amount of the stimulatory reagent is per 10⁶ cells of the estimated count of the target cells immobilized on the stationary phase. In any of the foregoing embodiments, the amount of the stimulatory reagent is per 10⁶ cells of the binding capacity of the stationary phase. In some embodiments, the amount of stimulatory reagent that is present in any of the described compositions containing the stimulatory reagent, e.g., those contacted to the target cells or added to the internal cavity, is any of those described in the foregoing embodiments.

In some embodiments, the stimulatory reagent contains a weight ratio of protein reagent:first binding agent (e.g., anti-CD3 binding agent):second binding agent (e.g., anti-CD28 binding agent) that is between about 10:1:1 and 2:1:1, inclusive. In some embodiments, the stimulatory reagent contains a weight ratio of protein reagent:first binding agent:second binding agent that is between about 8:1:1 and 2:1:1, inclusive. In some embodiments, the stimulatory reagent contains a weight ratio of protein reagent:first binding agent:second binding agent that is between about 8:1:1 and 4:1:1, inclusive. In some embodiments, the stimulatory reagent contains a weight ratio of protein reagent:first binding agent:second binding agent that is about 6:1:1. In some embodiments, 4 μg of the stimulatory reagent contains about 3 μg of protein reagent, 0.5 μg of anti-CD3 binding agent. and 0.5 μg of anti-CD28 binding agent.

In some embodiments, the incubating in the presence of the stimulatory reagent can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to stimulate the target cells.

In some embodiments, the incubating in the presence of the stimulatory reagent is carried out at room temperature (e.g., at or about 23° C.). In some embodiments, the incubating in the presence of the stimulatory reagent is carried out between about 30° C. and about 39° C., such as at or about 37° C. In some embodiments, the oxygen and carbon dioxide content is controlled using gas exchange.

In some embodiments, the incubating in the presence of the stimulatory reagent of target cells immobilized on the stationary phase facilitates downregulation of the cell surface molecule used for target cell selection (e.g., selection marker), in some instances resulting in spontaneous detachment or release of the target cell from the stationary phase. The release or detachment of the target cells can occur without any additional steps or reagents. In some aspects, the target cells can be collected using wash buffer that does not contain a competition agent to, e.g., facilitate detachment of the target cells from the stationary phase.

### C. Transduction

In some embodiments, the provided methods involve a step of transducing target cells. In some embodiments, the provided methods involve contacting target cells with the VBP reagent and viral particles under conditions for transducing the target cells. In some embodiments, the contacting is carried out by incubating target cells with the VBP reagent and the viral particles. In some embodiments, at least a portion of the incubating with the VBP reagent is carried out in the presence of the viral particles. In some embodiments, the target cells are incubated in the simultaneous presence of the viral particle and the VBP reagent. In some embodiments, the target cells are incubated in the simultaneous presence of the viral particle and the VBP reagent while immobilized on the stationary phase, e.g., by the selection agent bound to the selection marker expressed by the target cells.

In some embodiments, the viral particle is any as described herein, for instance any as described in Section V. In some embodiments, the viral particle contains a nucleic acid sequence encoding a recombinant protein. In some embodiments, the recombinant protein is an antigen receptor. In some embodiments, the recombinant protein is a chimeric antigen receptor (CAR), e.g., any as described herein. In some embodiments, the recombinant protein is an engineered T cell receptor (eTCR), e.g., any as described herein. In some embodiments, the incubating produces one or more transduced target cells. In some embodiments, the transduced target cells express the recombinant protein.

In some embodiments, the incubating is in the presence of between or between about 0.1 μg and 20 μg of the VBP reagent per 10⁶ cells of the target cells, of the target cells immobilized on the stationary phase, or of estimated cell counts of any of the foregoing. In some embodiments, the incubating is in the presence of between or between about 0.1 μg and 16 μg of the VBP reagent per 10⁶ cells of the target cells, of the target cells immobilized on the stationary phase, or of estimated cell counts of any of the foregoing. In some embodiments, the incubating is in the presence of between or between about 0.1 μg and 12 μg of the VBP reagent per 10⁶ cells of the target cells, of the target cells immobilized on the stationary phase, or of estimated cell counts of any of the foregoing. In some embodiments, the incubating is in the presence of between or between about 0.1 μg and 8 μg of the VBP reagent per 10⁶ cells of the target cells, of the target cells immobilized on the stationary phase, or of estimated cell counts of any of the foregoing. In some embodiments, the incubating is in the presence of between or between about 0.1 μg and 6 μg of the VBP reagent per 10⁶ cells of the target cells, of the target cells immobilized on the stationary phase, or of estimated cell counts of any of the foregoing. In some embodiments, the incubating is in the presence of between or between about 0.5 μg and 20 μg of the VBP reagent per 10⁶ cells of the target cells, of the target cells immobilized on the stationary phase, or of estimated cell counts of any of the foregoing. In some embodiments, the incubating is in the presence of between or between about 0.5 μg and 16 μg of the VBP reagent per 10⁶ cells of the target cells, of the target cells immobilized on the stationary phase, or of estimated cell counts of any of the foregoing. In some embodiments, the incubating is in the presence of between or between about 0.5 μg and 12 μg of the VBP reagent per 10⁶ cells of the target cells, of the target cells immobilized on the stationary phase, or of estimated cell counts of any of the foregoing. In some embodiments, the incubating is in the presence of between or between about 0.5 μg and 8 μg of the VBP reagent per 10⁶ cells of the target cells, of the target cells immobilized on the stationary phase, or of estimated cell counts of any of the foregoing. In some embodiments, the incubating is in the presence of between or between about 0.5 μg and 6 μg of the VBP reagent per 10⁶ cells of the target cells, of the target cells immobilized on the stationary phase, or of estimated cell counts of any of the foregoing. In some embodiments, the incubating is in the presence of between or between about 1 μg and 20 μg of the VBP reagent per 10⁶ cells of the target cells, of the target cells immobilized on the stationary phase, or of estimated cell counts of any of the foregoing. In some embodiments, the incubating is in the presence of between or between about 1 μg and 16 μg of the VBP reagent per 10⁶ cells of the target cells, of the target cells immobilized on the stationary phase, or of estimated cell counts of any of the foregoing. In some embodiments, the incubating is in the presence of between or between about 1 μg and 12 μg of the VBP reagent per 10⁶ cells of the target cells, of the target cells immobilized on the stationary phase, or of estimated cell counts of any of the foregoing. In some embodiments, the incubating is in the presence of between or between about 1 μg and 8 μg of the VBP reagent per 10⁶ cells of the target cells, of the target cells immobilized on the stationary phase, or of estimated cell counts of any of the foregoing. In some embodiments, the incubating is in the presence of between or between about 1 μg and 6 μg of the VBP reagent per 10⁶ cells of the target cells, of the target cells immobilized on the stationary phase, or of estimated cell counts of any of the foregoing. In some embodiments, the incubating is in the presence of between or between about 2 μg and 20 μg of the VBP reagent per 10⁶ cells of the target cells, of the target cells immobilized on the stationary phase, or of estimated cell counts of any of the foregoing. In some embodiments, the incubating is in the presence of between or between about 2 μg and 16 μg of the VBP reagent per 10⁶ cells of the target cells, of the target cells immobilized on the stationary phase, or of estimated cell counts of any of the foregoing. In some embodiments, the incubating is in the presence of between or between about 2 μg and 12 μg of the VBP reagent per 10⁶ cells of the target cells, of the target cells immobilized on the stationary phase, or of estimated cell counts of any of the foregoing. In some embodiments, the incubating is in the presence of between or between about 2 μg and 8 μg of the VBP reagent per 10⁶ cells of the target cells, of the target cells immobilized on the stationary phase, or of estimated cell counts of any of the foregoing. In some embodiments, the incubating is in the presence of between or between about 2 μg and 6 μg of the VBP reagent per 10⁶ cells of the target cells, of the target cells immobilized on the stationary phase, or of estimated cell counts of any of the foregoing. In some embodiments, the incubating is in the presence of between or between about 3 μg and 5 μg of the VBP reagent per 10⁶ cells of the target cells, of the target cells immobilized on the stationary phase, or of estimated cell counts of any of the foregoing. In some embodiments, the incubating is in the presence of between or between about 3.5 μg and 4.5 μg of the VBP reagent per 10⁶ cells of the target cells, of the target cells immobilized on the stationary phase, or of estimated cell counts of any of the foregoing. In some embodiments, the incubating is in the presence of about 3.5 μg of the VBP reagent per 10⁶ cells of the target cells, of the target cells immobilized on the stationary phase, or of estimated cell counts of any of the foregoing. In some embodiments, the incubating is in the presence of about 4 μg of the VBP reagent per 10⁶ cells of the target cells, of the target cells immobilized on the stationary phase, or of estimated cell counts of any of the foregoing. In some embodiments, the incubating is in the presence of about 4.5 μg of the VBP reagent per 10⁶ cells of the target cells, of the target cells immobilized on the stationary phase, or of estimated cell counts of any of the foregoing. In any of the foregoing embodiments, the amount of the VBP reagent is per 10⁶ cells of the target cells. In any of the foregoing embodiments, the amount of the VBP reagent is per 10⁶ cells of the estimated count of the target cells immobilized on the stationary phase. In any of the foregoing embodiments, the amount of the VBP reagent is per 10⁶ cells of the binding capacity of the stationary phase. In some embodiments, the amount of VBP reagent that is present in any of the described compositions containing the VBP reagent, e.g., those contacted to the target cells or added to the internal cavity, is any of those described in the foregoing embodiments.

In some embodiments, the VBP reagent contains a weight ratio of protein reagent:VBP that is between about 10:1 and 2:1, inclusive. In some embodiments, the VBP reagent contains a weight ratio of protein reagent:VBP that is between about 8:1 and 2:1, inclusive. In some embodiments, the VBP reagent contains a weight ratio of protein reagent:VBP that is between about 8:1 and 4:1, inclusive. In some embodiments, the VBP reagent contains a weight ratio of protein reagent:VBP that is about 6:1. In some embodiments, 3.5 μg of the VBP reagent contains about 3 μg of protein reagent and 0.5 μg of the VBP.

In some embodiments, the VBP reagent contains a weight ratio of protein reagent:VBP:binding agent (e.g., anti-CD4 or anti-CD8 binding agent) that is between about 10:1:1 and 2:1:1, inclusive. In some embodiments, the VBP reagent contains a weight ratio of protein reagent:VBP:binding agent (e.g., anti-CD4 or anti-CD8 binding agent) that is between about 8:1:1 and 2:1:1, inclusive. In some embodiments, the VBP reagent contains a weight ratio of protein reagent:VBP:binding agent (e.g., anti-CD4 or anti-CD8 binding agent) that is between about 8:1:1 and 4:1:1, inclusive. In some embodiments, the VBP reagent contains a weight ratio of protein reagent:VBP:binding agent (e.g., anti-CD4 or anti-CD8 binding agent) that is about 6:1:1. In some embodiments, 4 μg of the VBP reagent contains about 3 μg of protein reagent, 0.5 μg of the VBP, and 0.5 μg of the binding agent (e.g., anti-CD4 or anti-CD8 binding agent).

In some embodiments, the VBP reagent contains a weight ratio of protein reagent:VBP:first binding agent (e.g., anti-CD4 binding agent):second binding agent (e.g., anti-CD8 binding agent) that is between about 10:1:1:1 and 2:1:1:1, inclusive. In some embodiments, the VBP reagent contains a weight ratio of protein reagent:VBP:first binding agent (e.g., anti-CD4 binding agent):second binding agent (e.g., anti-CD8 binding agent) that is between about 8:1:1:1 and 2:1:1:1, inclusive. In some embodiments, the VBP reagent contains a weight ratio of protein reagent:VBP:first binding agent (e.g., anti-CD4 binding agent):second binding agent (e.g., anti-CD8 binding agent) that is between about 8:1:1:1 and 4:1:1:1, inclusive. In some embodiments, the VBP reagent contains a weight ratio of protein reagent:VBP:first binding agent (e.g., anti-CD4 binding agent):second binding agent (e.g., anti-CD8 binding agent) that is about 6:1:1:1. In some embodiments, 4.5 μg of the VBP reagent contains about 3 μg of protein reagent, 0.5 μg of the VBP, 0.5 μg of the first binding agent (e.g., anti-CD4 binding agent), and 0.5 μg of the second binding agent (e.g., anti-CD8 binding agent).

In some embodiments, the incubating is in the presence of between or between about 0.1 μL and 100 μL, inclusive, of a preparation of the viral particle per 1×10⁶ cells of target cells, of target cells immobilized on the stationary phase, or of estimated cell counts of any of the foregoing. In some embodiments, the incubating is in the presence of between or between about 0.5 μL and 50 μL, inclusive, of a preparation of the viral particle per 1×10⁶ cells of target cells, of target cells immobilized on the stationary phase, or of estimated cell counts of any of the foregoing. In some embodiments, the incubating is in the presence of between or between about 1 μL and 25 μL, inclusive, of a preparation of the viral particle per 1×10⁶ cells of target cells, of target cells immobilized on the stationary phase, or of estimated cell counts of any of the foregoing. In some embodiments, the incubating is in the presence of between or between about 2 μL and 10 μL, inclusive, of a preparation of the viral particle per 1×10⁶ cells of target cells, of target cells immobilized on the stationary phase, or of estimated cell counts of any of the foregoing. In some embodiments, the incubating is in the presence of between or between about 4 μL and 8 μL, inclusive, of a preparation of the viral particle per 1×10⁶ cells of target cells, of target cells immobilized on the stationary phase, or of estimated cell counts of any of the foregoing. In some embodiments, the incubating is in the presence of about 6 μL of a preparation of the viral particle per 1×10⁶ cells of target cells, of target cells immobilized on the stationary phase, or of estimated cell counts of any of the foregoing. In any of the foregoing embodiments, the amount of the viral particle preparation is per 10⁶ cells of the target cells. In any of the foregoing embodiments, the amount of the viral particle preparation is per 10⁶ cells of the estimated count of the target cells immobilized on the stationary phase. In any of the foregoing embodiments, the amount of the viral particle preparation is per 10⁶ cells of the binding capacity of the stationary phase. In some embodiments, the amount of viral particle preparation that is present in any of the described compositions containing the viral particle, e.g., those contacted to the target cells or added to the internal cavity, is any of those described in the foregoing embodiments.

In some embodiments, the preparation of the viral vector has a titer of between or between about 1×10⁶ TU/mL and 1×10⁹ TU/mL. In some embodiments, the preparation of the viral vector has a titer of between or between about 1×10⁶ TU/mL and 1×10⁸ TU/mL. In some embodiments, the preparation of the viral vector has a titer of between or between about 1×10⁶ TU/mL and 1×10⁷ TU/mL. In some embodiments, the preparation of the viral vector has a titer of between or between about 1×10⁷ TU/mL and 1×10⁹ TU/mL. In some embodiments, the preparation of the viral vector has a titer of between or between about 1×10⁷ TU/mL and 1×10⁸ TU/mL. In some embodiments, the preparation of the viral vector has a titer of between or between about 1×10⁸ TU/mL and 1×10⁹ TU/mL.

In some embodiments, the incubating is carried out in a cell medium. In some embodiments, the one or more compositions containing the VBP reagent and/or viral particle each contain a cell medium.

In some embodiments, the cell medium is a serum free medium. In some embodiments, the serum free medium is a defined or well-defined cell culture medium. In certain embodiments, the serum free medium is a controlled culture medium that has been processed, e.g., filtered, to remove inhibitors and/or growth factors. In some embodiments, the serum free medium contains proteins. In some embodiments, the serum-free medium contains serum albumin, hydrolysates, growth factors, hormones, carrier proteins, and/or attachment factors. In some embodiments, the serum free medium comprises glutamine.

In some embodiments, the cell medium contains one or more cytokines. In certain embodiments, the one or more cytokines are recombinant cytokines. In particular embodiments, the one or more cytokines are human recombinant cytokines. In certain embodiments, the one or more cytokines bind to receptors that are expressed by T cells. In particular embodiments, the one or more cytokines include a member of the 4-alpha-helix bundle family of cytokines. In some embodiments, members of the 4-alpha-helix bundle family of cytokines include interleukin-2 (IL-2), interleukin-4 (IL-4), interleukin-7 (IL-7), interleukin-9 (IL-9), interleukin 12 (IL-12), interleukin 15 (IL-15), granulocyte colony-stimulating factor (G-CSF), and granulocyte-macrophage colony-stimulating factor (GM-CSF). In some embodiments, the one or more cytokines include IL-15. In particular embodiments, the one or more cytokines include IL-7. In particular embodiments, the one or more cytokines include IL-2. In particular embodiments, the one or more cytokines are selected from IL-2, IL-15, and IL-7. In particular embodiments, the cell medium contains recombinant IL-2, IL-15, and IL-7.

In certain embodiments, the amount or concentration of the one or more cytokines are measured and/or quantified with International Units (IU). International units may be used to quantify vitamins, hormones, cytokines, vaccines, blood products, and similar biologically active substances. In some embodiments, IU are or include units of measure of the potency of biological preparations by comparison to an international reference standard of a specific weight and strength, e.g., WHO 1st International Standard for Human IL-2, 86/504. International Units are the only recognized and standardized method to report biological activity units that are published and are derived from an international collaborative research effort. In particular embodiments, the IU for population, sample, or source of a cytokine may be obtained through product comparison testing with an analogous WHO standard product. For example, in some embodiments, the IU/mg of a population, sample, or source of human recombinant IL-2, IL-7, or IL-15 is compared to the WHO standard IL-2 product (NIBSC code: 86/500), the WHO standard IL-17 product (NIBSC code: 90/530), and the WHO standard IL-15 product (NIBSC code: 95/554), respectively.

In particular embodiments, the ED50 of recombinant human IL-2 or IL-15 is equivalent to the concentration required for the half-maximal stimulation of cell proliferation (XTT cleavage) with CTLL-2 cells. In certain embodiments, the ED50 of recombinant human IL-7 is equivalent to the concentration required for the half-maximal stimulation for proliferation of PHA-activated human peripheral blood lymphocytes. Details relating to assays and calculations of IU for IL-2 are discussed in Wadhwa et al., Journal of Immunological Methods (2013), 379 (1-2): 1-7; and Gearing and Thorpe, Journal of Immunological Methods (1988), 114 (1-2): 3-9; details relating to assays and calculations of IU for IL-15 are discussed in Soman et al. Journal of Immunological Methods (2009) 348 (1 2): 83-94.

In some embodiments, the cell medium contains IL-2, e.g., human recombinant IL-2, at a concentration between 1 IU/mL and 500 IU/mL, between 10 IU/mL and 250 IU/mL, between 50 IU/mL and 200 IU/mL, between 50 IU/mL and 150 IU/mL, between 75 IU/mL and 125 IU/mL, between 100 IU/mL and 200 IU/mL, or between 10 IU/mL and 100 IU/mL. In particular embodiments, the cell medium contains recombinant IL-2 at a concentration at or at about 50 IU/mL, 60 IU/mL, 70 IU/mL, 80 IU/mL, 90 IU/mL, 100 IU/mL, 110 IU/mL, 120 IU/mL, 130 IU/mL, 140 IU/mL, 150 IU/mL, 160 IU/mL, 170 IU/mL, 180 IU/mL, 190 IU/mL, or 100 IU/mL. In some embodiments, the cell medium contains about 100 IU/mL of recombinant IL-2, e.g., human recombinant IL-2.

In some embodiments, the cell medium contains recombinant IL-7, e.g., human recombinant IL-7, at a concentration between 100 IU/mL and 2,000 IU/mL, between 500 IU/mL and 1,000 IU/mL, between 100 IU/mL and 500 IU/mL, between 500 IU/mL and 750 IU/mL, between 750 IU/mL and 1,000 IU/mL, or between 550 IU/mL and 650 IU/mL. In particular embodiments, the cell medium contains IL-7 at a concentration at or at about 50 IU/mL, 100 IU/mL, 150 IU/mL, 200 IU/mL, 250 IU/mL, 300 IU/mL, 350 IU/mL, 400 IU/mL, 450 IU/mL, 500 IU/mL, 550 IU/mL, 600 IU/mL, 650 IU/mL, 700 IU/mL, 750 IU/mL, 800 IU/mL, 750 IU/mL, 750 IU/mL, 750 IU/mL, or 1,000 IU/mL. In particular embodiments, the cell medium contains about 600 IU/mL of IL-7, e.g., human recombinant IL-7.

In some embodiments, the cell medium contains recombinant IL-15, e.g., human recombinant IL-15, at a concentration between 1 IU/mL and 500 IU/mL, between 10 IU/mL and 250 IU/mL, between 50 IU/mL and 200 IU/mL, between 50 IU/mL and 150 IU/mL, between 75 IU/mL and 125 IU/mL, between 100 IU/mL and 200 IU/mL, or between 10 IU/mL and 100 IU/mL. In particular embodiments, the cell medium contains recombinant IL-15 at a concentration at or at about 50 IU/mL, 60 IU/mL, 70 IU/mL, 80 IU/mL, 90 IU/mL, 100 IU/mL, 110 IU/mL, 120 IU/mL, 130 IU/mL, 140 IU/mL, 150 IU/mL, 160 IU/mL, 170 IU/mL, 180 IU/mL, 190 IU/mL, or 200 IU/mL. In some embodiments, the cell medium contains about 100 IU/mL of recombinant IL-15, e.g., human recombinant IL-15.

In some embodiments, following the initiation of the incubation in the presence of the viral particle, the target cells are incubated in the internal cavity of the chromatography. In some embodiments, the incubating is performed for, for about, or for less than one day. In some embodiments, the incubating is performed for, for about, or for less than, 24, 23, 22, 21, 20, 19, 18, 17, 16, 15, 14, 13, 12, 11, 10, 9, 8, 7, 6, 5, 4, 3, or 2 hours. In some embodiments, the incubating is performed for between or between about 2 to 24, 3 to 24, 4 to 24, 5, to 24, 6 to 24, 7 to 24, 8 to 24, 9 to 24, 10 to 24, 11 to 24, 12 to 24, 13 to 24, 14 to 24, 15 to 24, 16 to 24, 17 to 24, 18 to 24, 19 to 24, 20 to 24, 21 to 24, 22 to 24, 23 to 24, 2 to 23, 2 to 22, 2 to 21, 2 to 20, 2 to 19, 2 to 18, 2 to 17, 2 to 16, 2 to 15, 2 to 14, 2 to 13, 2 to 12, 2 to 11, 2 to 10, 2 to 9, 2 to 8, 2 to 7, 2 to 6, 2 to 5, 2 to 4, or 2 to 3 hours. In some embodiments, the incubating is performed for, for about, or for less than, 24 hours. In some embodiments, the incubating is performed for, for about, or for less than, 12 hours. In some embodiments, the incubating is performed for, for about, or for less than, 5 hours. In some embodiments, the incubating is performed for, for about, or for less than, 4 hours. In some embodiments, the incubating is performed for, for about, or for less than, 2 hours.

In some embodiments, the incubating is carried out at room temperature (e.g., at or about 23° C.). In some embodiments, the incubating is carried out between about 30° C. and about 39° C., such as at or about 37° C.

### D. Collection

In some embodiments, the provided methods involve collecting the one or more transduced target cells. In some embodiments, the one or more transduced target cells are collected from the chromatography column. In some embodiments, the collecting includes eluting the one or more transduced target cells from the chromatography column.

In some embodiments, the collecting is carried out at, at about, or at least 2, 3, 4, 5, 6, 7, 8, 9, 10, 11, 12, 13, 14, 15, 16, 17, 18, 19, 20, 21, 22, 23, or 24 hours after initiation of the incubation with the viral particle. In some embodiments, the collecting is carried out within or within about 2, 3, 4, 5, 6, 7, 8, 9, 10, 11, 12, 13, 14, 15, 16, 17, 18, 19, 20, 21, 22, 23, or 24 hours after initiation of the incubation with the viral particle. In some embodiments, the collecting is carried out about 2 to 24, 3 to 24, 4 to 24, 5, to 24, 6 to 24, 7 to 24, 8 to 24, 9 to 24, 10 to 24, 11 to 24, 12 to 24, 13 to 24, 14 to 24, 15 to 24, 16 to 24, 17 to 24, 18 to 24, 19 to 24, 20 to 24, 21 to 24, 22 to 24, 23 to 24, 2 to 23, 2 to 22, 2 to 21, 2 to 20, 2 to 19, 2 to 18, 2 to 17, 2 to 16, 2 to 15, 2 to 14, 2 to 13, 2 to 12, 2 to 11, 2 to 10, 2 to 9, 2 to 8, 2 to 7, 2 to 6, 2 to 5, 2 to 4, or 2 to 3 hours after initiation of the incubation with the viral particle. In some embodiments, the collecting is carried out about 2 to 24 hours after initiation of the incubation with the viral particle. In some embodiments, the collecting is carried out about 2 to 12 hours after initiation of the incubation with the viral particle. In some embodiments, the collecting is carried out about 2 to 6 hours after initiation of the incubation with the viral particle. In some embodiments, the collecting is carried out about 3 to 5 hours after initiation of the incubation with the viral particle. In some embodiments, the collecting is carried out about 4.5 hours after initiation of the incubation with the viral particle.

In some embodiments, the collecting involves adding a wash buffer to the chromatography column to collect the transduced target cells. In some embodiments, the wash buffer is a cell medium. In some embodiments, the cell medium contains one or more recombinant cytokines, e.g., any as described herein, for instance any as described in Section IV-C. In some embodiments, the one or more recombinant cytokines for the wash buffer are selected from IL-2, IL-15, and IL-7. In some embodiments, the wash buffer contains recombinant IL-2, IL-15, and IL-7. In some embodiments, the recombinant IL-2, IL-15, and IL-7 are at any of the concentrations described herein, for instance any as described in Section IV-C. In some embodiments, the cell medium is a serum free medium, e.g., any as described herein, for instance any as described in Section IV-C.

In some embodiments, the collecting can be performed without the addition of a competition agent to elute the transduced target cells from the stationary phase. In some embodiments, the cell medium does not contain a competition agent to elute the one or more transduced target cells from the stationary phase. In some embodiments, the cell medium contains a competition agent to elute the transduced target cells from the stationary phase.

In some embodiments, the competition agent facilitates detachment of the transduced target cells, e.g., any immobilized transduced target cells, from the stationary phase. In some embodiments, the competition agent disrupts the immobilization of the target cells on the stationary phase. In some embodiments, the competition agent disrupts the immobilization of the selection agent on the chromatography matrix of the stationary phase. For instance, in some embodiments, the stationary phase contains a molecule that is any of the streptavidin, avidin, streptavidin analog or mutein, or avidin analog or mutein molecules described herein, and the binding partner of the selection agent is any of the binding partners, e.g., streptavidin or avidin binding partners, such as streptavidin-binding peptides, described herein that reversibly binds to the molecule, for instance with reduced binding affinity compared to that of streptavidin to biotin, or such that the binding is disrupted in the presence of biotin. In some embodiments, the competition agent has higher binding affinity for the molecule than does the binding partner of the selection agent. In some embodiments, the competition agent disrupts the binding of the binding partner of the selection agent to the molecule. In some embodiments, the competition agent is biotin, e.g., D-biotin. In some embodiments, the competition agent is a biotin analog or derivative, e.g., any as described herein.

In some embodiments, the chromatography column and collection containers are connected in a closed system. In some embodiments, the closed system is sterile. In some embodiments, the selection, stimulation, transduction, and elution steps are performed by an automated system with minimal or no manual, such as human, operation or interference.

### E. Culture

In some embodiments, the provided methods involve further incubating, e.g., culturing, the collected transduced target cells. In some embodiments, the further incubation occurs outside of the chromatography column.

In some embodiments, the further incubation is effected under conditions to result in integration of the viral particle into a host genome of one or more of the target cells. It is within the level of a skilled artisan to assess or determine if the incubation has resulted in integration of viral particles into a host genome, and hence to empirically determine the conditions for a further incubation. In some embodiments, integration of a viral particle into a host genome can be assessed by measuring the level of expression of the recombinant protein encoded by the nucleic acid contained in the viral particle. A number of well-known methods for assessing expression level of recombinant molecules may be used, such as detection by affinity-based methods, e.g., immunoaffinity-based methods, e.g., in the context of cell surface proteins, such as by flow cytometry. In some examples, the expression is measured by detection of a transduction marker and/or reporter construct. In some embodiments, a nucleic acid encoding a truncated surface protein is included within the viral particle and used as a marker of expression.

In certain embodiments, the collected target cells are further incubated (e.g., cultured) after elution for, for about, or for at least 18 hours, 24 hours, 30 hours, 36 hours, 40 hours, 48 hours, 54 hours, 60 hours, 72 hours, 84 hours, 96 hours, or more than 96 hours. In some embodiments, the further incubating (e.g., culturing) is performed for an amount of time between 30 minutes and 2 hours, between 1 hour and 8 hours, between 6 hours and 12 hours, between 12 hours and 18 hours, between 16 hours and 24 hours, between 18 hours and 30 hours, between 24 hours and 48 hours, between 24 hours and 72 hours, between 42 hours and 54 hours, between 60 hours and 120 hours between 96 hours and 120 hours, between 90 hours and between 1 days and 7 days, between 3 days and 8 days, between 1 day and 3 days, between 4 days and 6 days, or between 4 days and 5 days following elution. In some embodiments, the further incubating is carried out for no more than 14 days. In some embodiments, the further incubating is carried out for no more than 12 days. In some embodiments, the further incubating is carried out for no more than 10 days. In some embodiments, the further incubating is carried out for no more than 8 days. In some embodiments, the further incubating is carried out for no more than 6 days. In some embodiments, the further incubating is carried out for no more than 5 days. In some embodiments, the further incubating is for or for about between 18 hours and 30 hours. In particular embodiments, the further incubating is for or for about 24 hours.

In some embodiments, the collected target cells are further incubated under conditions to maintain a target amount of carbon dioxide in the cell culture. In some aspects, the amount of carbon dioxide (CO₂) is between 10% and 0% (v/v) of said gas, such as between 8% and 2% (v/v) of said gas, for example an amount of or about 5% (v/v) CO₂.

In some embodiments, the collected target cells are further incubated in the presence of the same or a similar medium as was present during the transduction of the target cells. In some embodiments, the collected target cells are incubated in a medium having the same cytokines as the medium present during transduction of the target cells. In certain embodiments, the collected target cells are incubated in a medium having the same cytokines at the same concentrations as the medium present during transduction of the target cells.

In some embodiments, the further incubating is carried out in a cell medium containing one or more recombinant cytokines, e.g., any as described herein, for instance any as described in Section IV-C. In some embodiments, the one or more recombinant cytokines for the further incubating are selected from IL-2, IL-15, and IL-7. In some embodiments, the cell medium for the further incubating contains recombinant IL-2, IL-15, and IL-7. In some embodiments, the recombinant IL-2, IL-15, and IL-7 are at any of the concentrations described herein, for instance any as described in Section IV-C. In some embodiments, the cell medium is a serum free medium, e.g., any as described herein, for instance any as described in Section IV-C.

In some embodiments, the collected transduced cells are further incubated in the absence of recombinant cytokines.

In some embodiments, the collected transduced cells are further incubated in a basal medium. In some embodiments, the basal medium is without any recombinant cytokines. In some embodiments, the basal medium is serum-free. In some embodiments, the basal medium is free of serum derived from human. In some embodiments, the basal medium contains a mixture of inorganic salts, sugars, amino acids, and, optionally, vitamins, organic acids and/or buffers or other well known cell culture nutrients. In addition to nutrients, the medium can also help maintain pH and osmolality. A wide variety of commercially available basal media are well known to those skilled in the art, and include Dulbeccos' Modified Eagles Medium (DMEM), Roswell Park Memorial Institute Medium (RPMI), Iscove modified Dulbeccos' medium and Hams medium. In some embodiments, the basal medium is Iscove's Modified Dulbecco's Medium, RPMI-1640, or α-MEM.

In some embodiments, the basal medium is a balanced salt solution (e.g., PBS, DPBS, HBSS, EBSS). In some embodiments, the basal medium is selected from Dulbecco's Modified Eagle's Medium (DMEM), Minimal Essential Medium (MEM), Basal Medium Eagle (BME), F-10, F-12, RPMI 1640, Glasgow's Minimal Essential Medium (GMEM), alpha Minimal Essential Medium (alpha MEM), Iscove's Modified Dulbecco's Medium, and M199. In some embodiments, the base medium is a complex medium (e.g., RPMI-1640, IMDM). In some embodiments, the base medium is OpTmizer™ CTS™ T-Cell Expansion Basal Medium (ThermoFisher).

In certain embodiments, the basal medium is supplemented with additional additives. In some embodiments, the basal medium is not supplemented with any additional additives. Additives to cell culture media include nutrients, sugars, e.g., glucose, amino acids, vitamins, or additives such as ATP and NADH.

In some embodiments, the further incubating is carried out under conditions to induce expansion of the collected target cells. In particular embodiments, the cultivating conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to promote growth, division, and/or expansion of the collected target cells. In some embodiments, the further incubation is carried out for a time period until a desired or threshold density, concentration, or number of cells is achieved.

In some embodiments, the further incubating is carried out in a bioreactor. Examples of suitable bioreactors for the further incubating include GE Xuri W25, GE Xuri W5, Sartorius BioSTAT RM 20|50, Finesse SmartRocker Bioreactor Systems, and Pall XRS Bioreactor Systems. In some embodiments, the bioreactor is used to perfuse and/or mix the collected target cells during at least a portion of the further incubation step.

In some embodiments, the further incubating is carried out under conditions in which there is minimal or no further expansion of the collected one or more transduced target cells. In some embodiments, the collected target cells are not further incubated under conditions that increase the amount of cells during the further incubation. In some embodiments, the collected target cells are further incubated under conditions that may result in expansion, but the further incubating conditions are not carried out for purposes of expanding the collected target cells.

In some embodiments, the further incubation (e.g., culturing) occurs in an incubator. In some embodiments, the collected target cells are transferred into a container for the further incubation. In some embodiments, the container is a vial. In particular embodiments, the container is a bag. In some embodiments, the collected target cells are transferred into the container under closed or sterile conditions. In some embodiments, the container, e.g., the vial or bag, is then placed into an incubator for all or a portion of the incubation. In particular embodiments, the incubator is set at, at about, or at least 16° C., 24° C., or 35° C. In some embodiments, the incubator is set at 37° C., at about at 37° C., or at 37° C.±2° C., ±1° C., ±0.5° C., or ±0.1° C.

In certain embodiments, the further incubation (e.g., culturing) is performed under static conditions, such as conditions that do not involve centrifugation, shaking, rotating, rocking, or perfusion of media. In some embodiments, the further incubation (e.g., culturing) is performed under gentle mixing conditions, e.g., involving rocking.

### F. Harvest

In some embodiments, the provided methods involve harvesting the transduced target cells. In some embodiments, the harvesting is performed following the further incubating. In some embodiments, the harvesting is performed following the collecting of the transduced target cells and without any further incubating of the transduced target cells. In some embodiments, the harvested transduced target cells are formulated in a container, such as a bag or vial.

In some embodiments, the harvested transduced target cells are formulated for administration to a subject. In some embodiments, the harvested transduced target cells are formulated in a pharmaceutically acceptable buffer, which may, in some aspects, include a pharmaceutically acceptable carrier or excipient. In some embodiments, the harvested transduced target cells are formulated in the presence of a pharmaceutically acceptable excipient. Exemplary formulations are described in Section VII.

In some embodiments, the harvested transduced target cells are formulated for cryopreservation. In some embodiments, the harvested transduced target cells are formulated in the presence of a cryoprotectant. In some embodiments, the harvested transduced target cells are formulated with a cyropreservative solution that contains 1.0% to 30% DMSO solution, such as a 5% to 20% DMSO solution or a 5% to 10% DMSO solution. In some embodiments, the cryopreservation solution is or contains, for example, PBS containing 20% DMSO and 8% human serum albumin (HSA), or other suitable cell freezing medium. In some embodiments, the cryopreservative solution is or contains, for example, at least or about 7.5% DMSO. In some embodiments, the harvested transduced target cells are frozen, e.g., cryoprotected or cryopreserved, in media and/or solution with a final concentration of or of about 12.5%, 12.0%, 11.5%, 11.0%, 10.5%, 10.0%, 9.5%, 9.0%, 8.5%, 8.0%, 7.5%, 7.0% 6.5%, 6.0%, 5.5%, or 5.0% DMSO, or between 1% and 15%, between 6% and 12%, between 5% and 10%, or between 6% and 8% DMSO. In particular embodiments, the harvested transduced target cells are frozen, e.g., cryoprotected or cryopreserved, in media and/or solution with a final concentration of or of about 5.0%, 4.5%, 4.0%, 3.5%, 3.0%, 2.5%, 2.0%, 1.5%, 1.25%, 1.0%, 0.75%, 0.5%, or 0.25% HSA, or between 0.1% and −5%, between 0.25% and 4%, between 0.5% and 2%, or between 1% and 2% HSA.

### V. Viral Particles

In some embodiments, the viral particle contains a nucleic acid sequence encoding a recombinant protein. In some embodiments, the viral particle is a recombinant infectious virus particle. In some embodiments, the viral particle is a viral vector, such as a vector derived from simian virus 40 (SV40), adenoviruses, or adeno-associated virus (AAV). In some embodiments, the viral particle is a recombinant lentiviral vector or retroviral vector, such as a gamma-retroviral vector (see, e.g., Koste et al. (2014) Gene Therapy 2014 Apr. 3. doi: 10.1038/gt.2014.25; Carlens et al. (2000) Exp Hematol 28(10): 1137-46; Alonso-Camino et al. (2013) Mol Ther Nucl Acids 2, e93; Park et al., Trends Biotechnol. 2011 Nov. 29(11): 550-557. In some embodiments, the viral particle is a recombinant lentiviral vector.

In some embodiments, the retroviral vector has a long terminal repeat sequence (LTR), e.g., a retroviral vector derived from the Moloney murine leukemia virus (MoMLV), myeloproliferative sarcoma virus (MPSV), murine embryonic stem cell virus (MESV), murine stem cell virus (MSCV), spleen focus forming virus (SFFV), or adeno-associated virus (AAV). Many retroviral vectors are derived from murine retroviruses. In some embodiments, the retroviruses include those derived from any avian or mammalian cell source. The retroviruses can be amphotropic, meaning that they are capable of infecting host cells of several species, including humans. In one embodiment, the gene to be expressed replaces the retroviral gag, pol and/or env sequences. A number of illustrative retroviral systems have been described (e.g., U.S. Pat. Nos. 5,219,740; 6,207,453; 5,219,740; Miller and Rosman (1989) BioTechniques 7:980-990; Miller, A. D. (1990) Human Gene Therapy 1:5-14; Scarpa et al. (1991) Virology 180:849-852; Burns et al. (1993) Proc. Natl. Acad. Sci. USA 90:8033-8037; and Boris-Lawrie and Temin (1993) Cur. Opin. Genet. Develop. 3:102 109.

The viral vector genome can be constructed in a plasmid form that can be transfected into a packaging or producer cell line. In some embodiments, the nucleic acid encoding a recombinant protein, such as a recombinant receptor, is inserted or located in a region of the viral vector, such as in a non-essential region of the viral genome. In some embodiments, the nucleic acid is inserted into the viral genome in the place of certain viral sequences to produce a virus that is replication defective.

Any of a variety of known methods can be used to produce retroviral particles whose genome contains an RNA copy of the viral vector genome. In some embodiments, at least two components are involved in making a virus-based gene delivery system: first, packaging plasmids, encompassing the structural proteins as well as the enzymes necessary to generate a viral vector particle, and second, the viral vector itself, e.g., the genetic material to be transferred. Biosafety safeguards can be introduced in the design of one or both of these components.

In some embodiments, the packaging plasmid can contain all retroviral, such as HIV-1, proteins other than envelope proteins (Naldini et al., 1998). In other embodiments, viral vectors can lack additional viral genes, such as those that are associated with virulence, e.g., vpr, vif, vpu and nef, and/or Tat, a primary transactivator of HIV. In some embodiments, lentiviral vectors, such as HIV-based lentiviral vectors, contain only three genes of the parental virus: gag, pol and rev, which reduces or eliminates the possibility of reconstitution of a wild-type virus through recombination.

In some embodiments, the viral vector genome is introduced into a packaging cell line that contains all the components necessary to package viral genomic RNA, transcribed from the viral vector genome, into viral particles. Alternatively, the viral vector genome may contain one or more genes encoding viral components in addition to the one or more sequences, e.g., recombinant nucleic acids, of interest. In some aspects, in order to prevent replication of the genome in the target cell, however, endogenous viral genes required for replication are removed and provided separately in the packaging cell line.

In some embodiments, a packaging cell line is transfected with one or more plasmid vectors containing the components necessary to generate the particles. In some embodiments, a packaging cell line is transfected with a plasmid containing the viral vector genome, including the LTRs, the cis-acting packaging sequence and the sequence of interest, i.e. a nucleic acid encoding an antigen receptor, such as a CAR; and one or more helper plasmids encoding the virus enzymatic and/or structural components, such as Gag, pol and/or rev. In some embodiments, multiple vectors are utilized to separate the various genetic components that generate the retroviral vector particles. In some such embodiments, providing separate vectors to the packaging cell reduces the chance of recombination events that might otherwise generate replication competent viruses. In some embodiments, a single plasmid vector having all of the retroviral components can be used.

In some embodiments, the retroviral vector particle, such as lentiviral vector particle, is pseudotyped to increase the transduction efficiency of host cells. For example, a retroviral vector particle, such as a lentiviral vector particle, in some embodiments is pseudotyped with a VSV-G glycoprotein, which provides a broad cell host range extending the cell types that can be transduced. In some embodiments, a packaging cell line is transfected with a plasmid or polynucleotide encoding a non-native envelope glycoprotein, such as to include xenotropic, polytropic or amphotropic envelopes, such as Sindbis virus envelope, GALV or VSV-G.

In some embodiments, the packaging cell line provides the components, including viral regulatory and structural proteins, that are required in trans for the packaging of the viral genomic RNA into lentiviral vector particles. In some embodiments, the packaging cell line may be any cell line that is capable of expressing lentiviral proteins and producing functional lentiviral vector particles. In some aspects, suitable packaging cell lines include 293 (ATCC CCL X), 293T, HeLA (ATCC CCL 2), D17 (ATCC CCL 183), MDCK (ATCC CCL 34), BHK (ATCC CCL-10) and Cf2Th (ATCC CRL 1430) cells.

In some embodiments, the packaging cell line stably expresses the viral protein(s). For example, in some aspects, a packaging cell line containing the gag, pol, rev and/or other structural genes but without the LTR and packaging components can be constructed. In some embodiments, a packaging cell line can be transiently transfected with nucleic acid molecules encoding one or more viral proteins along with the viral vector genome containing a nucleic acid molecule encoding a heterologous protein, and/or a nucleic acid encoding an envelope glycoprotein.

In some embodiments, the viral vectors and the packaging and/or helper plasmids are introduced via transfection or infection into the packaging cell line. The packaging cell line can produce viral vector particles that contain the viral vector genome. Methods for transfection or infection are well known. Non-limiting examples include calcium phosphate, DEAE-dextran and lipofection methods, electroporation and microinjection.

When a recombinant plasmid and the retroviral LTR and packaging sequences are introduced into a special cell line (e.g., by calcium phosphate precipitation for example), the packaging sequences may permit the RNA transcript of the recombinant plasmid to be packaged into viral particles, which then may be secreted into the culture medium. The medium containing the recombinant retroviruses in some embodiments is then collected, optionally concentrated, and used for gene transfer. For example, in some aspects, after cotransfection of the packaging plasmids and the transfer vector to the packaging cell line, the viral vector particles are recovered from the culture medium and titered by standard methods used by those of skill in the art.

In some embodiments, a retroviral vector, such as a lentiviral vector, can be produced in a packaging cell line, such as an exemplary HEK 293T cell line, by introduction of plasmids to allow generation of lentiviral particles. In some embodiments, a packaging cell is transfected and/or contains a polynucleotide encoding gag and pol, and a polynucleotide encoding a recombinant receptor, such as an antigen receptor, for example, a CAR. In some embodiments, the packaging cell line is optionally and/or additionally transfected with and/or contains a polynucleotide encoding a rev protein. In some embodiments, the packaging cell line is optionally and/or additionally transfected with and/or contains a polynucleotide encoding a non-native envelope glycoprotein, such as VSV-G. In some such embodiments, approximately two days after transfection of cells, e.g. HEK 293T cells, the cell supernatant contains recombinant lentiviral vectors, which can be recovered and titered.

Recovered and/or produced retroviral vector particles can be used to transduce target cells using the methods as described. Once in the target cells, the viral RNA is reverse-transcribed, imported into the nucleus and stably integrated into the host genome. One or two days after the integration of the viral RNA, the expression of the recombinant protein, e.g. antigen receptor, such as CAR, can be detected.

In some embodiments, the vector is a viral vector, such as a retroviral vector. In some embodiments, the polynucleotide encoding the recombinant receptor and/or additional polypeptide(s) are introduced into the cell via retroviral or lentiviral vectors, or via transposons (see, e.g., Baum et al. (2006) Molecular Therapy: The Journal of the American Society of Gene Therapy. 13:1050-1063; Frecha et al. (2010) Molecular Therapy 18:1748 1757; and Hackett et al. (2010) Molecular Therapy 18:674-683).

In some embodiments, the one or more polynucleotide(s) or vector(s) encoding a recombinant receptor and/or additional polypeptide(s) are introduced into cells, e.g., T cells, prior to elution, cultivating, and/or expansion. This introduction of the polynucleotide(s) or vector(s) can be carried out with any suitable retroviral vector. In some embodiments, following engineering, resulting genetically engineered cells can be liberated from the initial stimulus (e.g., anti-CD3/anti-CD28 stimulus) and subsequently be stimulated in the presence of a second type of stimulus (e.g., via a de novo introduced recombinant receptor). This second type of stimulus may include an antigenic stimulus in form of a peptide/MHC molecule, the cognate (cross-linking) ligand of the genetically introduced receptor (e.g. natural antigen and/or ligand of a CAR) or any ligand (such as an antibody) that directly binds within the framework of the new receptor (e.g. by recognizing constant regions within the receptor). See, for example, Cheadle et al, “Chimeric antigen receptors for T-cell based therapy” Methods Mol Biol. 2012; 907:645-66 or Barrett et al., Chimeric Antigen Receptor Therapy for Cancer Annual Review of Medicine Vol. 65: 333-347 (2014).

In some cases, a vector may be used that does not require that the cells, e.g., T cells, are activated. In some such instances, the cells may be selected and/or transduced prior to activation.

In some embodiments, the polynucleotide encoding the recombinant receptor contains at least one promoter that is operatively linked to control expression of the recombinant receptor. In some examples, the polynucleotide contains two, three, or more promoters operatively linked to control expression of the recombinant receptor. In some embodiments, polynucleotide can contain regulatory sequences, such as transcription and translation initiation and termination codons, which are specific to the type of host (e.g., bacterium, fungus, plant, or animal) into which the polynucleotide is to be introduced, as appropriate and taking into consideration whether the polynucleotide is DNA- or RNA-based. In some embodiments, the polynucleotide can contain regulatory/control elements, such as a promoter, an enhancer, an intron, a polyadenylation signal, a Kozak consensus sequence, internal ribosome entry sites (IRES), a 2A sequence, and splice acceptor or donor. In some embodiments, the polynucleotide can contain a nonnative promoter operably linked to the nucleotide sequence encoding the recombinant receptor and/or one or more additional polypeptide(s). In some embodiments, the promoter is selected from among an RNA poll, pol II or pol III promoter. In some embodiments, the promoter is recognized by RNA polymerase II (e.g., a CMV, SV40 early region or adenovirus major late promoter). In another embodiment, the promoter is recognized by RNA polymerase III (e.g., a U6 or H1 promoter). In some embodiments, the promoter can be a non-viral promoter or a viral promoter, such as a cytomegalovirus (CMV) promoter, an SV40 promoter, an RSV promoter, and a promoter found in the long-terminal repeat of the murine stem cell virus. Other known promoters also are contemplated.

In some embodiments, the promoter is or comprises a constitutive promoter. Exemplary constitutive promoters include simian virus 40 early promoter (SV40), cytomegalovirus immediate-early promoter (CMV), human Ubiquitin C promoter (UBC), human elongation factor 1α promoter (EF1α), mouse phosphoglycerate kinase 1 promoter (PGK), and chicken β-Actin promoter coupled with CMV early enhancer (CAGG). In some embodiments, the constitutive promoter is a synthetic or modified promoter. In some embodiments, the promoter is or comprises an MND promoter, a synthetic promoter that contains the U3 region of a modified MoMuLV LTR with myeloproliferative sarcoma virus enhancer (see Challita et al. (1995) J. Virol. 69(2):748-755). In some embodiments, the promoter is a tissue-specific promoter. In another embodiment, the promoter is a viral promoter. In another embodiment, the promoter is a non-viral promoter. In some embodiments, exemplary promoters can include, but are not limited to, human elongation factor 1 alpha (EF1α) promoter or a modified form thereof or the MND promoter.

In another embodiment, the promoter is a regulated promoter (e.g., inducible promoter). In some embodiments, the promoter is an inducible promoter or a repressible promoter. In some embodiments, the promoter comprises a Lac operator sequence, a tetracycline operator sequence, a galactose operator sequence or a doxycycline operator sequence, or is an analog thereof or is capable of being bound by or recognized by a Lac repressor or a tetracycline repressor, or an analog thereof. In some embodiments, the polynucleotide does not include a regulatory element, e.g. promoter.

In some cases, the nucleic acid sequence encoding the recombinant receptor contains a signal sequence that encodes a signal peptide. In some aspects, the signal sequence may encode a signal peptide derived from a native polypeptide. In other aspects, the signal sequence may encode a heterologous or non-native signal peptide, such as the exemplary signal peptide of the GMCSFR alpha chain set forth in SEQ ID NO:40 and encoded by the nucleotide sequence set forth in SEQ ID NO:41. In some cases, the nucleic acid sequence encoding the recombinant receptor, e.g., chimeric antigen receptor (CAR) contains a signal sequence that encodes a signal peptide. Non-limiting exemplary signal peptides include, for example, the GMCSFR alpha chain signal peptide set forth in SEQ ID NO: 40 and encoded by the nucleotide sequence set forth in SEQ ID NO:40, or the CD8 alpha signal peptide set forth in SEQ ID NO:42.

In some embodiments, the polynucleotide contains a nucleic acid sequence encoding one or more additional polypeptides, e.g., one or more marker(s) and/or one or more effector molecules. In some embodiments, the one or more marker(s) includes a transduction marker, a surrogate marker and/or a resistance marker or selection marker. Among additional nucleic acid sequences introduced, e.g., encoding for one or more additional polypeptide(s), include nucleic acid sequences that can improve the efficacy of therapy, such as by promoting viability and/or function of transferred cells; nucleic acid sequences to provide a genetic marker for selection and/or evaluation of the cells, such as to assess in vivo survival or localization; nucleic acid sequences to improve safety, for example, by making the cell susceptible to negative selection in vivo as described by Lupton S. D. et al., Mol. and Cell Biol., 11:6 (1991); and Riddell et al., Human Gene Therapy 3:319-338 (1992); see also WO 1992008796 and WO 1994028143 describing the use of bifunctional selectable fusion genes derived from fusing a dominant positive selectable marker with a negative selectable marker, and U.S. Pat. No. 6,040,177.

In some embodiments, the marker is a transduction marker or a surrogate marker. A transduction marker or a surrogate marker can be used to detect cells that have been introduced with the polynucleotide, e.g., a polynucleotide encoding a recombinant receptor. In some embodiments, the transduction marker can indicate or confirm modification of a cell. In some embodiments, the surrogate marker is a protein that is made to be co-expressed on the cell surface with the recombinant receptor, e.g. CAR. In particular embodiments, such a surrogate marker is a surface protein that has been modified to have little or no activity. In certain embodiments, the surrogate marker is encoded on the same polynucleotide that encodes the recombinant receptor. In some embodiments, the nucleic acid sequence encoding the recombinant receptor is operably linked to a nucleic acid sequence encoding a marker, optionally separated by an internal ribosome entry site (IRES), or a nucleic acid encoding a self-cleaving peptide or a peptide that causes ribosome skipping, such as a 2A sequence. Extrinsic marker genes may in some cases be utilized in connection with engineered cell to permit detection or selection of cells and, in some cases, also to promote cell elimination and/or cell suicide.

Exemplary surrogate markers can include truncated forms of cell surface polypeptides, such as truncated forms that are non-functional and to not transduce or are not capable of transducing a signal or a signal ordinarily transduced by the full-length form of the cell surface polypeptide, and/or do not or are not capable of internalizing. Exemplary truncated cell surface polypeptides including truncated forms of growth factors or other receptors such as a truncated human epidermal growth factor receptor 2 (tHER2), a truncated epidermal growth factor receptor (tEGFR, exemplary tEGFR sequence set forth in SEQ ID NO: 43 or 44) or a prostate-specific membrane antigen (PSMA) or modified form thereof, such as a truncated PSMA (tPSMA). In some aspects, tEGFR may contain an epitope recognized by the antibody cetuximab (Erbitux®) or other therapeutic anti-EGFR antibody or binding molecule, which can be used to identify or select cells that have been engineered with the tEGFR construct and an encoded exogenous protein, and/or to eliminate or separate cells expressing the encoded exogenous protein. See U.S. Pat. No. 8,802,374 and Liu et al., Nature Biotech. 2016 April; 34(4): 430-434). In some aspects, the marker, e.g. surrogate marker, includes all or part (e.g., truncated form) of CD34, a NGFR, a CD19 or a truncated CD19, e.g., a truncated non-human CD19. An exemplary polypeptide for a truncated EGFR (e.g. tEGFR) comprises the sequence of amino acids set forth in SEQ ID NO: 43 or 44 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 43 or 44.

In some embodiments, the marker is or comprises a detectable protein, such as a fluorescent protein, such as green fluorescent protein (GFP), enhanced green fluorescent protein (EGFP), such as super-fold GFP (sfGFP), red fluorescent protein (RFP), such as tdTomato, mCherry, mStrawberry, AsRed2, DsRed or DsRed2, cyan fluorescent protein (CFP), blue green fluorescent protein (BFP), enhanced blue fluorescent protein (EBFP), and yellow fluorescent protein (YFP), and variants thereof, including species variants, monomeric variants, codon-optimized, stabilized and/or enhanced variants of the fluorescent proteins. In some embodiments, the marker is or comprises an enzyme, such as a luciferase, the lacZ gene from E. coli, alkaline phosphatase, secreted embryonic alkaline phosphatase (SEAP), chloramphenicol acetyl transferase (CAT). Exemplary light-emitting reporter genes include luciferase (luc), β-galactosidase, chloramphenicol acetyltransferase (CAT), β-glucuronidase (GUS) or variants thereof. In some aspects, expression of the enzyme can be detected by addition of a substrate that can be detected upon the expression and functional activity of the enzyme.

In some embodiments, the marker is a resistance maker or selection marker. In some embodiments, the resistance maker or selection marker is or comprises a polypeptide that confers resistance to exogenous agents or drugs. In some embodiments, the resistance marker or selection marker is an antibiotic resistance gene. In some embodiments, the resistance marker or selection marker is an antibiotic resistance gene confers antibiotic resistance to a mammalian cell. In some embodiments, the resistance marker or selection marker is or comprises a Puromycin resistance gene, a Hygromycin resistance gene, a Blasticidin resistance gene, a Neomycin resistance gene, a Geneticin resistance gene or a Zeocin resistance gene or a modified form thereof.

Any of the recombinant receptors and/or the additional polypeptide(s) described herein can be encoded by one or more polynucleotides containing one or more nucleic acid sequences encoding recombinant receptors, in any combinations, orientation or arrangements. For example, one, two, three or more polynucleotides can encode one, two, three or more different polypeptides, e.g., recombinant receptors or portions or components thereof, and/or one or more additional polypeptide(s), e.g., a marker and/or an effector molecule. In some embodiments, one polynucleotide contains a nucleic acid sequence encoding a recombinant receptor, e.g., CAR, or portion or components thereof, and a nucleic acid sequence encoding one or more additional polypeptide(s). In some embodiments, one vector or construct contains a nucleic acid sequence encoding a recombinant receptor, e.g., CAR, or portion or components thereof, and a separate vector or construct contains a nucleic acid sequence encoding one or more additional polypeptide(s). In some embodiments, the nucleic acid sequence encoding the recombinant receptor and the nucleic acid sequence encoding the one or more additional polypeptide(s) are operably linked to two different promoters. In some embodiments, the nucleic acid encoding the recombinant receptor is present upstream of the nucleic acid encoding the one or more additional polypeptide(s). In some embodiments, the nucleic acid encoding the recombinant receptor is present downstream of the nucleic acid encoding one or more additional polypeptide(s).

In certain cases, one polynucleotide contains nucleic acid sequences encode two or more different polypeptide chains, e.g., a recombinant receptor and one or more additional polypeptide(s), e.g., a marker and/or an effector molecule. In some embodiments, the nucleic acid sequences encoding two or more different polypeptide chains, e.g., a recombinant receptor and one or more additional polypeptide(s), are present in two separate polynucleotides. For example, two separate polynucleotides are provided, and each can be individually transferred or introduced into the cell for expression in the cell. In some embodiments, the nucleic acid sequences encoding the marker and the nucleic acid sequences encoding the recombinant receptor are present or inserted at different locations within the genome of the cell. In some embodiments, the nucleic acid sequences encoding the marker and the nucleic acid sequences encoding the recombinant receptor are operably linked to two different promoters.

In some embodiments, such as those where the polynucleotide contains a first and second nucleic acid sequence, the coding sequences encoding each of the different polypeptide chains can be operatively linked to a promoter, which can be the same or different. In some embodiments, the nucleic acid molecule can contain a promoter that drives the expression of two or more different polypeptide chains. In some embodiments, such nucleic acid molecules can be multicistronic (bicistronic or tricistronic, see e.g., U.S. Pat. No. 6,060,273). In some embodiments, the nucleic acid sequences encoding the recombinant receptor and the nucleic acid sequences encoding the one or more additional polypeptide(s) are operably linked to the same promoter and are optionally separated by an internal ribosome entry site (IRES), or a nucleic acid encoding a self-cleaving peptide or a peptide that causes ribosome skipping, such as a 2A element. For example, an exemplary marker, and optionally a ribosome skipping sequence sequence, can be any as disclosed in PCT Pub. No. WO2014031687.

In some embodiments, transcription units can be engineered as a bicistronic unit containing an IRES, which allows coexpression of gene products (e.g. encoding the recombinant receptor and the additional polypeptide) by a message from a single promoter. Alternatively, in some cases, a single promoter may direct expression of an RNA that contains, in a single open reading frame (ORF), two or three genes (e.g. encoding the marker and encoding the recombinant receptor) separated from one another by sequences encoding a self-cleavage peptide (e.g., 2A sequences) or a protease recognition site (e.g., furin). The ORF thus encodes a single polypeptide, which, either during (in the case of 2A) or after translation, is processed into the individual proteins. In some cases, the peptide, such as a T2A, can cause the ribosome to skip (ribosome skipping) synthesis of a peptide bond at the C-terminus of a 2A element, leading to separation between the end of the 2A sequence and the next peptide downstream (see, e.g., de Felipe, Genetic Vaccines and Ther. 2:13 (2004) and de Felipe et al. Traffic 5:616-626 (2004)). Various 2A elements are known. Examples of 2A sequences that can be used in the methods and system disclosed herein, without limitation, 2A sequences from the foot-and-mouth disease virus (F2A, e.g., SEQ ID NO: 45), equine rhinitis A virus (E2A, e.g., SEQ ID NO: 46), Thosea asigna virus (T2A, e.g., SEQ ID NO: 47 or 48), and porcine teschovirus-1 (P2A, e.g., SEQ ID NO: 49 or 50) as described in U.S. Patent Pub. No. 20070116690.

In some embodiments, the polynucleotide encoding the recombinant receptor and/or additional polypeptide is contained in a vector or can be cloned into one or more vector(s). In some embodiments, the one or more vector(s) can be used to transform or transfect a host cell, e.g., a cell for engineering. Exemplary vectors include vectors designed for introduction, propagation and expansion or for expression or both, such as plasmids and viral vectors. In some aspects, the vector is an expression vector, e.g., a recombinant expression vector. In some embodiments, the recombinant expression vectors can be prepared using standard recombinant DNA techniques.

In some embodiments, the vector can be a vector of the pUC series (Fermentas Life Sciences), the pBluescript series (Stratagene, LaJolla, Calif), the pET series (Novagen, Madison, Wis.), the pGEX series (Pharmacia Biotech, Uppsala, Sweden), or the pEX series (Clontech, Palo Alto, Calif.). In some cases, bacteriophage vectors, such as λG10, λGT11, λZapII (Stratagene), λEMBL4, and λNM1149, also can be used. In some embodiments, plant expression vectors can be used and include pBI01, pBI101.2, pBI101.3, pBI121 and pBIN19 (Clontech). In some embodiments, animal expression vectors include pEUK-Cl, pMAM and pMAMneo (Clontech).

### A. Recombinant Receptors

In some embodiments, the recombinant protein is an antigen receptor that is able to bind to a molecule present on the surface of a cell. Exemplary antigen receptors are described herein.

### 1. Chimeric Antigen Receptors (CARs)

In some embodiments, the recombinant protein is a chimeric antigen receptor (CAR). In some embodiments, chimeric receptors, such as a chimeric antigen receptors, contain one or more domains that combine a ligand-binding domain (e.g. antibody or antibody fragment) that provides specificity for a desired antigen (e.g., tumor antigen) with intracellular signaling domains. In some embodiments, the intracellular signaling domain is an activating intracellular domain portion, such as a T cell activating domain, providing a primary activation signal. In some embodiments, the intracellular signaling domain contains or additionally contains a costimulatory signaling domain to facilitate effector functions. In some embodiments, chimeric receptors when genetically engineered into immune cells can modulate T cell activity, and, in some cases, can modulate T cell differentiation or homeostasis, thereby resulting in genetically engineered cells with improved longevity, survival and/or persistence in vivo, such as for use in adoptive cell therapy methods.

Exemplary antigen receptors, including CARs, and methods for engineering and introducing such receptors into cells, include those described, for example, in international patent application publication numbers WO200014257, WO2013126726, WO2012/129514, WO2014031687, WO2013/166321, WO2013/071154, WO2013/123061, WO2016/0046724, WO2016/014789, WO2016/090320, WO2016/094304, WO2017/025038, WO2017/173256, U.S. patent application publication numbers US2002131960, US2013287748, US20130149337, U.S. Pat. Nos. 6,451,995, 7,446,190, 8,252,592, 8,339,645, 8,398,282, 7,446,179, 6,410,319, 7,070,995, 7,265,209, 7,354,762, 7,446,191, 8,324,353, 8,479,118, and 9,765,342, and European patent application number EP2537416, and/or those described by Sadelain et al., Cancer Discov. 2013 April; 3(4): 388-398; Davila et al. (2013) PLoS ONE 8(4): e61338; Turtle et al., Curr. Opin. Immunol., 2012 October; 24(5): 633-39; Wu et al., Cancer, 2012 Mar. 18(2): 160-75. In some aspects, the antigen receptors include a CAR as described in U.S. Pat. No. 7,446,190, and those described in International Patent Application Publication No.: WO/2014055668 A1. Examples of the CARs include CARs as disclosed in any of the aforementioned publications, such as WO2014031687, U.S. Pat. Nos. 8,339,645, 7,446,179, US 2013/0149337, U.S. Pat. Nos. 7,446,190, 8,389,282, Kochenderfer et al., 2013, Nature Reviews Clinical Oncology, 10, 267-276 (2013); Wang et al. (2012) J. Immunother. 35(9): 689-701; and Brentjens et al., Sci Transl Med. 2013 5(177). See also WO2014031687, U.S. Pat. Nos. 8,339,645, 7,446,179, US 2013/0149337, U.S. Pat. Nos. 7,446,190, and 8,389,282.

Exemplary antigen receptors, e.g., CARs, also include any described in Marofi et al., Stem Cell Res Ther 12: 81 (2021); Townsend et al., J Exp Clin Cancer Res 37: 163 (2018); Ma et al., Int J Biol Sci 15(12): 2548-2560 (2019); Zhao and Cao, Front Immunol 10: 2250 (2019); Han et al., J Cancer 12(2): 326-334 (2021); Specht et al., Cancer Res 79: 4 Supplement, Abstract P2-09-13; Byers et al., Journal of Clinical Oncology 37, no. 15_suppl (2019); Panowski et al., Cancer Res 79 (13 Supplement) 2326 (2019); and Sauer et al., Blood 134 (Supplement_1): 1932 (2019); or can contain any of the antibodies or antigen-binding fragments described in U.S. Pat. Nos. 8,153,765; 8,603,477, 8,008,450; U.S. Pub. No. US20120189622 or US20100260748; and International PCT Publication Nos. WO2006099875, WO2009080829, WO2012092612, WO2014210064.

Further exemplary antigen receptors, e.g., CARs, such as anti-BCMA CARs, include the CARs of idecabtagene vicleucel, ABECMA^{©}, BCMA02, JCARH125, JNJ-68284528 (LCAR-B38M; ciltacabtagene autoleucel; CARVYKTI™) (Janssen/Legend), P-BCMA-101 (Poseida), PBCAR269A (Poseida), P-BCMA-Allol (Poseida), Allo-715 (Pfizer/Allogene), CT053 (Carsgen), Descartes-08 (Cartesian), PHE885 (Novartis), ARI-002 (Hospital Clinic Barcelona, IDIBAPS), and CTX120 (CRISPR Therapeutics). In a particular embodiment, the CAR is the CAR of idecabtagene vicleucel cells. In a particular embodiment, the CAR is the CAR of ABECMA® cells (cells used in ABECMA® immunotherapy). In a particular embodiment, the CAR is the CAR of ciltacabtagene autoleucel cells. In a particular embodiment, the CAR is the CAR of CARVYKTI™ cells (cells used in CARVYKTI™ immunotherapy).

Exemplary antigen receptors, e.g., CARs, also include the CARs of FDA-approved products BREYANZI® (lisocabtagene maraleucel), TECARTUS™ (brexucabtagene autoleucel), KYIVIRIAH™ (tisagenlecleucel), and YESCARTA™ (axicabtagene ciloleucel), ABECMA® (idecabtagene vicleucel), and CARVYKTI™ (ciltacabtagene autoleucel). In some of any of the provided embodiments, the CAR is the CAR of BREYANZI® (lisocabtagene maraleucel), TECARTUS™ (brexucabtagene autoleucel), KYIVIRIAH™ (tisagenlecleucel), YESCARTA™ (axicabtagene ciloleucel), ABECMA® (idecabtagene vicleucel), or CARVYKTI™ (ciltacabtagene autoleucel). In some of any of the provided embodiments, the CAR is the CAR of BREYANZI® (lisocabtagene maraleucel, see Sehgal et al., 2020, Journal of Clinical Oncology 38:15_suppl, 8040; Teoh et al., 2019, Blood 134(Supplement_1):593; and Abramson et al., 2020, The Lancet 396(10254): 839-852). In some of any of the provided embodiments, the CAR is the CAR of TECARTUS™ (brexucabtagene autoleucel, see Mian and Hill, 2021, Expert Opin Biol Ther; 21(4):435-441; and Wang et al., 2021, Blood 138(Supplement 1):744). In some of any of the provided embodiments, the CAR is the CAR of KYIVIRIAH™ (tisagenlecleucel, see Bishop et al., 2022, N Engl J Med 386:629:639; Schuster et al., 2019, N Engl J Med 380:45-56; Halford et al., 2021, Ann Pharmacother 55(4):466-479; Mueller et al., 2021, Blood Adv. 5(23):4980-4991; and Fowler et al., 2022, Nature Medicine 28:325-332). In some of any of the provided embodiments, the CAR is the CAR of YESCARTA™ (axicabtagene ciloleucel, see Neelapu et al., 2017, N Engl J Med 377(26):2531-2544; Jacobson et al., 2021, The Lancet 23(1):P91-103; and Locke et al., 2022, N Engl J Med 386:640-654). In some of any of the provided embodiments, the CAR is the CAR of ABECMA® (idecabtagene vicleucel, see Raje et al., 2019, N Engl J Med 380:1726-1737; and Munshi et al., 2021, N Engl J Med 384:705-716). In some of any of the provided embodiments, the CAR is the CAR of CARVYKTI™ (ciltacabtagene autoleucel, see Berdeja et al., Lancet. 2021 Jul. 24; 398(10297):314-324; and Martin, Abstract #549 [Oral], presented at 2021 American Society of Hematology (ASH) Annual Meeting & Exposition)).

The chimeric receptors, such as CARs, generally include an extracellular antigen binding domain, such as a portion of an antibody molecule, generally a variable heavy (VH) chain region and/or variable light (VL) chain region of the antibody, e.g., an scFv antibody fragment.

In some embodiments, the antigen targeted by the receptor is a polypeptide. In some embodiments, it is a carbohydrate or other molecule. In some embodiments, the antigen is selectively expressed or overexpressed on cells of the disease or condition, e.g., the tumor or pathogenic cells, as compared to normal or non-targeted cells or tissues. In other embodiments, the antigen is expressed on normal cells and/or is expressed on the engineered cells.

In some embodiments, the antigen is or includes αvβ6 integrin (avb6 integrin), B cell maturation antigen (BCMA), B7-H3, B7-H6, carbonic anhydrase 9 (CA9, also known as CAIX or G250), a cancer-testis antigen, cancer/testis antigen 1B (CTAG, also known as NY-ESO-1 and LAGE-2), carcinoembryonic antigen (CEA), a cyclin, cyclin A2, C-C Motif Chemokine Ligand 1 (CCL-1), CD19, CD20, CD22, CD23, CD24, CD30, CD33, CD38, CD44, CD44v6, CD44v7/8, CD123, CD133, CD138, CD171, chondroitin sulfate proteoglycan 4 (CSPG4), epidermal growth factor protein (EGFR), type III epidermal growth factor receptor mutation (EGFR vIII), epithelial glycoprotein 2 (EPG-2), epithelial glycoprotein 40 (EPG-40), ephrinB2, ephrin receptor A2 (EPHa2), estrogen receptor, Fc receptor like 5 (FCRL5; also known as Fc receptor homolog 5 or FCRH5), fetal acetylcholine receptor (fetal AchR), a folate binding protein (FBP), folate receptor alpha, ganglioside GD2, O-acetylated GD2 (OGD2), ganglioside GD3, glycoprotein 100 (gp100), glypican-3 (GPC3), G Protein Coupled Receptor 5D (GPRC5D), Her2/neu (receptor tyrosine kinase erb-B2), Her3 (erb-B3), Her4 (erb-B4), erbB dimers, Human high molecular weight-melanoma-associated antigen (HMW-MAA), hepatitis B surface antigen, Human leukocyte antigen A1 (HLA-A1), Human leukocyte antigen A2 (HLA-A2), IL-22 receptor alpha(IL-22Rα), IL-13 receptor alpha 2 (IL-13Rα2), kinase insert domain receptor (kdr), kappa light chain, L1 cell adhesion molecule (L1-CAM), CE7 epitope of L1-CAM, Leucine Rich Repeat Containing 8 Family Member A (LRRC8A), Lewis Y, Melanoma-associated antigen (MAGE)-A1, MAGE-A3, MAGE-A6, MAGE-A10, mesothelin (MSLN), c-Met, murine cytomegalovirus (CMV), mucin 1 (MUC1), MUC16, natural killer group 2 member D (NKG2D) ligands, melan A (MART-1), neural cell adhesion molecule (NCAM), oncofetal antigen, Preferentially expressed antigen of melanoma (PRAME), progesterone receptor, a prostate specific antigen, prostate stem cell antigen (PSCA), prostate specific membrane antigen (PSMA), Receptor Tyrosine Kinase Like Orphan Receptor 1 (ROR1), survivin, Trophoblast glycoprotein (TPBG also known as 5T4), tumor-associated glycoprotein 72 (TAG72), Tyrosinase related protein 1 (TRP1, also known as TYRP 1 or gp75), Tyrosinase related protein 2 (TRP2, also known as dopachrome tautomerase, dopachrome delta-isomerase or DCT), vascular endothelial growth factor receptor (VEGFR), vascular endothelial growth factor receptor 2 (VEGFR2), Wilms Tumor 1 (WT-1), a pathogen-specific or pathogen-expressed antigen, or an antigen associated with a universal tag, and/or biotinylated molecules, and/or molecules expressed by HIV, HCV, HBV or other pathogens. Antigens targeted by the receptors in some embodiments include antigens associated with a B cell malignancy, such as any of a number of known B cell marker. In some embodiments, the antigen is or includes CD20, CD19, CD22, ROR1, CD45, CD21, CD5, CD33, Igkappa, Iglambda, CD79a, CD79b or CD30. In some embodiments, the antigen is or includes a pathogen-specific or pathogen-expressed antigen. In some embodiments, the antigen is a viral antigen (such as a viral antigen from HIV, HCV, HBV, etc.), bacterial antigens, and/or parasitic antigens.

Antigens targeted by the receptors in some embodiments include antigens associated with a B cell malignancy, such as any of a number of known B cell marker. In some embodiments, the antigen targeted by the receptor is CD20, CD19, CD22, ROR1, CD45, CD21, CD5, CD33, Igkappa, Iglambda, CD79a, CD79b or CD30.

In some embodiments, the antigen or antigen binding domain is CD19. In some embodiments, the scFv contains a VH and a VL derived from an antibody or an antibody fragment specific to CD19. In some embodiments, the antibody or antibody fragment that binds CD19 is a mouse derived antibody such as FMC63 and SJ25C1. In some embodiments, the antibody or antibody fragment is a human antibody, e.g., as described in U.S. Patent Publication No. US 2016/0152723. Exemplary antibody or antibody fragments that bind to CD19 are also described in WO 2014/031687, US 2016/0152723, and WO 2016/033570.

The term “antibody” herein is used in the broadest sense and includes polyclonal and monoclonal antibodies, including intact antibodies and functional (antigen-binding) antibody fragments, including fragment antigen binding (Fab) fragments, F(ab′)₂ fragments, Fab′ fragments, Fv fragments, recombinant IgG (rIgG) fragments, heavy chain variable (V_{H}) regions capable of specifically binding the antigen, single chain antibody fragments, including single chain variable fragments (scFv), and single domain antibodies (e.g., sdAb, sdFv, nanobody) fragments. The term encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific or trispecific, antibodies, diabodies, triabodies, and tetrabodies, tandem di-scFv, tandem tri-scFv. Unless otherwise stated, the term “antibody” should be understood to encompass functional antibody fragments thereof also referred to herein as “antigen-binding fragments.” The term also encompasses intact or full-length antibodies, including antibodies of any class or sub-class, including IgG and sub-classes thereof, IgM, IgE, IgA, and IgD.

The terms “complementarity determining region,” and “CDR,” synonymous with “hypervariable region” or “HVR,” are known in the art to refer to non-contiguous sequences of amino acids within antibody variable regions, which confer antigen specificity and/or binding affinity. In general, there are three CDRs in each heavy chain variable region (CDR-H1, CDR-H2, CDR-H3) and three CDRs in each light chain variable region (CDR-L1, CDR-L2, CDR-L3). “Framework regions” and “FR” are known in the art to refer to the non-CDR portions of the variable regions of the heavy and light chains. In general, there are four FRs in each full-length heavy chain variable region (FR-H1, FR-H2, FR-H3, and FR-H4), and four FRs in each full-length light chain variable region (FR-L1, FR-L2, FR-L3, and FR-L4).

The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme); Al-Lazikani et al., (1997) JMB 273, 927-948 (“Chothia” numbering scheme); MacCallum et al., J. Mol. Biol. 262:732-745 (1996), “Antibody-antigen interactions: Contact analysis and binding site topography,” J. Mol. Biol. 262, 732-745.” (“Contact” numbering scheme); Lefranc M P et al., “IMGT unique numbering for immunoglobulin and T cell receptor variable domains and Ig superfamily V-like domains,” Dev Comp Immunol, 2003 January; 27(1):55-77 (“IMGT” numbering scheme); Honegger A and Pluckthun A, “Yet another numbering scheme for immunoglobulin variable domains: an automatic modeling and analysis tool,” J Mol Biol, 2001 Jun. 8; 309(3):657-70, (“Aho” numbering scheme); and Martin et al., “Modeling antibody hypervariable loops: a combined algorithm,” PNAS, 1989, 86(23):9268-9272, (“AbM” numbering scheme).

The boundaries of a given CDR or FR may vary depending on the scheme used for identification. For example, the Kabat scheme is based on structural alignments, while the Chothia scheme is based on structural information. Numbering for both the Kabat and Chothia schemes is based upon the most common antibody region sequence lengths, with insertions accommodated by insertion letters, for example, “30a,” and deletions appearing in some antibodies. The two schemes place certain insertions and deletions (“indels”) at different positions, resulting in differential numbering. The Contact scheme is based on analysis of complex crystal structures and is similar in many respects to the Chothia numbering scheme. The AbM scheme is a compromise between Kabat and Chothia definitions based on that used by Oxford Molecular's AbM antibody modeling software.

Table 1, below, lists exemplary position boundaries of CDR-L1, CDR-L2, CDR-L3 and CDR-H1, CDR-H2, CDR-H3 as identified by Kabat, Chothia, AbM, and Contact schemes, respectively. For CDR-H1, residue numbering is listed using both the Kabat and Chothia numbering schemes. FRs are located between CDRs, for example, with FR-L1 located before CDR-L1, FR-L2 located between CDR-L1 and CDR-L2, FR-L3 located between CDR-L2 and CDR-L3 and so forth. It is noted that because the shown Kabat numbering scheme places insertions at H35A and H35B, the end of the Chothia CDR-H1 loop when numbered using the shown Kabat numbering convention varies between H32 and H34, depending on the length of the loop.

TABLE 1
Boundaries of CDRs according to various numbering schemes.

| CDR | Kabat | Chothia | AbM | Contact |
|---|---|---|---|---|
| CDR-L1 | L24--L34 | L24--L34 | L24--L34 | L30--L36 |
| CDR-L2 | L50--L56 | L50--L56 | L50--L56 | L46--L55 |
| CDR-L3 | L89--L97 | L89--L97 | L89--L97 | L89--L96 |
| CDR-H1 | H31--H35B | H26--H32 . . . 34 | H26--H35B | H30--H35B |
| (Kabat Numbering¹) | | | | |
| CDR-H1 | H31--H35 | H26--H32 | H26--H35 | H30--H35 |
| (Chothia Numbering²) | | | | |
| CDR-H2 | H50--H65 | H52--H56 | H50--H58 | H47--H58 |
| CDR-H3 | H95--H102 | H95--H102 | H95--H102 | H93--H101 |

¹Kabat et al. (1991), “Sequences of Proteins of Immunological Interest,” 5th Ed. Public
Health Service, National Institutes of Health, Bethesda, MD
²Al-Lazikani et al., (1997) JMB 273, 927-948

Thus, unless otherwise specified, a “CDR” or “complementary determining region,” or individual specified CDRs (e.g., CDR-H1, CDR-H2, CDR-H3), of a given antibody or region thereof, such as a variable region thereof, should be understood to encompass a (or the specific) complementary determining region as defined by any of the aforementioned schemes, or other known schemes. For example, where it is stated that a particular CDR (e.g., a CDR-H3) contains the amino acid sequence of a corresponding CDR in a given V_{H} or V_{L} region amino acid sequence, it is understood that such a CDR has a sequence of the corresponding CDR (e.g., CDR-H3) within the variable region, as defined by any of the aforementioned schemes, or other known schemes. In some embodiments, specific CDR sequences are specified. Exemplary CDR sequences of antibodies are described using various numbering schemes, although it is understood that an antibody can include CDRs as described according to any of the other aforementioned numbering schemes or other numbering schemes known to a skilled artisan.

Likewise, unless otherwise specified, a FR or individual specified FR(s) (e.g., FR-H1, FR-H2, FR-H3, FR-H4), of a given antibody or region thereof, such as a variable region thereof, should be understood to encompass a (or the specific) framework region as defined by any of the known schemes. In some instances, the scheme for identification of a particular CDR, FR, or FRs or CDRs is specified, such as the CDR as defined by the Kabat, Chothia, AbM or Contact method, or other known schemes. In other cases, the particular amino acid sequence of a CDR or FR is given.

The term “variable region” or “variable domain” refers to the domain of an antibody heavy or light chain that is involved in binding the antibody to antigen. The variable regions of the heavy chain and light chain (V_{H} and V_{L}, respectively) of a native antibody generally have similar structures, with each domain comprising four conserved framework regions (FRs) and three CDRs. (See, e.g., Kindt et al. Kuby Immunology, 6th ed., W.H. Freeman and Co., page 91 (2007). A single V_{H} or V_{L} domain may be sufficient to confer antigen-binding specificity. Furthermore, antibodies that bind a particular antigen may be isolated using a V_{H} or V_{L} domain from an antibody that binds the antigen to screen a library of complementary V_{L} or V_{H} domains, respectively. See, e.g., Portolano et al., J. Immunol. 150:880-887 (1993); Clarkson et al., Nature 352:624-628 (1991).

Among the antibodies included in the CARs are antibody fragments. An “antibody fragment” or “antigen-binding fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; heavy chain variable (V_{H}) regions, single-chain antibody molecules such as scFvs and single-domain antibodies comprising only the V_{H} region; and multispecific antibodies formed from antibody fragments. In some embodiments, the antigen-binding domain in the CARs is or comprises an antibody fragment comprising a variable heavy chain (V_{H}) and a variable light chain (V_{L}) region. In particular embodiments, the antibodies are single-chain antibody fragments comprising a heavy chain variable (V_{H}) region and/or a light chain variable (V_{L}) region, such as scFvs.

In some embodiments, the scFv is derived from FMC63. FMC63 generally refers to a mouse monoclonal IgG1 antibody raised against Nalm-1 and -16 cells expressing CD19 of human origin (Ling, N. R., et al. (1987). Leucocyte typing III. 302). In some embodiments, the FMC63 antibody comprises CDRH1 and H2 set forth in SEQ ID NOS: land 52, respectively, and CDRH3 set forth in SEQ ID NO: 53 or 54 and CDRL1 set forth in SEQ ID NO: 55 and CDR L2 set forth in SEQ ID NO: 55 or 57 and CDR L3 set forth in SEQ ID NO: 58 or 59. In some embodiments, the FMC63 antibody comprises the heavy chain variable region (V_{H}) comprising the amino acid sequence of SEQ ID NO: 60 and the light chain variable region (V_{L}) comprising the amino acid sequence of SEQ ID NO: 61.

In some embodiments, the scFv comprises a variable light chain containing the CDRL1 sequence of SEQ ID NO: 55, a CDRL2 sequence of SEQ ID NO: 56, and a CDRL3 sequence of SEQ ID NO: 58 and/or a variable heavy chain containing a CDRH1 sequence of SEQ ID NO: 51, a CDRH2 sequence of SEQ ID NO: 52, and a CDRH3 sequence of SEQ ID NO: 53. In some embodiments, the scFv comprises a variable heavy chain region set forth in SEQ ID NO:60 and a variable light chain region set forth in SEQ ID NO:61. In some embodiments, the variable heavy and variable light chains are connected by a linker. In some embodiments, the linker is set forth in SEQ ID NO: 62. In some embodiments, the scFv comprises, in order, a V_{H}, a linker, and a V_{L}. In some embodiments, the scFv comprises, in order, a V_{L}, a linker, and a V_{H}. In some embodiments, the scFv is encoded by a sequence of nucleotides set forth in SEQ ID NO: 63 or a sequence that exhibits at least 85%, 86%, 87%, 88%, 89%, 90%, 91%, 92%, 93%, 94%, 95%, 96%, 97%, 98%, or 99% sequence identity to SEQ ID NO: 63. In some embodiments, the scFv comprises the sequence of amino acids set forth in SEQ ID NO: 64 or a sequence that exhibits at least 85%, 86%, 87%, 88%, 89%, 90%, 91%, 92%, 93%, 94%, 95%, 96%, 97%, 98%, or 99% sequence identity to SEQ ID NO:64. In some embodiments, the scFv is that of BREYANZI® (lisocabtagene maraleucel). In some embodiments, the CAR is that of BREYANZI® (lisocabtagene maraleucel).

In some embodiments the scFv is derived from SJ25C1. SJ25C1 is a mouse monoclonal IgG1 antibody raised against Nalm-1 and -16 cells expressing CD19 of human origin (Ling, N. R., et al. (1987). Leucocyte typing III. 302). In some embodiments, the SJ25C1 antibody comprises CDRH1, H2 and H3 set forth in SEQ ID NOS: 65-67, respectively, and CDRL1, L2 and L3 sequences set forth in SEQ ID NOS:68-70, respectively. In some embodiments, the SJ25C1 antibody comprises the heavy chain variable region (V_{H}) comprising the amino acid sequence of SEQ ID NO: 71 and the light chain variable region (V_{L}) comprising the amino acid sequence of SEQ ID NO: 72.

In some embodiments, the scFv comprises a variable light chain containing the CDRL1 sequence of SEQ ID NO:73, a CDRL2 sequence of SEQ ID NO: 74, and a CDRL3 sequence of SEQ ID NO:75 and/or a variable heavy chain containing a CDRH1 sequence of SEQ ID NO:76, a CDRH2 sequence of SEQ ID NO:77, and a CDRH3 sequence of SEQ ID NO:78. In some embodiments, the scFv comprises a variable heavy chain region set forth in SEQ ID NO: 71 and a variable light chain region set forth in SEQ ID NO:72. In some embodiments, the variable heavy and variable light chain are connected by a linker. In some embodiments, the linker is set forth in SEQ ID NO:79. In some embodiments, the scFv comprises, in order, a V_{H}, a linker, and a V_{L}. In some embodiments, the scFv comprises, in order, a V_{L}, a linker, and a V_{H}. In some embodiments, the scFv comprises the sequence of amino acids set forth in SEQ ID NO:80 or a sequence that exhibits at least 85%, 86%, 87%, 88%, 89%, 90%, 91%, 92%, 93%, 94%, 95%, 96%, 97%, 98%, or 99% sequence identity to SEQ ID NO:80.

In some embodiments, the antigen or antigen binding domain is BCMA. In some embodiments, the scFv contains a VH and a VL derived from an antibody or an antibody fragment specific to BCMA. In some embodiments, the antibody or antibody fragment that binds BCMA is or contains a VH and a VL from an antibody or antibody fragment set forth in International Patent Applications, Publication Number WO 2016/090327 and WO 2016/090320.

In some embodiments, the antibody or antibody fragment that binds BCMA can be any anti-BCMA antibody described or derived from any anti-BCMA antibody described. See, e.g., Carpenter et al., Clin Cancer Res., 2013, 19(8):2048-2060; U.S. Pat. Nos. 9,034,324 9,765,342; U.S. Patent publication No. US2016/0046724, US20170183418; and International published PCT App. No. WO 2016090320, WO2016090327, WO2016094304, WO2016014565, WO106014789, WO2010104949, WO2017/025038, or WO2017173256. Any of such anti-BCMA antibodies or antigen-binding fragments can be used in the anti-BCMA CAR. In some embodiments, the anti-BCMA CAR contains an antigen-binding domain that is an scFv containing a variable heavy (V_{H}) and/or a variable light (V_{L}) region. In some embodiments, the scFv containing a variable heavy (V_{H}) and/or a variable light (V_{L}) region is derived from an antibody described in WO 2016090320 or WO2016090327. In some embodiments, the antigen or antigen binding domain is GPRC5D. In some embodiments, the scFv contains a VH and a VL derived from an antibody or an antibody fragment specific to GPRC5D. In some embodiments, the antibody or antibody fragment that binds GPRC5D is or contains a VH and a VL from an antibody or antibody fragment set forth in International Patent Applications, Publication Number WO 2016/090329, WO 2016/090312, and WO 2020/092854.

In some embodiments, the antibody or antibody fragment that binds BCMA includes a V_{H} and a V_{L} region, wherein the V_{H} region includes a CDR-H1 set forth in SEQ ID NO: 113, a CDR-H2 set forth in SEQ ID NO: 114, and a CDR-H3 set forth in SEQ ID NO:115, and the V_{L} region includes a CDR-L1 set forth in SEQ ID NO:116, a CDR-L2 set forth in SEQ ID NO:117, and a CDR-H3 set forth in SEQ ID NO:118. In some embodiments, the antibody or antibody fragment that binds BCMA includes a V_{H} region that has the sequence of amino acids set forth in SEQ ID NO: 119 or a sequence of amino acids that exhibits at least 90%, at least 91%, at least 92%, at least 93%, at least 94%, at least 95%, at least 96%, at least 97%, at least 98%, or at least 99% to SEQ ID NO: 119, and a V_{L} region that has the sequence of amino acids set forth in SEQ ID NO: 120 or a sequence of amino acids that exhibits at least 90%, at least 91%, at least 92%, at least 93%, at least 94%, at least 95%, at least 96%, at least 97%, at least 98%, or at least 99% to SEQ ID NO: 120. In some embodiments, the antibody or antibody fragment that binds BCMA includes a V_{H} region that has the sequence of amino acids set forth in SEQ ID NO: 119 and a V_{L} region that has the sequence of amino acids set forth in SEQ ID NO: 120. In some embodiments, the antibody or antibody fragment that binds BCMA is an scFv that has the sequence of amino acids set forth in SEQ ID NO: 121 or a sequence of amino acids that exhibits at least 90%, at least 91%, at least 92%, at least 93%, at least 94%, at least 95%, at least 96%, at least 97%, at least 98%, or at least 99% to SEQ ID NO:121. In some embodiments, the antibody or antibody fragment that binds BCMA is an scFv as set forth in SEQ ID NO: 121. In some embodiments, the scFv is that of ABECMA® (idecabtagene vicleucel). In some embodiments, the CAR has the sequence of amino acids set forth in SEQ ID NO: 122 or a sequence of amino acids that exhibits at least 90%, at least 91%, at least 92%, at least 93%, at least 94%, at least 95%, at least 96%, at least 97%, at least 98%, or at least 99% sequence identity to SEQ ID NO: 122. In some embodiments, the CAR is that of ABECMA® (idecabtagene vicleucel).

In some embodiments, the antibody or antibody fragment that binds BCMA includes a V_{H} and a V_{L} region, wherein the V_{H} region includes a CDR-H1 set forth in SEQ ID NO: 123, a CDR-H2 set forth in SEQ ID NO: 124, and a CDR-H3 set forth in SEQ ID NO:125, and the V_{L} region includes a CDR-L1 set forth in SEQ ID NO:126, a CDR-L2 set forth in SEQ ID NO: 127, and a CDR-H3 set forth in SEQ ID NO: 128. In some embodiments, the antibody or antibody fragment that binds BCMA includes a V_{H} region that has the sequence of amino acids set forth in SEQ ID NO: 129 or a sequence of amino acids that exhibits at least 90%, at least 91%, at least 92%, at least 93%, at least 94%, at least 95%, at least 96%, at least 97%, at least 98%, or at least 99% to SEQ ID NO: 129, and a V_{L} region that has the sequence of amino acids set forth in SEQ ID NO:130 or a sequence of amino acids that exhibits at least 90%, at least 91%, at least 92%, at least 93%, at least 94%, at least 95%, at least 96%, at least 97%, at least 98%, or at least 99% to SEQ ID NO: 130. In some embodiments, the antibody or antibody fragment that binds BCMA includes a V_{H} region that has the sequence of amino acids set forth in SEQ ID NO: 129 and a V_{L} region that has the sequence of amino acids set forth in SEQ ID NO: 130. In some embodiments, the antibody or antibody fragment that binds BCMA is an scFv that has the sequence of amino acids set forth in SEQ ID NO:131 or a sequence of amino acids that exhibits at least 90%, at least 91%, at least 92%, at least 93%, at least 94%, at least 95%, at least 96%, at least 97%, at least 98%, or at least 99% to SEQ ID NO:131. In some embodiments, the antibody or antibody fragment that binds BCMA is an scFv as set forth in SEQ ID NO: 131. In some embodiments, the scFv is that of orvacabtagene autoleucel. In some embodiments, the CAR has the sequence of amino acids set forth in SEQ ID NO: 132 or a sequence of amino acids that exhibits at least 90%, at least 91%, at least 92%, at least 93%, at least 94%, at least 95%, at least 96%, at least 97%, at least 98%, or at least 99% sequence identity to SEQ ID NO: 132. In some embodiments, the CAR is that of orvacabtagene autoleucel.

In some embodiments, the antigen is CD20. In some embodiments, the scFv contains a VH and a VL derived from an antibody or an antibody fragment specific to CD20. In some embodiments, the antibody or antibody fragment that binds CD20 is an antibody that is or is derived from Rituximab, such as is Rituximab scFv.

In some embodiments, the antigen is CD22. In some embodiments, the scFv contains a VH and a VL derived from an antibody or an antibody fragment specific to CD22. In some embodiments, the antibody or antibody fragment that binds CD22 is an antibody that is or is derived from m971, such as is m971 scFv.

In some embodiments, the antigen is ROR1. In some embodiments, the scFv contains a V_{H} and a V_{L} derived from an antibody or an antibody fragment specific to ROR1. In some embodiments, the antibody or antibody fragment that binds ROR1 is or contains a V_{H} and a V_{L} from an antibody or antibody fragment set forth in International Patent Applications, Publication Number WO 2014/031687, WO 2016/115559 and WO 2020/160050, the contents of each of which are incorporated by reference in their entirety.

In some embodiments, the antigen is FcRL5. In some embodiments, the scFv contains a V_{H} and a V_{L} derived from an antibody or an antibody fragment specific to FcRL5. In some embodiments, the antibody or antibody fragment that binds FcRL5 is or contains a V_{H} and a V_{L} from an antibody or antibody fragment set forth in International Patent Applications, Publication Number WO 2016/090337 and WO 2017/096120, the contents of each of which are incorporated by reference in their entirety.

In some embodiments, the antigen is mesothelin. In some embodiments, the scFv contains a V_{H} and a V_{L} derived from an antibody or an antibody fragment specific to mesothelin. In some embodiments, the antibody or antibody fragment that binds mesothelin is or contains a V_{H} and a V_{L} from an antibody or antibody fragment set forth in US2018/0230429, the contents of which are incorporated by reference in their entirety.

In some embodiments, the chimeric antigen receptor includes an extracellular portion containing an antibody or antibody fragment. In some aspects, the chimeric antigen receptor includes an extracellular portion containing the antibody or fragment and an intracellular signaling domain. In some embodiments, the antibody or fragment includes an scFv.

In some embodiments, the antibody portion of the recombinant receptor, e.g., CAR, further includes at least a portion of an immunoglobulin constant region, such as a hinge region, e.g., an IgG4 hinge region, and/or a CH1/CL and/or Fc region. In some embodiments, the constant region or portion is of a human IgG, such as IgG4 or IgG1. In some aspects, the portion of the constant region serves as a spacer region between the antigen-recognition component, e.g., scFv, and transmembrane domain. The spacer can be of a length that provides for increased responsiveness of the cell following antigen binding, as compared to in the absence of the spacer. Exemplary spacers include, but are not limited to, those described in Hudecek et al. (2013) Clin. Cancer Res., 19:3153, international patent application publication number WO2014031687, U.S. Pat. No. 8,822,647 or published app. No. US2014/0271635.

In some embodiments, the constant region or portion is of a human IgG, such as IgG4 or IgG1. In some embodiments, the spacer has the sequence ESKYGPPCPPCP (set forth in SEQ ID NO: 81), and is encoded by the sequence set forth in SEQ ID NO: 82. In some embodiments, the spacer has the sequence set forth in SEQ ID NO: 83. In some embodiments, the spacer has the sequence set forth in SEQ ID NO: 84. In some embodiments, the constant region or portion is of IgD. In some embodiments, the spacer has the sequence set forth in SEQ ID NO: 85. In some embodiments, the spacer has a sequence of amino acids that exhibits at least 85%, 86%, 87%, 88%, 89%, 90%, 91%, 92%, 93%, 94%, 95%, 96%, 97%, 98%, 99% or more sequence identity to any of SEQ ID NOS: 81, 83, 84 or 85. In some embodiments, the spacer has the sequence set forth in SEQ ID NOS: 86-94. In some embodiments, the spacer has a sequence of amino acids that exhibits at least 85%, 86%, 87%, 88%, 89%, 90%, 91%, 92%, 93%, 94%, 95%, 96%, 97%, 98%, 99% or more sequence identity to any of SEQ ID NOS: 86-94.

In some embodiments, the antigen receptor comprises an intracellular domain linked directly or indirectly to the extracellular domain. In some embodiments, the chimeric antigen receptor includes a transmembrane domain linking the extracellular domain and the intracellular signaling domain. In some embodiments, the intracellular signaling domain comprises an ITAM. For example, in some aspects, the antigen recognition domain (e.g. extracellular domain) generally is linked to one or more intracellular signaling components, such as signaling components that mimic activation through an antigen receptor complex, such as a TCR complex, in the case of a CAR, and/or signal via another cell surface receptor. In some embodiments, the chimeric receptor comprises a transmembrane domain linked or fused between the extracellular domain (e.g. scFv) and intracellular signaling domain. Thus, in some embodiments, the antigen-binding component (e.g., antibody) is linked to one or more transmembrane and intracellular signaling domains.

In one embodiment, a transmembrane domain that naturally is associated with one of the domains in the receptor, e.g., CAR, is used. In some instances, the transmembrane domain is selected or modified by amino acid substitution to avoid binding of such domains to the transmembrane domains of the same or different surface membrane proteins to minimize interactions with other members of the receptor complex.

The transmembrane domain in some embodiments is derived either from a natural or from a synthetic source. Where the source is natural, the domain in some aspects is derived from any membrane-bound or transmembrane protein. Transmembrane regions include those derived from (i.e. comprise at least the transmembrane region(s) of) the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137, CD154. Alternatively the transmembrane domain in some embodiments is synthetic. In some aspects, the synthetic transmembrane domain comprises predominantly hydrophobic residues such as leucine and valine. In some aspects, a triplet of phenylalanine, tryptophan and valine will be found at each end of a synthetic transmembrane domain. In some embodiments, the linkage is by linkers, spacers, and/or transmembrane domain(s). In some aspects, the transmembrane domain contains a transmembrane portion of CD28.

In some embodiments, the extracellular domain and transmembrane domain can be linked directly or indirectly. In some embodiments, the extracellular domain and transmembrane are linked by a spacer, such as any described herein. In some embodiments, the receptor contains extracellular portion of the molecule from which the transmembrane domain is derived, such as a CD28 extracellular portion.

Among the intracellular signaling domains are those that mimic or approximate a signal through a natural antigen receptor, a signal through such a receptor in combination with a costimulatory receptor, and/or a signal through a costimulatory receptor alone. In some embodiments, a short oligo- or polypeptide linker, for example, a linker of between 2 and 10 amino acids in length, such as one containing glycines and serines, e.g., glycine-serine doublet, is present and forms a linkage between the transmembrane domain and the cytoplasmic signaling domain of the CAR.

T cell activation is in some aspects described as being mediated by two classes of cytoplasmic signaling sequences: those that initiate antigen-dependent primary activation through the TCR (primary cytoplasmic signaling sequences), and those that act in an antigen-independent manner to provide a secondary or co-stimulatory signal (secondary cytoplasmic signaling sequences). In some aspects, the CAR includes one or both of such signaling components.

The receptor, e.g., the CAR, generally includes at least one intracellular signaling component or components. In some aspects, the CAR includes a primary cytoplasmic signaling sequence that regulates primary activation of the TCR complex. Primary cytoplasmic signaling sequences that act in a stimulatory manner may contain signaling motifs which are known as immunoreceptor tyrosine-based activation motifs or ITAMs. Examples of ITAM containing primary cytoplasmic signaling sequences include those derived from CD3 zeta chain, FcR gamma, CD3 gamma, CD3 delta and CD3 epsilon. In some embodiments, cytoplasmic signaling molecule(s) in the CAR contain(s) a cytoplasmic signaling domain, portion thereof, or sequence derived from CD3 zeta.

In some embodiments, the receptor includes an intracellular component of a TCR complex, such as a TCR CD3 chain that mediates T-cell activation and cytotoxicity, e.g., CD3 zeta chain. Thus, in some aspects, the antigen-binding portion is linked to one or more cell signaling modules. In some embodiments, cell signaling modules include CD3 transmembrane domain, CD3 intracellular signaling domains, and/or other CD3 transmembrane domains. In some embodiments, the receptor, e.g., CAR, further includes a portion of one or more additional molecules such as Fc receptor γ, CD8, CD4, CD25, or CD16. For example, in some aspects, the CAR or other chimeric receptor includes a chimeric molecule between CD3-zeta (CD3-ζ) or Fc receptor γ and CD8, CD4, CD25 or CD16.

In some embodiments, upon ligation of the CAR or other chimeric receptor, the cytoplasmic domain or intracellular signaling domain of the receptor activates at least one of the normal effector functions or responses of the immune cell, e.g., T cell engineered to express the CAR. For example, in some contexts, the CAR induces a function of a T cell such as cytolytic activity or T-helper activity, such as secretion of cytokines or other factors. In some embodiments, a truncated portion of an intracellular signaling domain of an antigen receptor component or costimulatory molecule is used in place of an intact immunostimulatory chain, for example, if it transduces the effector function signal. In some embodiments, the intracellular signaling domain or domains include the cytoplasmic sequences of the T cell receptor (TCR), and in some aspects also those of co-receptors that in the natural context act in concert with such receptors to initiate signal transduction following antigen receptor engagement.

In the context of a natural TCR, full activation generally requires not only signaling through the TCR, but also a costimulatory signal. Thus, in some embodiments, to promote full activation, a component for generating secondary or co-stimulatory signal is also included in the CAR. In other embodiments, the CAR does not include a component for generating a costimulatory signal. In some aspects, an additional CAR is expressed in the same cell and provides the component for generating the secondary or costimulatory signal.

In some embodiments, the chimeric antigen receptor contains an intracellular domain of a T cell costimulatory molecule. In some embodiments, the CAR includes a signaling domain and/or transmembrane portion of a costimulatory receptor, such as CD28, 4-1BB, OX40, DAP10, and ICOS. In some aspects, the same CAR includes both the activating and costimulatory components. In some embodiments, the chimeric antigen receptor contains an intracellular domain derived from a T cell costimulatory molecule or a functional variant thereof, such as between the transmembrane domain and intracellular signaling domain. In some aspects, the T cell costimulatory molecule is CD28 or 41BB.

In some embodiments, the activating domain is included within one CAR, whereas the costimulatory component is provided by another CAR recognizing another antigen. In some embodiments, the CARs include activating or stimulatory CARs, costimulatory CARs, both expressed on the same cell (see WO2014/055668). In some aspects, the cells include one or more stimulatory or activating CAR and/or a costimulatory CAR. In some embodiments, the cells further include inhibitory CARs (iCARs, see Fedorov et al., Sci. Transl. Medicine, 5(215) (December, 2013), such as a CAR recognizing an antigen other than the one associated with and/or specific for the disease or condition whereby an activating signal delivered through the disease-targeting CAR is diminished or inhibited by binding of the inhibitory CAR to its ligand, e.g., to reduce off-target effects.

In some embodiments, the two receptors induce, respectively, an activating and an inhibitory signal to the cell, such that ligation of one of the receptor to its antigen activates the cell or induces a response, but ligation of the second inhibitory receptor to its antigen induces a signal that suppresses or dampens that response. Examples are combinations of activating CARs and inhibitory CARs (iCARs). Such a strategy may be used, for example, to reduce the likelihood of off-target effects in the context in which the activating CAR binds an antigen expressed in a disease or condition but which is also expressed on normal cells, and the inhibitory receptor binds to a separate antigen which is expressed on the normal cells but not cells of the disease or condition.

In some aspects, the chimeric receptor is or includes an inhibitory CAR (e.g. iCAR) and includes intracellular components that dampen or suppress an immune response, such as an ITAM- and/or co stimulatory-promoted response in the cell. Exemplary of such intracellular signaling components are those found on immune checkpoint molecules, including PD-1, CTLA4, LAG3, BTLA, OX2R, TIM-3, TIGIT, LAIR-1, PGE2 receptors, EP2/4 Adenosine receptors including A2AR. In some aspects, the engineered cell includes an inhibitory CAR including a signaling domain of or derived from such an inhibitory molecule, such that it serves to dampen the response of the cell, for example, that induced by an activating and/or costimulatory CAR.

In certain embodiments, the intracellular signaling domain comprises a CD28 transmembrane and signaling domain linked to a CD3 (e.g., CD3-zeta) intracellular domain. In some embodiments, the intracellular signaling domain comprises a chimeric CD28 and CD137 (4-1BB, TNFRSF9) co-stimulatory domains, linked to a CD3 zeta intracellular domain.

In some embodiments, the CAR encompasses one or more, e.g., two or more, costimulatory domains and an activation domain, e.g., primary activation domain, in the cytoplasmic portion. Exemplary CARs include intracellular components of CD3-zeta, CD28, and 4-1BB.

In some embodiments, the antigen receptor further includes a marker and/or cells expressing the CAR or other antigen receptor further includes a surrogate marker, such as a cell surface marker, which may be used to confirm transduction or engineering of the cell to express the receptor. In some aspects, the marker includes all or part (e.g., truncated form) of CD34, a NGFR, or epidermal growth factor receptor, such as truncated version of such a cell surface receptor (e.g., tEGFR). In some embodiments, the nucleic acid encoding the marker is operably linked to a polynucleotide encoding for a linker sequence, such as a cleavable linker sequence, e.g., T2A. For example, a marker, and optionally a linker sequence, can be any as disclosed in published patent application No. WO2014031687. For example, the marker can be a truncated EGFR (tEGFR) that is, optionally, linked to a linker sequence, such as a T2A cleavable linker sequence.

An exemplary polypeptide for a truncated EGFR (e.g. tEGFR) comprises the sequence of amino acids set forth in SEQ ID NO: 43 or 16 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 43 or 44. An exemplary T2A linker sequence comprises the sequence of amino acids set forth in SEQ ID NO: 47 or 48 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 47 or 48.

In some embodiments, the marker is a molecule, e.g., cell surface protein, not naturally found on T cells or not naturally found on the surface of T cells, or a portion thereof. In some embodiments, the molecule is a non-self molecule, e.g., non-self protein, i.e., one that is not recognized as “self” by the immune system of the host into which the cells will be adoptively transferred.

In some embodiments, the marker serves no therapeutic function and/or produces no effect other than to be used as a marker for genetic engineering, e.g., for selecting cells successfully engineered. In other embodiments, the marker may be a therapeutic molecule or molecule otherwise exerting some desired effect, such as a ligand for a cell to be encountered in vivo, such as a costimulatory or immune checkpoint molecule to enhance and/or dampen responses of the cells upon adoptive transfer and encounter with ligand.

In some cases, CARs are referred to as first, second, and/or third generation CARs. In some aspects, a first generation CAR is one that solely provides a CD3-chain induced signal upon antigen binding; in some aspects, a second-generation CARs is one that provides such a signal and costimulatory signal, such as one including an intracellular signaling domain from a costimulatory receptor such as CD28 or CD137; in some aspects, a third generation CAR is one that includes multiple costimulatory domains of different costimulatory receptors.

For example, in some embodiments, the CAR contains an antibody, e.g., an antibody fragment, such as an scFv, specific to an antigen including any as described, a transmembrane domain that is or contains a transmembrane portion of CD28 or a functional variant thereof, and an intracellular signaling domain containing a signaling portion of CD28 or functional variant thereof and a signaling portion of CD3 zeta or functional variant thereof. In some embodiments, the CAR contains an antibody, e.g., antibody fragment, such as an scFv, specific to an antigen including any as described, a transmembrane domain that is or contains a transmembrane portion of CD28 or a functional variant thereof, and an intracellular signaling domain containing a signaling portion of a 4-1BB or functional variant thereof and a signaling portion of CD3 zeta or functional variant thereof. In some such embodiments, the receptor further includes a spacer containing a portion of an Ig molecule, such as a human Ig molecule, such as an Ig hinge, e.g. an IgG4 hinge, such as a hinge-only spacer.

In some embodiments, the transmembrane domain of the recombinant receptor, e.g., the CAR, is or includes a transmembrane domain of human CD28 (e.g. Accession No. P01747.1) or variant thereof, such as a transmembrane domain that comprises the sequence of amino acids set forth in SEQ ID NO: 95 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 95; in some embodiments, the transmembrane-domain containing portion of the recombinant receptor comprises the sequence of amino acids set forth in SEQ ID NO: 96 or a sequence of amino acids having at least at or about 85%, 86%, 87%, 88%, 89%, 90%, 91%, 92%, 93%, 94%, 95%, 96%, 97%, 98%, 99% or more sequence identity thereto.

In some embodiments, the intracellular signaling component(s) of the recombinant receptor, e.g. the CAR, contains an intracellular costimulatory signaling domain of human CD28 or a functional variant or portion thereof, such as a domain with an LL to GG substitution at positions 186-187 of a native CD28 protein. For example, the intracellular signaling domain can comprise the sequence of amino acids set forth in SEQ ID NO: 97 or 98 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 97 or 98. In some embodiments, the intracellular domain comprises an intracellular costimulatory signaling domain of 4-1BB (e.g. (Accession No. Q07011.1) or functional variant or portion thereof, such as the sequence of amino acids set forth in SEQ ID NO: 99 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 99.

In some embodiments, the intracellular signaling domain of the recombinant receptor, e.g. the CAR, comprises a human CD3 zeta stimulatory signaling domain or functional variant thereof, such as an 112 AA cytoplasmic domain of isoform 3 of human CD3t (Accession No.: P20963.2) or a CD3 zeta signaling domain as described in U.S. Pat. No. 7,446,190 or U.S. Pat. No. 8,911,993. For example, in some embodiments, the intracellular signaling domain comprises the sequence of amino acids as set forth in SEQ ID NO: 100, 101 or 102 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 100, 101 or 102.

In some aspects, the spacer contains only a hinge region of an IgG, such as only a hinge of IgG4 or IgG1, such as the hinge only spacer set forth in SEQ ID NO: 81. In other embodiments, the spacer is or contains an Ig hinge, e.g., an IgG4-derived hinge, optionally linked to a CH2 and/or CH3 domains. In some embodiments, the spacer is an Ig hinge, e.g., an IgG4 hinge, linked to CH2 and CH3 domains, such as set forth in SEQ ID NO: 84. In some embodiments, the spacer is an Ig hinge, e.g., an IgG4 hinge, linked to a CH3 domain only, such as set forth in SEQ ID NO: 83. In some embodiments, the spacer is or comprises a glycine-serine rich sequence or other flexible linker such as known flexible linkers.

For example, in some embodiments, the CAR includes an antibody such as an antibody fragment, including scFvs, a spacer, such as a spacer containing a portion of an immunoglobulin molecule, such as a hinge region and/or one or more constant regions of a heavy chain molecule, such as an Ig-hinge containing spacer, a transmembrane domain containing all or a portion of a CD28-derived transmembrane domain, a CD28-derived intracellular signaling domain, and a CD3 zeta signaling domain. In some embodiments, the CAR includes an antibody or fragment, such as scFv, a spacer such as any of the Ig-hinge containing spacers, a CD28-derived transmembrane domain, a 4-1BB-derived intracellular signaling domain, and a CD3 zeta-derived signaling domain.

Exemplary surrogate markers can include truncated forms of cell surface polypeptides, such as truncated forms that are non-functional and to not transduce or are not capable of transducing a signal or a signal ordinarily transduced by the full-length form of the cell surface polypeptide, and/or do not or are not capable of internalizing. Exemplary truncated cell surface polypeptides including truncated forms of growth factors or other receptors such as a truncated human epidermal growth factor receptor 2 (tHER2), a truncated epidermal growth factor receptor (tEGFR, exemplary tEGFR sequence set forth in 43 or 44) or a prostate-specific membrane antigen (PSMA) or modified form thereof. tEGFR may contain an epitope recognized by the antibody cetuximab (Erbitux®) or other therapeutic anti-EGFR antibody or binding molecule, which can be used to identify or select cells that have been engineered to express the tEGFR construct and an encoded exogenous protein, and/or to eliminate or separate cells expressing the encoded exogenous protein. See U.S. Pat. No. 8,802,374 and Liu et al., Nature Biotech. 2016 April; 34(4): 430-434). In some aspects, the marker, e.g. surrogate marker, includes all or part (e.g., truncated form) of CD34, a NGFR, a CD19 or a truncated CD19, e.g., a truncated non-human CD19, or epidermal growth factor receptor (e.g., tEGFR). In some embodiments, the marker is or comprises a fluorescent protein, such as green fluorescent protein (GFP), enhanced green fluorescent protein (EGFP), such as super-fold GFP (sfGFP), red fluorescent protein (RFP), such as tdTomato, mCherry, mStrawberry, AsRed2, DsRed or DsRed2, cyan fluorescent protein (CFP), blue green fluorescent protein (BFP), enhanced blue fluorescent protein (EBFP), and yellow fluorescent protein (YFP), and variants thereof, including species variants, monomeric variants, and codon-optimized and/or enhanced variants of the fluorescent proteins. In some embodiments, the marker is or comprises an enzyme, such as a luciferase, the lacZ gene from E. coli, alkaline phosphatase, secreted embryonic alkaline phosphatase (SEAP), chloramphenicol acetyl transferase (CAT). Exemplary light-emitting reporter genes include luciferase (luc), β-galactosidase, chloramphenicol acetyltransferase (CAT), β-glucuronidase (GUS) or variants thereof.

In some embodiments, the marker is a resistance marker or selection marker. In some embodiments, the resistance marker or selection marker is or comprises a polypeptide that confers resistance to exogenous agents or drugs. In some embodiments, the resistance marker or selection marker is an antibiotic resistance gene. In some embodiments, the resistance marker or selection marker is an antibiotic resistance gene confers antibiotic resistance to a mammalian cell. In some embodiments, the resistance marker or selection marker is or comprises a Puromycin resistance gene, a Hygromycin resistance gene, a Blasticidin resistance gene, a Neomycin resistance gene, a Geneticin resistance gene or a Zeocin resistance gene or a modified form thereof.

In some embodiments, the nucleic acid encoding the marker is operably linked to a polynucleotide encoding for a linker sequence, such as a cleavable linker sequence, e.g., a T2A. For example, a marker, and optionally a linker sequence, can be any as disclosed in PCT Pub. No. WO2014031687.

In some embodiments, nucleic acid molecules encoding such CAR constructs further includes a sequence encoding a T2A ribosomal skip element and/or a tEGFR sequence, e.g., downstream of the sequence encoding the CAR. In some embodiments, the sequence encodes a T2A ribosomal skip element set forth in SEQ ID NO: 47 or 48, or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 47 or 48.

In some embodiments, T cells expressing an antigen receptor (e.g. CAR) can also be generated to express a truncated EGFR (EGFRt) as a non-immunogenic selection epitope (e.g. by introduction of a construct encoding the CAR and EGFRt separated by a T2A ribosome switch to express two proteins from the same construct), which then can be used as a marker to detect such cells (see e.g. U.S. Pat. No. 8,802,374). In some embodiments, the sequence encodes an tEGFR sequence set forth in SEQ ID NO: 43 or 44, or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 43 or 44. In some cases, the peptide, such as T2A, can cause the ribosome to skip (ribosome skipping) synthesis of a peptide bond at the C-terminus of a 2A element, leading to separation between the end of the 2A sequence and the next peptide downstream (see, for example, de Felipe. Genetic Vaccines and Ther. 2:13 (2004) and deFelipe et al. Traffic 5:616-626 (2004)). Many 2A elements are known. Examples of 2A sequences that can be used herein include 2A sequences from the foot-and-mouth disease virus (F2A, e.g., SEQ ID NO: 45), equine rhinitis A virus (E2A, e.g., SEQ ID NO: 46), Thosea asigna virus (T2A, e.g., SEQ ID NO: 47 or 48), and porcine teschovirus-1 (P2A, e.g., SEQ ID NO: 49 or 50) as described in U.S. Patent Publication No. 20070116690.

The recombinant receptors, such as CARs, expressed by the cells administered to the subject generally recognize or specifically bind to a molecule that is expressed in, associated with, and/or specific for the disease or condition or cells thereof being treated. Upon specific binding to the molecule, e.g., antigen, the receptor generally delivers an immunostimulatory signal, such as an ITAM-transduced signal, into the cell, thereby promoting an immune response targeted to the disease or condition. For example, in some embodiments, the cells express a CAR that specifically binds to an antigen expressed by a cell or tissue of the disease or condition or associated with the disease or condition.

### 2. Chimeric Auto-Antibody Receptor (CAAR)

In some embodiments, the recombinant protein is a chimeric autoantibody receptor (CAAR). In some embodiments, the CAAR binds, e.g., specifically binds, or recognizes, an autoantibody. In some embodiments, a cell expressing the CAAR, such as a T cell engineered to express a CAAR, can be used to bind to and kill autoantibody-expressing cells, but not normal antibody expressing cells. In some embodiments, CAAR-expressing cells can be used to treat an autoimmune disease associated with expression of self-antigens, such as autoimmune diseases. In some embodiments, CAAR-expressing cells can target B cells that ultimately produce the autoantibodies and display the autoantibodies on their cell surfaces, mark these B cells as disease-specific targets for therapeutic intervention. In some embodiments, CAAR-expressing cells can be used to efficiently targeting and killing the pathogenic B cells in autoimmune diseases by targeting the disease-causing B cells using an antigen-specific chimeric autoantibody receptor. In some embodiments, the recombinant receptor is a CAAR, such as any described in U.S. Patent Application Pub. No. US 2017/0051035.

In some embodiments, the CAAR comprises an autoantibody binding domain, a transmembrane domain, and one or more intracellular signaling region or domain (also interchangeably called a cytoplasmic signaling domain or region). In some embodiments, the intracellular signaling region comprises an intracellular signaling domain. In some embodiments, the intracellular signaling domain is or comprises a primary signaling domain, a signaling domain that is capable of stimulating and/or inducing a primary activation signal in a T cell, a signaling domain of a T cell receptor (TCR) component (e.g. an intracellular signaling domain or region of a CD3-zeta (CD3ζ) chain or a functional variant or signaling portion thereof), and/or a signaling domain comprising an immunoreceptor tyrosine-based activation motif (ITAM).

In some embodiments, the autoantibody binding domain comprises an autoantigen or a fragment thereof. The choice of autoantigen can depend upon the type of autoantibody being targeted. For example, the autoantigen may be chosen because it recognizes an autoantibody on a target cell, such as a B cell, associated with a particular disease state, e.g. an autoimmune disease, such as an autoantibody-mediated autoimmune disease. In some embodiments, the autoimmune disease includes pemphigus vulgaris (PV). Exemplary autoantigens include desmoglein 1 (Dsg1) and Dsg3.

### 3. T Cell Receptors (TCRs)

In some embodiments, the recombinant protein is a T cell receptor (TCR). In some embodiments, the TCR recognizes an peptide epitope or T cell epitope of a target polypeptide, such as an antigen of a tumor, viral or autoimmune protein. In some embodiments, a “T cell receptor” or “TCR” is a molecule that contains a variable α and β chains (also known as TCRα and TCRβ, respectively) or a variable γ and δ chains (also known as TCRα and TCRβ, respectively), or antigen-binding portions thereof, and which is capable of specifically binding to a peptide bound to an MHC molecule. In some embodiments, the TCR is in the αβ form. Typically, TCRs that exist in αβ and γδ forms are generally structurally similar, but T cells expressing them may have distinct anatomical locations or functions. A TCR can be found on the surface of a cell or in soluble form. Generally, a TCR is found on the surface of T cells (or T lymphocytes) where it is generally responsible for recognizing antigens bound to major histocompatibility complex (MHC) molecules.

Unless otherwise stated, the term “TCR” should be understood to encompass full TCRs as well as antigen-binding portions or antigen-binding fragments thereof. In some embodiments, the TCR is an intact or full-length TCR, including TCRs in the αβ form or γδ form. In some embodiments, the TCR is an antigen-binding portion that is less than a full-length TCR but that binds to a specific peptide bound in an MHC molecule, such as binds to an MHC-peptide complex. In some cases, an antigen-binding portion or fragment of a TCR can contain only a portion of the structural domains of a full-length or intact TCR, but yet is able to bind the peptide epitope, such as MHC-peptide complex, to which the full TCR binds. In some cases, an antigen-binding portion contains the variable domains of a TCR, such as variable α chain and variable β chain of a TCR, sufficient to form a binding site for binding to a specific MHC-peptide complex. Generally, the variable chains of a TCR contain complementarity determining regions involved in recognition of the peptide, MHC and/or MHC-peptide complex.

In some embodiments, the variable domains of the TCR contain hypervariable loops, or complementarity determining regions (CDRs), which generally are the primary contributors to antigen recognition and binding capabilities and specificity. In some embodiments, a CDR of a TCR or combination thereof forms all or substantially all of the antigen-binding site of a given TCR molecule. The various CDRs within a variable region of a TCR chain generally are separated by framework regions (FRs), which generally display less variability among TCR molecules as compared to the CDRs (see, e.g., Jores et al., Proc. Nat'l Acad. Sci. U.S.A. 87:9138, 1990; Chothia et al., EMBO J. 7:3745, 1988; see also Lefranc et al., Dev. Comp. Immunol. 27:55, 2003). In some embodiments, CDR3 is the main CDR responsible for antigen binding or specificity, or is the most important among the three CDRs on a given TCR variable region for antigen recognition, and/or for interaction with the processed peptide portion of the peptide-MHC complex. In some contexts, the CDR1 of the alpha chain can interact with the N-terminal part of certain antigenic peptides. In some contexts, CDR1 of the beta chain can interact with the C-terminal part of the peptide. In some contexts, CDR2 contributes most strongly to or is the primary CDR responsible for the interaction with or recognition of the MHC portion of the MHC-peptide complex. In some embodiments, the variable region of the β-chain can contain a further hypervariable region (CDR4 or HVR4), which generally is involved in superantigen binding and not antigen recognition (Kotb (1995) Clinical Microbiology Reviews, 8:411-426).

In some embodiments, a TCR also can contain a constant domain, a transmembrane domain and/or a short cytoplasmic tail (see, e.g., Janeway et al., Immunobiology: The Immune System in Health and Disease, 3rd Ed., Current Biology Publications, p. 4:33, 1997). In some aspects, each chain of the TCR can possess one N-terminal immunoglobulin variable domain, one immunoglobulin constant domain, a transmembrane region, and a short cytoplasmic tail at the C-terminal end. In some embodiments, a TCR is associated with invariant proteins of the CD3 complex involved in mediating signal transduction.

In some embodiments, a TCR chain contains one or more constant domain. For example, the extracellular portion of a given TCR chain (e.g., α-chain or β-chain) can contain two immunoglobulin-like domains, such as a variable domain (e.g., Vα or Vβ; typically amino acids 1 to 116 based on Kabat numbering Kabat et al., “Sequences of Proteins of Immunological Interest, US Dept. Health and Human Services, Public Health Service National Institutes of Health, 1991, 5th ed.) and a constant domain (e.g., α-chain constant domain or Cα, typically positions 117 to 259 of the chain based on Kabat numbering or β chain constant domain or Cβ, typically positions 117 to 295 of the chain based on Kabat) adjacent to the cell membrane. For example, in some cases, the extracellular portion of the TCR formed by the two chains contains two membrane-proximal constant domains, and two membrane-distal variable domains, which variable domains each contain CDRs. The constant domain of the TCR may contain short connecting sequences in which a cysteine residue forms a disulfide bond, thereby linking the two chains of the TCR. In some embodiments, a TCR may have an additional cysteine residue in each of the α and β chains, such that the TCR contains two disulfide bonds in the constant domains.

In some embodiments, the TCR chains contain a transmembrane domain. In some embodiments, the transmembrane domain is positively charged. In some cases, the TCR chain contains a cytoplasmic tail. In some cases, the structure allows the TCR to associate with other molecules like CD3 and subunits thereof. For example, a TCR containing constant domains with a transmembrane region may anchor the protein in the cell membrane and associate with invariant subunits of the CD3 signaling device or complex. The intracellular tails of CD3 signaling subunits (e.g. CD3γ, CD3δ, CD3ε and CD3ζ chains) contain one or more immunoreceptor tyrosine-based activation motif or ITAM that are involved in the signaling capacity of the TCR complex.

In some embodiments, the TCR may be a heterodimer of two chains α and β (or optionally γ and δ) or it may be a single chain TCR construct. In some embodiments, the TCR is a heterodimer containing two separate chains (α and β chains or γ and δ chains) that are linked, such as by a disulfide bond or disulfide bonds.

In some embodiments, the TCR can be generated from a known TCR sequence(s), such as sequences of Vα,β chains, for which a substantially full-length coding sequence is readily available. Methods for obtaining full-length TCR sequences, including V chain sequences, from cell sources are well known. In some embodiments, nucleic acids encoding the TCR can be obtained from a variety of sources, such as by polymerase chain reaction (PCR) amplification of TCR-encoding nucleic acids within or isolated from a given cell or cells, or synthesis of publicly available TCR DNA sequences.

In some embodiments, the TCR is obtained from a biological source, such as from cells such as from a T cell (e.g. cytotoxic T cell), T-cell hybridomas or other publicly available source. In some embodiments, the T-cells can be obtained from in vivo isolated cells. In some embodiments, the TCR is a thymically selected TCR. In some embodiments, the TCR is a neoepitope-restricted TCR. In some embodiments, the T-cells can be a cultured T-cell hybridoma or clone. In some embodiments, the TCR or antigen-binding portion thereof or antigen-binding fragment thereof can be synthetically generated from knowledge of the sequence of the TCR.

In some embodiments, the TCR is generated from a TCR identified or selected from screening a library of candidate TCRs against a target polypeptide antigen, or target T cell epitope thereof. TCR libraries can be generated by amplification of the repertoire of Vα and Vβ from T cells isolated from a subject, including cells present in PBMCs, spleen or other lymphoid organ. In some cases, T cells can be amplified from tumor-infiltrating lymphocytes (TILs). In some embodiments, TCR libraries can be generated from CD4+ or CD8+ T cells. In some embodiments, the TCRs can be amplified from a T cell source of a normal of healthy subject, i.e. normal TCR libraries. In some embodiments, the TCRs can be amplified from a T cell source of a diseased subject, i.e. diseased TCR libraries. In some embodiments, degenerate primers are used to amplify the gene repertoire of Vα and Vβ, such as by RT-PCR in samples, such as T cells, obtained from humans. In some embodiments, scTv libraries can be assembled from naïve Vα and Vβ libraries in which the amplified products are cloned or assembled to be separated by a linker. Depending on the source of the subject and cells, the libraries can be HLA allele-specific. Alternatively, in some embodiments, TCR libraries can be generated by mutagenesis or diversification of a parent or scaffold TCR molecule. In some aspects, the TCRs are subjected to directed evolution, such as by mutagenesis, e.g., of the α or β chain. In some aspects, particular residues within CDRs of the TCR are altered. In some embodiments, selected TCRs can be modified by affinity maturation. In some embodiments, antigen-specific T cells may be selected, such as by screening to assess CTL activity against the peptide. In some aspects, TCRs, e.g. present on the antigen-specific T cells, may be selected, such as by binding activity, e.g., particular affinity or avidity for the antigen.

In some embodiments, the TCR or antigen-binding portion thereof is one that has been modified or engineered. In some embodiments, directed evolution methods are used to generate TCRs with altered properties, such as with higher affinity for a specific MHC-peptide complex. In some embodiments, directed evolution is achieved by display methods including, but not limited to, yeast display (Holler et al. (2003) Nat Immunol, 4, 55-62; Holler et al. (2000) Proc Natl Acad Sci USA, 97, 5387-92), phage display (Li et al. (2005) Nat Biotechnol, 23, 349-54), or T cell display (Chervin et al. (2008) J Immunol Methods, 339, 175-84). In some embodiments, display approaches involve engineering, or modifying, a known, parent or reference TCR. For example, in some cases, a wild-type TCR can be used as a template for producing mutagenized TCRs in which in one or more residues of the CDRs are mutated, and mutants with an desired altered property, such as higher affinity for a desired target antigen, are selected.

In some embodiments, peptides of a target polypeptide for use in producing or generating a TCR of interest are known or can be readily identified. In some embodiments, peptides suitable for use in generating TCRs or antigen-binding portions can be determined based on the presence of an HLA-restricted motif in a target polypeptide of interest, such as a target polypeptide described below. In some embodiments, peptides are identified using available computer prediction models. In some embodiments, for predicting MHC class I binding sites, such models include, but are not limited to, ProPred1 (Singh and Raghava (2001) Bioinformatics 17(12):1236-1237, and SYFPEITHI (see Schuler et al. (2007) Immunoinformatics Methods in Molecular Biology, 409(1): 75-93 2007). In some embodiments, the MHC-restricted epitope is HLA-A0201, which is expressed in approximately 39-46% of all Caucasians and therefore, represents a suitable choice of MHC antigen for use preparing a TCR or other MHC-peptide binding molecule.

HLA-A0201-binding motifs and the cleavage sites for proteasomes and immune-proteasomes using computer prediction models are known. For predicting MHC class I binding sites, such models include, but are not limited to, ProPred1 (described in more detail in Singh and Raghava, ProPred: prediction of HLA-DR binding sites. BIOINFORMATICS 17(12):1236-1237 2001), and SYFPEITHI (see Schuler et al. SYFPEITHI, Database for Searching and T-Cell Epitope Prediction. in Immunoinformatics Methods in Molecular Biology, vol 409(1): 75-93 2007).

In some embodiments, the TCR or antigen binding portion thereof may be a recombinantly produced natural protein or mutated form thereof in which one or more property, such as binding characteristic, has been altered. In some embodiments, a TCR may be derived from one of various animal species, such as human, mouse, rat, or other mammal. A TCR may be cell-bound or in soluble form. In some embodiments, the TCR is in cell-bound form expressed on the surface of a cell.

In some embodiments, the TCR is a full-length TCR. In some embodiments, the TCR is an antigen-binding portion. In some embodiments, the TCR is a dimeric TCR (dTCR). In some embodiments, the TCR is a single-chain TCR (sc-TCR). In some embodiments, a dTCR or scTCR have the structures as described in WO 03/020763, WO 04/033685, WO2011/044186.

In some embodiments, the TCR contains a sequence corresponding to the transmembrane sequence. In some embodiments, the TCR does contain a sequence corresponding to cytoplasmic sequences. In some embodiments, the TCR is capable of forming a TCR complex with CD3. In some embodiments, any of the TCRs, including a dTCR or scTCR, can be linked to signaling domains that yield an active TCR on the surface of a T cell. In some embodiments, the TCR is expressed on the surface of cells.

In some embodiments a dTCR contains a first polypeptide wherein a sequence corresponding to a TCR α chain variable region sequence is fused to the N terminus of a sequence corresponding to a TCR α chain constant region extracellular sequence, and a second polypeptide wherein a sequence corresponding to a TCR β chain variable region sequence is fused to the N terminus a sequence corresponding to a TCR β chain constant region extracellular sequence, the first and second polypeptides being linked by a disulfide bond. In some embodiments, the bond can correspond to the native inter-chain disulfide bond present in native dimeric αβ TCRs. In some embodiments, the interchain disulfide bonds are not present in a native TCR. For example, in some embodiments, one or more cysteines can be incorporated into the constant region extracellular sequences of dTCR polypeptide pair. In some cases, both a native and a non-native disulfide bond may be desirable. In some embodiments, the TCR contains a transmembrane sequence to anchor to the membrane.

In some embodiments, a dTCR contains a TCR α chain containing a variable α domain, a constant α domain and a first dimerization motif attached to the C-terminus of the constant α domain, and a TCR β chain comprising a variable β domain, a constant β domain and a first dimerization motif attached to the C-terminus of the constant β domain, wherein the first and second dimerization motifs easily interact to form a covalent bond between an amino acid in the first dimerization motif and an amino acid in the second dimerization motif linking the TCR α chain and TCR β chain together.

In some embodiments, the TCR is a scTCR. Typically, a scTCR can be generated using methods known, See e.g., Soo Hoo, W. F. et al. PNAS (USA) 89, 4759 (1992); Wülfing, C. and Plückthun, A., J. Mol. Biol. 242, 655 (1994); Kurucz, I. et al. PNAS (USA) 90 3830 (1993); International published PCT Nos. WO 96/13593, WO 96/18105, WO99/60120, WO99/18129, WO 03/020763, WO2011/044186; and Schlueter, C. J. et al. J. Mol. Biol. 256, 859 (1996). In some embodiments, a scTCR contains an introduced non native disulfide interchain bond to facilitate the association of the TCR chains (see e.g. International published PCT No. WO 03/020763). In some embodiments, a scTCR is a non-disulfide linked truncated TCR in which heterologous leucine zippers fused to the C-termini thereof facilitate chain association (see e.g. International published PCT No. WO99/60120). In some embodiments, a scTCR contain a TCRα variable domain covalently linked to a TCRβ variable domain via a peptide linker (see e.g., International published PCT No. WO99/18129).

In some embodiments, a scTCR contains a first segment constituted by an amino acid sequence corresponding to a TCR α chain variable region, a second segment constituted by an amino acid sequence corresponding to a TCR β chain variable region sequence fused to the N terminus of an amino acid sequence corresponding to a TCR β chain constant domain extracellular sequence, and a linker sequence linking the C terminus of the first segment to the N terminus of the second segment.

In some embodiments, a scTCR contains a first segment constituted by an α chain variable region sequence fused to the N terminus of an α chain extracellular constant domain sequence, and a second segment constituted by a β chain variable region sequence fused to the N terminus of a sequence β chain extracellular constant and transmembrane sequence, and, optionally, a linker sequence linking the C terminus of the first segment to the N terminus of the second segment.

In some embodiments, a scTCR contains a first segment constituted by a TCR β chain variable region sequence fused to the N terminus of a β chain extracellular constant domain sequence, and a second segment constituted by an α chain variable region sequence fused to the N terminus of a sequence α chain extracellular constant and transmembrane sequence, and, optionally, a linker sequence linking the C terminus of the first segment to the N terminus of the second segment.

In some embodiments, the linker of a scTCRs that links the first and second TCR segments can be any linker capable of forming a single polypeptide strand, while retaining TCR binding specificity. In some embodiments, the linker sequence may, for example, have the formula -P-AA-P- wherein P is proline and AA represents an amino acid sequence wherein the amino acids are glycine and serine. In some embodiments, the first and second segments are paired so that the variable region sequences thereof are orientated for such binding. Hence, in some cases, the linker has a sufficient length to span the distance between the C terminus of the first segment and the N terminus of the second segment, or vice versa, but is not too long to block or reduces bonding of the scTCR to the target ligand. In some embodiments, the linker can contain from 10 to 45 amino acids or from about 10 to about 45 amino acids, such as 10 to 30 amino acids or 26 to 41 amino acids residues, for example 29, 30, 31 or 32 amino acids. In some embodiments, the linker has the formula -PGGG-(SGGGG)5-P- wherein P is proline, G is glycine and S is serine (SEQ ID NO:38). In some embodiments, the linker has the sequence GSADDAKKDAAKKDGKS (SEQ ID NO:39).

In some embodiments, the scTCR contains a covalent disulfide bond linking a residue of the immunoglobulin region of the constant domain of the α chain to a residue of the immunoglobulin region of the constant domain of the β chain. In some embodiments, the interchain disulfide bond in a native TCR is not present. For example, in some embodiments, one or more cysteines can be incorporated into the constant region extracellular sequences of the first and second segments of the scTCR polypeptide. In some cases, both a native and a non-native disulfide bond may be desirable.

In some embodiments of a dTCR or scTCR containing introduced interchain disulfide bonds, the native disulfide bonds are not present. In some embodiments, the one or more of the native cysteines forming a native interchain disulfide bonds are substituted to another residue, such as to a serine or alanine. In some embodiments, an introduced disulfide bond can be formed by mutating non-cysteine residues on the first and second segments to cysteine. Exemplary non-native disulfide bonds of a TCR are described in published International PCT No. WO2006/000830.

In some embodiments, the TCR or antigen-binding fragment thereof exhibits an affinity with an equilibrium binding constant for a target antigen of between or between about 10-5 and 10-12 M and all individual values and ranges therein. In some embodiments, the target antigen is an MHC-peptide complex or ligand.

In some embodiments, nucleic acid or nucleic acids encoding a TCR, such as α and β chains, can be amplified by PCR, cloning or other suitable means and cloned into a suitable expression vector or vectors. The expression vector can be any suitable recombinant expression vector, and can be used to transform or transfect any suitable host. Suitable vectors include those designed for propagation and expansion or for expression or both, such as plasmids and viruses.

In some embodiments, the vector can a vector of the pUC series (Fermentas Life Sciences), the pBluescript series (Stratagene, LaJolla, Calif.), the pET series (Novagen, Madison, Wis.), the pGEX series (Pharmacia Biotech, Uppsala, Sweden), or the pEX series (Clontech, Palo Alto, Calif.). In some cases, bacteriophage vectors, such as λG10, λGT11, λZapII (Stratagene), λEMBL4, and λNM1149, also can be used. In some embodiments, plant expression vectors can be used and include pBI01, pBI101.2, pBI101.3, pBI121 and pBIN19 (Clontech). In some embodiments, animal expression vectors include pEUK-Cl, pMAM and pMAMneo (Clontech). In some embodiments, a viral vector is used, such as a retroviral vector.

In some embodiments, the recombinant expression vectors can be prepared using standard recombinant DNA techniques. In some embodiments, vectors can contain regulatory sequences, such as transcription and translation initiation and termination codons, which are specific to the type of host (e.g., bacterium, fungus, plant, or animal) into which the vector is to be introduced, as appropriate and taking into consideration whether the vector is DNA- or RNA-based. In some embodiments, the vector can contain a nonnative promoter operably linked to the nucleotide sequence encoding the TCR or antigen-binding portion (or other MHC-peptide binding molecule). In some embodiments, the promoter can be a non-viral promoter or a viral promoter, such as a cytomegalovirus (CMV) promoter, an SV40 promoter, an RSV promoter, and a promoter found in the long-terminal repeat of the murine stem cell virus. Other known promoters also are contemplated.

In some embodiments, to generate a vector encoding a TCR, the α and β chains are PCR amplified from total cDNA isolated from a T cell clone expressing the TCR of interest and cloned into an expression vector. In some embodiments, the α and β chains are cloned into the same vector. In some embodiments, the α and β chains are cloned into different vectors. In some embodiments, the generated α and β chains are incorporated into a retroviral, e.g. lentiviral, vector.

### VI. Stationary Phases, Kits, and Other Articles

In some embodiments, the provided methods involve the use of chromatography matrices, chromatography columns, and stationary phases. Such chromatography matrices, chromatography columns, and stationary phases include any as described herein, for instance here in Section VI. In some embodiments, the provided kits, stationary phases, and articles of manufacture include any combination of any of the VBPs, VBP reagents, selection agents, selection reagents, chromatography matrices, stationary phases, and chromatography columns described herein, for instance here in Section VI.

In some embodiments, the provided kits can be used for purifying viral particles. In some embodiments, the provided stationary phases can be used for purifying viral particles. In some embodiments, the provided articles of manufacture can be used for purifying viral particles.

In some embodiments, the provided kits can be used for transducing cells, e.g., the on-column transduction of cells, for instance according to any of the described methods. In some embodiments, the provided stationary phases can be used for transducing cells, e.g., the on-column transduction of cells, for instance according to any of the described methods. In some embodiments, the provided articles of manufacture can be used for transducing cells, e.g., the on-column transduction of cells, for instance according to any of the described methods. In some embodiments, the cells are any of the target cells described herein. In some embodiments, the cells are transduced with a viral particle.

Exemplary viral particles are described in Section V. In some embodiments, the viral particle is any as described herein. In some embodiments, the kit contains any of the viral particles described herein.

In some embodiments, the kit contains a chromatography matrix suitable for viral purification using column chromatography. In some embodiments, the kit contains a stationary phase suitable for viral purification using column chromatography. In some embodiments, the article of manufacture contains a stationary phase suitable for viral purification using column chromatography. In some embodiments, the stationary phase contains a chromatography matrix that is suitable for viral purification using column chromatography.

In some embodiments, the kit contains a chromatography matrix suitable for cell separation using column chromatography. In some embodiments, the kit contains a stationary phase suitable for cell separation using column chromatography. In some embodiments, the stationary phase contains a chromatography matrix suitable for cell separation using column chromatography.

Exemplary chromatography matrices are described in Section VI-A. In some embodiments, the chromatography matrix is any as described herein.

In some embodiments, the kit contains any of the provided VBPs. In some embodiments, the VBP is capable of being immobilized on the chromatography matrix. In some embodiments, the VBP is immobilized on the chromatography matrix.

In some embodiments, the stationary phase contains any of the provided VBPs. In some embodiments, the VBP is immobilized on the chromatography matrix of the stationary phase.

In some embodiments, the kit contains any of the provided VBP reagents. In some embodiments, the VBP reagent is capable of being immobilized on the chromatography matrix. In some embodiments, the VBP reagent is immobilized on the chromatography matrix.

In some embodiments, the stationary phase contains any of the provided VBP reagents. In some embodiments, the VBP reagent is immobilized on the chromatography matrix of the stationary phase.

In some embodiments, the kit contains a selection reagent. In some embodiments, the stationary phase contains a selection reagent. In some embodiments, the selection reagent contains a molecule or a plurality of molecules of streptavidin, avidin, a streptavidin analog or mutein, or an avidin analog or mutein. In some embodiments, the molecule is any of the streptavidin, avidin, streptavidin analog or mutein, or avidin analog or mutein molecules described herein, for instance in Section II-A.

In some embodiments, the selection reagent is capable of being immobilized on the chromatography matrix. In some embodiments, the selection reagent is immobilized on the chromatography matrix. In some embodiments, the selection reagent is immobilized on the chromatography matrix of the stationary phase.

In some embodiments, the kit contains a selection agent. In some embodiments, the stationary phase contains a selection agent. In some embodiments, the selection agent specifically binds to a selection marker expressed on a target cell. In some embodiments, the selection agent is any of the binding agents described herein, e.g., in Section II-B, as well as any described in Section IV-A.

In some embodiments, the selection agent is capable of being immobilized on the chromatography matrix. In some embodiments, the selection agent is immobilized on the chromatography matrix. In some embodiments, the selection agent is immobilized on the chromatography matrix of the stationary phase.

Methods for immobilizing the VBP, VBP reagent, selection agent, or selection reagent on the chromatography matrix can be identified and selected by one of ordinary skill in the art. For instance, crosslinkers such as those described in Section I can be used for conjugating the VBP, VBP reagent, selection agent, or selection reagent to the chromatography matrix. In addition, in some instances, materials of the chromatography matrix, such as resins, can be activated in order to form covalent bonds with ligands containing amine, thiol, or hydroxyl groups. Such activated materials include epoxy-activated materials, such as epoxy-activated agarose, which is commercially available.

In some embodiments, the VBP is immobilized to the chromatography matrix via binding to the selection reagent immobilized on the chromatography matrix. In some embodiments, the binding partner of the VBP is capable of binding to the selection reagent, e.g., the molecule of the selection reagent that is streptavidin, avidin, a streptavidin analog or mutein, or an avidin analog or mutein. In some embodiments, the binding partner of the VBP is bound to the molecule. In some embodiments, the binding partner of the VBP is reversibly bound to the molecule.

In some embodiments, the selection agent is immobilized to the chromatography matrix via binding to the selection reagent immobilized on the chromatography matrix. In some embodiments, the binding partner of the selection agent is capable of binding to the selection reagent, e.g., the molecule of the selection reagent that is streptavidin, avidin, a streptavidin analog or mutein, or an avidin analog or mutein. In some embodiments, the binding partner of the selection agent is bound to the molecule. In some embodiments, the binding partner of the selection agent is reversibly bound to the molecule.

In some embodiments, the kit contains a chromatography column. In some embodiments, the provided articles of manufacture contain a chromatography column. Exemplary chromatography columns include columns, e.g., those suitable for flow-through of a liquid sample, containers suitable for bidirectional flow, pipette tips, and tubes.

In some embodiments, the chromatography matrix is contained in the chromatography column. In some embodiments, the stationary phase is contained in the chromatography column.

In some aspects, the binding capacity of a stationary phase affects how much stationary phase is needed in order to select a certain number of target moieties, e.g., target cells such as T cells. The binding capacity, e.g., the number of target cells that can be immobilized per mL of the stationary phase, can be used to determine or control the number of immobilized target cells. In some aspects, the binding capacity of a stationary phase can be used to standardize the reagent amount, e.g., amount of VBP reagent or stimulatory reagent, used in a single column. In some embodiments, 1 mL of the stationary phase is capable of accommodating up to 0.1 billion±0.025 billion cells. In some embodiments, the stationary phase is or is about 5 mL, 10 mL, 15 mL, 20 mL, 25 mL, 30 mL, 35 mL, or 40 mL. In some embodiments, the stationary phase is or is about 10 mL and is capable of accommodating up to 1 billion±0.25 billion cells. In some embodiments, the stationary phase is or is about 20 mL and is capable of accommodating up to 2 billion±0.5 billion cells. In some embodiments, the stationary phase is or is about 40 mL and is capable of accommodating between about 3 billion and about 5 billion cells.

In some embodiments, the stationary phase has a binding capacity of between or between about 0.5 billion and 5 billion cells. In some embodiments, the stationary phase has a binding capacity of between or between about 0.5 billion and 4 billion cells. In some embodiments, the stationary phase has a binding capacity of between or between about 0.5 billion and 3 billion cells. In some embodiments, the stationary phase has a binding capacity of between or between about 0.5 billion and 2 billion cells. In some embodiments, the stationary phase has a binding capacity of between or between about 1 billion and 5 billion cells. In some embodiments, the stationary phase has a binding capacity of between or between about 1 billion and 4 billion cells. In some embodiments, the stationary phase has a binding capacity of between or between about 1 billion and 3 billion cells. In some embodiments, the stationary phase has a binding capacity of between or between about 1 billion and 2 billion cells, inclusive. In some embodiments, the stationary phase is 20 mL. In some embodiments, the stationary phase has a binding capacity of 2 billion±0.5 billion cells.

In some embodiments, the binding capacity of the stationary phase is the maximum number of target cells (e.g., CD3+ T cells, CD4+ T cells, or CD8+ T cells) bound to the stationary phase at given solvent and cell concentration conditions, when an excess of target cells are loaded onto the stationary phase. In some embodiments, the binding capacity is or is about 100 million±25 million target cells (e.g., T cells) per mL of stationary phase.

In some embodiments, the static binding capacity is the maximum amount of cells capable of being immobilized on the stationary phase, e.g., at certain solvent and cell concentration conditions. In some embodiments, the static binding capacity of the stationary phase ranges between about 75 million and about 125 million target cells per mL of stationary phase. In some embodiments, the static binding capacity of the stationary phase ranges between about 50 million and about 100 million target cells per mL of stationary phase. In some embodiments, the static binding capacity is or is about 100 million±25 million target cells (e.g., T cells) per mL of stationary phase. In some embodiments, the static binding capacity of the stationary phase disclosed herein ranges between about 75 million and about 125 million target cells per mL of stationary phase. In some embodiments, the static binding capacity of the stationary phase is between about 10 million and about 20 million, between about 20 million and about 30 million, between about 30 million and about 40 million, between about 40 million and about 50 million, between about 50 million and about 60 million, between about 60 million and about 70 million, between about 70 million and about 80 million, between about 80 million and about 90 million, between about 90 million and about 100 million, between about 110 million and about 120 million, between about 120 million and about 130 million, between about 130 million and about 140 million, between about 140 million and about 150 million, between about 150 million and about 160 million, between about 160 million and about 170 million, between about 170 million and about 180 million, between about 180 million and about 190 million, or between about 190 million and about 200 million target cells per mL of stationary phase.

In some embodiments, the binding capacity of the stationary phase is the number of target cells (e.g., CD3+ T cells, CD4+ T cells, or CD8+ T cells) that bind to the stationary phase under given flow conditions before a significant breakthrough of unbound target cells occurs. In one aspect, the binding capacity of the stationary phase is a dynamic binding capacity, e.g., the binding capacity under operating conditions in a packed chromatography column during sample application. In some embodiments, the dynamic binding capacity is determined by loading a sample containing a known concentration of the target cells and monitoring the flow-through, and the target cells will bind the stationary phase to a certain break point before unbound target cells will flow through the column. In some embodiments, the dynamic binding capacity is or is about 100 million±25 million target cells (e.g., T cells) per mL of stationary phase. In some embodiments, the dynamic binding capacity of the stationary phase is between or is between about 75 million and about 125 million target cells per mL of stationary phase. In some embodiments, the dynamic binding capacity of the stationary phase ranges between about 50 million and about 100 million target cells per mL of stationary phase. In some embodiments, the dynamic binding capacity of the stationary phase is between about 10 million and about 20 million, between about 20 million and about 30 million, between about 30 million and about 40 million, between about 40 million and about 50 million, between about 50 million and about 60 million, between about 60 million and about 70 million, between about 70 million and about 80 million, between about 80 million and about 90 million, between about 90 million and about 100 million, between about 110 million and about 120 million, between about 120 million and about 130 million, between about 130 million and about 140 million, between about 140 million and about 150 million, between about 150 million and about 160 million, between about 160 million and about 170 million, between about 170 million and about 180 million, between about 180 million and about 190 million, or between about 190 million and about 200 million target cells per mL of stationary phase.

In some embodiments, any of the foregoing binding capacity values can be that of a chromatography matrix described herein, for instance any of the chromatography matrices to which is immobilized a VBP, VBP reagent, or selection agent as described herein.

### A. Chromatography Matrices

In some embodiments, the chromatography matrix includes a non-magnetic material or non-magnetizable material. In some embodiments, the chromatography matrix is void of any magnetically attractable matter.

In some embodiments, the chromatography matrix includes a monolithic matrix. In some embodiments, the chromatography matrix includes a membrane matrix. In some embodiments, the chromatography matrix includes a particulate matrix. In some embodiments, the chromatography matrix includes a beaded matrix.

In some embodiments, the chromatography matrix is essentially innocuous, e.g., is not detrimental to the health or viability of cells added to the chromatography matrix.

In some embodiments, the chromatography matrix includes derivatized silica or a crosslinked gel. In some embodiments, the crosslinked gel is based on a natural polymer, for instance a polysaccharide. In some embodiments, the polysaccharide is crosslinked. Examples of a polysaccharide matrix include an agarose gel (for example, Superflow™ agarose or a Sepharose® material such as Superflow™ Sepharose® that is commercially available in different bead and pore sizes) or a gel of crosslinked dextrans. Further examples include a particulate cross-linked agarose matrix to which dextran is covalently bonded, for instance that is commercially available (in various bead sizes and with various pore sizes) as Sephadex® or Superdex®, both available from GE Healthcare. Further examples include Sephacryl® which is also available in different bead and pore sizes from GE Healthcare.

In some embodiments, the crosslinked gel is based on a synthetic polymer. In some embodiments, the synthetic polymer is a polymer that has polar monomer units and which is therefore in itself polar. In some embodiments, the synthetic polymer is hydrophilic. Examples of synthetic polymers include polyacrylamides, a styrene-divinylbenzene gel, and a copolymer of an acrylate and a diol or of an acrylamide and a diol. An illustrative example is a polymethacrylate gel, commercially available as a Fractogel®. A further example is a copolymer of ethylene glycol and methacrylate, commercially available as a Toyopearl®. In some embodiments, the chromatography matrix includes natural and synthetic polymer components, such as a composite matrix or a composite or a co-polymer of a polysaccharide and agarose, e.g., a polyacrylamide/agarose composite, or of a polysaccharide and N,N′-methylenebisacrylamide. An illustrative example of a copolymer of a dextran and N,N′-methylenebisacrylamide is the Sephacryl® series of material. A derivatized silica may include silica particles that are coupled to a synthetic or to a natural polymer. Examples of such embodiments include polysaccharide grafted silica, polyvinylpyrrolidone grafted silica, polyethylene oxide grafted silica, poly(2-hydroxyethylaspartamide) silica, and poly(N-isopropylacrylamide) grafted silica.

In some embodiments, the chromatography matrix includes a particulate matrix. In some embodiments, the chromatography matrix includes a polymeric resin, metal oxide, metalloid oxide, or mixed oxide. In some embodiments, particulates of the particulate matrix have a mean particle size of between or between about 5 μm and 600 μm, 5 μm and 400 μm, 5 μm and 200 μm, 5 μm and 150 μm, 5 μm and 125 μm, 5 μm and 100 μm, 5 μm and 75 μm, 5 μm and 50 μm, 5 μm and 25 μm, 25 μm and 600 μm, 25 μm and 400 μm, 25 μm and 200 μm, 25 μm and 150 μm, 25 μm and 125 μm, 25 μm and 100 μm, 25 μm and 75 μm, 25 μm and 50 μm, 50 μm and 600 μm, 50 μm and 400 μm, 50 μm and 200 μm, 50 μm and 150 μm, 50 μm and 125 μm, 50 μm and 100 μm, 50 μm and 75 μm, 75 μm and 600 μm, 75 μm and 400 μm, 75 μm and 200 μm, 75 μm and 150 μm, 75 μm and 125 μm, 75 μm and 100 μm, 100 μm and 600 μm, 100 μm and 400 μm, 100 μm and 200 μm, 100 μm and 150 μm, 100 μm and 125 μm, 125 μm and 600 μm, 125 μm and 400 μm, 125 μm and 200 μm, 125 μm and 150 μm, 150 μm and 600 μm, 150 μm and 400 μm, 150 μm and 200 μm, 200 μm and 600 μm, 200 μm and 400 μm, or 400 μm and 600 μm, each inclusive. In some embodiments, the chromatography resin beads are between or between about 50 μm and 150 μm in diameter, inclusive. In some embodiments, the chromatography resin beads are between or between about 75 μm and 125 μm in diameter, inclusive. In some embodiments, the chromatography resin beads are between or between about 90 μm and 110 μm in diameter, inclusive.

In some embodiments, the chromatography matrix includes a chromatography resin. In some embodiments, the chromatography matrix includes chromatography resin beads, such as those commercially available as CytoSorb® (CytoSorbents™). In some embodiments, the resin includes a polystyrene resin. In some embodiments, the chromatography resin beads are between or between about 5 μm and 600 μm, 5 μm and 400 μm, 5 μm and 200 μm, 5 μm and 150 μm, 5 μm and 125 μm, 5 μm and 100 μm, 5 μm and 75 μm, 5 μm and 50 μm, 5 μm and 25 μm, 25 μm and 600 μm, 25 μm and 400 μm, 25 μm and 200 μm, 25 μm and 150 μm, 25 μm and 125 μm, 25 μm and 100 μm, 25 μm and 75 μm, 25 μm and 50 μm, 50 μm and 600 μm, 50 μm and 400 μm, 50 μm and 200 μm, 50 μm and 150 μm, 50 μm and 125 μm, 50 μm and 100 μm, 50 μm and 75 μm, 75 μm and 600 μm, 75 μm and 400 μm, 75 μm and 200 μm, 75 μm and 150 μm, 75 μm and 125 μm, 75 μm and 100 μm, 100 μm and 600 μm, 100 μm and 400 μm, 100 μm and 200 μm, 100 μm and 150 μm, 100 μm and 125 μm, 125 μm and 600 μm, 125 μm and 400 μm, 125 μm and 200 μm, 125 μm and 150 μm, 150 μm and 600 μm, 150 μm and 400 μm, 150 μm and 200 μm, 200 μm and 600 μm, 200 μm and 400 μm, or 400 μm and 600 μm in diameter, each inclusive. In some embodiments, the chromatography resin beads are between or between about 50 μm and 150 μm in diameter, inclusive. In some embodiments, the chromatography resin beads are between or between about 75 μm and 125 μm in diameter, inclusive. In some embodiments, the chromatography resin beads are between or between about 90 μm and 110 μm in diameter, inclusive.

In some embodiments, the chromatography matrix contains magnetically attractable matter, such as one or more magnetically attractable particles or a ferrofluid. Magnetically attractable particles may contain diamagnetic, ferromagnetic, paramagnetic, or superparamagnetic material. Superparamagnetic material responds to a magnetic field with an induced magnetic field without a resulting permanent magnetization. Magnetic particles based on iron oxide are commercially available as, for example, Dynabeads® from Dynal Biotech, magnetic MicroBeads from Miltenyi Biotec, and magnetic porous glass beads from CPG Inc., as well as from various other sources, such as Roche Applied Science, BIOCLON, BioSource International Inc., micromod, AMBION, Merck, Bangs Laboratories, Polysciences, or Novagen Inc. Magnetic nanoparticles based on superparamagnetic Co and FeCo, as well as ferromagnetic Co nanocrystals, have been described by, for example, Hütten, A. et al. (J. Biotech. (2004), 112, 47-63).

### VII. Compositions

Also provided herein are compositions, including pharmaceutical compositions and formulations, containing the cells, such as CAR-expressing or TCR-expressing cells, produced by any of the methods described herein, including any of the provided methods for transducing cells. Also provided herein are methods of using and uses of the compositions, such as in the treatment of diseases, conditions, and disorders in which the antigen targeted by the CAR or TCR, is expressed, or in detection, diagnostic, and prognostic methods.

In some embodiments, the term “pharmaceutical formulation” refers to a preparation which is in such form as to permit the biological activity of an active ingredient contained therein to be effective, and which contains no additional components which are unacceptably toxic to a subject to which the formulation would be administered.

In some embodiments, a “pharmaceutically acceptable carrier” refers to an ingredient in a pharmaceutical formulation, other than an active ingredient, which is nontoxic to a subject. A pharmaceutically acceptable carrier can include a buffer, excipient, stabilizer, or preservative.

In some aspects, the choice of carrier is determined in part by the particular cell or agent and/or by the method of administration. Accordingly, there are a variety of suitable formulations. For example, the pharmaceutical composition can contain preservatives. Suitable preservatives may include methylparaben, propylparaben, sodium benzoate, and benzalkonium chloride. In some aspects, a mixture of two or more preservatives is used. The preservative or mixtures thereof can be present in an amount of about 0.0001% to about 2% by weight of the total composition. Carriers are described by, e.g., Remington's Pharmaceutical Sciences 16th edition, Osol, A. Ed. (1980). Pharmaceutically acceptable carriers are generally nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

Buffering agents in some aspects are included in the compositions. Suitable buffering agents include citric acid, sodium citrate, phosphoric acid, potassium phosphate, and various other acids and salts. In some aspects, a mixture of two or more buffering agents is used. The buffering agent or mixtures thereof can be present in an amount of about 0.001% to about 4% by weight of the total composition. Methods for preparing administrable pharmaceutical compositions are known. Exemplary methods are described in more detail in, for example, Remington: The Science and Practice of Pharmacy, Lippincott Williams & Wilkins; 21st ed. (May 1, 2005).

The formulation or composition may also contain more than one active ingredient useful for the particular indication, disease, or condition being prevented or treated with the cells or agents, where the respective activities do not adversely affect one another. Such active ingredients are suitably present in combination in amounts that are effective for the purpose intended. Thus, in some embodiments, the pharmaceutical composition further includes other pharmaceutically active agents or drugs, such as chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, vincristine, etc. In some embodiments, the agents or cells are administered in the form of a salt, e.g., a pharmaceutically acceptable salt. Suitable pharmaceutically acceptable acid addition salts include those derived from mineral acids, such as hydrochloric, hydrobromic, phosphoric, metaphosphoric, nitric, and sulphuric acids, and organic acids, such as tartaric, acetic, citric, malic, lactic, fumaric, benzoic, glycolic, gluconic, succinic, and arylsulphonic acids, for example, p-toluenesulphonic acid.

The pharmaceutical composition in some embodiments contains agents or cells in amounts effective to treat or prevent the disease or condition, such as a therapeutically effective or prophylactically effective amount. Therapeutic or prophylactic efficacy in some embodiments is monitored by periodic assessment of treated subjects. For repeated administrations over several days or longer, depending on the condition, the treatment is repeated until a desired suppression of disease symptoms occurs. However, other dosage regimens may be useful and can be determined. The desired dosage can be delivered by a single bolus administration of the composition, by multiple bolus administrations of the composition, or by continuous infusion administration of the composition.

The agents or cells can be administered by any suitable means, for example by bolus infusion, by injection, e.g., intravenous or subcutaneous injections, intraocular injection, periocular injection, subretinal injection, intravitreal injection, trans-septal injection, subscleral injection, intrachoroidal injection, intracameral injection, subconjectval injection, subconjuntival injection, sub-Tenon's injection, retrobulbar injection, peribulbar injection, or posterior juxtascleral delivery. In some embodiments, they are administered by parenteral, intrapulmonary, and intranasal, and, if desired for local treatment, intralesional administration. Parenteral infusions include intramuscular, intravenous, intraarterial, intraperitoneal, or subcutaneous administration. In some embodiments, a given dose is administered by a single bolus administration of the cells or agent. In some embodiments, it is administered by multiple bolus administrations of the cells or agent, for example, over a period of no more than 3 days, or by continuous infusion administration of the cells or agent.

For the prevention or treatment of disease, the appropriate dosage may depend on the type of disease to be treated, the type of agent or agents, the type of cells or recombinant receptors, the severity and course of the disease, whether the agent or cells are administered for preventive or therapeutic purposes, previous therapy, the subject's clinical history and response to the agent or the cells, and the discretion of the attending physician. The compositions are in some embodiments suitably administered to the subject at one time or over a series of treatments.

The cells or agents may be administered using standard administration techniques, formulations, and/or devices. Provided are formulations and devices, such as syringes and vials, for storage and administration of the compositions. With respect to cells, administration can be autologous or heterologous. For example, immunoresponsive cells or progenitors can be obtained from one subject, and administered to the same subject or a different, compatible subject. Peripheral blood derived immunoresponsive cells or their progeny (e.g., in vivo, ex vivo or in vitro derived) can be administered via localized injection, including catheter administration, systemic injection, localized injection, intravenous injection, or parenteral administration. When administering a therapeutic composition (e.g., a pharmaceutical composition containing a genetically modified immunoresponsive cell or an agent that treats or ameliorates symptoms of neurotoxicity), it will generally be formulated in a unit dosage injectable form (solution, suspension, emulsion).

Formulations include those for oral, intravenous, intraperitoneal, subcutaneous, pulmonary, transdermal, intramuscular, intranasal, buccal, sublingual, or suppository administration. In some embodiments, the agent or cell populations are administered parenterally. In some embodiments, the term “parenteral” includes intravenous, intramuscular, subcutaneous, rectal, vaginal, and intraperitoneal administration. In some embodiments, the agent or cell populations are administered to a subject using peripheral systemic delivery by intravenous, intraperitoneal, or subcutaneous injection.

Compositions in some embodiments are provided as sterile liquid preparations, e.g., isotonic aqueous solutions, suspensions, emulsions, dispersions, or viscous compositions, which may in some aspects be buffered to a selected pH. Liquid preparations can be easier to prepare than gels, other viscous compositions, and solid compositions. Additionally, liquid compositions can be more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol) and suitable mixtures thereof.

Sterile injectable solutions can be prepared by incorporating the agent or cells in a solvent, such as in admixture with a suitable carrier, diluent, or excipient such as sterile water, physiological saline, glucose, dextrose, or the like.

The formulations to be used for in vivo administration are generally sterile. Sterility may be readily accomplished by, e.g., filtration through sterile filtration membranes.

### VIII. Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

As used herein, the singular forms “a,” “an,” and “the” include plural referents unless the context clearly dictates otherwise. For example, “a” or “an” means “at least one” or “one or more.” It is understood that aspects and variations described herein include “consisting” and/or “consisting essentially of” aspects and variations.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that smaller ranges between each intervening value between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

The term “about” as used herein refers to the usual error range for the respective value readily known. Reference to “about” a value or parameter herein includes (and describes) embodiments that are directed to that value or parameter per se. For example, description referring to “about X” includes description of “X”.

As used herein, a statement that a cell or population of cells is “positive” for a particular marker refers to the detectable presence on or in the cell of a particular marker, typically a surface marker. When referring to a surface marker, the term refers to the presence of surface expression as detected by flow cytometry, for example by staining with an antibody that specifically binds to the marker and detecting said antibody, wherein the staining is detectable by flow cytometry at a level substantially above the staining detected carrying out the same procedure with an isotype-matched control under otherwise identical conditions, and/or at a level substantially similar to that for a cell known to be positive for the marker, and/or at a level substantially higher than that for a cell known to be negative for the marker.

As used herein, a statement that a cell or population of cells is “negative” for a particular marker refers to the absence of substantial detectable presence on or in the cell of a particular marker, typically a surface marker. When referring to a surface marker, the term refers to the absence of surface expression as detected by flow cytometry, for example by staining with an antibody that specifically binds to the marker and detecting said antibody, wherein the staining is not detected by flow cytometry at a level substantially above the staining detected carrying out the same procedure with an isotype-matched control under otherwise identical conditions, and/or at a level substantially lower than that for a cell known to be positive for the marker, and/or at a level substantially similar as compared to that for a cell known to be negative for the marker.

As used herein, a composition refers to any mixture of two or more products, substances, or compounds, including cells. It may be a solution, a suspension, liquid, powder, a paste, aqueous, non-aqueous or any combination thereof.

As used herein, a “subject” is a mammal, such as a human or other animal, and typically is human.

### IX. Exemplary Embodiments

Among the provided embodiments are:
1. A protein comprising two heparin-binding domains and a streptavidin-binding partner, wherein the streptavidin-binding partner is connected to one of the heparin-binding domains.
2. The protein of embodiment 1, wherein the protein comprises between 2 and 10 heparin-binding domains, 2 and 9 heparin-binding domains, 2 and 8 heparin-binding domains, 2 and 7 heparin-binding domains, 2 and 6 heparin-binding domains, 2 and 5 heparin-binding domains, 2 and 4 heparin-binding domains, or 2 and 3 heparin-binding domains, each inclusive.
3. The protein of embodiment 1 or embodiment 2, wherein the protein comprises no more than two heparin-binding domains.
4. A protein set forth by the formula (heparin-binding domain)ₙ-(streptavidin-binding partner), wherein n is between 2 and 10, inclusive.
5. The protein of embodiment 4, wherein n is between 2 and 9, 2 and 8, 2 and 7, 2 and 6, 2 and 5, 2 and 4, or 2 and 3, each inclusive.
6. The protein of embodiment 4 or embodiment 5, wherein n is 2.
7. The protein of any one of embodiments 1-6, wherein the heparin-binding domains are different from one another.
8. The protein of any one of embodiments 1-6, wherein the heparin-binding domains are identical to one another.
9. The protein of any one of embodiments 1-8, wherein one, optionally each, of the heparin-binding domains comprises an amino acid sequence that binds to heparin and exhibits at least 85% sequence identity to a heparin-binding domain of fibronectin.
10. The protein of any one of embodiments 1-9, wherein one, optionally each, of the heparin-binding domains comprises the amino acid sequence of a heparin-binding domain of fibronectin.
11. The protein of embodiment 9 or embodiment 10, wherein the heparin-binding domain of fibronectin is heparin-binding domain II of fibronectin (FN12-14).
12. The protein of any one of embodiments 1-11, wherein one, optionally each, of the heparin-binding domains comprises an amino acid sequence selected from X-B-B-X-B-X (SEQ ID NO: 137), X-B-B-B-X-X-B-X (SEQ ID NO: 138), X-B-X-B-B-X (SEQ ID NO: 139), and X-B-X-X-B-B-B-X (SEQ ID NO: 140), wherein each X is independently selected from any hydrophobic amino acid, and each B is independently selected from any basic amino acid.
13. The protein of any one of embodiments 1-12, wherein one, optionally each, of the heparin-binding domains comprises the amino acid sequence X-B-B-X-B-X (SEQ ID NO: 137), wherein each X is independently selected from any hydrophobic amino acid, and each B is independently selected from any basic amino acid.
14. The protein of embodiment 12 or embodiment 13, wherein each B is independently selected from arginine and lysine.
15. The protein of any one of embodiments 1-14, wherein one, optionally each, of the heparin-binding domains comprises the amino acid sequence set forth in SEQ ID NO: 141.
16. The protein of any one of embodiments 1-15, wherein one, optionally each, of the heparin-binding domains comprises the amino acid sequence set forth in SEQ ID NO: 150.
17. The protein of any one of embodiments 1-16, wherein one, optionally each, of the heparin-binding domains comprises the amino acid sequence set forth in SEQ ID NO: 142.
18. The protein of any one of embodiments 1-17, wherein one, optionally each, of the heparin-binding domains comprises an amino acid sequence that binds to heparin and exhibits at least 85% sequence identity to the amino acid sequence set forth in SEQ ID NO: 133.
19. The protein of any one of embodiments 1-18, wherein one, optionally each, of the heparin-binding domains comprises the amino acid sequence set forth in SEQ ID NO: 133.
20. The protein of any one of embodiments 1-19, wherein one, optionally each, of the heparin-binding domains consists of the amino acid sequence set forth in SEQ ID NO: 133.
21. The protein of any one of embodiments 1-20, wherein the heparin-binding domains are connected to one another via a peptide linker.
22. The protein of embodiment 21, wherein the peptide linker is a GS-linker.
23. The protein of embodiment 21 or embodiment 22, wherein the peptide linker comprises the amino acid sequence set forth in SEQ ID NO: 134.
24. The protein of any one of embodiments 21-23, wherein the peptide linker consists of the amino acid sequence set forth in SEQ ID NO: 134.
25. The protein of any one of embodiments 1-24, wherein the streptavidin-binding partner is directly connected to one of the heparin-binding domains.
26. The protein of any one of embodiments 1-25, wherein the streptavidin-binding partner is directly connected to the C-terminus of one of the heparin-binding domains.
27. A protein comprising a heparin-binding domain and a streptavidin-binding partner, wherein the streptavidin-binding partner is connected to the heparin-binding domain.
28. The protein of any one of embodiments 1-27, wherein the streptavidin-binding partner is at the C-terminus of the protein.
29. The protein of any one of embodiments 1-28, wherein the streptavidin-binding partner binds to a molecule that is streptavidin, avidin, a streptavidin analog or mutein, or an avidin analog or mutein.
30. The protein of any one of embodiments 1-29, wherein the streptavidin-binding partner binds to a molecule that is streptavidin or a streptavidin mutein.
31. The protein of embodiment 29 or embodiment 30, wherein the streptavidin-binding partner binds to a biotin-binding site of the molecule.
32. The protein of any one of embodiments 29-31, wherein the streptavidin mutein comprises the amino acid sequence set forth in any of SEQ ID NO: 3-6, 27, 28, 104, 105, and 163.
33. The protein of any one of embodiments 1-32, wherein the streptavidin-binding partner comprises biotin, a biotin analog or derivative, or a streptavidin-binding peptide.
34. The protein of any one of embodiments 1-33, wherein the streptavidin-binding partner comprises a streptavidin-binding peptide.
35. The protein of any one of embodiments 1-34, wherein the streptavidin-binding partner is a streptavidin-binding peptide.
36. The protein of any one of embodiments 33-35, wherein the streptavidin-binding peptide comprises the amino acid sequence set forth SEQ ID NO: 7 or SEQ ID NO: 8.
37. The protein of any one of embodiments 33-36, wherein the streptavidin-binding peptide comprises a sequential arrangement of two streptavidin-binding modules.
38. The protein of embodiment 37, wherein the streptavidin-binding modules are separated from one another by no more than 50 amino acids.
39. The protein of embodiment 37 or embodiment 38, wherein one, optionally each, of the streptavidin-binding modules comprises an amino acid sequence independently selected from the amino acid sequences set forth in SEQ ID NO: 7 and SEQ ID NO: 8.
40. The protein of any one of embodiments 37-39, wherein each of the streptavidin-binding modules comprises the amino acid sequence set forth in SEQ ID NO: 8.
41. The protein of any one of embodiments 33-40, wherein the streptavidin-binding peptide comprises the amino acid sequence set forth in any of SEQ ID NO: 15-19.
42. The protein of any one of embodiments 33-41, wherein the streptavidin-binding peptide consists of the amino acid sequence set forth in any of SEQ ID NO: 15-19.
43. The protein of any one of embodiments 33-42, wherein the streptavidin-binding peptide comprises the amino acid sequence set forth in SEQ ID NO: 16.
44. The protein of any one of embodiments 33-43, wherein the streptavidin-binding peptide consists of the amino acid sequence set forth in SEQ ID NO: 16.
45. The protein of any one of embodiments 1-44, wherein the protein comprises the amino acid sequence set forth in SEQ ID NO: 135.
46. The protein of any one of embodiments 1-45, wherein the protein consists of the amino acid sequence set forth in SEQ ID NO: 135.
47. A heparin-binding reagent comprising (i) a protein reagent comprising one or a plurality of a molecule that is streptavidin, avidin, a streptavidin analog or mutein, or an avidin analog or mutein, and (ii) the protein of any one of embodiments 1-46, wherein the streptavidin-binding partner of the protein is bound to at least one of the one or plurality of molecules.
48. The heparin-binding reagent of embodiment 47, wherein the heparin-binding reagent is capable of being immobilized on a solid support.
49. The heparin-binding reagent of embodiment 47, wherein the heparin-binding reagent is in soluble form.
50. The heparin-binding reagent of any one of embodiments 47-49, wherein the heparin-binding reagent comprises a plurality of the molecule, and the streptavidin-binding partner is bound to at least one of the plurality of molecules.
51. The heparin-binding reagent of embodiment 50, wherein the protein reagent is an oligomer of the plurality of molecules.
52. The heparin-binding reagent of embodiment 50 or embodiment 51, wherein the plurality of molecules comprises between or between about 500 and 5000 tetramers, 1000 and 4000 tetramers, or 2000 and 3000 tetramers, each inclusive, of the molecule of the protein reagent or a mixture of molecules each independently selected from streptavidin, avidin, a streptavidin analog or mutein, or an avidin analog or mutein.
53. The heparin-binding reagent of any one of embodiments 50-52, wherein the plurality of molecules comprises about 2500 tetramers of the molecule of the protein reagent or a mixture of molecules each independently selected from streptavidin, avidin, a streptavidin analog or mutein, or an avidin analog or mutein.
54. The heparin-binding reagent of any one of embodiments 47-53, wherein the molecule of the protein reagent is streptavidin.
55. The heparin-binding reagent of any one of embodiments 47-53, wherein the molecule of the protein reagent is a streptavidin mutein.
56. The heparin-binding reagent of any one of embodiments 47-53 and 55,
wherein the streptavidin mutein comprises the amino acid sequence IGAR (SEQ ID NO: 143) or VTAR (SEQ ID NO: 144) at sequence positions corresponding to positions 44 to 47 of the sequence of amino acids set forth in SEQ ID NO: 1.
57. The heparin-binding reagent of any one of embodiments 47-53, 55, and 56, wherein the streptavidin mutein begins N-terminally in the region of amino acid positions 10 to 16 of SEQ ID NO: 1 and terminates C-terminally in the region of amino acid positions 133 to 142 of SEQ ID NO: 1.
58. A heparin-binding reagent comprising a protein reagent and the protein of any one of embodiments 1-46, wherein:
the protein reagent is an oligomer of a plurality of a molecule that is a streptavidin mutein, wherein the streptavidin mutein (i) comprises the amino acid sequence IGAR (SEQ ID NO: 143) or VTAR (SEQ ID NO: 144) at sequence positions corresponding to positions 44 to 47 of the sequence of amino acids set forth in SEQ ID NO: 1, (ii) begins N-terminally in the region of amino acid positions 10 to 16 of SEQ ID NO: 1, and (iii) terminates C-terminally in the region of amino acid positions 133 to 142 of SEQ ID NO: 1; and
the streptavidin-binding partner of the protein (i) is a streptavidin-binding peptide comprising the amino acid sequence set forth in SEQ ID NO: 7 or SEQ ID NO: 8, and (ii) is bound to at least one of the plurality of molecules.
59. The heparin-binding reagent of embodiment 58, wherein the plurality of molecules comprises between or between about 500 and 5000 tetramers, 1000 and 4000 tetramers, or 2000 and 3000 tetramers, each inclusive, of the molecule of the protein reagent.
60. The heparin-binding reagent of embodiment 58 or embodiment 59, wherein the plurality of molecules comprises about 2500 tetramers of the molecule of the protein reagent.
61. The heparin-binding reagent of any one of embodiments 47-53 and 55-60, wherein the streptavidin mutein further comprises the residues Glu117, Gly120, and Tyr121 with reference to positions of the sequence of amino acids set forth in SEQ ID NO: 1.
62. The heparin-binding reagent of any one of embodiments 47-53 and 55-61, wherein the streptavidin mutein comprises the sequence of amino acids set forth in any of SEQ ID NOs: 3-6, 27, 28, 104, and 105.
63. The heparin-binding reagent of any one of embodiments 47-53, 55-60, and 62, wherein the streptavidin mutein comprises the sequence of amino acids set forth in SEQ ID NO: 6.
64. The heparin-binding reagent of any one of embodiments 50-63, wherein molecules of the plurality of molecules are crosslinked to one another by a polysaccharide or a bifunctional linker.
65. The heparin-binding reagent of any one of embodiments 50-64, wherein molecules of the plurality of molecules are crosslinked to one another by an amine-to-thiol crosslinker.
66. The heparin-binding reagent of any one of embodiments 47-65, wherein the streptavidin-binding partner is bound to a biotin-binding site of the at least one molecule.
67. The heparin-binding reagent of any one of embodiments 47-66, wherein the streptavidin-binding partner is reversibly bound to the at least one molecule.
68. The heparin-binding reagent of any one of embodiments 47-67, wherein the binding affinity of the streptavidin-binding partner to the at least one molecule is reduced compared to the binding affinity of biotin to streptavidin.
69. The heparin-binding reagent of any one of embodiments 47-68, wherein the binding of the streptavidin-binding partner to the at least one molecule is disrupted by the presence of biotin.
70. The heparin-binding reagent of any one of embodiments 47-69, wherein the heparin-binding reagent further comprises one or more binding agents that are each bound to the protein reagent.
71. The heparin-binding reagent of embodiment 70, wherein one, optionally each, of the one or more binding agents is reversibly bound to the protein reagent.
72. The heparin-binding reagent of embodiment 70 or embodiment 71, wherein each of the one or more binding agents comprises a streptavidin-binding partner that is bound to at least one of the plurality of molecules.
73. The heparin-binding reagent of embodiment 72, wherein the streptavidin-binding partner of one, optionally each, of the one or more binding agents is bound to a biotin-binding site of the at least one molecule.
74. The heparin-binding reagent of embodiment 72 or embodiment 73, wherein the streptavidin-binding partner of one, optionally each, of the one or more binding agents comprises biotin, a biotin analog, or a streptavidin-binding peptide.
75. The heparin-binding reagent of any one of embodiments 72-74, wherein the streptavidin-binding partner of one, optionally each, of the one or more binding agents is reversibly bound to the at least one molecule.
76. The heparin-binding reagent of any one of embodiments 72-75, wherein the binding affinity of the streptavidin-binding partner of one, optionally each, of the one or more binding agents to the at least one molecule is reduced compared to the binding affinity of biotin to streptavidin.
77. The heparin-binding reagent of any one of embodiments 72-76, wherein the binding of the streptavidin-binding partner of one, optionally each, of the one or more binding agents to the at least one molecule is disrupted by the presence of biotin.
78. The heparin-binding reagent of any one of embodiments 72-77, wherein the at least one molecule is streptavidin, and the streptavidin-binding partner of one, optionally each, of the one or more binding agents comprises a biotin analog or a streptavidin-binding peptide.
79. The heparin-binding reagent of any one of embodiments 72-78, wherein the streptavidin-binding partner of one, optionally each, of the one or more binding agents comprises a streptavidin-binding peptide.
80. The heparin-binding reagent of any one of embodiments 72-79, wherein the streptavidin-binding partner of one, optionally each, of the one or more binding agents consists of a streptavidin-binding peptide.
81. The heparin-binding reagent of any one of embodiments 74-80, wherein the streptavidin-binding peptide of one, optionally each, of the one or more binding agents comprises an amino acid sequence independently selected from the amino acid sequences set forth in SEQ ID NO: 7 or SEQ ID NO: 8.
82. The heparin-binding reagent of any one of embodiments 74-81, wherein the streptavidin-binding peptide of one, optionally each, of the one or more binding agents comprises a sequential arrangement of two streptavidin-binding modules.
83. The heparin-binding reagent of embodiment 82, wherein the streptavidin-binding modules are separated from one another by no more than 50 amino acids.
84. The heparin-binding reagent of embodiment 82 or embodiment 83, wherein one, optionally each, of the streptavidin-binding modules comprises an amino acid sequence independently selected from the amino acid sequences set forth in SEQ ID NO: 7 and SEQ ID NO: 8.
85. The heparin-binding reagent of any one of embodiments 82-84, wherein each of the streptavidin-binding modules comprises the amino acid sequence set forth in SEQ ID NO: 8.
86. The heparin-binding reagent of any one of embodiments 74-85, wherein the streptavidin-binding peptide of one, optionally each, of the one or more binding agents comprises an amino acid sequence independently selected from the amino acid sequences set forth in SEQ ID NO: 15-19.
87. The heparin-binding reagent of any one of embodiments 74-86, wherein the streptavidin-binding peptide of one, optionally each, of the one or more binding agents consists of an amino acid sequence independently selected from the amino acid sequences set forth in SEQ ID NO: 15-19.
88. The heparin-binding reagent of any one of embodiments 74-87, wherein the streptavidin-binding peptide of one, optionally each, of the one or more binding agents comprises the amino acid sequence set forth in SEQ ID NO: 16.
89. The heparin-binding reagent of any one of embodiments 74-88, wherein the streptavidin-binding peptide of one, optionally each, of the one or more binding agents consists of the amino acid sequence set forth in SEQ ID NO: 16.
90. The heparin-binding reagent of any one of embodiments 70-89, wherein one, optionally each, of the one or more binding agents comprises an antibody or an antibody fragment.
91. The heparin-binding reagent of any one of embodiments 70-90, wherein one, optionally each, of the one or more binding agents comprises an antibody fragment.
92. The heparin-binding reagent of embodiment 90 or embodiment 91, wherein the antibody fragment is a monovalent antibody fragment.
93. The heparin-binding reagent of any one of embodiments 90-92, wherein the antibody fragment is a Fab.
94. The heparin-binding reagent of any one of embodiments 70-93, wherein one of the one or more binding agents is a binding agent that binds to a molecule expressed on the surface of a target cell.
95. The heparin-binding reagent of embodiment 94, wherein the target cell is an immune cell.
96. The heparin-binding reagent of embodiment 94 or embodiment 95, wherein the target cell is a T cell.
97. The heparin-binding reagent of any one of embodiments 94-96, wherein the molecule expressed on the surface of the target cell is a member of a TCR/CD3 complex.
98. The heparin-binding reagent of any one of embodiments 94-97, wherein the molecule expressed on the surface of the target cell is CD3.
99. The heparin-binding reagent of any one of embodiments 94-96, wherein the molecule expressed on the surface of the target cell is a costimulatory molecule.
100. The heparin-binding reagent of any one of embodiments 94-96, wherein the molecule expressed on the surface of the target cell is a co-receptor.
101. The heparin-binding reagent of any one of embodiments 94-100, wherein the binding agent is a first binding agent, the molecule expressed on the surface of the target cell is a first molecule, and one of the one or more binding agents is a second binding agent that binds to a second molecule expressed on the surface of the target cell.
102. The heparin-binding reagent of embodiment 101, wherein the second molecule expressed on the surface of the target cell is a costimulatory molecule.
103. The heparin-binding reagent of any one of embodiments 99, 101, and 102, wherein the costimulatory molecule is CD28, CD90 (Thy-1), CD95 (Apo−/Fas), CD137 (4-1BB), CD154 (CD40L), ICOS, LAT, CD27, OX40, or HVEM.
104. The heparin-binding reagent of any one of embodiments 101-103, wherein the first binding agent comprises the streptavidin-binding partner and an anti-CD3 antibody or antibody fragment, and the second binding agent comprises the streptavidin-binding partner and an anti-CD28 antibody or antibody fragment.
105. The heparin-binding reagent of any one of embodiments 101-104, wherein the first binding agent comprises the streptavidin-binding partner and an anti-CD3 Fab, and the second binding agent comprises the streptavidin-binding partner and an anti-CD28 Fab.
106. The heparin-binding reagent of embodiment 101, wherein the second molecule expressed on the surface of the target cell is a co-receptor.
107. The heparin-binding reagent of any one of embodiments 100-103 and 106, wherein the co-receptor is CD4 or CD8.
108. The heparin-binding reagent of any one of embodiments 100, 101, 106, and 107, wherein the first binding agent comprises the streptavidin-binding partner and an anti-CD4 antibody or antibody fragment, and the second binding agent comprises the streptavidin-binding partner and an anti-CD8 antibody or antibody fragment.
109. The heparin-binding reagent of any one of embodiments 100, 101, and 106-108, wherein the first binding agent comprises the streptavidin-binding partner and an anti-CD4 Fab, and the second binding agent comprises the streptavidin-binding partner and an anti-CD8 Fab.
110. A method for purifying viral particles, comprising:
(a) adding a sample comprising viral particles to an internal cavity of a chromatography column, wherein the internal cavity comprises a stationary phase comprising a chromatography matrix and the protein of any one of embodiments 1-46, and the protein is immobilized on the chromatography matrix, thereby immobilizing a viral particle from the sample on the stationary phase; and
(b) eluting the viral particle from the internal cavity.
111. The method of embodiment 110, wherein the eluting comprises disrupting the binding between the protein and the viral particle.
112. The method of embodiment 110 or embodiment 111, wherein the protein is reversibly bound to the chromatography matrix.
113. The method of any one of embodiments 110-112, wherein the eluting comprises disrupting the binding between the protein and the chromatography matrix.
114. The method of any one of embodiments 110-113, wherein the protein is immobilized on the chromatography matrix via a selection reagent comprising a molecule that is streptavidin, avidin, a streptavidin analog or mutein, or an avidin analog or mutein, wherein the selection reagent is immobilized on the chromatography matrix, and the streptavidin-binding partner of the protein is bound to the molecule.
115. The method of embodiment 114, wherein the streptavidin-binding partner is bound to a biotin-binding site of the molecule of the selection reagent.
116. The method of embodiment 114 or embodiment 115, wherein the streptavidin-binding partner is reversibly bound to the molecule of the selection reagent.
117. The method of any one of embodiments 114-116, wherein the molecule of the selection reagent is a streptavidin mutein.
118. The method of any one of embodiments 114-117, wherein the streptavidin mutein comprises the amino acid sequence IGAR (SEQ ID NO: 143) or VTAR (SEQ ID NO: 144) at sequence positions corresponding to positions 44 to 47 of the sequence of amino acids set forth in SEQ ID NO: 1.
119. The method of any one of embodiments 114-118, wherein the streptavidin mutein begins N-terminally in the region of amino acid positions 10 to 16 of SEQ ID NO: 1 and terminates C-terminally in the region of amino acid positions 133 to 142 of SEQ ID NO: 1.
120. The method of any one of embodiments 114-119, wherein the streptavidin mutein comprises the sequence of amino acids set forth in any of SEQ ID NOs: 3-6, 27, 28, 104, and 105.
121. The method of any one of embodiments 114-120, wherein the streptavidin mutein comprises the sequence of amino acids set forth in SEQ ID NO: 6.
122. The method of any one of embodiments 110-121, wherein the eluting comprises adding, to the internal cavity, a composition comprising a substance that disrupts the immobilization of the viral particle on the stationary phase.
123. The method of embodiment 122, wherein the substance comprises biotin or a biotin analog.
124. The method of embodiment 122 or embodiment 123, wherein the substance comprises biotin.
125. A method for transducing cells, comprising incubating one or more target cells in the simultaneous presence of a viral particle and the heparin-binding reagent of any one of embodiments 47-109, thereby producing a transduced target cell.
126. The method of embodiment 125, wherein at least a portion of the incubating occurs in an internal cavity of a chromatography column.
127. The method of embodiment 125 or embodiment 126, wherein the one or more target cells are immobilized on a solid support during at least a portion of the incubating.
128. The method of embodiment 127, wherein the solid support is a stationary phase for column chromatography.
129. The method of embodiment 128, wherein the stationary phase is comprised in an internal cavity of a chromatography column during the at least a portion of the incubating.
130. A method for on-column transduction of cells, comprising incubating, in an internal cavity of a chromatography column, one or more target cells in the simultaneous presence of a viral particle and the heparin-binding reagent of any one of embodiments 47-109, thereby producing a transduced target cell, wherein the internal cavity comprises a stationary phase, and the one or more target cells are immobilized on the stationary phase during at least a portion of the incubation.
131. The method of any one of embodiments 128-130, wherein prior to the incubating, the method comprises adding a sample comprising a plurality of the target cells to the stationary phase, thereby immobilizing the one or more target cells on the stationary phase.
132. The method of any one of embodiments 128-131, wherein the stationary phase comprises a selection agent that specifically binds to a selection marker expressed on the surface of the one or more target cells, wherein specific binding of the selection agent to the selection marker effects the immobilization of the one or more target cells on the stationary phase.
133. A method for on-column transduction of cells, comprising:
(a) adding a sample comprising a plurality of target cells to an internal cavity of a chromatography column, wherein the internal cavity comprises a stationary phase comprising a selection agent that specifically binds to a selection marker expressed on the surface of one or more of the plurality of target cells, thereby immobilizing the one or more target cells on the stationary phase; and
(b) incubating the one or more target cells in the simultaneous presence of a viral particle and the heparin-binding reagent of any one of embodiments 47-109, thereby producing a transduced target cell, wherein the one or more target cells are immobilized on the stationary phase during at least a portion of the incubation.
134. The method of any one of embodiments 125-133, wherein prior to the incubating, the method comprises contacting the one or more target cells with one or more compositions each comprising one or both of the heparin-binding reagent and the viral particle.
135. The method of any one of embodiments 128-134, wherein prior to the incubating, the method comprises adding, to the stationary phase, one or more compositions each comprising one or both of the heparin-binding reagent and the viral particle, thereby contacting the one or more target cells with the one or more compositions.
136. The method of embodiment 135, wherein the one or more compositions are a first composition comprising the heparin-binding reagent and a second, separate composition comprising the viral particle.
137. The method of embodiment 136, wherein the one or more target cells are simultaneously contacted with the first composition and the second composition.
138. The method of embodiment 135, wherein the one or more compositions is a composition comprising both of the heparin-binding reagent and the viral particle.
139. The method of embodiment 138, further comprising mixing the heparin-binding reagent and the viral particle to form the composition comprising both of the heparin-binding reagent and the viral particle.
140. The method of any one of embodiments 125-139, wherein prior to the incubating, the one or more target cells are incubated in the presence of one or more binding agents, one of the one or more binding agents being a binding agent that binds to a molecule expressed on the surface of the one or more target cells.
141. The method of any one of embodiments 125-140, wherein at least a portion, optionally all, of the incubating is further in the presence of one or more binding agents, one of the one or more binding agents being a binding agent that binds to a molecule expressed on the surface of the one or more target cells.
142. The method of embodiment 140 or embodiment 141, wherein the one or more binding agents are comprised in a second reagent comprising a second protein reagent comprising one or a plurality of a molecule that is streptavidin, avidin, a streptavidin analog or mutein, or an avidin analog or mutein, wherein the one or more binding agents are each bound to the second protein reagent of the second reagent.
143. The method of embodiment 142, wherein one, optionally each, of the one or more binding agents is reversibly bound to the second protein reagent.
144. The method of embodiment 142 or embodiment 143, wherein each of the one or more binding agents comprises a streptavidin-binding partner that is bound to at least one of the plurality of molecules of the second protein reagent.
145. The method of any one of embodiments 142-144, wherein the second reagent is a stimulatory reagent.
146. The method of any one of embodiments 142-145, wherein the method comprises contacting the one or more target cells with a composition comprising the second reagent.
147. The method of any one of embodiments 142-146, wherein the method comprises adding, to the stationary phase, a composition comprising the second reagent, thereby contacting the one or more target cells with the composition comprising the second reagent.
148. The method of embodiment 146 or embodiment 147, wherein the one or more target cells are simultaneously contacted with the composition comprising the second reagent and the one or more compositions each comprising one or both of the heparin-binding reagent and the viral particle.
149. The method of any one of embodiments 142-145, wherein the composition comprising both of the heparin-binding reagent and the viral particle further comprises the second reagent.
150. The method of embodiment 140 or embodiment 141, wherein the heparin-binding reagent comprises the one or more binding agents, wherein the one or more binding agents are each bound to the protein reagent of the heparin-binding reagent.
151. The method of embodiment 150, wherein one, optionally each, of the one or more binding agents is reversibly bound to the protein reagent.
152. The method of embodiment 150 or embodiment 151, wherein each of the one or more binding agents comprises a streptavidin-binding partner that is bound to at least one of the plurality of molecules of the protein reagent.
153. The method of any one of embodiments 140-152, wherein the streptavidin-binding partner of one, optionally each, of the one or more binding agents comprises biotin, a biotin analog, or a streptavidin-binding peptide.
154. The method of any one of embodiments 140-153, wherein the streptavidin-binding partner of one, optionally each, of the one or more binding agents comprises a streptavidin-binding peptide.
155. The method of embodiment 153 or embodiment 154, wherein the streptavidin-binding peptide of one, optionally each, of the one or more binding agents comprises the amino acid sequence set forth in any of SEQ ID NO: 7, 8, and 15-19.
156. The method of any one of embodiments 153-155, wherein the streptavidin-binding peptide of one, optionally each, of the one or more binding agents comprises the amino acid sequence set forth in SEQ ID NO: 16.
157. The method of any one of embodiments 140-156, wherein one, optionally each, of the one or more binding agents comprises an antibody or an antibody fragment.
158. The method of any one of embodiments 140-157, wherein one, optionally each, of the one or more binding agents comprises an antibody fragment.
159. The method of embodiment 157 or embodiment 158, wherein the antibody fragment is a monovalent antibody fragment.
160. The method of any one of embodiments 157-159, wherein the antibody fragment is a Fab.
161. The method of any one of embodiments 140-160, wherein the binding agent binds to the molecule expressed on the one or more target cells and thereby provides a primary activation signal to the one or more target cells.
162. The method of any one of embodiments 125-161, wherein the one or more target cells are immune cells.
163. The method of any one of embodiments 125-162, wherein the one or more target cells are T cells.
164. The method of any one of embodiments 140-163, wherein the molecule expressed on the surface of the one or more target cells is a member of a TCR/CD3 complex.
165. The method of any one of embodiments 140-164, wherein the molecule expressed on the surface of the one or more target cells is CD3.
166. The method of any one of embodiments 140-160, wherein the molecule expressed on the surface of the one or more target cells is a costimulatory molecule.
167. The method of embodiment 166, wherein the binding agent binds to the costimulatory molecule and thereby provides a costimulatory signal to the one or more target cells.
168. The method of any one of embodiments 140-160, wherein the molecule expressed on the surface of the one or more target cells is a co-receptor.
169. The method of any one of embodiments 140-168, wherein the binding agent is a first binding agent, the molecule expressed on the surface of the one or more target cells is a first molecule, and one of the one or more binding agents is a second binding agent that binds to a second molecule expressed on the surface of the one or more target cells.
170. The method of embodiment 169, wherein the second molecule expressed on the surface of the one or more target cells is a costimulatory molecule.
171. The method of embodiment 170, wherein the second binding agent binds to the costimulatory molecule and thereby provides a costimulatory signal to the one or more target cells.
172. The method of any one of embodiments 166, 167, and 169-171, wherein the costimulatory molecule is CD28, CD90 (Thy-1), CD95 (Apo−/Fas), CD137 (4-1BB), CD154 (CD40L), ICOS, LAT, CD27, OX40, or HVEM.
173. The method of any one of embodiments 169-172, wherein the first binding agent comprises a streptavidin-binding partner and an anti-CD3 antibody or antibody fragment, and the second binding agent comprises a streptavidin-binding partner and an anti-CD28 antibody or antibody fragment.
174. The method of any one of embodiments 169-173, wherein the first binding agent comprises a streptavidin-binding partner and an anti-CD3 Fab, and the second binding agent comprises a streptavidin-binding partner and an anti-CD28 Fab.
175. The method of embodiment 169, wherein the second molecule expressed on the surface of the target cell is a co-receptor.
176. The method of any one of embodiments 168, 169, and 175, wherein the co-receptor is CD4 or CD8.
177. The method of any one of embodiments 168, 169, 175, and 176, wherein the first binding agent comprises a streptavidin-binding partner and an anti-CD4 antibody or antibody fragment, and the second binding agent comprises a streptavidin-binding partner and an anti-CD8 antibody or antibody fragment.
178. The method of any one of embodiments 168, 169, and 175-177, wherein the first binding agent comprises a streptavidin-binding partner and an anti-CD4 Fab, and the second binding agent comprises a streptavidin-binding partner and an anti-CD8 Fab.
179. The method of any one of embodiments 131-178, wherein the incubating is initiated within or within about 10 minutes, within or within about 20 minutes, within or within about 30 minutes, within or within about 45 minutes, within or within about 60 minutes, within or within about 90 minutes, or within or within about 120 minutes after adding the sample to the internal cavity.
180. The method of any one of embodiments 125-179, wherein the incubating is carried out in a cell media.
181. The method of embodiment 180, wherein the cell media is a serum free media.
182. The method of embodiment 180 or embodiment 181, wherein the cell media comprises a recombinant cytokine.
183. The method of embodiment 182, wherein the recombinant cytokine is selected from IL-2, IL-15, and IL-7.
184. The method of any one of embodiments 180-183, wherein the cell media comprises recombinant IL-2, IL-15, and IL-7.
185. The method of any one of embodiments 125-184, wherein the incubating is in the presence of between or between about 0.1 μg and 20 μg, inclusive; between or between about 0.1 μg and 12 μg, inclusive; or between or between about 0.5 μg and 8 μg, inclusive, of the heparin-binding reagent; each per 10⁶ cells of the one or more target cells or of an estimated cell count thereof.
186. The method of any one of embodiments 125-185, wherein the incubating is in the presence of between or between about 3 μg and 5 μg, inclusive, of the heparin-binding reagent per 10⁶ cells of the one or more target cells or of an estimated cell count thereof.
187. The method of any one of embodiments 134-186, wherein the composition of the one or more compositions that comprises the heparin-binding reagent comprises between or between about 0.1 μg and 20 μg, inclusive; between or between about 0.1 μg and 12 μg, inclusive; or between or between about 0.5 μg and 8 μg, inclusive, of the heparin-binding reagent; each per 10⁶ cells of the one or more target cells or of an estimated cell count thereof.
188. The method of any one of embodiments 134-187, wherein the composition of the one or more compositions that comprises the heparin-binding reagent comprises between or between about 3 μg and 5 μg, inclusive, of the heparin-binding reagent per 10⁶ cells of the one or more target cells or of an estimated cell count thereof.
189. The method of any one of embodiments 125-188, wherein the incubating is in the presence of between or between about 0.1 μL and 100 μL, inclusive; between or between about 0.5 μL and 50 μL, inclusive; between or between about 1 μL and 25 μL, inclusive; or between or between about 2 μL and 10 μL, inclusive, of a preparation of the viral particle per 10⁶ cells of the one or more target cells or of an estimated cell count thereof.
190. The method of any one of embodiments 125-189, wherein the incubating is in the presence of about 6 μL per 10⁶ cells of the one or more target cells or of an estimated cell count thereof.
191. The method of any one of embodiments 134-190, wherein the composition of the one or more compositions that comprises the viral particle comprises between or between about 0.1 μL and 100 μL, inclusive; between or between about 0.5 μL and 50 μL, inclusive; between or between about 1 μL and 25 μL, inclusive; or between or between about 2 μL and 10 μL, inclusive, of a preparation of the viral particle per 10⁶ cells of the one or more target cells or of an estimated cell count thereof.
192. The method of any one of embodiments 134-191, wherein the composition of the one or more compositions that comprises the viral particle comprises about 6 μL per 10⁶ cells of the one or more target cells or of an estimated cell count thereof.
193. The method of any one of embodiments 189-192, wherein the preparation of the viral particle has a titer of between or between about 1×10⁶ TU/mL and 1×10⁹ TU/mL, between or between about 1×10⁶ TU/mL and 1×10⁸ TU/mL, between or between about 1×10⁶ TU/mL and 1×10⁷ TU/mL, between or between about 1×10⁷ TU/mL and 1×10⁹ TU/mL, between or between about 1×10⁷ TU/mL and 1×10⁸ TU/mL, or between or between about 1×10⁸ TU/mL and 1×10⁹ TU/mL.
194. The method of any one of embodiments 128-193, wherein the stationary phase has a binding capacity of between or between about 0.5 billion and 5 billion cells, 0.5 billion and 4 billion cells, 0.5 billion and 3 billion cells, 0.5 billion and 2 billion cells, 1 billion and 5 billion cells, 1 billion and 4 billion cells, 1 billion and 3 billion cells, or 1 billion and 2 billion cells, each inclusive.
195. The method of any one of embodiments 128-194, wherein the stationary phase has a binding capacity of between or between about 1 billion and 2 billion cells, inclusive.
196. The method of any one of embodiments 125-195, wherein at least a portion, optionally all, of the incubating is carried out at a temperature between about 35° C. and about 39° C.
197. The method of any one of embodiments 125-196, wherein at least a portion, optionally all, of the incubating is carried out at a temperature of or of about 37° C.
198. The method of any one of embodiments 125-197, further comprising collecting the transduced target cell.
199. The method of embodiment 198, wherein the collecting comprises eluting the transduced target cell from the chromatography column.
200. The method of embodiment 198 or embodiment 199, the collecting is carried out within 24 hours, 22 hours, 20 hours, 18 hours, 16 hours, 16 hours, 14 hours, 12 hours, 10 hours, 9 hours, 8 hours, 7 hours, 6 hours, or 5 hours after the initiation of the incubating.
201. The method of any one of embodiments 198-200, wherein the collecting is carried out between or between about 2 hours and 24 hours, 2 hours and 22 hours, 2 hours and 20 hours, 2 hours and 18 hours, 2 hours and 16 hours, 2 hours and 14 hours, 2 hours and 12 hours, 2 hours and 10 hours, 2 hours and 9 hours, 2 hours and 8 hours, 2 hours and 7 hours, 2 hours and 6 hours, 2 hours and 5 hours, 3 hours and 6 hours, 3 hours and 5 hours, 4 hours and 6 hours, or 4 hours and 5 hours, each inclusive, after the initiation of the incubating.
202. The method of any one of embodiments 198-201, wherein the collecting is carried out at or about 4.5 hours after the initiation of the incubating.
203. The method of any one of embodiments 198-202, wherein the collecting comprises adding a wash buffer to the chromatography column to collect the transduced target cell.
204. The method of embodiment 203, wherein the wash buffer comprises a cell media.
205. The method of embodiment 203 or embodiment 204, wherein the wash buffer does not comprise a competition agent to elute the transduced target cell from the stationary phase.
206. The method of embodiment 203 or embodiment 204, wherein the wash buffer comprises a competition agent to elute the transduced target cell from the stationary phase.
207. The method of embodiment 205 or embodiment 206, wherein the competition agent facilitates detachment of the one or more target cells from the stationary phase.
208. The method of any one of embodiments 205-207, wherein the competition agent comprises biotin or a biotin analog.
209. The method of any one of embodiments 205-208, wherein the competition agent comprises biotin.
210. The method of any one of embodiments 198-209, comprising further incubating the collected transduced target cell.
211. The method of any one of embodiments 132-210, wherein the stationary phase comprises a chromatography matrix, and the selection agent is immobilized on the chromatography matrix.
212. The method of embodiment 211, wherein the selection agent is immobilized on the chromatography matrix via a selection reagent comprising a molecule that is streptavidin, avidin, a streptavidin analog or mutein, or an avidin analog or mutein, wherein the selection reagent is immobilized on the chromatography matrix, and the selection agent comprises a streptavidin-binding partner that is bound to the molecule.
213. The method of embodiment 212, wherein the streptavidin-binding partner of the selection agent is bound to a biotin-binding site of the molecule of the selection reagent.
214. The method of embodiment 212 or embodiment 213, wherein the molecule of the selection reagent is a streptavidin mutein.
215. The method of any one of embodiments 212-214, wherein the streptavidin mutein comprises the amino acid sequence IGAR (SEQ ID NO: 143) or VTAR (SEQ ID NO: 144) at sequence positions corresponding to positions 44 to 47 of the sequence of amino acids set forth in SEQ ID NO: 1.
216. The method of any one of embodiments 212-215, wherein the streptavidin mutein begins N-terminally in the region of amino acid positions 10 to 16 of SEQ ID NO: 1 and terminates C-terminally in the region of amino acid positions 133 to 142 of SEQ ID NO: 1.
217. The method of any one of embodiments 212-216, wherein the streptavidin mutein comprises the sequence of amino acids set forth in any of SEQ ID NOs: 3-6, 27, 28, 104, 105, and 163.
218. The method of any one of embodiments 212-217, wherein the streptavidin mutein comprises the sequence of amino acids set forth in SEQ ID NO: 6.
219. The method of any one of embodiments 212-218, wherein the streptavidin-binding partner of the selection agent comprises biotin, a biotin analog, or a streptavidin-binding peptide.
220. The method of any one of embodiments 212-219, wherein the binding of the streptavidin-binding partner of the selection agent to the molecule of the selection reagent is reversible.
221. The method of any one of embodiments 212-220, wherein the streptavidin-binding partner of the selection agent comprises a streptavidin-binding peptide.
222. The method of any one of embodiments 219-221, wherein the streptavidin-binding peptide of the selection agent comprises the amino acid sequence set forth in any of SEQ ID NO: 7, 8, and 15-19.
223. The method of any one of embodiments 219-222, wherein the streptavidin-binding peptide comprises the amino acid sequence set forth in SEQ ID NO: 16.
224. The method of any one of embodiments 132-223, wherein the selection agent comprises an antibody or antibody fragment that binds to the selection marker.
225. The method of any one of embodiments 132-224, wherein the selection agent comprises an antibody fragment that binds to the selection marker.
226. The method of embodiment 224 or embodiment 225, wherein the antibody fragment is a monovalent antibody fragment.
227. The method of any one of embodiments 224-226, wherein the antibody fragment is a Fab.
228. The method of any one of embodiments 132-227, wherein the selection marker is a T cell coreceptor or a member of a T cell antigen receptor complex.
229. The method of any one of embodiments 132-228, wherein the selection marker is selected from the group consisting of CD3, CD4, CD8, CD45RA, CD27, CD28, and CCR7.
230. The method of any one of embodiments 132-229, wherein the selection marker is CD3.
231. The method of any one of embodiments 125-230, wherein the one or more target cells are primary cells from a human subject.
232. The method of any one of embodiments 131-231, wherein the sample is an apheresis or leukapheresis product.
233. The method of any one of embodiments 125-232, wherein the viral particle comprises a nucleic acid sequence encoding a recombinant protein.
234. The method of embodiment 233, wherein the recombinant protein is an antigen receptor.
235. The method of embodiment 233 or embodiment 234, wherein the recombinant protein is a chimeric antigen receptor (CAR).
236. The method of embodiment 233 or embodiment 234, wherein the recombinant protein is a T cell receptor (TCR).
237. The method of any one of embodiments 125-236, wherein the viral particle is a viral vector.
238. The method of any one of embodiments 198-237, further comprising harvesting the collected transduced target cell.
239. The method of embodiment 238, wherein the harvesting is carried out after the further incubating.
240. The method of embodiment 238 or embodiment 239, further comprising formulating the harvested transduced target cell for cryopreservation or administration to a subject.
241. The method of any one of embodiments 238-240, wherein the harvested transduced target cell is formulated in the presence of a pharmaceutically acceptable excipient or a cryoprotectant.
242. The method of any one of embodiments 125-241, wherein one, optionally all, of the steps of the method is carried out in a closed system.
243. The method of any one of embodiments 125-242, wherein one, optionally all, of the steps of the method is automated.
244. A kit for purifying viral particles, comprising the protein of any one of embodiments 1-46, a chromatography matrix suitable for viral purification using column chromatography, and a selection reagent, the selection reagent comprising a molecule that is streptavidin, avidin, a streptavidin analog or mutein, or an avidin analog or mutein, wherein the selection reagent is capable of being immobilized on the chromatography matrix.
245. The kit of embodiment 244, wherein the streptavidin-binding partner of the protein is bound to the molecule.
246. A kit for transducing cells, comprising the heparin-binding reagent of any one of embodiments 47-109, a chromatography matrix suitable for cell separation using column chromatography, and a selection agent that specifically binds to a selection marker expressed on the surface of a target cell, wherein the selection agent is capable of being immobilized on the chromatography matrix.
247. The kit of embodiment 246, further comprising a viral particle.
248. The kit of embodiment 246 or embodiment 247, further comprising a stimulatory reagent.
249. The kit of any one of embodiments 246-248, wherein the selection agent is immobilized on the chromatography matrix.
250. The kit of any one of embodiments 246-249, wherein the selection agent comprises a streptavidin-binding partner.
251. The kit of any one of embodiments 246-250, further comprising a selection reagent, the selection reagent comprising a molecule that is streptavidin, avidin, a streptavidin analog or mutein, or an avidin analog or mutein, wherein the selection reagent is capable of being immobilized on the chromatography matrix.
252. The kit of any one of embodiments 244, 245, and 251, wherein the selection reagent is immobilized on the chromatography matrix.
253. The kit of embodiment 251 or embodiment 252, wherein the streptavidin-binding partner of the selection agent is bound to the molecule.
254. The kit of any one of embodiments 244-253, further comprising a chromatography column.
255. The kit of embodiment 254, wherein the chromatography matrix is comprised in an internal cavity of the chromatography column.
256. A stationary phase for purifying viral particles, comprising the protein of any one of embodiments 1-46, a chromatography matrix suitable for viral purification using column chromatography, and a selection reagent, the selection reagent comprising a molecule that is streptavidin, avidin, a streptavidin analog or mutein, or an avidin analog or mutein, wherein the selection agent is immobilized on the chromatography matrix, and the streptavidin-binding partner of the protein is bound to the molecule.
257. An article of manufacture for purifying viral particles, comprising the stationary phase of embodiment 256 and a chromatography column, wherein the stationary phase is comprised in an internal cavity of the chromatography column.
258. A viral particle purified by the method of any of embodiments 110-124.
259. A target cell transduced by the method of any of embodiments 125-243.

### X. Examples

The following examples are included for illustrative purposes only and are not intended to limit the scope of the invention.

### Example 1: Generation of Viral Binding Protein (VBP)

A viral binding protein (VBP) containing multiple viral binding domains was generated. The VBP (SEQ ID NO: 135) contained two viral binding domains, each viral binding domain containing the sequence of heparin binding domain II of fibronectin (FN12-14, SEQ ID NO: 133). The two viral binding domains were connected with a GS-linker (SEQ ID NO: 134), and for use as an affinity tag, a streptavidin-binding peptide sequence (Twin-Strep-Tag®, SEQ ID NO: 16) was added to the C-terminus of the second viral binding domain. Sequence identifiers for the VBP are summarized in Table E1.

A sequence encoding the linker-connected viral binding domains of the VBP was in silico designed and ordered as a DNA String. Subsequent cloning was performed with appropriate restriction enzymes to insert the designed sequence into an inducible pASG-IBA102 vector (IBA Lifesciences GmbH) encoding a C-terminal Twin-Strep-Tag®. DNA was purified with a Plasmid Miniprep kit (Promega Corporation). The E. coli BL21 bacteria strain was used for protein expression using a standard cytoplasmic expression protocol, with protein expression induced by anhydrotetracycline through an integrated tetracycline promotor. Bacteria were lysed via sonication, and whole cell lysate was purified using a StrepTactin® XT gravity flow column (IBA Lifesciences GmbH), resulted in pure target protein.

TABLE E1
Sequence Identifiers (SEQ ID NO) for Viral Binding
Protein (VBP) and Subsequences Thereof

| Viral Binding | Linker | Viral Binding | Affinity | Full Sequence |
|---|---|---|---|---|
| Domain 1 (aa) | (aa) | Domain 2 (aa) | Tag (aa) | of VBP (aa) |
| 133 | 134 | 133 | 16 | 135 |

The VBP was tested for viral binding. To do so, the VBP was immobilized on an affinity chromatography column, to which a sample containing lentiviral particles was applied.

To prepare the affinity chromatography column, a StrepTactin® Superflow® column was used (0.2 mL resin bed, 3 mg binding capacity per mL; IBA Lifesciences GmbH). To saturate and functionalize the column, 0.6 mg of the VBP in PBS was loaded by gravity flow, after which the column was equilibrated and washed once with PBS.

To test for viral binding, samples containing GFP-encoding lentiviral particles were applied to columns that had been functionalized with or without VBP. Flow through (FT) fractions were collected, after which elution fractions containing viral binding protein and potentially bound virus were eluted by addition of biotin-containing buffer. FT and elution fractions were then used to transduce T cells. As a positive control, T cells were transduced with unprocessed virus, and as a negative control, T cells were mock transduced. Five days after transduction, cells were assessed for GFP expression by flow cytometry.

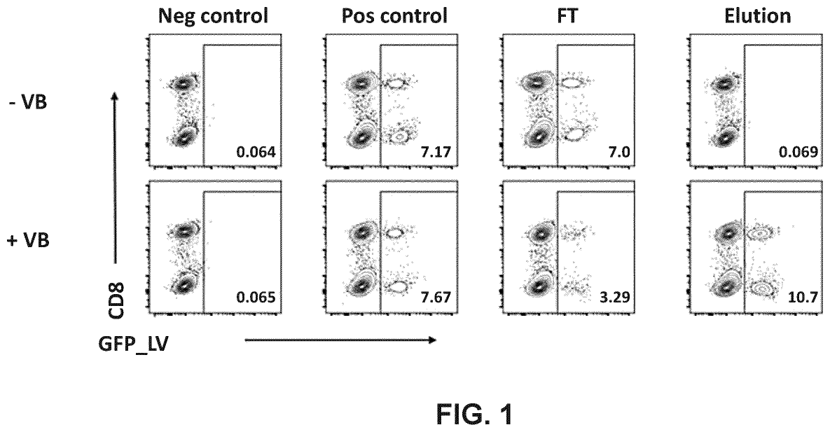

FIG. 1 shows GFP expression of T cells five days after transduction using FT or elution fractions collected from columns functionalized with viral binding protein (+VB) or columns functionalized without viral binding protein (−VB). Results shown are for cells pre-gated for living lymphocytes. As shown in FIG. 1, T cells transduced with FT fractions of −VB samples showed 7% CD8−CD8+GFP+ cells, which was similar to levels for the positive control. In contrast, T cells transduced with FT fractions of +VB samples showed 3.29% CD8−CD8+GFP+ cells, indicating depletion of virus in FT fractions when columns were functionalized with VBP. T cells transduced with elution fractions of −VB samples showed levels of CD8-CD8+GFP+ cells comparable to those of the negative control, indicating that virus was not retained on columns that had not been functionalized with VBP. T cells transduced with elution fractions of +VB samples showed 10.7% CD8-CD8+GFP+ cells.

Together, these results indicate that the VBP can be used to isolate viral particles using column chromatography. These results also demonstrate that the VBP and isolated viral particles can be functionally eluted without affecting the ability of the isolated viral particles to transduce cells.

### Example 2: On-Column Transduction of T Cells in the Presence of Viral Binding Protein (VBP)

The viral binding protein (VBP) described in Example 1 was tested for its effects on the on-column transduction of cells. To do so, the VBP was multimerized using an oligomeric streptavidin mutein reagent, and T cells immobilized on an affinity chromatography column were transduced in the presence of lentiviral particles and the VBP-functionalized reagent.

An anti-CD3 affinity chromatography column was prepared by adding, to a streptavidin mutein-coated resin as described in Example 1, 1.8 mg of an anti-CD3 Fab fragment. The anti-CD3 Fab fragment was derived from the CD3 binding monoclonal antibody produced by the hybridoma cell line OKT3 (ATCC® CRL-8001™; see also U.S. Pat. No. 4,361,549), and contained the heavy chain variable domain (SEQ ID NO: 31) and light chain variable domain (SEQ ID NO: 32) of the anti-CD3 antibody OKT3 described in Arakawa et al., J. Biochem. 120, 657-662 (1996). The anti-CD3 Fab fragments were individually fused at the carboxy-terminus of their heavy chain to a streptavidin-binding peptide sequence (Twin-Strep-Tag®, SEQ ID NO: 16) containing a sequential arrangement of two streptavidin-binding modules. The peptide-tagged Fab fragments were recombinantly produced (see International Patent App. Pub. Nos. WO 2013/011011 and WO 2013/124474).

For stimulating cells, an anti-CD3/anti-CD28 stimulatory reagent was prepared using an oligomeric streptavidin mutein reagent produced as described in WO 2018/197949 (see also Poltorak et al., Scientific Reports (2020)). The oligomeric streptavidin mutein reagent had an average hydrodynamic radius of 90-120 nm and contained an average of 2000-2800 streptavidin mutein tetramers. The oligomeric streptavidin mutein reagent was mixed at room temperature with the anti-CD3 Fab fragments described above as well as with anti-CD28 Fab fragments also individually fused at the carboxy-terminus of their heavy chain to a streptavidin-binding peptide sequence (Twin-Strep-Tag®, SEQ ID NO: 16). The anti-CD28 Fab fragments were derived from antibody CD28.3 (deposited as a synthetic single chain Fv construct under GenBank Accession No. AF451974.1; see also Vanhove et al., BLOOD, 15 Jul. 2003, Vol. 102, No. 2, pages 564-570) and contained the heavy chain variable domain (SEQ ID NO: 33) and the light chain variable domain (SEQ ID NO: 34) of the anti-CD28 antibody CD28.3.

For VBP multimerization, a separate VBP-functionalized reagent was prepared. To do so, 1.5 mg of the oligomeric streptavidin mutein reagent (produced as described in WO 2018/197949) was mixed with 0.25 mg of VBP at room temperature.

For T cell isolation, stimulation, and transduction, a fresh apheresis sample from a human donor was loaded onto the anti-CD3 functionalized column. Following loading and washing, the T cells while immobilized on-column were incubated in the presence of an activation-transduction buffer. The activation-transduction buffer included a serum-free medium containing the following: recombinant IL-2, IL-15, and IL-7; 2 mg of the anti-CD3/anti-CD28 stimulatory reagent; 6 μL/1×10⁶ cells of lentiviral particles encoding an exemplary anti-BCMA CAR; and 1.75 mg of the VBP-functionalized reagent. Approximately 0.5×10⁹ cells were expected to be captured per column.

Following addition of the activation-transduction buffer, the affinity chromatography column was heated and incubated at 37° C. for 4.5 hours. Following incubation, during which time cells spontaneously detached from the column, cells were eluted using a serum-free medium containing recombinant IL-12, IL-15, and IL-7 only (without the need to add biotin or other competition substance to release the immobilized cells). Following elution, the cells were adjusted to a seeding cell concentration of 3×10⁶ cells/mL by addition of a serum-free medium containing recombinant IL-2, IL-15, and IL-7 and then underwent further culturing at 37° C. in an incubator. After five days of culture, CAR expression was assessed by flow cytometry using an anti-idiotypic antibody against the CAR in order to determine transduction efficiency.

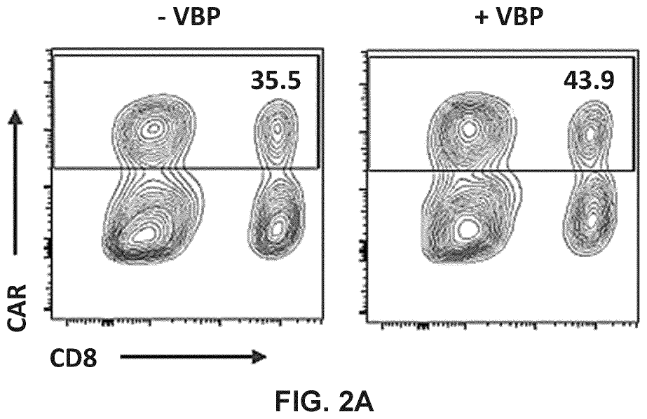

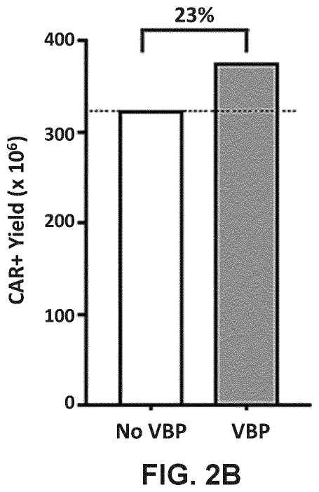

FIG. 2A and FIG. 2B show CAR expression of T cells following transduction in the presence (+VBP) or absence (−VBP) of VBP-functionalized reagent and five days of culture. Results shown are for cells pre-gated for living, single lymphocytes. As shown in FIG. 2A, T cells transduced in the presence of VBP-functionalized oligomeric reagent showed increased transduction efficiency (43.9% CD8−CD8+CAR+ T cells) compared to T cells transduced in the absence of VBP-functionalized reagent (35.5% CD8−CD8+CAR+ T cells). As shown in FIG. 2B, increased transduction efficiency for T cells transduced in the presence of VBP-functionalized reagent resulted in 23% higher yield of CAR T cells.

Together, these results indicate that the presence of multimerized VBP during transduction can increase transduction efficiency.

### Example 3: Assessment of Anti-CD4/Anti-CD8 Viral Binding Protein (VBP)-Functionalized Reagent for On-Column Transduction

The viral binding protein (VBP)-functionalized reagent described in Example 2 was further functionalized with anti-CD4 and anti-CD8 Fab fragments and tested for its effect on the on-column transduction of cells. T cells immobilized on an affinity chromatography column were transduced with lentiviral particles in the presence of an anti-CD3/anti-CD28 stimulatory reagent and additionally with or without oligomeric streptavidin mutein reagent that was further functionalized with or without VBP and with or without anti-CD4 and anti-CD8 Fab fragments.

To do so, an anti-CD3 affinity chromatography column, an anti-CD3/anti-CD28 stimulatory reagent, and a separate VBP-functionalized reagent were prepared as described in Example 2. To prepare a separate anti-CD4/anti-CD8 VBP-functionalized reagent, 0.3 mg of the oligomeric streptavidin mutein reagent described in Example 2 was mixed at room temperature with 50 μg of anti-CD4 Fab fragments, 50 μg of anti-CD8 Fab fragments, and 50 μg of the VBP. For comparison, an anti-CD4/anti-CD8 reagent without VBP was also prepared using 0.3 mg of the oligomeric streptavidin mutein reagent, 50 μg of anti-CD4 Fab fragments, and 50 μg of anti-CD8 Fab fragments.

The anti-CD4 Fab fragment was derived from antibody 13B8.2 (see U.S. Pat. No. 7,482,000, U.S. Patent Appl. No. US2014/0295458, and International Patent Application No. WO2013/124474; and Bes, C, et al. J Biol Chem 278, 14265-14273 (2003)). The fragment contained the heavy chain variable domain set forth by SEQ ID NO: 29 and the light chain variable domain set forth by SEQ ID NO: 30. The anti-CD8 Fab fragment was derived from antibody OKT8 (ATCC CRL-8014) and contained the heavy chain variable domain set forth by SEQ ID NO: 36 and the light chain variable domain set forth by SEQ ID NO: 37. The anti-CD4 and anti-CD8 Fab fragments were individually fused at the carboxy-terminus of their heavy chain to a streptavidin-binding peptide sequence (Twin-Strep-Tag®, SEQ ID NO: 16) containing a sequential arrangement of two streptavidin-binding modules. The peptide-tagged Fab fragments were recombinantly produced (see International Patent App. Pub. Nos. WO 2013/011011 and WO 2013/124474).

For T-cell isolation, stimulation, and transduction, a fresh apheresis sample was loaded onto the anti-CD3 functionalized column. Post loading and washing, the immobilized T cells were incubated in the presence of activation-transduction buffer containing the following: recombinant IL-2, IL-15, and IL-7; 0.4 mg of the anti-CD3/anti-CD28 stimulatory reagent; 6 μL/1×10⁶ cells of lentiviral particles encoding an exemplary anti-BCMA CAR; and 0.45 mg of (i) the anti-CD4/anti-CD8 VBP-functionalized reagent, (ii) the anti-CD4/anti-CD8 reagent (without VBP), (iii) the VBP-functionalized reagent (without anti-CD4/anti-CD8 Fab fragments), or (iv) the oligomeric streptavidin mutein reagent (without functionalization). Approximately 100×10⁶ cells were expected to be captured per column.

Following addition of the activation-transduction buffer, the affinity chromatography column was heated and incubated at 37° C. for 4.5 hours. Following incubation, during which time cells spontaneously detached from the column, cells were eluted using a serum-free medium containing recombinant IL-12, IL-15, and IL-7 only (without the need to add biotin or other competition substance to release the immobilized cells). Following elution, the cells were adjusted to a seeding cell concentration of 3×10⁶ cells/mL by addition of a serum-free medium containing recombinant IL-2, IL-15, and IL-7 and then underwent further culturing at 37° C. in an incubator. After five days of culture, CAR expression was assessed by flow cytometry using an anti-idiotypic antibody against the CAR in order to determine transduction efficiency.

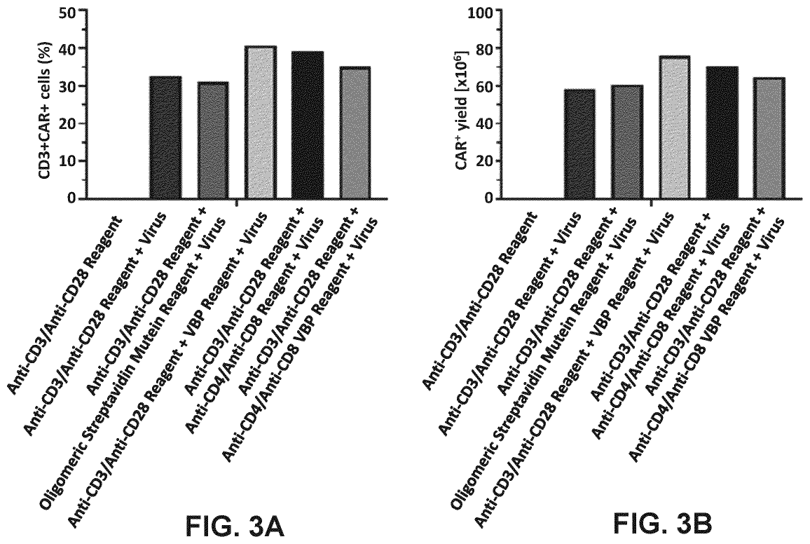

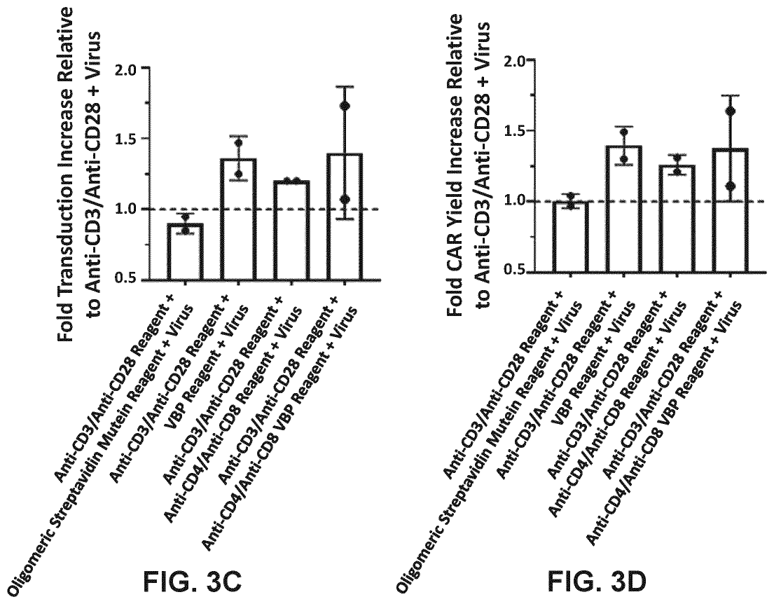

FIG. 3A shows CAR expression of CD3+ T cells following transduction. As shown in FIG. 3A, the percentage of CD3+CAR+ T cells was increased following on-column transduction in the presence of the VBP-functionalized reagent, the anti-CD4/anti-CD8 reagent, and the anti-CD4/anti-CD8 VBP-functionalized reagent, relative to that achieved using the anti-CD3/anti-CD8 stimulatory reagent alone or in combination with the non-functionalized oligomeric streptavidin mutein reagent. Similar results are shown in FIG. 3B, which shows transduction efficiency in terms of CAR T cell yield (cell count).

FIG. 3C demonstrates transduction fold-increases relative to that achieved using the lentiviral particles in the presence of only the anti-CD3/anti-CD28 stimulatory reagent. The non-functionalized oligomeric streptavidin mutein reagent did not result in increased transduction. In contrast, the presence of the VBP-functionalized reagent, the anti-CD4/anti-CD8 reagent, and the anti-CD4/anti-CD8 VBP-functionalized reagent improved transduction efficiency. Similar results are shown in FIG. 3D for fold-increases in CAR T cell yield.

Together, these results further support that the presence of multimerized VBP during transduction, including in reagents further functionalized with binding agents against T cell surface markers (e.g., CD4 and CD8), can improve transduction efficiency and downstream CAR T cell yield following on-column activation and transduction.

### Example 4: Assessment of Anti-CD3/Anti-CD28 Viral Binding Protein (VBP)-Functionalized Stimulatory Reagent for On-Column Transduction

The anti-CD3/anti-CD28 stimulatory reagent described in Example 2 was further functionalized with the viral binding protein (VBP) described in Example 1 and tested for its effect on the on-column transduction of cells. T cells immobilized on an affinity chromatography column were transduced with lentiviral particles in the presence of the anti-CD3/anti-CD28 VBP-functionalized stimulatory reagent.

To do so, an anti-CD3 affinity chromatography column was prepared as described in Example 2. To prepare the anti-CD3/anti-CD28 VBP-functionalized stimulatory reagent, 0.3 mg of the oligomeric streptavidin mutein reagent described in Example 2 was mixed at room temperature with 50 μg of anti-CD3 Fab fragments, 50 μg of anti-CD28 Fab fragments, and 50 μg of the VBP. For comparison, an anti-CD3/anti-CD28 stimulatory reagent without the VBP was prepared as described in Example 2. Both reagents used the same anti-CD3 and anti-CD28 Fab fragments described in Example 2.

For T-cell isolation, stimulation, and transduction, a fresh apheresis sample was loaded onto the anti-CD3 functionalized column. Post loading and washing, the immobilized T cells were incubated in the presence of activation-transduction buffer containing the following: recombinant IL-2, IL-15, and IL-7; 6 μL/1×10⁶ cells of lentiviral particles encoding an exemplary anti-BCMA CAR; and 0.45 mg of (i) the anti-CD3/anti-CD28 VBP-functionalized stimulatory reagent or (ii) the anti-CD3/anti-CD28 stimulatory reagent without VBP. Approximately 100×10⁶ cells were expected to be captured per column.

Following addition of the activation-transduction buffer, the affinity chromatography column was heated and incubated at 37° C. for 4.5 hours. Following incubation, during which time cells spontaneously detached from the column, cells were eluted using a serum-free medium containing recombinant IL-12, IL-15, and IL-7 only (without the need to add biotin or other competition substance to release the immobilized cells). Following elution, the cells were adjusted to a seeding cell concentration of 3×10⁶ cells/mL by addition of a serum-free medium containing recombinant IL-2, IL-15, and IL-7 and then underwent further culturing at 37° C. in an incubator. After five days of culture, CAR expression was assessed by flow cytometry using an anti-idiotypic antibody against the CAR in order to determine transduction efficiency.

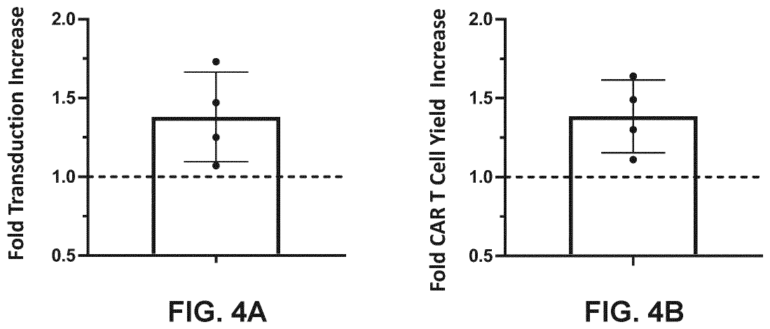

FIG. 4A shows the fold increase in transduction efficiency following transduction in the presence of the anti-CD3/anti-CD28 VBP-functionalized stimulatory reagent, relative to in the presence of the anti-CD3/anti-CD28 stimulatory reagent without VBP. As shown in FIG. 4A, transduction efficiency was increased following on-column transduction in the presence of the anti-CD3/anti-CD28 VBP-functionalized stimulatory reagent. Similar results are shown in FIG. 4B, which shows the fold increase in CAR T cell yield.

Together, these results further support that the presence of multimerized VBP during transduction can improve transduction efficiency and downstream CAR T cell yield following on-column activation and transduction. As shown here, these effects can be achieved using a single stimulatory (e.g., anti-CD3/anti-CD28) reagent that has been further functionalized with the VBP.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

SEQUENCES

| No. | Sequence | Description |
|---|---|---|
| 1 | DPSKDSKAQVSAAEAGITGTWYNQLGSTFIVTAGADGAL | Streptavidin |
| | TGTYESAVGNAESRYVLTGRYDSAPATDGSGTALGWTVA | (UniProt No. |
| | WKNNYRNAHSATTWSGQYVGGAEARINTQWLLTSGTTEA | P22629) |
| | NAWKSTLVGHDTFTKVKPSAASIDAAKKAGVNNGNPLDA | Species: |
| | VQQ | Streptomyces |
| | | avidinii |
| 2 | MEAGITGTWYNQLGSTFIVTAGADGALTGTYESAVGNAE | Minimal |
| | SRYVLTGRYDSAPATDGSGTALGWTVAWKNNYRNAHSAT | streptavidin |
| | TWSGQYVGGAEARINTQWLLTSGTTEANAWKSTLVGHDT | |
| | FTKVKPSAAS | |
| 3 | DPSKDSKAQVSAAEAGITGTWYNQLGSTFIVTAGADGAL | Mutein |
| | TGTYVTARGNAESRYVLTGRYDSAPATDGSGTALGWTVA | Streptavidin Val⁴⁴- |
| | WKNNYRNAHSATTWSGQYVGGAEARINTQWLLTSGTTEA | Thr⁴⁵-Ala⁴⁶-Arg⁴⁷ |
| | NAWKSTLVGHDTFTKVKPSAASIDAAKKAGVNNGNPLDA | |
| | VQQ | |
| 4 | MEAGITGTWYNQLGSTFIVTAGADGALTGTYVTARGNAE | Mutein |
| | SRYVLTGRYDSAPATDGSGTALGWTVAWKNNYRNAHSAT | Streptavidin Val⁴⁴- |
| | TWSGQYVGGAEARINTQWLLTSGTTEANAWKSTLVGHDT | Thr⁴⁵-Ala⁴⁶-Arg⁴⁷ |
| | FTKVKPSAAS | |
| 5 | DPSKDSKAQVSAAEAGITGTWYNQLGSTFIVTAGADGAL | Mutein |
| | TGTYIGARGNAESRYVLTGRYDSAPATDGSGTALGWTVA | Streptavidin Ile⁴⁴- |
| | WKNNYRNAHSATTWSGQYVGGAEARINTQWLLTSGTTEA | Gly⁴⁵-Ala⁴⁶-Arg⁴⁷ |
| | NAWKSTLVGHDTFTKVKPSAASIDAAKKAGVNNGNPLDA | |
| | VQQ | |
| 6 | MEAGITGTWYNQLGSTFIVTAGADGALTGTYIGARGNAE | Mutein |
| | SRYVLTGRYDSAPATDGSGTALGWTVAWKNNYRNAHSAT | Streptavidin Ile⁴⁴- |
| | TWSGQYVGGAEARINTQWLLTSGTTEANAWKSTLVGHDT | Gly⁴⁵-Ala⁴⁶-Arg⁴⁷ |
| | FTKVKPSAAS | |
| 7 | WRHPQFGG | Streptavidin- |
| | | binding peptide, |
| | | Strep-tag® |
| 8 | WSHPQFEK | Strep-tag® II |
| 9 | His-Pro-Xaa | Streptavidin- |
| | | Binding peptide |
| | | Xaa is selected |
| | | from Gln, Asp, and |
| | | Met |
| 10 | His-Pro-Gln-Phe | Streptavidin- |
| | | binding peptide |
| 11 | Xaa₁-Xaa₂-His-Pro-Gln-Phe-Xaa₃-Xaa₄ | Streptavidin- |
| | | binding peptide |
| | | Xaa₁ is Trp, Lys or |
| | | Arg; |
| | | Xaa₂ is any amino |
| | | acid; |
| | | Xaa₃ is Gly or Glu |
| | | Xaa₄ is Gly, Lys or |
| | | Arg |
| 12 | Trp-Xaa₁-His-Pro-Gln-Phe-Xaa₂-Xaa₃ | Streptavidin- |
| | | binding peptide |
| | | Xaa₁ is any amino |
| | | acid; |
| | | Xaa₂ is Gly or Glu |
| | | Xaa₃ is Gly, Lys or |
| | | Arg |
| 13 | Trp-Ser-His-Pro-Gln-Phe-Glu-Lys-(Xaa)ₙ- | Sequential modules |
| | Trp-Ser-His-Pro-Gln-Phe-Glu-Lys | of streptavidin- |
| | | binding peptide |
| | | Xaa is any amino |
| | | acid; |
| | | n is 8 |
| 14 | Trp-Ser-His-Pro-Gln-Phe-Glu-Lys- | Sequential modules |
| | (GlyGlyGlySer)n-Trp-Ser-His-Pro- | of streptavidin- |
| | Gln-Phe-Glu-Lys | binding peptide |
| | | n is 2 |
| 15 | SAWSHPQFEKGGGSGGGSGGGSWSHPQFEK | Twin-Strep-tag |
| 16 | SAWSHPQFEKGGGSGGGSGGSAWSHPQFEK | Twin-Strep-tag |
| 17 | WSHPQFEKGGGSGGGSGGGSWSHPQFEK | Twin-Strep-tag |
| 18 | WSHPQFEKGGGSGGGSWSHPQFEK | Twin-Strep-tag |
| 19 | WSHPQFEKGGGSGGGSGGSAWSHPQFEK | Twin-Strep-tag |
| 20 | Tyr-Pro-Tyr-Asp-Val-Pro-Asp-Tyr-Ala | HA-tag |
| 21 | Tyr-Thr-Asp-Ile-Glu-Met-Asn-Arg-Leu- | VSV-G-tag |
| | Gly-Lys | |
| 22 | Gln-Pro-Glu-Leu-Ala-Pro-Glu-Asp-Pro- | HSV-tag |
| | Glu-Asp | |
| 23 | Ala-Ser-Met-Thr-Gly-Gly-Gln-Gln-Met- | T7 epitope |
| | Gly | |
| 24 | Gln-Pro-Glu-Leu-Ala-Pro-Glu-Asp-Pro- | HSV epitope |
| | Glu-Asp | |
| 25 | Glu-Gln-Lys-Leu-Ile-Ser-Glu-Glu-Asp- | Myc epitope |
| | Leu | |
| 26 | Gly-Lys-Pro-Ile-Pro-Asn-Pro-Leu-Leu- | V5-tag |
| | Gly-Leu-Asp-Ser-Thr | |
| 27 | EAGITGTWYNQLGSTFIVTAGADGALTGTYVTARGNAES | Mutein |
| | RYVLTGRYDSAPATDGSGTALGWTVAWKNNYRNAHSATT | Streptavidin Val⁴⁴- |
| | WSGQYVGGAEARINTQWLLTSGTTEENAGYSTLVGHDTF | Thr⁴⁵-Ala⁴⁶-Arg⁴⁷ |
| | TKVKPSAAS | and Glu¹¹⁷, Gly¹²⁰ |
| | | Tyr¹²¹ (mutein m1- |
| | | 9) |
| 28 | DPSKDSKAQVSAAEAGITGTWYNQLGSTFIVTAGADGAL | Mutein |
| | TGTYVTARGNAESRYVLTGRYDSAPATDGSGTALGWTVA | Streptavidin Val⁴⁴- |
| | WKNNYRNAHSATTWSGQYVGGAEARINTQWLLTSGTTEE | Thr⁴⁵-Ala⁴⁶-Arg⁴⁷ |
| | NAGYSTLVGHDTFTKVKPSAAS | and Glu¹¹⁷, Gly¹²⁰ |
| | | Tyr¹²¹ (mutein m1- |
| | | 9) |
| 29 | AMQVQLKQSGPGLVQPSQSLSITCTVSGFSLTTFGVHWV | Variable Heavy |
| | RQSPGKGLEWLGVIWASGITDYNVPFMSRLSITKDNSKS | chain of Fab |
| | QVFFKLNSLQPDDTAIYYCAKNDPGTGFAYWGQGTLVTV | fragment m13B8.2 |
| | SAGSTKGPSVFPLAPSSKSTSGGTAALGCLVKDYFPEPV | |
| | TVSWNSGALTSGVHTFPAVLQSSGLYSLSSVVTVPSSSL | |
| | GTQTYICNVNHKPSNTKVDKKVEPKSCGSAWSHPQFEKG | |
| | GGSGGGSGGSAWSHPQFEK | |
| 30 | AMDIQMTQSPASLSASVGETVTFTCRASEMIYSYLAWYQ | Variable Light |
| | QKQGKSPQLLVHDAKTLAEGVPSRFSGGGSGTQFSLKIN | chain of Fab |
| | TLQPEDFGTYYCQAHYGNPPTFGGGTKLEIKRGIAAPSV | Fragment m13B8.2 |
| | FIFPPSDEQLKSGTASVVCLLNNFYPREAKVQWKVDNAL | |
| | QSGNSQESVTEQDSKDSTYSLSSTLTLSKADYEKHKVYA | |
| | CEVTHQGLSSPVTKSFNRGECGS | |
| 31 | GlnValGlnLeuGlnGlnSerGlyAlaGluLeuAlaArg | Variable Heavy |
| | ProGlyAlaSerValLysMetSerCysLysAlaSerGly | chain of anti-CD3 |
| | TyrThrPheThrArgTyrThrMetHisTrpValLysGln | antibody OKT3 |
| | ArgProGlyGlnGlyLeuGluTrpIleGlyTyrIleAsn | |
| | ProSerArgGlyTyrThrAsnTyrAsnGlnLysPheLys | |
| | AspLysAlaThrLeuThrThrAspLysSerSerSerThr | |
| | AlaTyrMetGlnLeuSerSerLeuThrSerGluAspSer | |
| | AlaValTyrTyrCysAlaArgTyrTyrAspAspHisTyr | |
| | CysLeuAspTyrTrpGlyGlnGlyThrThrLeuThrVal | |
| | SerSer | |
| 32 | GlnIleValLeuThrGlnSerProAlaIleMetSerAla | Variable Light |
| | SerProGlyGluLysVa1ThrMetThrCysSerAlaSer | chain of anti-CD3 |
| | SerSerValSerTyrMetAsnTrpTyrGlnGlnLysSer | antibody OKT3 |
| | GlyThrSerProLysArgTrpIleTyrAspThrSerLys | |
| | LeuAlaSerGlyValProAlaHisPheArgGlySerGly | |
| | SerGlyThrSerTyrSerLeuThrIleSerGlyMetGlu | |
| | AlaGluAspAlaAlaThrTyrTyrCysGlnGlnTrpSer | |
| | SerAsnProPheThrPheGlySerGlyThrLysLeuGlu | |
| | IleAsn | |
| 33 | LeuGlnGlnSerGlyAlaGluLeuValLysProGlyAla | Variable Heavy |
| | SerValArgLeuSerCysLysAlaSerGlyTyrThrPhe | chain of anti-CD28 |
| | ThrGluTyrIleIleHisTrpIleLysLeuArgSerGly | antibody CD28.3 |
| | GlnGlyLeuGluTrpIleGlyTrpPheTyrProGlySer | |
| | AsnAspIleGlnTyrAsnAlaLysPheLysGlyLysAla | |
| | ThrLeuThrAlaAspLysSerSerSerThrValTyrMet | |
| | GluLeuThrGlyLeuThrSerGluAspSerAlaValTyr | |
| | PheCysAlaArgArgAspAspPheSerGlyTyrAspAla | |
| | LeuProTyrTrpGlyGlnGlyThrMetValThrVal | |
| 34 | AspIleGlnMetThrGlnSerProAlaSerLeuSerVal | Variable Light |
| | SerValGlyGluThrValThrIleThrCysArgThrAsn | chain of anti-CD28 |
| | GluAsnIleTyrSerAsnLeuAlaTrpTyrGlnGlnLys | antibody CD28.3 |
| | GlnGlyLysSerProGlnLeuLeuIleTyrAlaAlaThr | |
| | HisLeuValGluGlyValProSerArgPheSerGlySer | |
| | GlySerGlyThrGlnTyrSerLeuLysIleThrSerLeu | |
| | GlnSerGluAspPheGlyAsnTyrTyrCysGlnHisPhe | |
| | TrpGlyThrProCysThrPheGlyGlyGlyThrLysLeu | |
| | GluIleLysArg | |
| 35 | His-Asn-His-Arg-His-Lys-His-Gly-Gly-Gly | MAT tag |
| | -Cys | |
| 36 | GlnValGlnLeuValGlnSerGlyAlaGluValLysLys | Variable Heavy |
| | ProGlyAla1ThrValLysIleSerCysLysValSerGly | chain of huOKT8 |
| | PheAsnIleLysAspThrTyrIleHisTrpValGlnGln | |
| | AlaProGlyLysGlyLeuGluTrpMetGlyArgIleAsp | |
| | ProAlaAsnAspAsnThrLeuTyrAlaSerLysPheGln | |
| | GlyArgValThrIleThrAlaAspThrSerThrAspThr | |
| | AlaTyr65MetGluLeuSerSerLeuArgSerGluAsp | |
| | ThrAlaValTyrTyrCysAlaArgGlyTyrGlyTyrTyr | |
| | ValPheAspHisTrpGlyGlnGlyThrLeuValThrVal | |
| | SerSer | |
| 37 | AspValGlnIleThrGlnSerProSerSerLeuSerAla | Variable Light |
| | SerValGlyAspArgValThrIleThrCysArgThrSer | chain of huOKT8 |
| | ArgSerIleSerGlnTyrLeuAlaTrpTyrGlnGlnLys | |
| | ProGlyLysValProLysLeuLeuIleTyrSerGlySer | |
| | ThrLeuGlnSerGlyValProSerArgPheSerGlySer | |
| | GlySerGlyThrAspPheThrLeuThrIleSerSerLeu | |
| | GlnProGluAspValAlaThrTyrTyrCysGlnGlnHis | |
| | AsnGluAsnProLeuThrPheGlyGlyGlyThrLysVal | |
| | GluIleLys | |
| 38 | -PGGG-(SGGGG)₅-P- | Linker |
| | | P is proline, G is |
| | | glycine and S is |
| | | serine |
| 39 | GSADDAKKDAAKKDGKS | Linker |
| 40 | MLLLVTSLLLCELPHPAFLLIP | GMCSFR alpha |
| | | chain (amino acid) |
| 41 | atgcttctcctggtgacaagccttctgctctgtgagtta | GMCSFR alpha |
| | ccacacccagcattcctcctgatccca | chain (nucleic acid) |
| 42 | MALPVTALLLPLALLLHA | CD8 alpha signal |
| | | peptide |
| 43 | MLLLVTSLLLCELPHPAFLLIPRKVCNGIGIGEFKDSLS | truncated |
| | INATNIKHFKNCTSISGDLHILPVAFRGDSFTHTPPLDP | epidermal growth |
| | QELDILKTVKEITGFLLIQAWPENRTDLHAFENLEIIRG | factor receptor |
| | RTKQHGQFSLAVVSLNITSLGLRSLKEISDGDVIISGNK | (tEGFR) |
| | NLCYANTINWKKLFGTSGQKTKIISNRGENSCKATGQVC | |
| | HALCSPEGCWGPEPRDCVSCRNVSRGRECVDKCNLLEGE | |
| | PREFVENSECIQCHPECLPQAMNITCTGRGPDNCIQCAH | |
| | YIDGPHCVKTCPAGVMGENNTLVWKYADAGHVCHLCHPN | |
| | CTYGCTGPGLEGCPTNGPKIPSIATGMVGALLLLLVVAL | |
| | GIGLFM | |
| 44 | RKVCNGIGIGEFKDSLSINATNIKHFKNCTSISGDLHIL | truncated |
| | PVAFRGDSFTHTPPLDPQELDILKTVKEITGFLLIQAWP | epidermal growth |
| | ENRTDLHAFENLEIIRGRTKQHGQFSLAVVSLNITSLGL | factor receptor |
| | RSLKEISDGDVIISGNKNLCYANTINWKKLFGTSGQKTK | (tEGFR) |
| | IISNRGENSCKATGQVCHALCSPEGCWGPEPRDCVSCRN | |
| | VSRGRECVDKCNLLEGEPREFVENSECIQCHPECLPQAM | |
| | NITCTGRGPDNCIQCAHYIDGPHCVKTCPAGVMGENNTL | |
| | VWKYADAGHVCHLCHPNCTYGCTGPGLEGCPTNGPKIPS | |
| | IATGMVGALLLLLVVALGIGLFM | |
| 45 | VKQTLNFDLLKLAGDVESNPGP | F2A |
| 46 | QCTNYALLKLAGDVESNPGP | E2A |
| 47 | LEGGGEGRGSLLTCGDVEENPGPR | T2A |
| 48 | EGRGSLLTCGDVEENPGP | T2A |
| 49 | GSGATNFSLLKQAGDVEENPGP | P2A |
| 50 | ATNFSLLKQAGDVEENPGP | P2A |
| 51 | DYGVS | CDR H1 |
| 52 | VIWGSETTYYNSALKS | CDR H2 |
| 53 | YAMDYWG | CDR H3 |
| 54 | HYYYGGSYAMDY | CDR H3 |
| 55 | RASQDISKYLN | CDR L1 |
| 56 | SRLHSGV | CDR L2 |
| 57 | HTSRLHS | CDR L2 |
| 58 | GNTLPYTFG | CDR L3 |
| 59 | QQGNTLPYT | CDR L3 |
| 60 | EVKLQESGPGLVAPSQSLSVTCTVSGVSLPDYGVSWIRQ | VH |
| | PPRKGLEWLGVIWGSETTYYNSALKSRLTIIKDNSKSQV | |
| | FLKMNSLQTDDTAIYYCAKHYYYGGSYAMDYWGQGTSVT | |
| | VSS | |
| 61 | DIQMTQTTSSLSASLGDRVTISCRASQDISKYLNWYQQK | VL |
| | PDGTVKLLIYHTSRLHSGVPSRFSGSGSGTDYSLTISNL | |
| | EQEDIATYFCQQGNTLPYTFGGGTKLEIT | |
| 62 | GSTSGSGKPGSGEGSTKG | Linker |
| 63 | gacatccagatgacccagaccacctccagcctgagcgcc | Sequence encoding |
| | agcctgggcgaccgggtgaccatcagctgccgggccagc | scFv |
| | caggacatcagcaagtacctgaactggtatcagcagaag | |
| | cccgacggcaccgtcaagctgctgatctaccacaccagc | |
| | cggctgcacagcggcgtgcccagccggtttagcggcagc | |
| | ggctccggcaccgactacagcctgaccatctccaacctg | |
| | gaacaggaagatatcgccacctacttttgccagcagggc | |
| | aacacactgccctacacctttggcggcggaacaaagctg | |
| | gaaatcaccggcagcacctccggcagcggcaagcctggc | |
| | agcggcgagggcagcaccaagggcgaggtgaagctgcag | |
| | gaaagcggccctggcctggtggcccccagccagagcctg | |
| | agcgtgacctgcaccgtgagcggcgtgagcctgcccgac | |
| | tacggcgtgagctggatccggcagccccccaggaagggc | |
| | ctggaatggctgggcgtgatctggggcagcgagaccacc | |
| | tactacaacagegccctgaagagccggctgaccatcatc | |
| | aaggacaacagcaagagccaggtgttcctgaagatgaac | |
| | agcctgcagaccgacgacaccgccatctactactgcgcc | |
| | aagcactactactacggcggcagctacgccatggactac | |
| | tggggccagggcaccagcgtgaccgtgagcagc | |
| 64 | DIQMTQTTSSLSASLGDRVTISCRASQDISKYLNWYQQK | scFv |
| | PDGTVKLLIYHTSRLHSGVPSRFSGSGSGTDYSLTISNL | |
| | EQEDIATYFCQQGNTLPYTFGGGTKLEITGSTSGSGKPG | |
| | SGEGSTKGEVKLQESGPGLVAPSQSLSVTCTVSGVSLPD | |
| | YGVSWIRQPPRKGLEWLGVIWGSETTYYNSALKSRLTII | |
| | KDNSKSQVFLKMNSLQTDDTAIYYCAKHYYYGGSYAMDY | |
| | WGQGTSVTVSS | |
| 65 | SYWMN | CDR H1 |
| 66 | QIYPGDGDTNYNGKFKG | CDR H2 |
| 67 | KTISSVVDFYFDY | CDR H3 |
| 68 | QQYNRYPYT | CDR L3 |
| 69 | SYWMN | CDR H1 |
| 70 | QIYPGDGDTNYNGKFKG | CDR H2 |
| 71 | EVKLQQSGAELVRPGSSVKISCKASGYAFSSYWMNWVKQ | VH |
| | RPGQGLEWIGQIYPGDGDTNYNGKFKGQATLTADKSSST | |
| | AYMQLSGLTSEDSAVYFCARKTISSVVDFYFDYWGQGTT | |
| | VTVSS | |
| 72 | DIELTQSPKFMSTSVGDRVSVTCKASQNVGTNVAWYQQK | VL |
| | PGQSPKPLIYSATYRNSGVPDRFTGSGSGTDFTLTITNV | |
| | QSKDLADYFCQQYNRYPYTSGGGTKLEIKR | |
| 73 | KASQNVGTNVA | CDR L1 |
| 74 | SATYRNS | CDR L2 |
| 75 | QQYNRYPYT | CDR L3 |
| 76 | SYWMN | CDR H1 |
| 77 | QIYPGDGDTNYNGKFKG | CDR H2 |
| 78 | KTISSVVDFYFDY | CDR H3 |
| 79 | GGGGSGGGGSGGGGS | Linker |
| 80 | EVKLQQSGAELVRPGSSVKISCKASGYAFSSYWMNWVKQ | scFv |
| | RPGQGLEWIGQIYPGDGDTNYNGKFKGQATLTADKSSST | |
| | AYMQLSGLTSEDSAVYFCARKTISSVVDFYFDYWGQGTT | |
| | VTVSSGGGGSGGGGSGGGGSDIELTQSPKFMSTSVGDRV | |
| | SVTCKASQNVGTNVAWYQQKPGQSPKPLIYSATYRNSGV | |
| | PDRFTGSGSGTDFTLTITNVQSKDLADYFCQQYNRYPYT | |
| | SGGGTKLEIKR | |
| 81 | ESKYGPPCPPCP | spacer (IgG4hinge) |
| | | (aa) |
| | | Species: Homo |
| | | sapiens |
| 82 | GAATCTAAGTACGGACCGCCCTGCCCCCCTTGCCCT | spacer (IgG4hinge) |
| | | (nt) |
| | | Species: Homo |
| | | sapiens |
| 83 | ESKYGPPCPPCPGQPREPQVYTLPPSQEEMTKNQVSLTC | Hinge-CH3 spacer |
| | LVKGFYPSDIAVEWESNGQPENNYKTTPPVLDSDGSFFL | Species: Homo |
| | YSRLTVDKSRWQEGNVFSCSVMHEALHNHYTQKSLSLSL | sapiens |
| | GK | |
| 84 | ESKYGPPCPPCPAPEFLGGPSVFLFPPKPKDTLMISRTP | Hinge-CH2-CH3 |
| | EVTCVVVDVSQEDPEVQFNWYVDGVEVHNAKTKPREEQF | spacer |
| | NSTYRVVSVLTVLHQDWLNGKEYKCKVSNKGLPSSIEKT | Species: Homo |
| | ISKAKGQPREPQVYTLPPSQEEMTKNQVSLTCLVKGFYP | sapiens |
| | SDIAVEWESNGQPENNYKTTPPVLDSDGSFFLYSRLTVD | |
| | KSRWQEGNVFSCSVMHEALHNHYTQKSLSLSLGK | |
| 85 | RWPESPKAQASSVPTAQPQAEGSLAKATTAPATTRNTGR | IgD-hinge-Fc |
| | GGEEKKKEKEKEEQEERETKTPECPSHTQPLGVYLLTPA | Species: Homo |
| | VQDLWLRDKATFTCFVVGSDLKDAHLTWEVAGKVPTGGV | sapiens |
| | EEGLLERHSNGSQSQHSRLTLPRSLWNAGTSVTCTLNHP | |
| | SLPPQRLMALREPAAQAPVKLSLNLLASSDPPEAASWLL | |
| | CEVSGFSPPNILLMWLEDQREVNTSGFAPARPPPQPGST | |
| | TFWAWSVLRVPAPPSPQPATYTCVVSHEDSRTLLNASRS | |
| | LEVSYVTDH | |
| 86 | GluValValValLysTyrGlyProProCysProProCys | Exemplary IgG |
| | Pro | Hinge |
| 87 | X₁PPX₂P | Exemplary IgG |
| | X₁ is glycine, | Hinge |
| | cysteine or arginine | |
| | X₂ is cysteine or threonine | |
| 88 | GluProLysSerCysAspLysThrHisThrCysProPro | Exemplary IgG |
| | CysPro | Hinge |
| 89 | GluArgLysCysCysValGluCysProProCysPro | Exemplary IgG |
| | | Hinge |
| 90 | ELKTPLGDTHTCPRCPEPKSCDTPPPCPRCPEPKSCDTP | Exemplary IgG |
| | PPCPRCPEPKSCDTPPPCPRCP | Hinge |
| 91 | GluSerLysTyrGlyProProCysProSerCysPro | Exemplary IgG |
| | | Hinge |
| 92 | GluSerLysTyrGlyProProCysProProCysPro | Exemplary IgG |
| | | Hinge |
| 93 | TyrGlyProProCysProProCysPro | Exemplary IgG |
| | | Hinge |
| 94 | LysTyrGlyProProCysProProCysPro | Exemplary IgG |
| | | Hinge |
| 95 | FWVLVVVGGVLACYSLLVTVAFIIFWV | CD28 (amino acids |
| | | 153-179 of |
| | | Accession No. |
| | | P10747) |
| | | Species: Homo |
| | | sapiens |
| 96 | IEVMYPPPYLDNEKSNGTIIHVKGKHLCPSPLFPGPSKP | CD28 (amino acids |
| | FWVLVVVGGVLACYSLLVTVAFIIFWV | 114-179 of |
| | | Accession No. |
| | | P10747) |
| | | Species: Homo |
| | | sapiens |
| 97 | RSKRSRLLHSDYMNMTPRRPGPTRKHYQPYAPPRDFAAY | CD28 (amino acids |
| | RS | 180-220 of |
| | | P10747) |
| | | Species: Homo |
| | | sapiens |
| 98 | RSKRSRGGHSDYMNMTPRRPGPTRKHYQPYAPPRDFAAY | CD28 (LL to GG) |
| | RS | Species: Homo |
| | | sapiens |
| 99 | KRGRKKLLYIFKQPFMRPVQTTQEEDGCSCRFPEEEEGG | 4-1BB (amino |
| | CEL | acids 214-255 of |
| | | Q07011.1) |
| | | Species: Homo |
| | | sapiens |
| 100 | RVKFSRSADAPAYQQGQNQLYNELNLGRREEYDVLDKRR | CD3 zeta |
| | GRDPEMGGKPRRKNPQEGLYNELQKDKMAEAYSEIGMKG | Species: Homo |
| | ERRRGKGHDGLYQGLSTATKDTYDALHMQALPPR | sapiens |
| 101 | RVKFSRSAEPPAYQQGQNQLYNELNLGRREEYDVLDKRR | CD3 zeta |
| | GRDPEMGGKPRRKNPQEGLYNELQKDKMAEAYSEIGMKG | Species: Homo |
| | ERRRGKGHDGLYQGLSTATKDTYDALHMQALPPR | sapiens |
| 102 | RVKFSRSADAPAYKQGQNQLYNELNLGRREEYDVLDKRR | CD3 zeta |
| | GRDPEMGGKPRRKNPQEGLYNELQKDKMAEAYSEIGMKG | Species: Homo |
| | ERRRGKGHDGLYQGLSTATKDTYDALHMQALPPR | sapiens |
| 103 | EAGITGTWYNQLGSTFIVTAGADGALTGTYESAVGNAES | Minimal |
| | RYVLTGRYDSAPATDGSGTALGWTVAWKNNYRNAHSATT | streptavidin |
| | WSGQYVGGAEARINTQWLLTSGTTEANAWKSTLVGHDTF | without initial |
| | TKVKPSAAS | methionine |
| 104 | EAGITGTWYNQLGSTFIVTAGADGALTGTYVTARGNAES | Mutein |
| | RYVLTGRYDSAPATDGSGTALGWTVAWKNNYRNAHSATT | Streptavidin Val⁴⁴- |
| | WSGQYVGGAEARINTQWLLTSGTTEANAWKSTLVGHDTF | Thr⁴⁵-Ala⁴⁶-Arg⁴⁷ |
| | TKVKPSAAS | without initial |
| | | methionine |
| 105 | EAGITGTWYNQLGSTFIVTAGADGALTGTYIGARGNAES | Mutein |
| | RYVLTGRYDSAPATDGSGTALGWTVAWKNNYRNAHSATT | Streptavidin Ile44- |
| | WSGQYVGGAEARINTQWLLTSGTTEANAWKSTLVGHDTF | Gly45-Ala-46- |
| | TKVKPSAAS | Arg47 without |
| | | initial methionine |
| 106 | ASTKGPSVFPLAPCSRSTSESTAALGCLVKDYFPEPVTV | Human IgG2 Fc |
| | SWNSGALTSGVHTFPAVLQSSGLYSLSSVVTVPSSNFGT | (Uniprot P01859) |
| | QTYTCNVDHKPSNTKVDKTVERKCCVECPPCPAPPVAGP | Species: Homo |
| | SVFLFPPKPKDTLMISRTPEVTCVVVDVSHEDPEVQFNW | sapiens |
| | YVDGVEVHNAKTKPREEQFNSTFRVVSVLTVVHQDWLNG | |
| | KEYKCKVSNKGLPAPIEKTISKTKGQPREPQVYTLPPSR | |
| | EEMTKNQVSLTCLVKGFYPSDISVEWESNGQPENNYKTT | |
| | PPMLDSDGSFFLYSKLTVDKSRWQQGNVFSCSVMHEALH | |
| | NHYTQKSLSLSPGK | |
| 107 | ASTKGPSVFPLAPCSRSTSESTAALGCLVKDYFPEPVTV | Human IgG4 Fc |
| | SWNSGALTSGVHTFPAVLQSSGLYSLSSVVTVPSSSLGT | (Uniprot P01861) |
| | KTYTCNVDHKPSNTKVDKRVESKYGPPCPSCPAPEFLGG | Species: Homo |
| | PSVFLFPPKPKDTLMISRTPEVTCVVVDVSQEDPEVQFN | sapiens |
| | WYVDGVEVHNAKTKPREEQFNSTYRVVSVLTVLHQDWLN | |
| | GKEYKCKVSNKGLPSSIEKTISKAKGQPREPQVYTLPPS | |
| | QEEMTKNQVSLTCLVKGFYPSDIAVEWESNGQPENNYKT | |
| | TPPVLDSDGSFFLYSRLTVDKSRWQEGNVFSCSVMHEAL | |
| | HNHYTQKSLSLSLGK | |
| 108 | GVQVETISPGDGRTFPKRGQTCVVHYTGMLEDGKKMDSS | FKBP |
| | RDRNKPFKFMLGKQEVIRGWEEGVAQMSVGQRAKLTISP | |
| | DYAYGATGHPGIIPPHATLVFDVELLKLE | |
| 109 | GVQVETISPGDGRTFPKRGQTCVVHYTGMLEDGKKVDSS | FKBP12v36 |
| | RDRNKPFKFMLGKQEVIRGWEEGVAQMSVGQRAKLTISP | |
| | DYAYGATGHPGIIPPHATLVFDVELLKLE | |
| 110 | MGSNKSKPKDASQRRR | Modified acylation |
| | | motif |
| 111 | Met-Gly-Cys-Xaa-Cys | dual acylation |
| | | motif |
| 112 | Cys-Ala-Ala-Xaa | acylation region |
| 113 | DYSIN | CDR-H1 |
| 114 | WINTETREPAYAYDFRG | CDR-H2 |
| 115 | DYSYAMDY | CDR-H3 |
| 116 | RASESVTILGSHLIH | CDR-L1 |
| 117 | LASNVQT | CDR-L2 |
| 118 | LQSRTIPRT | CDR-L3 |
| 119 | QIQLVQSGPELKKPGETVKISCKASGYTFTDYSINWVKR | Variable heavy |
| | APGKGLKWMGWINTETREPAYAYDFRGRFAFSLETSAST | (VH) Anti-BCMA |
| | AYLQINNLKYEDTATYFCALDYSYAMDYWGQGTSVTVSS | |
| 120 | DIVLTQSPPSLAMSLGKRATISCRASESVTILGSHLIHW | Variable light (VL) |
| | YQQKPGQPPTLLIQLASNVQTGVPARFSGSGSRTDFTLT | Anti-BCMA |
| | IDPVEEDDVAVYYCLQSRTIPRTFGGGTKLEIK | |
| 121 | DIVLTQSPPSLAMSLGKRATISCRASESVTILGSHLIHW | scFv |
| | YQQKPGQPPTLLIQLASNVQTGVPARFSGSGSRTDFTLT | |
| | IDPVEEDDVAVYYCLQSRTIPRTFGGGTKLEIKGSTSGS | |
| | GKPGSGEGSTKGQIQLVQSGPELKKPGETVKISCKASGY | |
| | TFTDYSINWVKRAPGKGLKWMGWINTETREPAYAYDFRG | |
| | RFAFSLETSASTAYLQINNLKYEDTATYFCALDYSYAMD | |
| | YWGQGTSVTVSS | |
| 122 | DIVLTQSPPSLAMSLGKRATISCRASESVTILGSHLIHW | anti-BCMA CAR |
| | YQQKPGQPPTLLIQLASNVQTGVPARFSGSGSRTDFTLT | |
| | IDPVEEDDVAVYYCLQSRTIPRTFGGGTKLEIKGSTSGS | |
| | GKPGSGEGSTKGQIQLVQSGPELKKPGETVKISCKASGY | |
| | TFTDYSINWVKRAPGKGLKWMGWINTETREPAYAYDFRG | |
| | RFAFSLETSASTAYLQINNLKYEDTATYFCALDYSYAMD | |
| | YWGQGTSVTVSSAAATTTPAPRPPTPAPTIASQPLSLRP | |
| | EACRPAAGGAVHTRGLDFACDIYIWAPLAGTCGVLLLSL | |
| | VITLYCKRGRKKLLYIFKQPFMRPVQTTQEEDGCSCRFP | |
| | EEEEGGCELRVKFSRSADAPAYQQGQNQLYNELNLGRRE | |
| | EYDVLDKRRGRDPEMGGKPRRKNPQEGLYNELQKDKMAE | |
| | AYSEIGMKGERRRGKGHDGLYQGLSTATKDTYDALHMQA | |
| | LPPR | |
| 123 | DYYVY | CDR-H1 |
| 124 | WINPNSGGTNYAQKFQG | CDR-H2 |
| 125 | SQRDGYMDY | CDR-H3 |
| 126 | TGTSSDVG | CDR-L1 |
| 127 | EDSKRPS | CDR-L2 |
| 128 | SSNTRSSTLV | CDR-L3 |
| 129 | EVQLVQSGAEMKKPGASLKLSCKASGYTFIDYYVYWMRQ | Variable heavy |
| | APGQGLESMGWINPNSGGTNYAQKFQGRVTMTRDTSIST | (VH) Anti-BCMA |
| | AYMELSRLRSDDTAMYYCARSQRDGYMDYWGQGTLVTVS | |
| | S | |
| 130 | QSALTQPASVSASPGQSIAISCTGTSSDVGWYQQHPGKA | Variable light (VL) |
| | PKLMIYEDSKRPSGVSNRFSGSKSGNTASLTISGLQAED | Anti-BCMA |
| | EADYYCSSNTRSSTLVFGGGTKLTVLG | |
| 131 | QSALTQPASVSASPGQSIAISCTGTSSDVGWYQQHPGKA | scFv |
| | PKLMIYEDSKRPSGVSNRFSGSKSGNTASLTISGLQAED | |
| | EADYYCSSNTRSSTLVFGGGTKLTVLGSRGGGGSGGGGS | |
| | GGGGSLEMAEVQLVQSGAEMKKPGASLKLSCKASGYTFI | |
| | DYYVYWMRQAPGQGLESMGWINPNSGGTNYAQKFQGRVT | |
| | MTRDTSISTAYMELSRLRSDDTAMYYCARSQRDGYMDYW | |
| | GQGTLVTVSS | |
| 132 | QSALTQPASVSASPGQSIAISCTGTSSDVGWYQQHPGKA | anti-BCMA CAR |
| | PKLMIYEDSKRPSGVSNRFSGSKSGNTASLTISGLQAED | |
| | EADYYCSSNTRSSTLVFGGGTKLTVLGSRGGGGSGGGGS | |
| | GGGGSLEMAEVQLVQSGAEMKKPGASLKLSCKASGYTFI | |
| | DYYVYWMRQAPGQGLESMGWINPNSGGTNYAQKFQGRVT | |
| | MTRDTSISTAYMELSRLRSDDTAMYYCARSQRDGYMDYW | |
| | GQGTLVTVSSESKYGPPCPPCPAPPVAGPSVFLFPPKPK | |
| | DTLMISRTPEVTCVVVDVSQEDPEVQFNWYVDGVEVHNA | |
| | KTKPREEQFQSTYRVVSVLTVLHQDWLNGKEYKCKVSNK | |
| | GLPSSIEKTISKAKGQPREPQVYTLPPSQEEMTKNQVSL | |
| | TCLVKGFYPSDIAVEWESNGQPENNYKTTPPVLDSDGSF | |
| | FLYSRLTVDKSRWQEGNVFSCSVMHEALHNHYTQKSLSL | |
| | SLGKMFWVLVVVGGVLACYSLLVTVAFIIFWVKRGRKKL | |
| | LYIFKQPFMRPVQTTQEEDGCCRFPEEEEGGCELRVKFS | |
| | RSADAPAYQQGQNQLYNELNLGRREEYDVLDKRRGRDPE | |
| | MGGKPRRKNPQEGLYNELQKDKMAEAYSEIGMKGERRRG | |
| | KGHDGLYQGLSTATKDTYDALHMQALPPR | |
| 133 | AIPAPTDLKFTQVTPTSLSAQWTPPNVQLTGYRVRVTPK | Heparin binding |
| | EKTGPMKEINLAPDSSSVVVSGLMVATKYEVSVYALKDT | domain II of |
| | LTSRPAQGVVTTLENVSPPRRARVTDATETTITISWRTK | fibronectin (FN12- |
| | TETITGFQVDAVPANGQTPIQRTIKPDVRSYTITGLQPG | 14) |
| | TDYKIYLYTLNDNARSSPVVIDASTAIDAPSNLRFLATT | |
| | PNSLLVSWQPPRARITGYIIKYEKPGSPPREVVPRPRPG | |
| | VTEATITGLEPGTEYTIYVIALKNNQKSEPLIGRKKT | |
| 134 | GGGGSGGGGS | Linker |
| 135 | MAIPAPTDLKFTQVTPTSLSAQWTPPNVQLTGYRVRVTP | Viral binding |
| | KEKTGPMKEINLAPDSSSVVVSGLMVATKYEVSVYALKD | protein |
| | TLTSRPAQGVVTTLENVSPPRRARVTDATETTITISWRT | |
| | KTETITGFQVDAVPANGQTPIQRTIKPDVRSYTITGLQP | |
| | GTDYKIYLYTLNDNARSSPVVIDASTAIDAPSNLRFLAT | |
| | TPNSLLVSWQPPRARITGYIIKYEKPGSPPREVVPRPRP | |
| | GVTEATITGLEPGTEYTIYVIALKNNQKSEPLIGRKKTG | |
| | GGGSGGGGSAIPAPTDLKFTQVTPTSLSAQWTPPNVQLT | |
| | GYRVRVTPKEKTGPMKEINLAPDSSSVVVSGLMVATKYE | |
| | VSVYALKDTLTSRPAQGVVTTLENVSPPRRARVTDATET | |
| | TITISWRTKTETITGFQVDAVPANGQTPIQRTIKPDVRS | |
| | YTITGLQPGTDYKIYLYTLNDNARSSPVVIDASTAIDAP | |
| | SNLRFLATTPNSLLVSWQPPRARITGYIIKYEKPGSPPR | |
| | EVVPRPRPGVTEATITGLEPGTEYTIYVIALKNNQKSEP | |
| | LIGRKKTSAWSHPQFEKGGGGSGGGGSGGSAWSHPQFEK | |
| 136 | atggcgattccggcgccgaccgatctgaaatttacccag | Sequence encoding |
| | gtgaccccgaccagcctgagcgcgcagtggaccccgccg | viral binding |
| | aacgtgcagctgaccggctatcgcgtgcgcgtgaccccg | protein |
| | aaagaaaaaaccggcccgatgaaagaaattaacctggcg | |
| | ccggatagcagcagcgtggtggtgagcggcctgatggtg | |
| | gcgaccaaatatgaagtgagcgtgtatgcgctgaaagat | |
| | accctgaccagccgcccggcgcagggcgtggtgaccacc | |
| | ctggaaaacgtgagcccgccgcgccgcgcgcgcgtgacc | |
| | gatgegaccgaaaccaccattaccattagctggegcacc | |
| | aaaaccgaaaccattaccggctttcaggtggatgcggtg | |
| | ccggcgaacggccagaccccgattcagcgcaccattaaa | |
| | ccggatgtgcgcagctataccattaccggcctgcagccg | |
| | ggcaccgattataaaatttatctgtataccctgaacgat | |
| | aacgcgcgcagcagcccggtggtgattgatgcgagcacc | |
| | gcgattgatgcgccgagcaacctgcgctttctggcgacc | |
| | accccgaacagcctgctggtgagctggcagccgccgcgc | |
| | gcgcgcattaccggctatattattaaatatgaaaaaccg | |
| | ggcagcccgccgcgcgaagtggtgccgcgcccgcgcccg | |
| | ggcgtgaccgaagcgaccattaccggcctggaaccgggc | |
| | accgaatataccatttatgtgattgcgctgaaaaacaac | |
| | cagaaaagcgaaccgctgattggccgcaaaaaaaccggc | |
| | ggcggcggcagcggcggcggcggcagcgcgattccggcg | |
| | ccgaccgatctgaaatttacccaggtgaccccgaccagc | |
| | ctgagcgcgcagtggaccccgccgaacgtgcagctgacc | |
| | ggctatcgcgtgcgcgtgaccccgaaagaaaaaaccggc | |
| | ccgatgaaagaaattaacctggcgccggatagcagcagc | |
| | gtggtggtgagcggcctgatggtggcgaccaaatatgaa | |
| | gtgagcgtgtatgcgctgaaagataccctgaccagccgc | |
| | ccggcgcagggcgtggtgaccaccctggaaaacgtgagc | |
| | ccgccgcgccgcgcgcgcgtgaccgatgcgaccgaaacc | |
| | accattaccattagctggogcaccaaaaccgaaaccatt | |
| | accggctttcaggtggatgcggtgccggcgaacggccag | |
| | accccgattcagcgcaccattaaaccggatgtgcgcagc | |
| | tataccattaccggcctgcagccgggcaccgattataaa | |
| | atttatctgtataccctgaacgataacgcgcgcagcagc | |
| | ccggtggtgattgatgcgagcaccgcgattgatgcgccg | |
| | agcaacctgcgctttctggcgaccaccccgaacagcctg | |
| | ctggtgagctggcagccgccgcgcgcgcgcattaccggc | |
| | tatattattaaatatgaaaaaccgggcagcccgccgcgc | |
| | gaagtggtgccgcgcccgcgcccgggcgtgaccgaagcg | |
| | accattaccggcctggaaccgggcaccgaatataccatt | |
| | tatgtgattgcgctgaaaaacaaccagaaaagcgaaccg | |
| | ctgattggccgcaaaaaaaccagcgcgtggagccatccg | |
| | cagtttgaaaaaggcggcggcggcagcggcggcggcggc | |
| | agcggcggcagcgcgtggagccatccgcagtttgaaaaa | |
| 137 | X-B-B-X-B-X | Heparin-Binding |
| | | Sequence |
| | | Each X is |
| | | independently |
| | | selected from any |
| | | amino acid |
| | | Each B is |
| | | independently |
| | | selected from any |
| | | basic amino acid |
| 138 | X-B-B-B-X-X-B-X | Heparin-Binding |
| | | Sequence |
| | | Each X is |
| | | independently |
| | | selected from any |
| | | amino acid |
| | | Each B is |
| | | independently |
| | | selected from any |
| | | basic amino acid |
| 139 | X-B-X-B-B-X | Heparin-Binding |
| | | Sequence |
| | | Each X is |
| | | independently |
| | | selected from any |
| | | amino acid |
| | | Each B is |
| | | independently |
| | | selected from any |
| | | basic amino acid |
| 140 | X-B-X-X-B-B-B-X | Heparin-Binding |
| | | Sequence |
| | | Each X is |
| | | independently |
| | | selected from any |
| | | amino acid |
| | | Each B is |
| | | independently |
| | | selected from any |
| | | basic amino acid |
| 141 | PRRARV | Heparin-Binding |
| | | Sequence |
| 142 | LIGRKK | Heparin-Binding |
| | | Sequence |
| 143 | IGAR | Sequence of mutein |
| | | streptavidin at |
| | | sequence positions |
| | | corresponding to |
| | | positions 44 to 47 |
| | | of streptavidin |
| 144 | VTAR | Sequence of mutein |
| | | streptavidin at |
| | | sequence positions |
| | | corresponding to |
| | | positions 44 to 47 |
| | | of streptavidin |
| 145 | GS | Linker |
| 146 | GGS | Linker |
| 147 | GGSGGS | Linker |
| 148 | GGSGGSGGS | Linker |
| 149 | GGGGS | Linker |
| 150 | KNNQKSE | Heparin-Binding |
| | | Sequence |
| 151 | LIGRKKT | Heparin-Binding |
| | | Sequence |
| 152 | PRRGRV | Heparin-Binding |
| | | Sequence |
| 153 | LIGKKKT | Heparin-Binding |
| | | Sequence |
| 154 | LIGRKTG | Heparin-Binding |
| | | Sequence |
| 155 | LIGRKTG | Heparin-Binding |
| | | Sequence |
| 156 | LIGRKTG | Heparin-Binding |
| | | Sequence |
| 157 | LIGRKFD | Heparin-Binding |
| | | Sequence |
| 158 | LIGRKFE | Heparin-Binding |
| | | Sequence |
| 159 | LIGRKFD | Heparin-Binding |
| | | Sequence |
| 160 | LIGRKYD | Heparin-Binding |
| | | Sequence |
| 161 | LIGRKFN | Heparin-Binding |
| | | Sequence |
| 162 | AEAGITGTWYNQLGSTFIVTAGADGALTGTYESAVGNAE | Minimal |
| | SRYVLTGRYDSAPATDGSGTALGWTVAWKNNYRNAHSAT | streptavidin |
| | TWSGQYVGGAEARINTQWLLTSGTTEANAWKSTLVGHDT | |
| | FTKVKPSAAS | |
| 163 | MEAGITGTWYNQLGSTFIVTAGADGALTGTYVTARGNA | Mutein |
| | ESRYVLTGRYDSAPATDGSGTALGWTVAWKNNYRNAHS | Streptavidin Val⁴⁴- |
| | ATTWSGQYVGGAEARINTQWLLTSGTTEENAGYSTLVG | Thr⁴⁵-Ala⁴⁶-Arg⁴⁷ |
| | HDTFTKVKPSAAS | and Glu¹¹⁷, Gly¹²⁰, |
| | | Tyr¹²¹ (mutein |
| | | m1-9) |
| 164 | Trp-Ser-His-Pro-Gln-Phe-Glu-Lys-(Xaa)ₙ- | Sequential modules |
| | Trp-Ser-His-Pro-Gln-Phe-Glu-Lys | of streptavidin- |
| | | binding peptide |
| | | Xaa is any amino |
| | | acid; |
| | | n is 12 |

1. A protein comprising two heparin-binding domains and a streptavidin-binding partner, wherein the streptavidin-binding partner is connected to one of the heparin-binding domains.

2. The protein of claim 1, wherein the protein comprises between 2 and 10 heparin-binding domains, 2 and 9 heparin-binding domains, 2 and 8 heparin-binding domains, 2 and 7 heparin-binding domains, 2 and 6 heparin-binding domains, 2 and 5 heparin-binding domains, 2 and 4 heparin-binding domains, or 2 and 3 heparin-binding domains, each inclusive.

3. The protein of claim 1 or claim 2, wherein the protein comprises no more than two heparin-binding domains.

4. A protein set forth by the formula (heparin-binding domain)ₙ-(streptavidin-binding partner), wherein n is between 2 and 10, inclusive.

5. The protein of claim 4, wherein n is between 2 and 9, 2 and 8, 2 and 7, 2 and 6, 2 and 5, 2 and 4, or 2 and 3, each inclusive.

6. The protein of claim 4 or claim 5, wherein n is 2.

7. The protein of any one of claims 1-6, wherein the heparin-binding domains are different from one another.

8. The protein of any one of claims 1-6, wherein the heparin-binding domains are identical to one another.

9. The protein of any one of claims 1-8, wherein one, optionally each, of the heparin-binding domains comprises an amino acid sequence that binds to heparin and exhibits at least 85% sequence identity to a heparin-binding domain of fibronectin.

10. The protein of any one of claims 1-9, wherein one, optionally each, of the heparin-binding domains comprises the amino acid sequence of a heparin-binding domain of fibronectin.

11. The protein of claim 9 or claim 10, wherein the heparin-binding domain of fibronectin is heparin-binding domain II of fibronectin (FN12-14).

12. The protein of any one of claims 1-11, wherein one, optionally each, of the heparin-binding domains comprises an amino acid sequence selected from X-B-B-X-B-X (SEQ ID NO: 137), X-B-B-B-X-X-B-X (SEQ ID NO: 138), X-B-X-B-B-X (SEQ ID NO: 139), and X-B-X-X-B-B-B-X (SEQ ID NO: 140), wherein each X is independently selected from any hydrophobic amino acid, and each B is independently selected from any basic amino acid.

13. The protein of any one of claims 1-12, wherein one, optionally each, of the heparin-binding domains comprises the amino acid sequence X-B-B-X-B-X (SEQ ID NO: 137), wherein each X is independently selected from any hydrophobic amino acid, and each B is independently selected from any basic amino acid.

14. The protein of claim 12 or claim 13, wherein each B is independently selected from arginine and lysine.

15. The protein of any one of claims 1-14, wherein one, optionally each, of the heparin-binding domains comprises the amino acid sequence set forth in SEQ ID NO: 141.

16. The protein of any one of claims 1-15, wherein one, optionally each, of the heparin-binding domains comprises the amino acid sequence set forth in SEQ ID NO: 150.

17. The protein of any one of claims 1-16, wherein one, optionally each, of the heparin-binding domains comprises the amino acid sequence set forth in SEQ ID NO: 142.

18. The protein of any one of claims 1-17, wherein one, optionally each, of the heparin-binding domains comprises an amino acid sequence that binds to heparin and exhibits at least 85% sequence identity to the amino acid sequence set forth in SEQ ID NO: 133.

19. The protein of any one of claims 1-18, wherein one, optionally each, of the heparin-binding domains comprises the amino acid sequence set forth in SEQ ID NO: 133.

20. The protein of any one of claims 1-19, wherein one, optionally each, of the heparin-binding domains consists of the amino acid sequence set forth in SEQ ID NO: 133.

21. The protein of any one of claims 1-20, wherein the heparin-binding domains are connected to one another via a peptide linker.

22. The protein of claim 21, wherein the peptide linker is a GS-linker.

23. The protein of claim 21 or claim 22, wherein the peptide linker comprises the amino acid sequence set forth in SEQ ID NO: 134.

24. The protein of any one of claims 21-23, wherein the peptide linker consists of the amino acid sequence set forth in SEQ ID NO: 134.

25. The protein of any one of claims 1-24, wherein the streptavidin-binding partner is directly connected to one of the heparin-binding domains.

26. The protein of any one of claims 1-25, wherein the streptavidin-binding partner is directly connected to the C-terminus of one of the heparin-binding domains.

27. A protein comprising a viral-binding domain and a streptavidin-binding partner, wherein the streptavidin-binding partner is connected to the viral-binding domain.

28. The protein of any one of claims 1-27, wherein the streptavidin-binding partner is at the C-terminus of the protein.

29. The protein of any one of claims 1-28, wherein the streptavidin-binding partner binds to a molecule that is streptavidin, avidin, a streptavidin analog or mutein, or an avidin analog or mutein.

30. The protein of any one of claims 1-29, wherein the streptavidin-binding partner binds to a molecule that is streptavidin or a streptavidin mutein.

31. The protein of claim 29 or claim 30, wherein the streptavidin-binding partner binds to a biotin-binding site of the molecule.

32. The protein of any one of claims 29-31, wherein the streptavidin mutein comprises the amino acid sequence set forth in any of SEQ ID NO: 3-6, 27, 28, 104, 105, and 163.

33. The protein of any one of claims 1-32, wherein the streptavidin-binding partner comprises biotin, a biotin analog or derivative, or a streptavidin-binding peptide.

34. The protein of any one of claims 1-33, wherein the streptavidin-binding partner comprises a streptavidin-binding peptide.

35. The protein of any one of claims 1-34, wherein the streptavidin-binding partner is a streptavidin-binding peptide.

36. The protein of any one of claims 33-35, wherein the streptavidin-binding peptide comprises the amino acid sequence set forth SEQ ID NO: 7 or SEQ ID NO: 8.

37. The protein of any one of claims 33-36, wherein the streptavidin-binding peptide comprises a sequential arrangement of two streptavidin-binding modules.

38. The protein of claim 37, wherein the streptavidin-binding modules are separated from one another by no more than 50 amino acids.

39. The protein of claim 37 or claim 38, wherein one of the streptavidin-binding modules comprises an amino acid sequence selected from the amino acid sequences set forth in SEQ ID NO: 7 and SEQ ID NO: 8, optionally wherein each of the streptavidin-binding modules comprises an amino acid sequence independently selected from the amino acid sequences set forth in SEQ ID NO: 7 and SEQ ID NO: 8.

40. The protein of any one of claims 37-39, wherein each of the streptavidin-binding modules comprises the amino acid sequence set forth in SEQ ID NO: 8.

41. The protein of any one of claims 33-40, wherein the streptavidin-binding peptide comprises the amino acid sequence set forth in any of SEQ ID NO: 15-19.

42. The protein of any one of claims 33-41, wherein the streptavidin-binding peptide consists of the amino acid sequence set forth in any of SEQ ID NO: 15-19.

43. The protein of any one of claims 33-42, wherein the streptavidin-binding peptide comprises the amino acid sequence set forth in SEQ ID NO: 16.

44. The protein of any one of claims 33-43, wherein the streptavidin-binding peptide consists of the amino acid sequence set forth in SEQ ID NO: 16.

45. The protein of any one of claims 1-44, wherein the protein comprises the amino acid sequence set forth in SEQ ID NO: 135.

46. The protein of any one of claims 1-45, wherein the protein consists of the amino acid sequence set forth in SEQ ID NO: 135.

47. A heparin-binding reagent comprising (i) a protein reagent comprising one or a plurality of a molecule that is streptavidin, avidin, a streptavidin analog or mutein, or an avidin analog or mutein, and (ii) the protein of any one of claims 1-46, wherein the streptavidin-binding partner of the protein is bound to at least one of the one or plurality of molecules.

48. The heparin-binding reagent of claim 47, wherein the heparin-binding reagent is capable of being immobilized on a solid support.

49. The heparin-binding reagent of claim 47, wherein the heparin-binding reagent is in soluble form.

50. The heparin-binding reagent of any one of claims 47-49, wherein the heparin-binding reagent comprises a plurality of the molecule, and the streptavidin-binding partner is bound to at least one of the plurality of molecules.

51. The heparin-binding reagent of claim 50, wherein the protein reagent is an oligomer of the plurality of molecules.

52. The heparin-binding reagent of claim 50 or claim 51, wherein the plurality of molecules comprises between or between about 500 and 5000 tetramers, 1000 and 4000 tetramers, or 2000 and 3000 tetramers, each inclusive, of the molecule of the protein reagent or a mixture of molecules each independently selected from streptavidin, avidin, a streptavidin analog or mutein, or an avidin analog or mutein.

53. The heparin-binding reagent of any one of claims 50-52, wherein the plurality of molecules comprises about 2500 tetramers of the molecule of the protein reagent or a mixture of molecules each independently selected from streptavidin, avidin, a streptavidin analog or mutein, or an avidin analog or mutein.

54. The heparin-binding reagent of any one of claims 47-53, wherein the molecule of the protein reagent is streptavidin.

55. The heparin-binding reagent of any one of claims 47-53, wherein the molecule of the protein reagent is a streptavidin mutein.

56. The heparin-binding reagent of any one of claims 47-53 and 55, wherein the streptavidin mutein comprises the amino acid sequence IGAR (SEQ ID NO: 143) or VTAR (SEQ ID NO: 144) at sequence positions corresponding to positions 44 to 47 of the sequence of amino acids set forth in SEQ ID NO: 1.

57. The heparin-binding reagent of any one of claims 47-53, 55, and 56, wherein the streptavidin mutein begins N-terminally in the region of amino acid positions 10 to 16 of SEQ ID NO: 1 and terminates C-terminally in the region of amino acid positions 133 to 142 of SEQ ID NO: 1.

58. A heparin-binding reagent comprising a protein reagent and the protein of any one of claims 1-46, wherein:
the protein reagent is an oligomer of a plurality of a molecule that is a streptavidin mutein, wherein the streptavidin mutein (i) comprises the amino acid sequence IGAR (SEQ ID NO: 143) or VTAR (SEQ ID NO: 144) at sequence positions corresponding to positions 44 to 47 of the sequence of amino acids set forth in SEQ ID NO: 1, (ii) begins N-terminally in the region of amino acid positions 10 to 16 of SEQ ID NO: 1, and (iii) terminates C-terminally in the region of amino acid positions 133 to 142 of SEQ ID NO: 1; and
the streptavidin-binding partner of the protein (i) is a streptavidin-binding peptide comprising the amino acid sequence set forth in SEQ ID NO: 7 or SEQ ID NO: 8, and (ii) is bound to at least one of the plurality of molecules.

59. The heparin-binding reagent of claim 58, wherein the plurality of molecules comprises between or between about 500 and 5000 tetramers, 1000 and 4000 tetramers, or 2000 and 3000 tetramers, each inclusive, of the molecule of the protein reagent.

60. The heparin-binding reagent of claim 58 or claim 59, wherein the plurality of molecules comprises about 2500 tetramers of the molecule of the protein reagent.

61. The heparin-binding reagent of any one of claims 47-53 and 55-60, wherein the streptavidin mutein further comprises the residues Glu117, Gly120, and Tyr121 with reference to positions of the sequence of amino acids set forth in SEQ ID NO: 1.

62. The heparin-binding reagent of any one of claims 47-53 and 55-61, wherein the streptavidin mutein comprises the sequence of amino acids set forth in any of SEQ ID NOs: 3-6, 27, 28, 104, and 105.

63. The heparin-binding reagent of any one of claims 47-53, 55-60, and 62, wherein the streptavidin mutein comprises the sequence of amino acids set forth in SEQ ID NO: 6.

64. The heparin-binding reagent of any one of claims 50-63, wherein molecules of the plurality of molecules are crosslinked to one another by a polysaccharide or a bifunctional linker.

65. The heparin-binding reagent of any one of claims 50-64, wherein molecules of the plurality of molecules are crosslinked to one another by an amine-to-thiol crosslinker.

66. The heparin-binding reagent of any one of claims 47-65, wherein the streptavidin-binding partner is bound to a biotin-binding site of the at least one molecule.

67. The heparin-binding reagent of any one of claims 47-66, wherein the streptavidin-binding partner is reversibly bound to the at least one molecule.

68. The heparin-binding reagent of any one of claims 47-67, wherein the binding affinity of the streptavidin-binding partner to the at least one molecule is reduced compared to the binding affinity of biotin to streptavidin.

69. The heparin-binding reagent of any one of claims 47-68, wherein the binding of the streptavidin-binding partner to the at least one molecule is disrupted by the presence of biotin.

70. The heparin-binding reagent of any one of claims 47-69, wherein the heparin-binding reagent further comprises one or more binding agents that are each bound to the protein reagent.

71. The heparin-binding reagent of claim 70, wherein one, optionally each, of the one or more binding agents is reversibly bound to the protein reagent.

72. The heparin-binding reagent of claim 70 or claim 71, wherein each of the one or more binding agents comprises a streptavidin-binding partner that is bound to at least one of the plurality of molecules.

73. The heparin-binding reagent of claim 72, wherein the streptavidin-binding partner of one, optionally each, of the one or more binding agents is bound to a biotin-binding site of the at least one molecule.

74. The heparin-binding reagent of claim 72 or claim 73, wherein the streptavidin-binding partner of one, optionally each, of the one or more binding agents comprises biotin, a biotin analog or derivative, or a streptavidin-binding peptide.

75. The heparin-binding reagent of any one of claims 72-74, wherein the streptavidin-binding partner of one, optionally each, of the one or more binding agents is reversibly bound to the at least one molecule.

76. The heparin-binding reagent of any one of claims 72-75, wherein the binding affinity of the streptavidin-binding partner of one, optionally each, of the one or more binding agents to the at least one molecule is reduced compared to the binding affinity of biotin to streptavidin.

77. The heparin-binding reagent of any one of claims 72-76, wherein the binding of the streptavidin-binding partner of one, optionally each, of the one or more binding agents to the at least one molecule is disrupted by the presence of biotin.

78. The heparin-binding reagent of any one of claims 72-77, wherein the at least one molecule is streptavidin, and the streptavidin-binding partner of one, optionally each, of the one or more binding agents comprises a biotin analog or derivative or a streptavidin-binding peptide.

79. The heparin-binding reagent of any one of claims 72-78, wherein the streptavidin-binding partner of one, optionally each, of the one or more binding agents comprises a streptavidin-binding peptide.

80. The heparin-binding reagent of any one of claims 72-79, wherein the streptavidin-binding partner of one, optionally each, of the one or more binding agents consists of a streptavidin-binding peptide.

81. The heparin-binding reagent of any one of claims 74-80, wherein the streptavidin-binding peptide of one of the one or more binding agents comprises an amino acid sequence selected from the amino acid sequences set forth in SEQ ID NO: 7 and SEQ ID NO: 8, optionally wherein the streptavidin-binding peptide of each of the one or more binding agents comprises an amino acid sequence independently selected from the amino acid sequences set forth in SEQ ID NO: 7 and SEQ ID NO: 8.

82. The heparin-binding reagent of any one of claims 74-81, wherein the streptavidin-binding peptide of one, optionally each, of the one or more binding agents comprises a sequential arrangement of two streptavidin-binding modules.

83. The heparin-binding reagent of claim 82, wherein the streptavidin-binding modules are separated from one another by no more than 50 amino acids.

84. The heparin-binding reagent of claim 82 or claim 83, wherein one of the streptavidin-binding modules comprises an amino acid sequence selected from the amino acid sequences set forth in SEQ ID NO: 7 and SEQ ID NO: 8, optionally wherein each of the streptavidin-binding modules comprises an amino acid sequence independently selected from the amino acid sequences set forth in SEQ ID NO: 7 and SEQ ID NO: 8.

85. The heparin-binding reagent of any one of claims 82-84, wherein each of the streptavidin-binding modules comprises the amino acid sequence set forth in SEQ ID NO: 8.

86. The heparin-binding reagent of any one of claims 74-85, wherein the streptavidin-binding peptide of one of the one or more binding agents comprises an amino acid sequence selected from the amino acid sequences set forth in SEQ ID NO: 15-19, optionally wherein the streptavidin-binding peptide of each of the one or more binding agents comprises an amino acid sequence independently selected from the amino acid sequences set forth in SEQ ID NO: 15-19.

87. The heparin-binding reagent of any one of claims 74-86, wherein the streptavidin-binding peptide of one of the one or more binding agents consists of an amino acid sequence selected from the amino acid sequences set forth in SEQ ID NO: 15-19, optionally wherein the streptavidin-binding peptide of each of the one or more binding agents consists of an amino acid sequence independently selected from the amino acid sequences set forth in SEQ ID NO: 15-19.

88. The heparin-binding reagent of any one of claims 74-87, wherein the streptavidin-binding peptide of one, optionally each, of the one or more binding agents comprises the amino acid sequence set forth in SEQ ID NO: 16.

89. The heparin-binding reagent of any one of claims 74-88, wherein the streptavidin-binding peptide of one, optionally each, of the one or more binding agents consists of the amino acid sequence set forth in SEQ ID NO: 16.

90. The heparin-binding reagent of any one of claims 70-89, wherein one, optionally each, of the one or more binding agents comprises an antibody or an antibody fragment.

91. The heparin-binding reagent of any one of claims 70-90, wherein one, optionally each, of the one or more binding agents comprises an antibody fragment.

92. The heparin-binding reagent of claim 90 or claim 91, wherein the antibody fragment is a monovalent antibody fragment.

93. The heparin-binding reagent of any one of claims 90-92, wherein the antibody fragment is a Fab.

94. The heparin-binding reagent of any one of claims 70-93, wherein one of the one or more binding agents is a binding agent that binds to a molecule expressed on the surface of a target cell.

95. The heparin-binding reagent of claim 94, wherein the target cell is an immune cell.

96. The heparin-binding reagent of claim 94 or claim 95, wherein the target cell is a T cell.

97. The heparin-binding reagent of any one of claims 94-96, wherein the molecule expressed on the surface of the target cell is a member of a TCR/CD3 complex.

98. The heparin-binding reagent of any one of claims 94-97, wherein the molecule expressed on the surface of the target cell is CD3.

99. The heparin-binding reagent of any one of claims 94-96, wherein the molecule expressed on the surface of the target cell is a costimulatory molecule.

100. The heparin-binding reagent of any one of claims 94-96, wherein the molecule expressed on the surface of the target cell is a co-receptor.

101. The heparin-binding reagent of any one of claims 94-100, wherein the binding agent is a first binding agent, the molecule expressed on the surface of the target cell is a first molecule, and one of the one or more binding agents is a second binding agent that binds to a second molecule expressed on the surface of the target cell.

102. The heparin-binding reagent of claim 101, wherein the second molecule expressed on the surface of the target cell is a costimulatory molecule.

103. The heparin-binding reagent of any one of claims 99, 101, and 102, wherein the costimulatory molecule is CD28, CD90 (Thy-1), CD95 (Apo−/Fas), CD137 (4-1BB), CD154 (CD40L), ICOS, LAT, CD27, OX40, or HVEM.

104. The heparin-binding reagent of any one of claims 101-103, wherein the first binding agent comprises the streptavidin-binding partner and an anti-CD3 antibody or antibody fragment, and the second binding agent comprises the streptavidin-binding partner and an anti-CD28 antibody or antibody fragment.

105. The heparin-binding reagent of any one of claims 101-104, wherein the first binding agent comprises the streptavidin-binding partner and an anti-CD3 Fab, and the second binding agent comprises the streptavidin-binding partner and an anti-CD28 Fab.

106. The heparin-binding reagent of claim 101, wherein the second molecule expressed on the surface of the target cell is a co-receptor.

107. The heparin-binding reagent of any one of claims 100-103 and 106, wherein the co-receptor is CD4 or CD8.

108. The heparin-binding reagent of any one of claims 100, 101, 106, and 107, wherein the first binding agent comprises the streptavidin-binding partner and an anti-CD4 antibody or antibody fragment, and the second binding agent comprises the streptavidin-binding partner and an anti-CD8 antibody or antibody fragment.

109. The heparin-binding reagent of any one of claims 100, 101, and 106-108, wherein the first binding agent comprises the streptavidin-binding partner and an anti-CD4 Fab, and the second binding agent comprises the streptavidin-binding partner and an anti-CD8 Fab.

110. A method for purifying viral particles, comprising:
(a) adding a sample comprising viral particles to an internal cavity of a chromatography column, wherein the internal cavity comprises a stationary phase comprising a chromatography matrix and the protein of any one of claims 1-46, and the protein is immobilized on the chromatography matrix, thereby immobilizing a viral particle from the sample on the stationary phase; and
(b) eluting the viral particle from the internal cavity.

111. The method of claim 110, wherein the eluting comprises disrupting the binding between the protein and the viral particle.

112. The method of claim 110 or claim 111, wherein the protein is reversibly bound to the chromatography matrix.

113. The method of any one of claims 110-112, wherein the eluting comprises disrupting the binding between the protein and the chromatography matrix.

114. The method of any one of claims 110-113, wherein the protein is immobilized on the chromatography matrix via a selection reagent comprising a molecule that is streptavidin, avidin, a streptavidin analog or mutein, or an avidin analog or mutein, wherein the selection reagent is immobilized on the chromatography matrix, and the streptavidin-binding partner of the protein is bound to the molecule.

115. The method of claim 114, wherein the streptavidin-binding partner is bound to a biotin-binding site of the molecule of the selection reagent.

116. The method of claim 114 or claim 115, wherein the streptavidin-binding partner is reversibly bound to the molecule of the selection reagent.

117. The method of any one of claims 114-116, wherein the molecule of the selection reagent is a streptavidin mutein.

118. The method of any one of claims 114-117, wherein the streptavidin mutein comprises the amino acid sequence IGAR (SEQ ID NO: 143) or VTAR (SEQ ID NO: 144) at sequence positions corresponding to positions 44 to 47 of the sequence of amino acids set forth in SEQ ID NO: 1.

119. The method of any one of claims 114-118, wherein the streptavidin mutein begins N-terminally in the region of amino acid positions 10 to 16 of SEQ ID NO: 1 and terminates C-terminally in the region of amino acid positions 133 to 142 of SEQ ID NO: 1.

120. The method of any one of claims 114-119, wherein the streptavidin mutein comprises the sequence of amino acids set forth in any of SEQ ID NOs: 3-6, 27, 28, 104, and 105.

121. The method of any one of claims 114-120, wherein the streptavidin mutein comprises the sequence of amino acids set forth in SEQ ID NO: 6.

122. The method of any one of claims 110-121, wherein the eluting comprises adding, to the internal cavity, a composition comprising a substance that disrupts the immobilization of the viral particle on the stationary phase.

123. The method of claim 122, wherein the substance comprises biotin or a biotin analog or derivative.

124. The method of claim 122 or claim 123, wherein the substance comprises biotin.

125. A method for transducing cells, comprising incubating one or more target cells in the simultaneous presence of a viral particle and the heparin-binding reagent of any one of claims 47-109, thereby producing a transduced target cell.

126. The method of claim 125, wherein at least a portion of the incubating occurs in an internal cavity of a chromatography column.

127. The method of claim 125 or claim 126, wherein the one or more target cells are immobilized on a solid support during at least a portion of the incubating.

128. The method of claim 127, wherein the solid support is a stationary phase for column chromatography.

129. The method of claim 128, wherein the stationary phase is comprised in an internal cavity of a chromatography column during the at least a portion of the incubating.

130. A method for on-column transduction of cells, comprising incubating, in an internal cavity of a chromatography column, one or more target cells in the simultaneous presence of a viral particle and the heparin-binding reagent of any one of claims 47-109, thereby producing a transduced target cell, wherein the internal cavity comprises a stationary phase, and the one or more target cells are immobilized on the stationary phase during at least a portion of the incubation.

131. The method of any one of claims 128-130, wherein prior to the incubating, the method comprises adding a sample comprising a plurality of the target cells to the stationary phase, thereby immobilizing the one or more target cells on the stationary phase.

132. The method of any one of claims 128-131, wherein the stationary phase comprises a selection agent that specifically binds to a selection marker expressed on the surface of the one or more target cells, wherein specific binding of the selection agent to the selection marker effects the immobilization of the one or more target cells on the stationary phase.

133. A method for on-column transduction of cells, comprising:
(a) adding a sample comprising a plurality of target cells to an internal cavity of a chromatography column, wherein the internal cavity comprises a stationary phase comprising a selection agent that specifically binds to a selection marker expressed on the surface of one or more of the plurality of target cells, thereby immobilizing the one or more target cells on the stationary phase; and
(b) incubating the one or more target cells in the simultaneous presence of a viral particle and the heparin-binding reagent of any one of claims 47-109, thereby producing a transduced target cell, wherein the one or more target cells are immobilized on the stationary phase during at least a portion of the incubation.

134. The method of any one of claims 125-133, wherein prior to the incubating, the method comprises contacting the one or more target cells with one or more compositions each comprising one or both of the heparin-binding reagent and the viral particle.

135. The method of any one of claims 128-134, wherein prior to the incubating, the method comprises adding, to the stationary phase, one or more compositions each comprising one or both of the heparin-binding reagent and the viral particle, thereby contacting the one or more target cells with the one or more compositions.

136. The method of claim 135, wherein the one or more compositions are a first composition comprising the heparin-binding reagent and a second, separate composition comprising the viral particle.

137. The method of claim 136, wherein the one or more target cells are simultaneously contacted with the first composition and the second composition.

138. The method of claim 135, wherein the one or more compositions is a composition comprising both of the heparin-binding reagent and the viral particle.

139. The method of claim 138, wherein the method comprises mixing the heparin-binding reagent and the viral particle to form the composition comprising both of the heparin-binding reagent and the viral particle.

140. The method of any one of claims 125-139, wherein prior to the incubating, the one or more target cells are incubated in the presence of one or more binding agents, one of the one or more binding agents being a binding agent that binds to a molecule expressed on the surface of the one or more target cells.

141. The method of any one of claims 125-140, wherein at least a portion, optionally all, of the incubating is further in the presence of one or more binding agents, one of the one or more binding agents being a binding agent that binds to a molecule expressed on the surface of the one or more target cells.

142. The method of claim 140 or claim 141, wherein the one or more binding agents are comprised in a second reagent comprising a second protein reagent comprising one or a plurality of a molecule that is streptavidin, avidin, a streptavidin analog or mutein, or an avidin analog or mutein, wherein the one or more binding agents are each bound to the second protein reagent of the second reagent.

143. The method of claim 142, wherein one, optionally each, of the one or more binding agents is reversibly bound to the second protein reagent.

144. The method of claim 142 or claim 143, wherein each of the one or more binding agents comprises a streptavidin-binding partner that is bound to at least one of the plurality of molecules of the second protein reagent.

145. The method of any one of claims 142-144, wherein the second reagent is a stimulatory reagent.

146. The method of any one of claims 142-145, wherein the method comprises contacting the one or more target cells with a composition comprising the second reagent.

147. The method of any one of claims 142-146, wherein the method comprises adding, to the stationary phase, a composition comprising the second reagent, thereby contacting the one or more target cells with the composition comprising the second reagent.

148. The method of claim 146 or claim 147, wherein the one or more target cells are simultaneously contacted with the composition comprising the second reagent and the one or more compositions each comprising one or both of the heparin-binding reagent and the viral particle.

149. The method of any one of claims 142-145, wherein the composition comprising both of the heparin-binding reagent and the viral particle further comprises the second reagent.

150. The method of claim 140 or claim 141, wherein the heparin-binding reagent comprises the one or more binding agents, wherein the one or more binding agents are each bound to the protein reagent of the heparin-binding reagent.

151. The method of claim 150, wherein one, optionally each, of the one or more binding agents is reversibly bound to the protein reagent.

152. The method of claim 150 or claim 151, wherein each of the one or more binding agents comprises a streptavidin-binding partner that is bound to at least one of the plurality of molecules of the protein reagent.

153. The method of any one of claims 140-152, wherein the streptavidin-binding partner of one, optionally each, of the one or more binding agents comprises biotin, a biotin analog or derivative, or a streptavidin-binding peptide.

154. The method of any one of claims 140-153, wherein the streptavidin-binding partner of one, optionally each, of the one or more binding agents comprises a streptavidin-binding peptide.

155. The method of claim 153 or claim 154, wherein the streptavidin-binding peptide of one, optionally each, of the one or more binding agents comprises the amino acid sequence set forth in any of SEQ ID NO: 7, 8, and 15-19.

156. The method of any one of claims 153-155, wherein the streptavidin-binding peptide of one, optionally each, of the one or more binding agents comprises the amino acid sequence set forth in SEQ ID NO: 16.

157. The method of any one of claims 140-156, wherein one, optionally each, of the one or more binding agents comprises an antibody or an antibody fragment.

158. The method of any one of claims 140-157, wherein one, optionally each, of the one or more binding agents comprises an antibody fragment.

159. The method of claim 157 or claim 158, wherein the antibody fragment is a monovalent antibody fragment.

160. The method of any one of claims 157-159, wherein the antibody fragment is a Fab.

161. The method of any one of claims 140-160, wherein the binding agent binds to the molecule expressed on the one or more target cells and thereby provides a primary activation signal to the one or more target cells.

162. The method of any one of claims 125-161, wherein the one or more target cells are immune cells.

163. The method of any one of claims 125-162, wherein the one or more target cells are T cells.

164. The method of any one of claims 140-163, wherein the molecule expressed on the surface of the one or more target cells is a member of a TCR/CD3 complex.

165. The method of any one of claims 140-164, wherein the molecule expressed on the surface of the one or more target cells is CD3.

166. The method of any one of claims 140-160, 162, and 163, wherein the molecule expressed on the surface of the one or more target cells is a costimulatory molecule.

167. The method of claim 166, wherein the binding agent binds to the costimulatory molecule and thereby provides a costimulatory signal to the one or more target cells.

168. The method of any one of claims 140-160, 162, and 163, wherein the molecule expressed on the surface of the one or more target cells is a co-receptor.

169. The method of any one of claims 140-168, wherein the binding agent is a first binding agent, the molecule expressed on the surface of the one or more target cells is a first molecule, and one of the one or more binding agents is a second binding agent that binds to a second molecule expressed on the surface of the one or more target cells.

170. The method of claim 169, wherein the second molecule expressed on the surface of the one or more target cells is a costimulatory molecule.

171. The method of claim 170, wherein the second binding agent binds to the costimulatory molecule and thereby provides a costimulatory signal to the one or more target cells.

172. The method of any one of claims 166, 167, and 169-171, wherein the costimulatory molecule is CD28, CD90 (Thy-1), CD95 (Apo−/Fas), CD137 (4-1BB), CD154 (CD40L), ICOS, LAT, CD27, OX40, or HVEM.

173. The method of any one of claims 169-172, wherein the first binding agent comprises a streptavidin-binding partner and an anti-CD3 antibody or antibody fragment, and the second binding agent comprises a streptavidin-binding partner and an anti-CD28 antibody or antibody fragment.

174. The method of any one of claims 169-173, wherein the first binding agent comprises a streptavidin-binding partner and an anti-CD3 Fab, and the second binding agent comprises a streptavidin-binding partner and an anti-CD28 Fab.

175. The method of claim 169, wherein the second molecule expressed on the surface of the target cell is a co-receptor.

176. The method of any one of claims 168, 169, and 175, wherein the co-receptor is CD4 or CD8.

177. The method of any one of claims 168, 169, 175, and 176, wherein the first binding agent comprises a streptavidin-binding partner and an anti-CD4 antibody or antibody fragment, and the second binding agent comprises a streptavidin-binding partner and an anti-CD8 antibody or antibody fragment.

178. The method of any one of claims 168, 169, and 175-177, wherein the first binding agent comprises a streptavidin-binding partner and an anti-CD4 Fab, and the second binding agent comprises a streptavidin-binding partner and an anti-CD8 Fab.

179. The method of any one of claims 131-178, wherein the incubating is initiated within or within about 10 minutes, within or within about 20 minutes, within or within about 30 minutes, within or within about 45 minutes, within or within about 60 minutes, within or within about 90 minutes, or within or within about 120 minutes after adding the sample to the internal cavity.

180. The method of any one of claims 125-179, wherein the incubating is carried out in a cell medium.

181. The method of claim 180, wherein the cell media is a serum free medium.

182. The method of claim 180 or claim 181, wherein the cell medium comprises a recombinant cytokine.

183. The method of claim 182, wherein the recombinant cytokine is selected from IL-2, IL-15, and IL-7.

184. The method of any one of claims 180-183, wherein the cell medium comprises recombinant IL-2, IL-15, and IL-7.

185. The method of any one of claims 125-184, wherein the incubating is in the presence of between or between about 0.1 μg and 20 μg, inclusive; between or between about 0.1 μg and 12 μg, inclusive; or between or between about 0.5 μg and 8 μg, inclusive, of the heparin-binding reagent; each per 10⁶ cells of the one or more target cells or of an estimated cell count thereof.

186. The method of any one of claims 125-185, wherein the incubating is in the presence of between or between about 3 μg and 5 μg, inclusive, of the heparin-binding reagent per 10⁶ cells of the one or more target cells or of an estimated cell count thereof.

187. The method of any one of claims 134-186, wherein the composition of the one or more compositions that comprises the heparin-binding reagent comprises between or between about 0.1 μg and 20 μg, inclusive; between or between about 0.1 μg and 12 μg, inclusive; or between or between about 0.5 μg and 8 μg, inclusive, of the heparin-binding reagent; each per 10⁶ cells of the one or more target cells or of an estimated cell count thereof.

188. The method of any one of claims 134-187, wherein the composition of the one or more compositions that comprises the heparin-binding reagent comprises between or between about 3 μg and 5 μg, inclusive, of the heparin-binding reagent per 10⁶ cells of the one or more target cells or of an estimated cell count thereof.

189. The method of any one of claims 125-188, wherein the incubating is in the presence of between or between about 0.1 μL and 100 μL, inclusive; between or between about 0.5 μL and 50 μL, inclusive; between or between about 1 μL and 25 μL, inclusive; or between or between about 2 μL and 10 μL, inclusive, of a preparation of the viral particle per 10⁶ cells of the one or more target cells or of an estimated cell count thereof.

190. The method of any one of claims 125-189, wherein the incubating is in the presence of about 6 μL per 10⁶ cells of the one or more target cells or of an estimated cell count thereof.

191. The method of any one of claims 134-190, wherein the composition of the one or more compositions that comprises the viral particle comprises between or between about 0.1 μL and 100 μL, inclusive; between or between about 0.5 μL and 50 μL, inclusive; between or between about 1 μL and 25 μL, inclusive; or between or between about 2 μL and 10 μL, inclusive, of a preparation of the viral particle per 10⁶ cells of the one or more target cells or of an estimated cell count thereof.

192. The method of any one of claims 134-191, wherein the composition of the one or more compositions that comprises the viral particle comprises about 6 μL per 10⁶ cells of the one or more target cells or of an estimated cell count thereof.

193. The method of any one of claims 189-192, wherein the preparation of the viral particle has a titer of between or between about 1×10⁶ TU/mL and 1×10⁹ TU/mL, between or between about 1×10⁶ TU/mL and 1×10⁸ TU/mL, between or between about 1×10⁶ TU/mL and 1×10⁷ TU/mL, between or between about 1×10⁷ TU/mL and 1×10⁹ TU/mL, between or between about 1×10⁷ TU/mL and 1×10⁸ TU/mL, or between or between about 1×10⁸ TU/mL and 1×10⁹ TU/mL.

194. The method of any one of claims 128-193, wherein the stationary phase has a binding capacity of between or between about 0.5 billion and 5 billion cells, 0.5 billion and 4 billion cells, 0.5 billion and 3 billion cells, 0.5 billion and 2 billion cells, 1 billion and 5 billion cells, 1 billion and 4 billion cells, 1 billion and 3 billion cells, or 1 billion and 2 billion cells, each inclusive.

195. The method of any one of claims 128-194, wherein the stationary phase has a binding capacity of between or between about 1 billion and 2 billion cells, inclusive.

196. The method of any one of claims 125-195, wherein at least a portion, optionally all, of the incubating is carried out at a temperature between about 35° C. and about 39° C.

197. The method of any one of claims 125-196, wherein at least a portion, optionally all, of the incubating is carried out at a temperature of or of about 37° C.

198. The method of any one of claims 125-197, wherein the method comprises collecting the transduced target cell.

199. The method of claim 198, wherein the collecting comprises eluting the transduced target cell from the chromatography column.

200. The method of claim 198 or claim 199, the collecting is carried out within 24 hours, 22 hours, 20 hours, 18 hours, 16 hours, 16 hours, 14 hours, 12 hours, 10 hours, 9 hours, 8 hours, 7 hours, 6 hours, or 5 hours after the initiation of the incubating.

201. The method of any one of claims 198-200, wherein the collecting is carried out between or between about 2 hours and 24 hours, 2 hours and 22 hours, 2 hours and 20 hours, 2 hours and 18 hours, 2 hours and 16 hours, 2 hours and 14 hours, 2 hours and 12 hours, 2 hours and 10 hours, 2 hours and 9 hours, 2 hours and 8 hours, 2 hours and 7 hours, 2 hours and 6 hours, 2 hours and 5 hours, 3 hours and 6 hours, 3 hours and 5 hours, 4 hours and 6 hours, or 4 hours and 5 hours, each inclusive, after the initiation of the incubating.

202. The method of any one of claims 198-201, wherein the collecting is carried out at or about 4.5 hours after the initiation of the incubating.

203. The method of any one of claims 198-202, wherein the collecting comprises adding a wash buffer to the chromatography column to collect the transduced target cell.

204. The method of claim 203, wherein the wash buffer comprises a cell medium.

205. The method of claim 203 or claim 204, wherein the wash buffer does not comprise a competition agent to elute the transduced target cell from the stationary phase.

206. The method of claim 203 or claim 204, wherein the wash buffer comprises a competition agent to elute the transduced target cell from the stationary phase.

207. The method of claim 205 or claim 206, wherein the competition agent facilitates detachment of the one or more target cells from the stationary phase.

208. The method of any one of claims 205-207, wherein the competition agent comprises biotin or a biotin analog or derivative.

209. The method of any one of claims 205-208, wherein the competition agent comprises biotin.

210. The method of any one of claims 198-209, wherein the method comprises further incubating the collected transduced target cell.

211. The method of any one of claims 132-210, wherein the stationary phase comprises a chromatography matrix, and the selection agent is immobilized on the chromatography matrix.

212. The method of claim 211, wherein the selection agent is immobilized on the chromatography matrix via a selection reagent comprising a molecule that is streptavidin, avidin, a streptavidin analog or mutein, or an avidin analog or mutein, wherein the selection reagent is immobilized on the chromatography matrix, and the selection agent comprises a streptavidin-binding partner that is bound to the molecule.

213. The method of claim 212, wherein the streptavidin-binding partner of the selection agent is bound to a biotin-binding site of the molecule of the selection reagent.

214. The method of claim 212 or claim 213, wherein the molecule of the selection reagent is a streptavidin mutein.

215. The method of any one of claims 212-214, wherein the streptavidin mutein comprises the amino acid sequence IGAR (SEQ ID NO: 143) or VTAR (SEQ ID NO: 144) at sequence positions corresponding to positions 44 to 47 of the sequence of amino acids set forth in SEQ ID NO: 1.

216. The method of any one of claims 212-215, wherein the streptavidin mutein begins N-terminally in the region of amino acid positions 10 to 16 of SEQ ID NO: 1 and terminates C-terminally in the region of amino acid positions 133 to 142 of SEQ ID NO: 1.

217. The method of any one of claims 212-216, wherein the streptavidin mutein comprises the sequence of amino acids set forth in any of SEQ ID NOs: 3-6, 27, 28, 104, 105, and 163.

218. The method of any one of claims 212-217, wherein the streptavidin mutein comprises the sequence of amino acids set forth in SEQ ID NO: 6.

219. The method of any one of claims 212-218, wherein the streptavidin-binding partner of the selection agent comprises biotin, a biotin analog or derivative, or a streptavidin-binding peptide.

220. The method of any one of claims 212-219, wherein the binding of the streptavidin-binding partner of the selection agent to the molecule of the selection reagent is reversible.

221. The method of any one of claims 212-220, wherein the streptavidin-binding partner of the selection agent comprises a streptavidin-binding peptide.

222. The method of any one of claims 219-221, wherein the streptavidin-binding peptide of the selection agent comprises the amino acid sequence set forth in any of SEQ ID NO: 7, 8, and 15-19.

223. The method of any one of claims 219-222, wherein the streptavidin-binding peptide comprises the amino acid sequence set forth in SEQ ID NO: 16.

224. The method of any one of claims 132-223, wherein the selection agent comprises an antibody or antibody fragment that binds to the selection marker.

225. The method of any one of claims 132-224, wherein the selection agent comprises an antibody fragment that binds to the selection marker.

226. The method of claim 224 or claim 225, wherein the antibody fragment is a monovalent antibody fragment.

227. The method of any one of claims 224-226, wherein the antibody fragment is a Fab.

228. The method of any one of claims 132-227, wherein the selection marker is a T cell coreceptor or a member of a T cell antigen receptor complex.

229. The method of any one of claims 132-228, wherein the selection marker is selected from the group consisting of CD3, CD4, CD8, CD45RA, CD27, CD28, and CCR7.

230. The method of any one of claims 132-229, wherein the selection marker is CD3.

231. The method of any one of claims 125-230, wherein the one or more target cells are primary cells from a human subject.

232. The method of any one of claims 131-231, wherein the sample is an apheresis or leukapheresis product.

233. The method of any one of claims 125-232, wherein the viral particle comprises a nucleic acid sequence encoding a recombinant protein.

234. The method of claim 233, wherein the recombinant protein is an antigen receptor.

235. The method of claim 233 or claim 234, wherein the recombinant protein is a chimeric antigen receptor (CAR).

236. The method of claim 233 or claim 234, wherein the recombinant protein is a T cell receptor (TCR).

237. The method of any one of claims 125-236, wherein the viral particle is a viral vector.

238. The method of any one of claims 198-237, wherein the method comprises harvesting the collected transduced target cell.

239. The method of claim 238, wherein the harvesting is carried out after the further incubating.

240. The method of claim 238 or claim 239, wherein the method comprises formulating the harvested transduced target cell for cryopreservation or administration to a subject.

241. The method of any one of claims 238-240, wherein the harvested transduced target cell is formulated in the presence of a pharmaceutically acceptable excipient or a cryoprotectant.

242. The method of any one of claims 125-241, wherein one, optionally all, of the steps of the method is carried out in a closed system.

243. The method of any one of claims 125-242, wherein one, optionally all, of the steps of the method is automated.

244. A kit for purifying viral particles, comprising the protein of any one of claims 1-46, a chromatography matrix suitable for viral purification using column chromatography, and a selection reagent, the selection reagent comprising a molecule that is streptavidin, avidin, a streptavidin analog or mutein, or an avidin analog or mutein, wherein the selection reagent is capable of being immobilized on the chromatography matrix.

245. The kit of claim 244, wherein the streptavidin-binding partner of the protein is bound to the molecule.

246. A kit for transducing cells, comprising the heparin-binding reagent of any one of claims 47-109, a chromatography matrix suitable for cell separation using column chromatography, and a selection agent that specifically binds to a selection marker expressed on the surface of a target cell, wherein the selection agent is capable of being immobilized on the chromatography matrix.

247. The kit of claim 246, wherein the kit comprises a viral particle.

248. The kit of claim 246 or claim 247, wherein the kit comprises a stimulatory reagent.

249. The kit of any one of claims 246-248, wherein the selection agent is immobilized on the chromatography matrix.

250. The kit of any one of claims 246-249, wherein the selection agent comprises a streptavidin-binding partner.

251. The kit of any one of claims 246-250, wherein the kit comprises a selection reagent, the selection reagent comprising a molecule that is streptavidin, avidin, a streptavidin analog or mutein, or an avidin analog or mutein, wherein the selection reagent is capable of being immobilized on the chromatography matrix.

252. The kit of any one of claims 244, 245, and 251, wherein the selection reagent is immobilized on the chromatography matrix.

253. The kit of claim 251 or claim 252, wherein the streptavidin-binding partner of the selection agent is bound to the molecule.

254. The kit of any one of claims 244-253, wherein the kit comprises a chromatography column.

255. The kit of claim 254, wherein the chromatography matrix is comprised in an internal cavity of the chromatography column.

256. A stationary phase for purifying viral particles, comprising the protein of any one of claims 1-46, a chromatography matrix suitable for viral purification using column chromatography, and a selection reagent, the selection reagent comprising a molecule that is streptavidin, avidin, a streptavidin analog or mutein, or an avidin analog or mutein, wherein the selection agent is immobilized on the chromatography matrix, and the streptavidin-binding partner of the protein is bound to the molecule.

257. An article of manufacture for purifying viral particles, comprising the stationary phase of claim 256 and a chromatography column, wherein the stationary phase is comprised in an internal cavity of the chromatography column.

258. A viral particle purified by the method of any of claims 110-124.

259. A target cell transduced by the method of any of claims 125-243.
